(19) United States
(12) Patent Application Publication
(10) Pub. No.: US 2025/0194420 A1
(43) Pub. Date: Jun. 12, 2025

(54) COMPOUND AND ORGANIC ELECTROLUMINESCENT DEVICE COMPRISING THE SAME

(71) Applicant: DuPont Specialty Materials Korea Ltd., Chungcheongnam-do (KR)

(72) Inventors: Eun-Joung CHOI, Gyeonggi-do (KR); HaeYeon KIM, Gyeonggi-do (KR); Hyo-Soon PARK, Gyeonggi-do (KR); Hee-Ryong KANG, Gyeonggi-do (KR); Soo-Yong LEE, Gyeonggi-do (KR); So-Mi PARK, Gyeonggi-do (KR); So-Young JUNG, Gyeonggi-do (KR); Jung-Hwa LEE, Gyeonggi-do (KR); Seung-Hyun YOON, Gyeonggi-do (KR); Yoo-Jin DOH, Gyeonggi-do (KR); Sung-Woo JANG, Gyeonggi-do (KR)

(21) Appl. No.: 18/973,260

(22) Filed: Dec. 9, 2024

(30) Foreign Application Priority Data
Dec. 12, 2023 (KR) 10-2023-0179230
Oct. 10, 2024 (KR) 10-2024-0137283
Nov. 6, 2024 (KR) 10-2024-0156278

(51) Int. Cl. H10K 85/60; C07D 471/04; H10K 50/13

(52) U.S. Cl. CPC H10K 85/6572; C07D 471/04; H10K 85/654; H10K 50/131

(57) ABSTRACT

The present disclosure relates to a compound and an organic electroluminescent device comprising the same. By including the compound according to the present disclosure as an organic electroluminescent material, an organic electroluminescent device exhibiting low driving voltage and/or high power efficiency and/or long lifespan characteristics can be provided.

### TECHNICAL FIELD

The present disclosure relates to a compound and an organic electroluminescent device comprising the same.

### BACKGROUND ART

An organic electroluminescent device (OLED) is a self-light-emitting display device which has advantages in that it provides a wider viewing angle, a greater contrast ratio, and a faster response time. In such an OLED, holes from the anode and electrons from the cathode are injected into a light-emitting layer by the application of electric voltage, and excitons having high energy are produced by the recombination of the holes and electrons. The organic light-emitting compound moves into an excited state due to the energy and emits light from energy when the organic light-emitting compound returns to the ground state from the excited state.

Unlike red and green high-efficiency phosphorescent materials which have already been commercialized among the light-emitting materials of the OLED, it has been pointed out that a blue phosphorescent material is not suitable for long-term use such as several years or more, since the blue phosphorescent material has a short lifespan and high driving voltage, and thus a fluorescent material is used. As such, conventional materials have not been able to satisfy the light-emitting characteristics of the OLED, and thus development of an OLED including an organic electroluminescent material having excellent performance is required.

Korean Patent Application Laid-Open No. 2015-0121394 discloses an organic electroluminescent device comprising a quinazoline derivative substituted with a heterocyclic group containing at least one nitrogen atom as an electron transport layer material, and Korean Patent Application Laid-Open No. 2017-0105040 discloses a light-emitting device comprising a phenanthroline derivative as an electron transport layer material. However, said references do not specifically disclose an organic electroluminescent device comprising a phenanthroline derivative as an N-type charge generation layer material according to the present disclosure.

### DISCLOSURE OF THE INVENTION

### Technical Problem

The object of the present disclosure is, firstly, to provide a compound capable of producing an organic electroluminescent device having low driving voltage and/or high power efficiency and/or long lifespan characteristics, and an organic electroluminescent material comprising the same; and secondly, to provide an organic electroluminescent device comprising the compound and the organic electroluminescent material.

### Solution to the Problem

As a result of intensive studies to solve the technical problem above, the present inventors found that the aforementioned objective can be achieved by a compound of the following Formula 1, thereby completing the present invention.

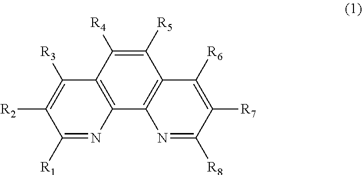

In Formula 1,
R₁ represents hydrogen, deuterium, halogen, a substituted or unsubstituted phenyl, a substituted or unsubstituted biphenyl, a substituted or unsubstituted terphenyl, a substituted or unsubstituted (C₁-C₃₀)alkyl, or a substituted or unsubstituted (3- to 30-membered)heteroaryl;
R₂ to R₈ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀)aliphatic ring and (C₆-C₃₀)aromatic ring or -L-HAr; or may be linked to the adjacent substituents to form a ring(s);
provided that at least one of R₂ to R₈ is -L-HAr;
L represents a single bond, a substituted or unsubstituted (C₆-C₃₀)arylene, or a substituted or unsubstituted (3- to 30-membered)heteroarylene; and
HAr represents a substituted or unsubstituted (3- to 30-membered)heteroaryl containing at least one nitrogen atom;
provided that when R₄ or R₅ is -L-HAr, the compounds in which HAr is quinazoline are excluded.

### Advantageous Effects of the Invention

By using the compound according to the present disclosure and an organic electroluminescent material comprising the same, an organic electroluminescent device having low driving voltage and/or high power efficiency and/or long lifespan characteristics can be prepared.

### MODE FOR THE INVENTION

Hereinafter, the present disclosure will be described in detail. However, the following description is intended to explain the invention, and is not meant in any way to restrict the scope of the invention.

The present disclosure relates to a compound represented by Formula 1, an organic electroluminescent material comprising the compound, and an organic electroluminescent device comprising the organic electroluminescent material.

The term “(organic electroluminescent) compound” in the present disclosure means a compound that may be used in an organic electroluminescent device, and may be comprised in any material layer constituting an organic electroluminescent device, as necessary.

Herein, “organic electroluminescent material” means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron-blocking material, a light-emitting material (containing host and dopant materials), an electron buffer material, a hole-blocking material, an electron transport material, or an electron injection material, etc.

Herein, “(C₁-C₃₀)alkyl(ene)” is meant to be a linear or branched alkyl having 1 to 30 carbon atoms constituting the chain, in which the number of carbon atoms is preferably 1 to 20, more preferably 1 to 10. The above alkyl may include methyl, ethyl, n-propyl, isopropyl, n-butyl, isobutyl, tert-butyl, sec-butyl, etc. Herein, the “(C₃-C₃₀)cycloalkyl(ene)” is meant to be a mono- or polycyclic hydrocarbon having 3 to 30 ring backbone carbon atoms, in which the number of carbon atoms is preferably 3 to 20, more preferably 3 to 7. The above cycloalkyl may include cyclopropyl, cyclobutyl, cyclopentyl, cyclohexyl, cyclopentylmethyl, cyclohexylmethyl, etc. The “(3- to 7-membered)heterocycloalkyl” in the present disclosure is meant to be a cycloalkyl having 3 to 7 ring backbone atoms, preferably 5 to 7 ring backbone atoms and at least one heteroatom selected from the group consisting of B, N, O, S, Si, and P, preferably O, S, and N, and includes tetrahydrofuran, pyrrolidine, thiolan, tetrahydropyran, etc. The “(C₆-C₃₀)aryl(ene)” in the present disclosure is meant to be a monocyclic or fused ring radical derived from an aromatic hydrocarbon having 6 to 30 ring backbone carbon atoms, in which the number of the ring backbone carbon atoms is preferably 6 to 20, more preferably 6 to 15, and may be partially saturated and may comprise a spiro structure. Examples of the aryl specifically include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluorene]yl, spiro[fluorene-benzofluorene]yl, azulenyl, tetramethyl-dihydrophenanthrenyl, etc. More specifically, the aryl may be o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc. The “(3- to 30-membered)heteroaryl(ene)” in the present disclosure is an aryl having 3 to 30 ring backbone atoms including at least one heteroatom selected from the group consisting of B, N, O, S, Si, P, Se, and Ge in which the number of the ring backbone carbon atoms is preferably 3 to 30, more preferably 5 to 20. The number of the heteroatoms in the heteroaryl is preferably 1 to 4. The above heteroaryl may be a monocyclic ring, or a fused ring condensed with at least one benzene ring, and may be partially saturated. Also, the above heteroaryl or heteroarylene herein may be one formed by linking at least one heteroaryl or aryl group to a heteroaryl group via a single bond(s), and may include a spiro structure. Examples of the heteroaryl specifically may include a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, dibenzoselenophenyl, benzofuroquinolinyl, benzofuroquinazolinyl, benzofuronaphthiridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzoperimidinyl, indolocarbazolyl, indenocarbazolyl, etc. More specifically, the heteroaryl may be 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Additionally, “heteroaryl(ene)” can be classified into a heteroaryl(ene) with electronic properties and a heteroaryl(ene) with hole properties. A heteroaryl(ene) with electronic properties is a substituent with relatively abundant electrons in the parent nucleus, and, for example, it may be a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. A heteroaryl(ene) which has hole properties is a substituent with a relative lack of electrons in the parent nucleus, and, for example, it may be a substituted or unsubstituted carbazolyl, a substituted or unsubstituted dibenzofuranyl, or a substituted or unsubstituted dibenzothiophenyl. Herein, the term “a fused ring of (C₃-C₃₀) aliphatic ring and (C₆-C₃₀) aromatic ring” means a ring formed by fusing at least one aliphatic ring having 3 to 30 ring backbone carbon atoms in which the number of carbon atoms is preferably 3 to 25, more preferably 3 to 18, and at least one aromatic ring having 6 to 30 ring backbone carbon atoms in which the number of carbon atoms is preferably 6 to 25, more preferably 6 to 18. For example, the fused ring may be a fused ring of at least one benzene and at least one cyclohexane, or a fused ring of at least one naphthalene and at least one cyclopentane, etc. Herein, the carbon atoms in the fused ring of (C₃-C₃₀) aliphatic ring and (C₆-C₃₀) aromatic ring may be replaced with at least one heteroatom selected from B, N, O, S, Si, and P, preferably at least one heteroatom selected from N, O, and S. The “halogen” in the present disclosure includes F, Cl, Br, and I.

In addition, “ortho-” (“o-”), “meta-” (“m-”), and “para-” (“p-”) are meant to signify the substitution position of all substituents. An ortho-configuration describes a compound with substituents which are adjacent to each other, e.g., at the 1 and 2 positions on benzene. A meta-configuration indicates the next substitution position of the immediately adjacent substitution position, e.g., a compound with substituents at the 1 and 3 positions on benzene. A para-configuration indicates the next substitution position from the meta-position, e.g., a compound with substituents at the 1 and 4 positions on benzene.

Herein, the term “a ring formed in linking to an adjacent substituent” means a substituted or unsubstituted (3- to 30-membered) mono- or polycyclic, alicyclic, aromatic ring, or a combination thereof, formed by linking or fusing two or more adjacent substituents, and preferably this may be a substituted or unsubstituted (5- to 25-membered) mono- or polycyclic, alicyclic, aromatic ring, or a combination thereof. Further, the formed ring may include at least one heteroatom selected from the group consisting of B, N, O, S, Si, and P, preferably N, O, and S. According to one embodiment of the present disclosure, the number of atoms in the ring skeleton is 5 to 20; according to another embodiment of the present disclosure, the number of atoms in the ring skeleton is 5 to 15. In one embodiment, the fused ring may be, for example, a substituted or unsubstituted dibenzothiophene ring, a substituted or unsubstituted dibenzofuran ring, a substituted or unsubstituted naphthalene ring, a substituted or unsubstituted phenanthrene ring, a substituted or unsubstituted fluorene ring, a substituted or unsubstituted benzofluorene ring, a substituted or unsubstituted benzothiophene ring, a substituted or unsubstituted benzofuran ring, a substituted or unsubstituted indole ring, a substituted or unsubstituted indene ring, a substituted or unsubstituted benzene ring, or a substituted or unsubstituted carbazole ring, etc.

In addition, the term “substituted” in the expression “substituted or unsubstituted” means that a hydrogen atom in a certain functional group is replaced with another atom or functional group, i.e., a substituent. Unless otherwise specified, the substituents may not be limited to hydrogen at positions where the substituents may be substituted, and when two or more hydrogen atoms are each replaced with a substituent in a functional group, the substituents may be the same as or different from each other. For example, “a substituent to which two or more substituents are connected” may be pyridine-triazine. That is, pyridine-triazine may be heteroaryl or may be interpreted as one substituent in which two heteroaryls are connected. Preferably, the substituted alkyl, the substituted alkenyl, the substituted aryl(ene), the substituted heteroaryl(ene), the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, and the substituted fused ring of aliphatic ring and aromatic ring in the formulas of the present disclosure each independently are substituted with at least one selected from the group consisting of deuterium, halogen, cyano, carboxyl, nitro, hydroxyl, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, (5- to 30-membered)heteroaryl unsubstituted or substituted with (C₆-C₃₀)aryl, (C₆-C₃₀)aryl unsubstituted or substituted with (5- to 30-membered)heteroaryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, a fused ring of (C₃-C₃₀)aliphatic ring and (C₆-C₃₀)aromatic ring, amino, mono- or di(C₁-C₃₀)alkylamino, a substituted or unsubstituted mono- or di(C₆-C₃₀)arylamino, (C₁-C₃₀)alkyl(C₆-C₃₀)arylamino, mono- or di(3- to 30-membered)heteroarylamino, (C₁-C₃₀)alkyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, (C₆-C₃₀)arylphosphinyl, di(C₆-C₃₀)arylboronyl, di(C₁-C₃₀)alkylboronyl, (C₁-C₃₀)alkyl(C₆-C₃₀)arylboronyl, (C₆-C₃₀)ar(C₁-C₃₀)alkyl, and (C₁-C₃₀)alkyl(C₆-C₃₀)aryl.

When a substituent is not shown in the chemical formula or the compound structure of the present disclosure, it may signify that all positions that may be present as substituents are hydrogen or deuterium. That is, in the case of deuterium, an isotope of hydrogen, some of the hydrogen atoms may be deuterium, which is an isotope, and in this case, the content of deuterium may be 0% to 100%. In the case where the substituent is not shown in the chemical formula or the compound structure of the present disclosure, when deuterium is not explicitly excluded, such as when the content of deuterium is 0%, the content of hydrogen is 100%, and all substituents are hydrogen, hydrogen and deuterium may be mixed and used in the compound. The deuterium is an element having a deuteron composed of one proton and one neutron as an atomic nucleus, which is one of the isotopes of hydrogen, and may be represented as hydrogen-2, and the element symbol may be D or ²H. Isotopes have the same atomic number (Z) but different mass numbers (A), and may also be interpreted as elements with the same number of protons but different numbers of neutrons.

Herein, “combinations thereof” signifies that one or more components of the corresponding list are combined to form a known or chemically stable arrangement that a person skilled in the art could conceive of from the corresponding list. For example, alkyl and deuterium may be combined to form partially or entirely deuterated alkyl groups; halogen and alkyl may be combined to form halogenated alkyl substituents; and halogen, alkyl, and aryl may be combined to form halogenated arylalkyl. For example, preferred combinations of substituents may include up to 50 atoms excluding hydrogen and deuterium, or include up to 40 atoms excluding hydrogen and deuterium, or include up to 30 atoms excluding hydrogen and deuterium, or in many cases, preferred combinations of substituents may include up to 20 atoms excluding hydrogen and deuterium.

In the formula of the present disclosure, when multiple substituents are indicated by the same symbol, each of these substituents represented by the same symbol may be the same as or different from one another.

Hereinafter, the compound according to one embodiment will be described.

The compound according to one embodiment of the present disclosure is represented by the following Formula 1.

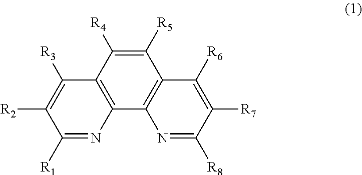

In Formula 1,
R₁ represents hydrogen, deuterium, halogen, a substituted or unsubstituted phenyl, a substituted or unsubstituted biphenyl, a substituted or unsubstituted terphenyl, a substituted or unsubstituted (C₁-C₃₀)alkyl, or a substituted or unsubstituted (3- to 30-membered)heteroaryl;
R₂ to R₈ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀)aliphatic ring and (C₆-C₃₀)aromatic ring, or -L-HAr; or may be linked to the adjacent substituents to form a ring(s);
provided that at least one of R₂ to R₈ is -L-HAr;
L represents a single bond, a substituted or unsubstituted (C₆-C₃₀)arylene, or a substituted or unsubstituted (3- to 30-membered)heteroarylene; and
HAr represents a substituted or unsubstituted (3- to 30-membered)heteroaryl containing at least one nitrogen atom;
provided that when R₄ or R₅ is -L-HAr, the compounds in which HAr is quinazoline are excluded.

In one embodiment, R₁ may be hydrogen, halogen, a substituted or unsubstituted phenyl, a substituted or unsubstituted biphenyl, a substituted or unsubstituted terphenyl, a substituted or unsubstituted (C₁-C₁₀)alkyl, or a substituted or unsubstituted (5- to 30-membered)heteroaryl, preferably hydrogen, halogen, phenyl unsubstituted or substituted with (C₁-C₁₀)alkyl or (5- to 30-membered)heteroaryl, a substituted or unsubstituted p-biphenyl, a substituted or unsubstituted o-biphenyl, a substituted or unsubstituted m-terphenyl, a substituted or unsubstituted o-terphenyl, (C₁-C₁₀)alkyl unsubstituted or substituted with deuterium, or a substituted or unsubstituted (5- to 25-membered)heteroaryl, more preferably hydrogen, halogen, phenyl unsubstituted or substituted with (C₁-C₄)alkyl or (5- to 25-membered)heteroaryl, unsubstituted p-biphenyl, unsubstituted m-biphenyl, unsubstituted o-biphenyl, unsubstituted m-terphenyl, unsubstituted o-terphenyl, (C₁-C₄)alkyl unsubstituted or substituted with deuterium, or a substituted or unsubstituted (5- to 18-membered)heteroaryl. For example, R₁ may be hydrogen, F, phenyl unsubstituted or substituted with tert-butyl or benzotriazolyl, unsubstituted p-biphenyl, unsubstituted m-biphenyl, unsubstituted o-biphenyl, unsubstituted m-terphenyl, unsubstituted o-terphenyl, methyl substituted with deuterium, tert-butyl unsubstituted or substituted with deuterium, a substituted or unsubstituted pyridyl, a substituted or unsubstituted pyrimidinyl, a substituted or unsubstituted isoquinolyl, a substituted or unsubstituted quinolyl, a substituted or unsubstituted dibenzofuranyl, a substituted or unsubstituted dibenzothiophenyl, or a substituted or unsubstituted benzotriazolyl.

In one embodiment, R₂ to R₈ each independently may be hydrogen, a substituted or unsubstituted (C₆-C₃₀)aryl, or -L-HAr, and at least one of R₂ to R₈ may be -L-HAr, preferably hydrogen, a substituted or unsubstituted (C₆-C₂₅)aryl, or -L-HAr, more preferably hydrogen, a substituted or unsubstituted (C₆-C₁₈)aryl, or -L-HAr. For example, R₂ to R₈ each independently may be hydrogen, a substituted or unsubstituted phenyl, or -L-HAr.

According to one embodiment, the compound represented by Formula 1 may be represented by any one of the following Formulas 1-1 to 1-7.

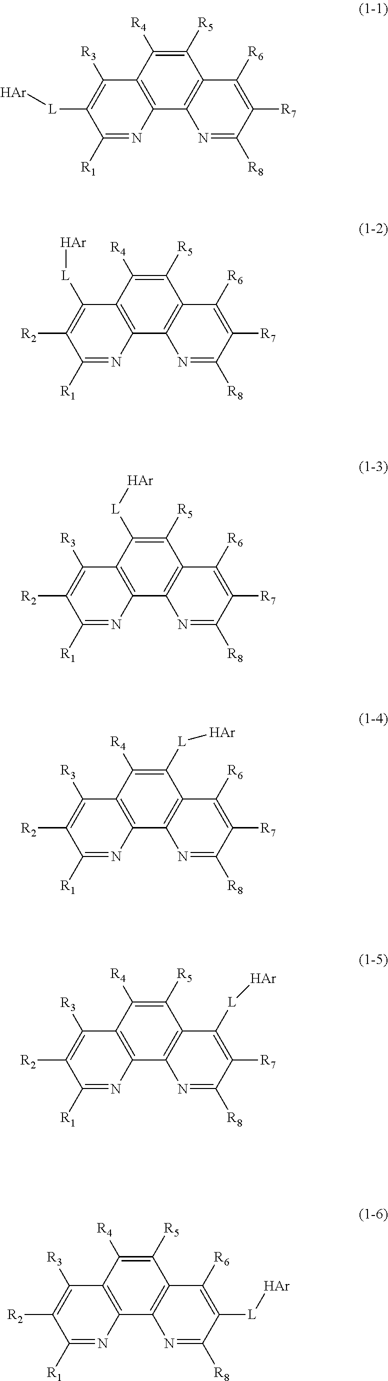

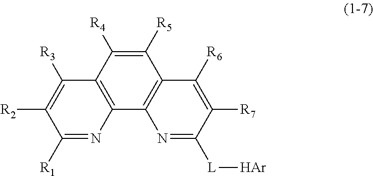

In Formulas 1-1 to 1-7,
R₁, L, and HAr are as defined in Formula 1; and
R₂ to R₈ each independently represent hydrogen, deuterium, a substituted or unsubstituted (C₆-C₃₀)aryl, or a substituted or unsubstituted (3- to 30-membered)heteroaryl.

In one embodiment, L may be a single bond or a substituted or unsubstituted (C₆-C₃₀)arylene, preferably a single bond or a substituted or unsubstituted (C₆-C₂₅)arylene, more preferably a single bond or a substituted or unsubstituted (C₆-C₁₈)arylene. For example, L may be a single bond, phenylene unsubstituted or substituted with at least one phenyl, a substituted or unsubstituted biphenylene, or a substituted or unsubstituted naphthylene.

In one embodiment, HAr may be a substituted or unsubstituted (5- to 30-membered)heteroaryl containing at least one nitrogen atom, preferably (5- to 25-membered)heteroaryl containing at least one nitrogen atom and unsubstituted or substituted with (C₆-C₃₀)aryl or (5- to 30-membered)heteroaryl, more preferably (5- to 18-membered)heteroaryl containing at least two nitrogen atoms and unsubstituted or substituted with (C₆-C₂₅)aryl or (5- to 25-membered)heteroaryl. For example, HAr may be quinazolinyl unsubstituted or substituted with at least one phenyl or at least one pyridyl, quinoxalinyl unsubstituted or substituted with at least one phenyl or at least one pyridyl, or benzotriazolyl unsubstituted or substituted with phenyl or dimethylfluorenyl.

In one embodiment, HAr may be represented by the following Formula 1-a or 1-b.

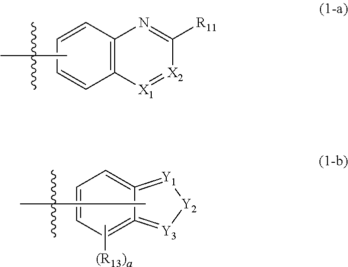

In Formulas 1-a and 1-b,

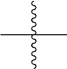

represents a site connected to L;
X₁ and X₂ each independently represent N or CR₁₂;
Y₁ to Y₃ each independently represent N or NR₁₄;
R₁₁ and R₁₂ each independently represent hydrogen, deuterium, a substituted or unsubstituted (C₆-C₃₀)aryl, or a substituted or unsubstituted (3- to 30-membered)heteroaryl;
R₁₃ and R₁₄ each independently represent hydrogen, deuterium, a substituted or unsubstituted (C₆-C₃₀)aryl, a substituted or unsubstituted (3- to 30-membered)heteroaryl, or a site directly connected to L; and
a represents an integer of 1 to 4, when a is an integer of 2 or more, each of R₁₃ may be the same as or different from each other.

In one embodiment, X₁ may be N, and X₂ may be CR₁₂.

In one embodiment, X₁ may be CR₁₂, and X₂ may be N.

In one embodiment, R₁₁ and R₁₂ each independently may be a substituted or unsubstituted (C₆-C₃₀)aryl or a substituted or unsubstituted (5- to 30-membered)heteroaryl, preferably a substituted or unsubstituted (C₆-C₂₅)aryl or a substituted or unsubstituted (5- to 25-membered)heteroaryl, more preferably a substituted or unsubstituted (C₆-C₁₈)aryl or a substituted or unsubstituted (5- to 18-membered)heteroaryl. For example, R₁₁ and R₁₂ each independently may be phenyl unsubstituted or substituted with methyl substituted with deuterium, a substituted or unsubstituted naphthyl, a substituted or unsubstituted p-biphenyl, a substituted or unsubstituted m-biphenyl, a substituted or unsubstituted phenanthrenyl, a substituted or unsubstituted pyridyl, a substituted or unsubstituted dibenzofuranyl, or a substituted or unsubstituted dibenzothiophenyl.

In one embodiment, Y₁ and Y₃ may be N, and Y₂ may be NR₁₄, wherein R₁₄ may be a substituted or unsubstituted (C₆-C₃₀)aryl.

In one embodiment, Y₁ and Y₃ may be N, and Y₂ may be NR₁₄, wherein R₁₄ may be a site directly connected to L.

In one embodiment, R₁₃ and R₁₄ each independently may be hydrogen, deuterium, or a substituted or unsubstituted (C₆-C₃₀)aryl, preferably hydrogen, deuterium, or a substituted or unsubstituted (C₆-C₂₅)aryl, and more preferably hydrogen, deuterium, or a substituted or unsubstituted (C₆-C₁₈)aryl. For example, R₁₃ and R₁₄ each independently may be hydrogen, deuterium, a substituted or unsubstituted phenyl, a substituted or unsubstituted naphthyl, or a substituted or unsubstituted fluorenyl.

According to one embodiment, the compound represented by Formula 1 may be more specifically illustrated by the following compounds, but is not limited thereto:

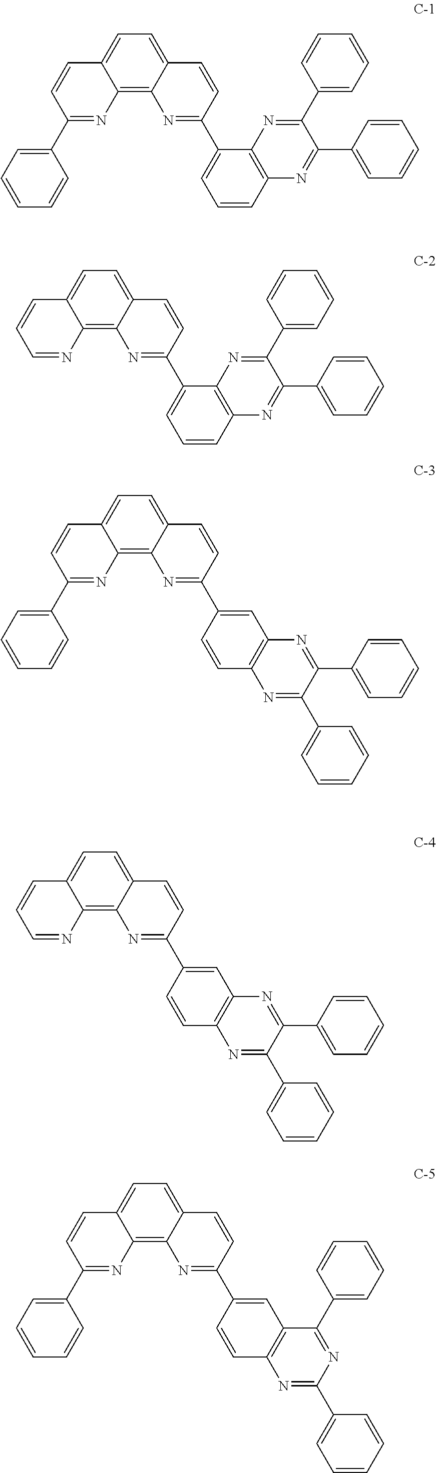 [SMILES: c1ccc(-c2ccc3ccc4ccc(-c5cccc6nc(-c7ccccc7)c(-c7ccccc7)nc56)nc4c3n2)cc1 ; c1ccc(-c2nc3cccc(-c4ccc5ccc6cccnc6c5n4)c3nc2-c2ccccc2)cc1 ; c1ccc(-c2ccc3ccc4ccc(-c5ccc6nc(-c7ccccc7)c(-c7ccccc7)nc6c5)nc4c3n2)cc1 ; c1ccc(-c2nc3ccc(-c4ccc5ccc6cccnc6c5n4)cc3nc2-c2ccccc2)cc1 ; c1ccc(-c2ccc3ccc4ccc(-c5ccc6nc(-c7ccccc7)nc(-c7ccccc7)c6c5)nc4c3n2)cc1]

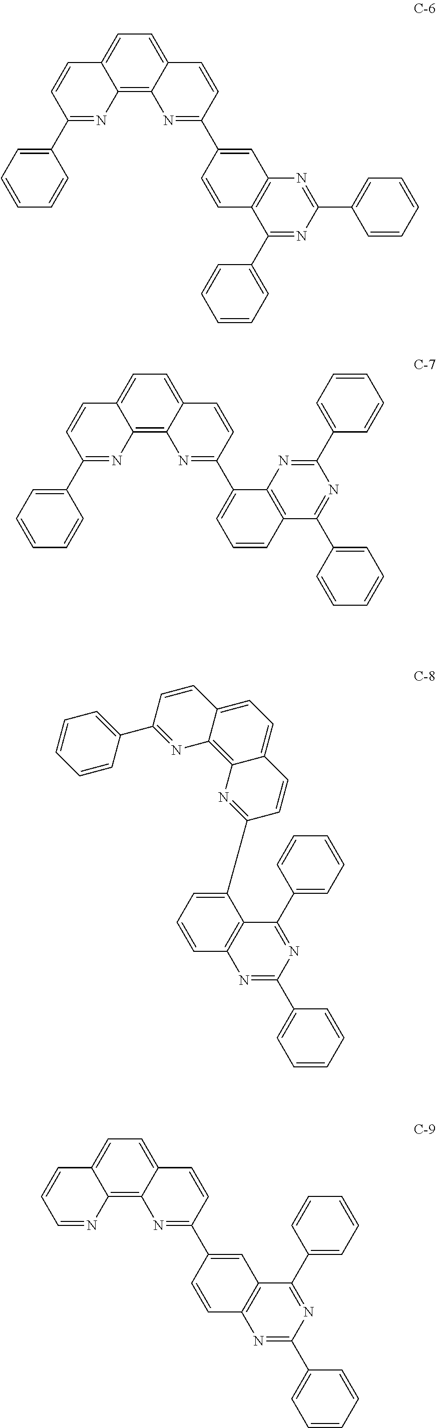 [SMILES: c1ccc(-c2ccc3ccc4ccc(-c5ccc6c(-c7ccccc7)nc(-c7ccccc7)nc6c5)nc4c3n2)cc1 ; c1ccc(-c2ccc3ccc4ccc(-c5cccc6c(-c7ccccc7)nc(-c7ccccc7)nc56)nc4c3n2)cc1 ; c1ccc(-c2ccc3ccc4ccc(-c5cccc6nc(-c7ccccc7)nc(-c7ccccc7)c56)nc4c3n2)cc1 ; c1ccc(-c2nc(-c3ccccc3)c3cc(-c4ccc5ccc6cccnc6c5n4)ccc3n2)cc1]

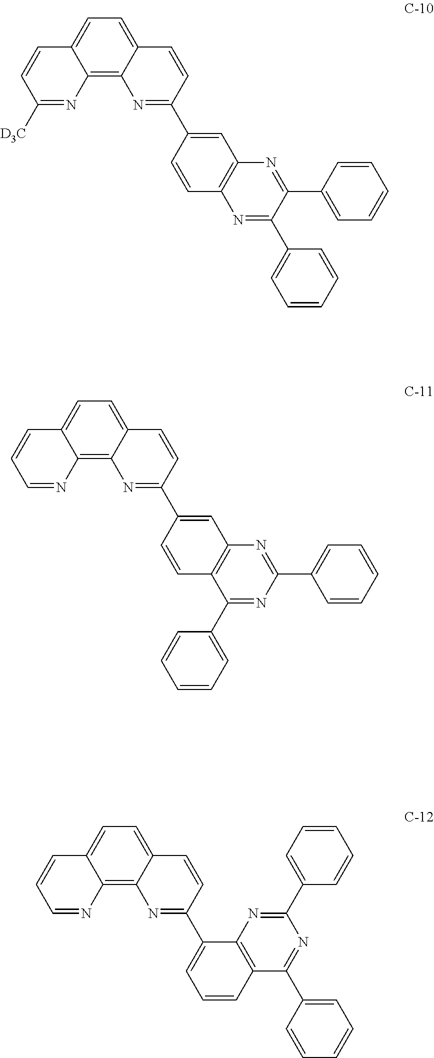 [SMILES: [2H]C([2H])([2H])c1ccc2ccc3ccc(-c4ccc5nc(-c6ccccc6)c(-c6ccccc6)nc5c4)nc3c2n1 ; c1ccc(-c2nc(-c3ccccc3)c3ccc(-c4ccc5ccc6cccnc6c5n4)cc3n2)cc1 ; c1ccc(-c2nc(-c3ccccc3)c3cccc(-c4ccc5ccc6cccnc6c5n4)c3n2)cc1]

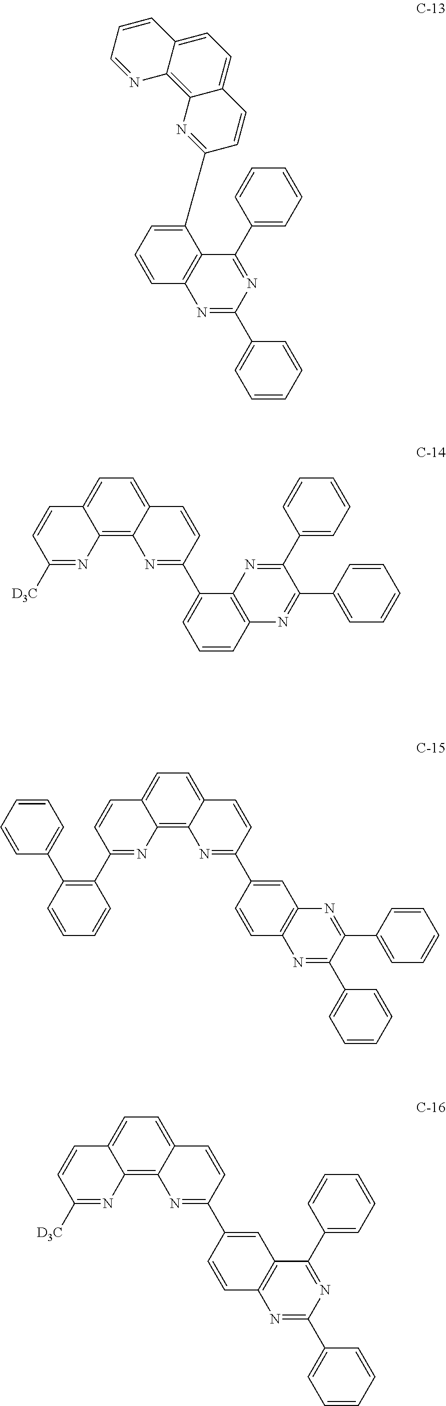 [SMILES: c1ccc(-c2nc(-c3ccccc3)c3c(-c4ccc5ccc6cccnc6c5n4)cccc3n2)cc1 ; [2H]C([2H])([2H])c1ccc2ccc3ccc(-c4cccc5nc(-c6ccccc6)c(-c6ccccc6)nc45)nc3c2n1 ; c1ccc(-c2ccccc2-c2ccc3ccc4ccc(-c5ccc6nc(-c7ccccc7)c(-c7ccccc7)nc6c5)nc4c3n2)cc1 ; [2H]C([2H])([2H])c1ccc2ccc3ccc(-c4ccc5nc(-c6ccccc6)nc(-c6ccccc6)c5c4)nc3c2n1]

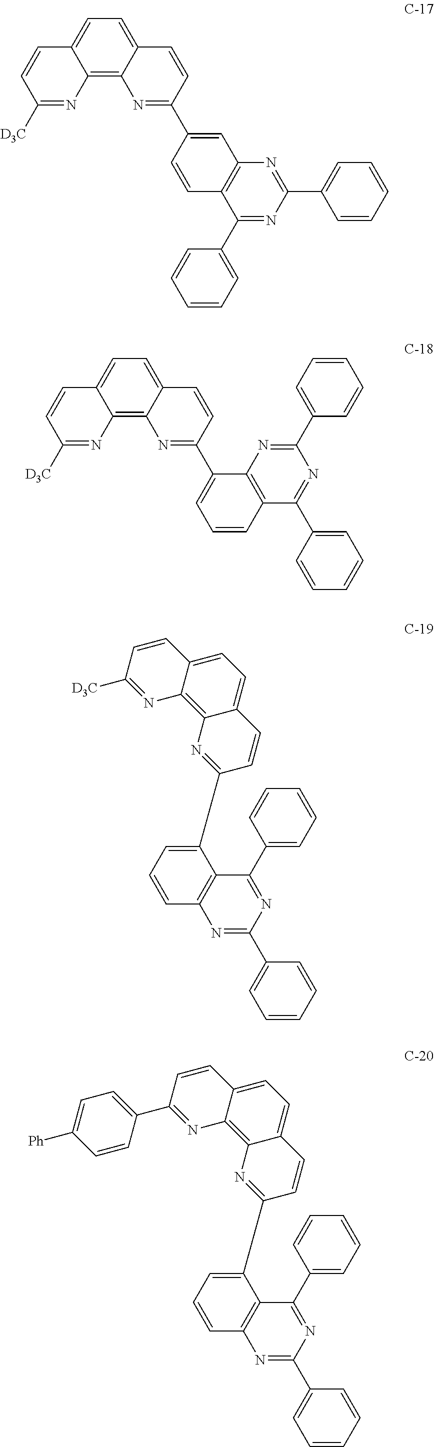 [SMILES: [2H]C([2H])([2H])c1ccc2ccc3ccc(-c4ccc5c(-c6ccccc6)nc(-c6ccccc6)nc5c4)nc3c2n1 ; [2H]C([2H])([2H])c1ccc2ccc3ccc(-c4cccc5c(-c6ccccc6)nc(-c6ccccc6)nc45)nc3c2n1 ; [2H]C([2H])([2H])c1ccc2ccc3ccc(-c4cccc5nc(-c6ccccc6)nc(-c6ccccc6)c45)nc3c2n1 ; c1ccc(-c2ccc(-c3ccc4ccc5ccc(-c6cccc7nc(-c8ccccc8)nc(-c8ccccc8)c67)nc5c4n3)cc2)cc1]

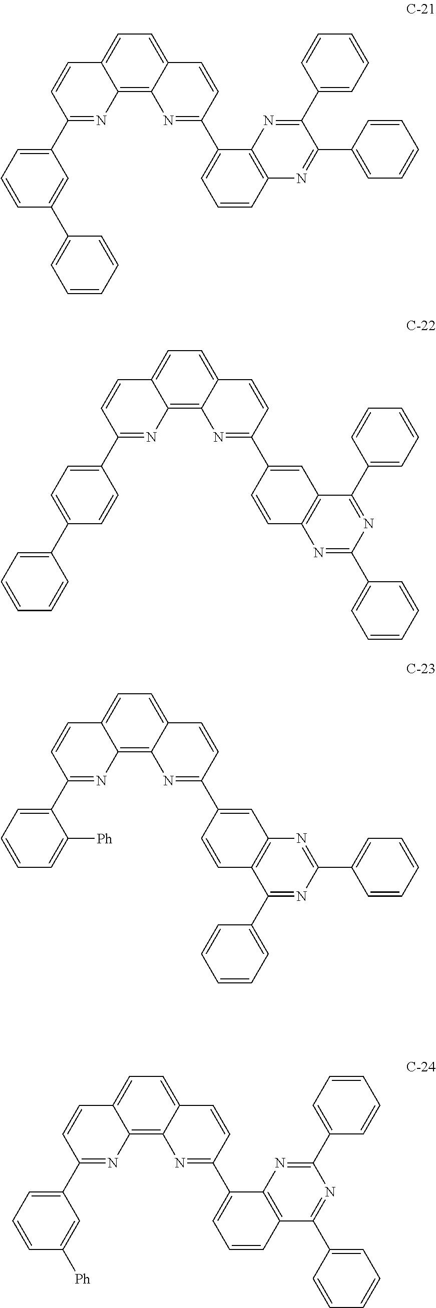 [SMILES: c1ccc(-c2cccc(-c3ccc4ccc5ccc(-c6cccc7nc(-c8ccccc8)c(-c8ccccc8)nc67)nc5c4n3)c2)cc1 ; c1ccc(-c2ccc(-c3ccc4ccc5ccc(-c6ccc7nc(-c8ccccc8)nc(-c8ccccc8)c7c6)nc5c4n3)cc2)cc1 ; c1ccc(-c2nc(-c3ccccc3)c3ccc(-c4ccc5ccc6ccc(-c7ccccc7-c7ccccc7)nc6c5n4)cc3n2)cc1 ; c1ccc(-c2cccc(-c3ccc4ccc5ccc(-c6cccc7c(-c8ccccc8)nc(-c8ccccc8)nc67)nc5c4n3)c2)cc1]

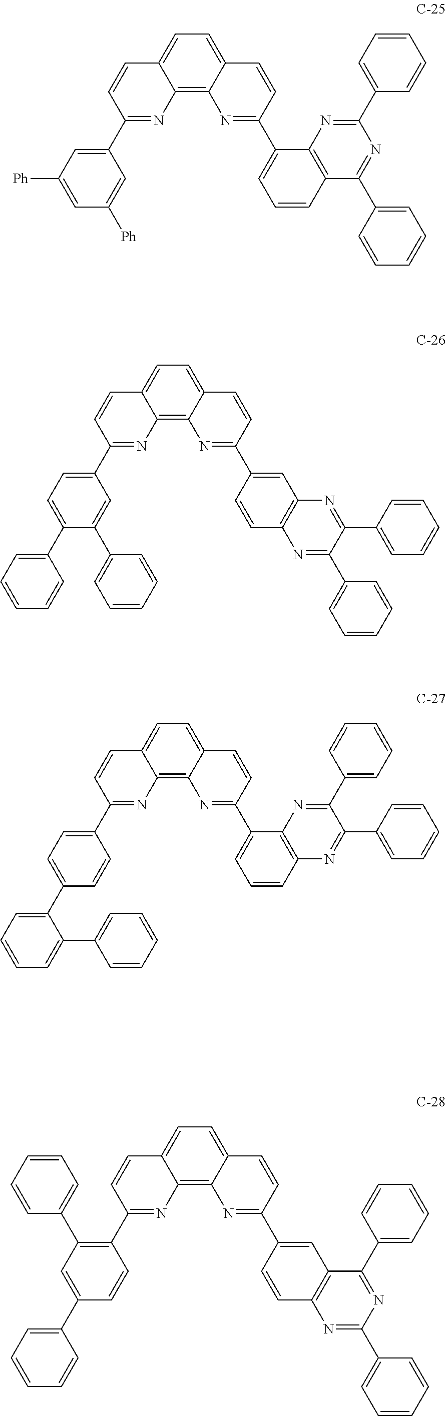 [SMILES: c1ccc(-c2cc(-c3ccccc3)cc(-c3ccc4ccc5ccc(-c6cccc7c(-c8ccccc8)nc(-c8ccccc8)nc67)nc5c4n3)c2)cc1 ; c1ccc(-c2ccc(-c3ccc4ccc5ccc(-c6ccc7nc(-c8ccccc8)c(-c8ccccc8)nc7c6)nc5c4n3)cc2-c2ccccc2)cc1 ; c1ccc(-c2ccccc2-c2ccc(-c3ccc4ccc5ccc(-c6cccc7nc(-c8ccccc8)c(-c8ccccc8)nc67)nc5c4n3)cc2)cc1 ; c1ccc(-c2ccc(-c3ccc4ccc5ccc(-c6ccc7nc(-c8ccccc8)nc(-c8ccccc8)c7c6)nc5c4n3)c(-c3ccccc3)c2)cc1]

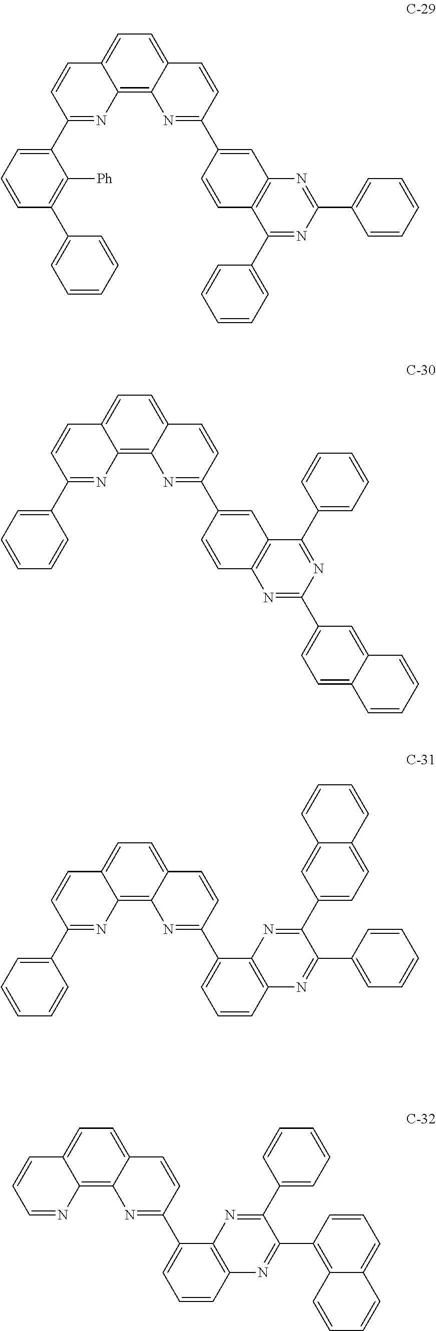 [SMILES: c1ccc(-c2nc(-c3ccccc3)c3ccc(-c4ccc5ccc6ccc(-c7cccc(-c8ccccc8)c7-c7ccccc7)nc6c5n4)cc3n2)cc1 ; c1ccc(-c2ccc3ccc4ccc(-c5ccc6nc(-c7ccc8ccccc8c7)nc(-c7ccccc7)c6c5)nc4c3n2)cc1 ; c1ccc(-c2ccc3ccc4ccc(-c5cccc6nc(-c7ccccc7)c(-c7ccc8ccccc8c7)nc56)nc4c3n2)cc1 ; c1ccc(-c2nc3c(-c4ccc5ccc6cccnc6c5n4)cccc3nc2-c2cccc3ccccc23)cc1]

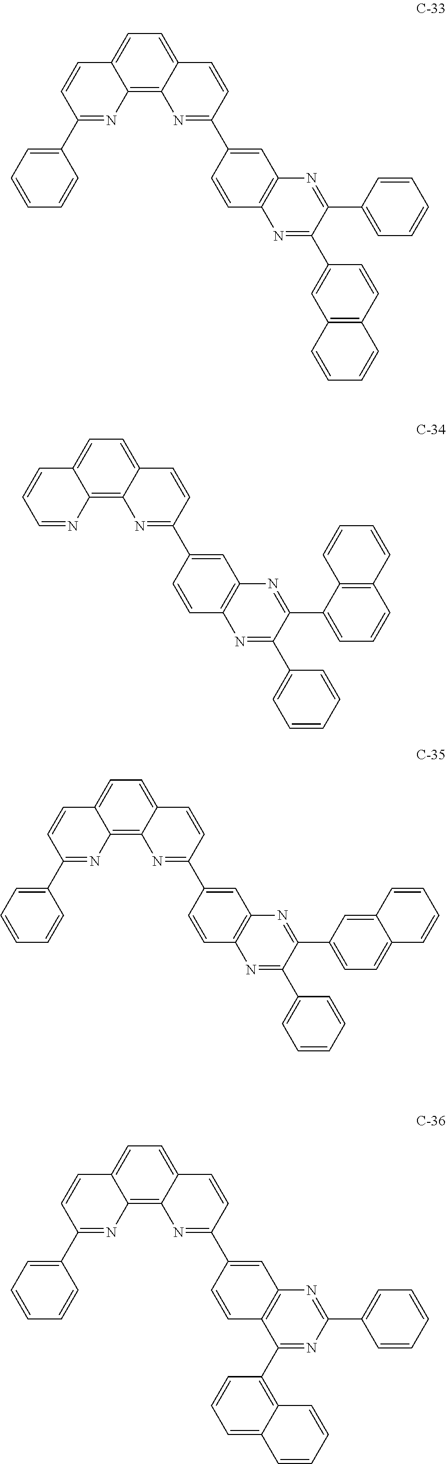 [SMILES: c1ccc(-c2ccc3ccc4ccc(-c5ccc6nc(-c7ccc8ccccc8c7)c(-c7ccccc7)nc6c5)nc4c3n2)cc1 ; c1ccc(-c2nc3ccc(-c4ccc5ccc6cccnc6c5n4)cc3nc2-c2cccc3ccccc23)cc1 ; c1ccc(-c2ccc3ccc4ccc(-c5ccc6nc(-c7ccccc7)c(-c7ccc8ccccc8c7)nc6c5)nc4c3n2)cc1 ; c1ccc(-c2ccc3ccc4ccc(-c5ccc6c(-c7cccc8ccccc78)nc(-c7ccccc7)nc6c5)nc4c3n2)cc1]

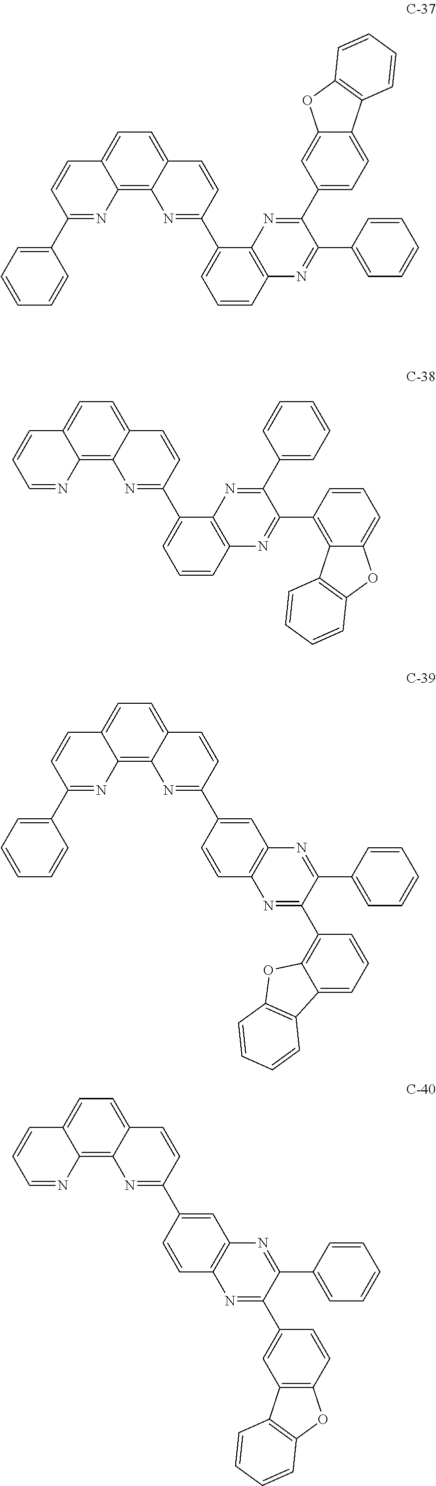 [SMILES: c1ccc(-c2ccc3ccc4ccc(-c5cccc6nc(-c7ccccc7)c(-c7ccc8c(c7)oc7ccccc78)nc56)nc4c3n2)cc1 ; c1ccc(-c2nc3c(-c4ccc5ccc6cccnc6c5n4)cccc3nc2-c2cccc3oc4ccccc4c23)cc1 ; c1ccc(-c2ccc3ccc4ccc(-c5ccc6nc(-c7cccc8c7oc7ccccc78)c(-c7ccccc7)nc6c5)nc4c3n2)cc1 ; c1ccc(-c2nc3cc(-c4ccc5ccc6cccnc6c5n4)ccc3nc2-c2ccc3oc4ccccc4c3c2)cc1]

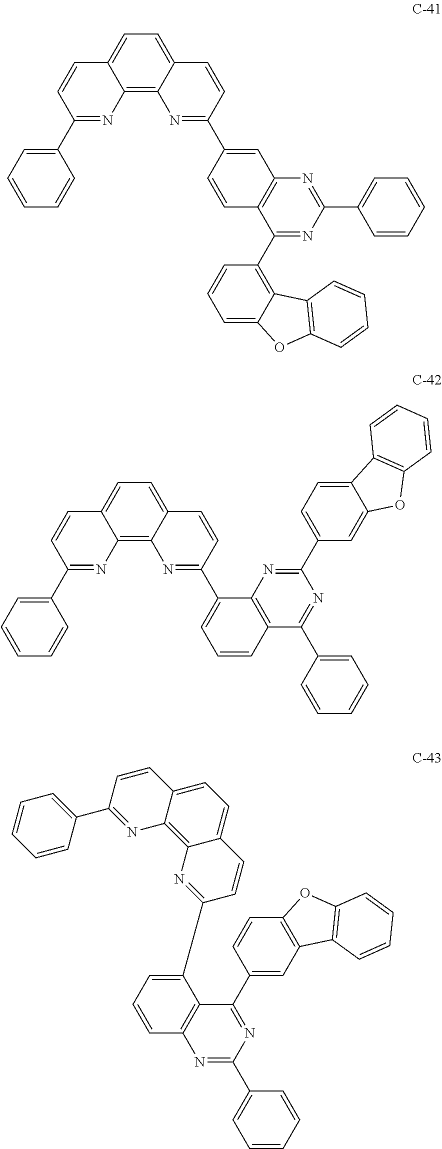 [SMILES: c1ccc(-c2ccc3ccc4ccc(-c5ccc6c(-c7cccc8oc9ccccc9c78)nc(-c7ccccc7)nc6c5)nc4c3n2)cc1 ; c1ccc(-c2ccc3ccc4ccc(-c5cccc6c(-c7ccccc7)nc(-c7ccc8c(c7)oc7ccccc78)nc56)nc4c3n2)cc1 ; c1ccc(-c2ccc3ccc4ccc(-c5cccc6nc(-c7ccccc7)nc(-c7ccc8oc9ccccc9c8c7)c56)nc4c3n2)cc1]

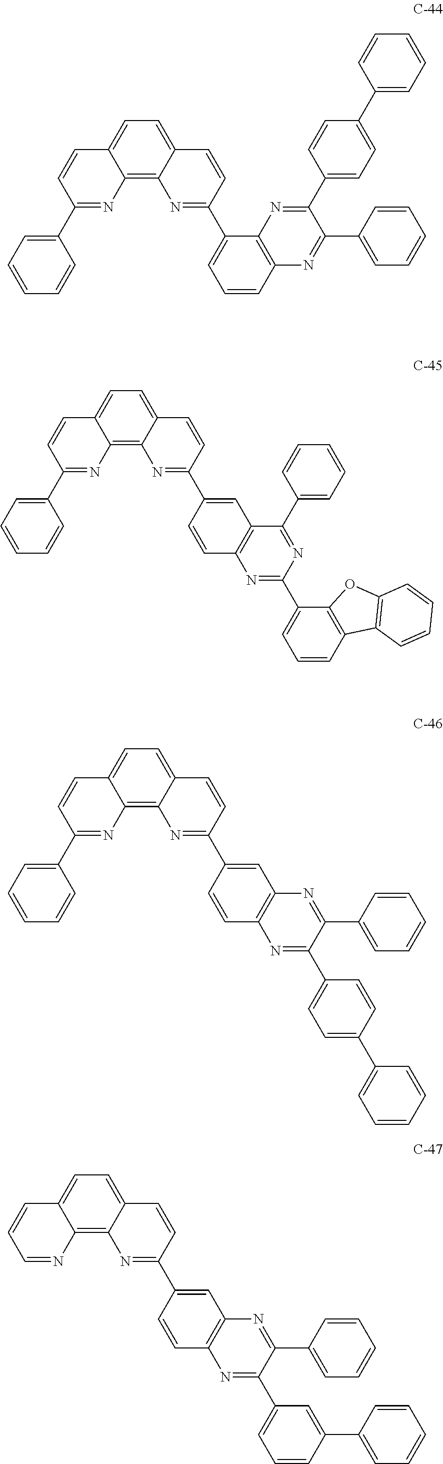 [SMILES: c1ccc(-c2ccc(-c3nc4c(-c5ccc6ccc7ccc(-c8ccccc8)nc7c6n5)cccc4nc3-c3ccccc3)cc2)cc1 ; c1ccc(-c2ccc3ccc4ccc(-c5ccc6nc(-c7cccc8c7oc7ccccc78)nc(-c7ccccc7)c6c5)nc4c3n2)cc1 ; c1ccc(-c2ccc(-c3nc4ccc(-c5ccc6ccc7ccc(-c8ccccc8)nc7c6n5)cc4nc3-c3ccccc3)cc2)cc1 ; c1ccc(-c2cccc(-c3nc4ccc(-c5ccc6ccc7cccnc7c6n5)cc4nc3-c3ccccc3)c2)cc1]

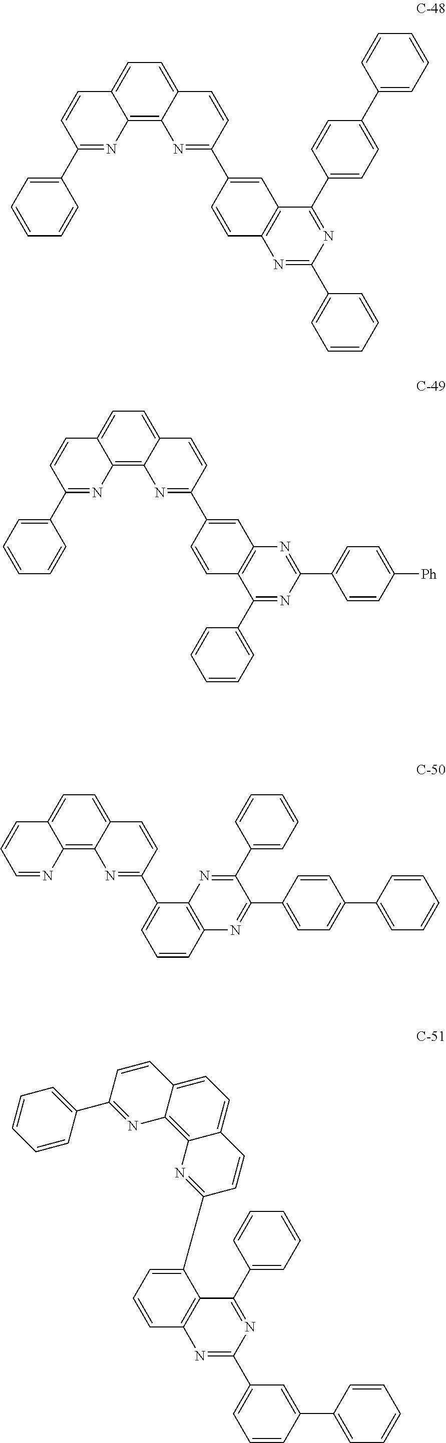 [SMILES: c1ccc(-c2ccc(-c3nc(-c4ccccc4)nc4ccc(-c5ccc6ccc7ccc(-c8ccccc8)nc7c6n5)cc34)cc2)cc1 ; c1ccc(-c2ccc(-c3nc(-c4ccccc4)c4ccc(-c5ccc6ccc7ccc(-c8ccccc8)nc7c6n5)cc4n3)cc2)cc1 ; c1ccc(-c2ccc(-c3nc4cccc(-c5ccc6ccc7cccnc7c6n5)c4nc3-c3ccccc3)cc2)cc1 ; c1ccc(-c2cccc(-c3nc(-c4ccccc4)c4c(-c5ccc6ccc7ccc(-c8ccccc8)nc7c6n5)cccc4n3)c2)cc1]

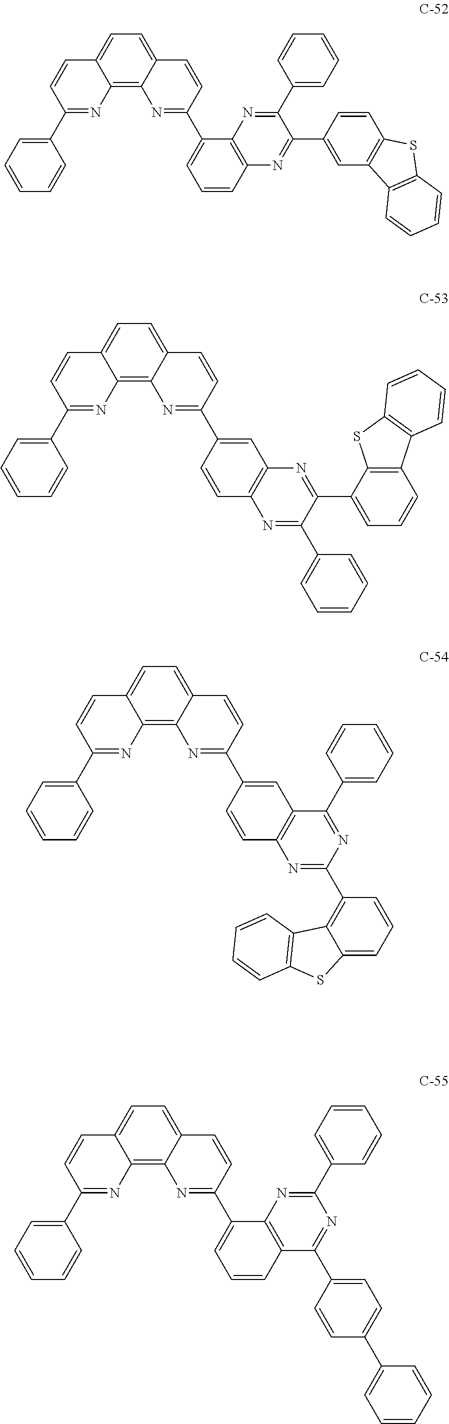 [SMILES: c1ccc(-c2ccc3ccc4ccc(-c5cccc6nc(-c7ccc8sc9ccccc9c8c7)c(-c7ccccc7)nc56)nc4c3n2)cc1 ; c1ccc(-c2ccc3ccc4ccc(-c5ccc6nc(-c7ccccc7)c(-c7cccc8c7sc7ccccc78)nc6c5)nc4c3n2)cc1 ; c1ccc(-c2ccc3ccc4ccc(-c5ccc6nc(-c7cccc8sc9ccccc9c78)nc(-c7ccccc7)c6c5)nc4c3n2)cc1 ; c1ccc(-c2ccc(-c3nc(-c4ccccc4)nc4c(-c5ccc6ccc7ccc(-c8ccccc8)nc7c6n5)cccc34)cc2)cc1]

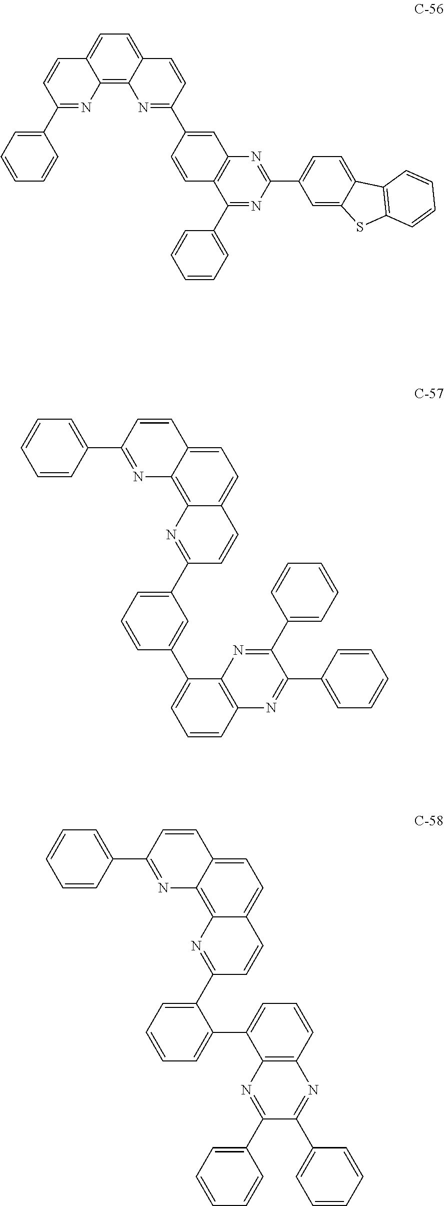 [SMILES: c1ccc(-c2ccc3ccc4ccc(-c5ccc6c(-c7ccccc7)nc(-c7ccc8c(c7)sc7ccccc78)nc6c5)nc4c3n2)cc1 ; c1ccc(-c2ccc3ccc4ccc(-c5cccc(-c6cccc7nc(-c8ccccc8)c(-c8ccccc8)nc67)c5)nc4c3n2)cc1 ; c1ccc(-c2ccc3ccc4ccc(-c5ccccc5-c5cccc6nc(-c7ccccc7)c(-c7ccccc7)nc56)nc4c3n2)cc1]

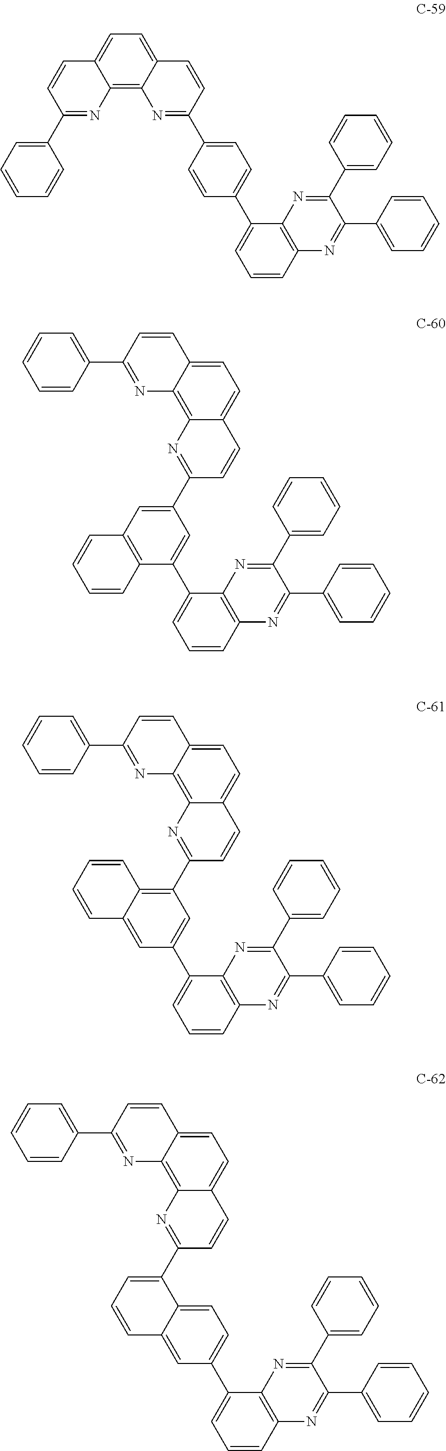 [SMILES: c1ccc(-c2ccc3ccc4ccc(-c5ccc(-c6cccc7nc(-c8ccccc8)c(-c8ccccc8)nc67)cc5)nc4c3n2)cc1 ; c1ccc(-c2ccc3ccc4ccc(-c5cc(-c6cccc7nc(-c8ccccc8)c(-c8ccccc8)nc67)c6ccccc6c5)nc4c3n2)cc1 ; c1ccc(-c2ccc3ccc4ccc(-c5cc(-c6cccc7nc(-c8ccccc8)c(-c8ccccc8)nc67)cc6ccccc56)nc4c3n2)cc1 ; c1ccc(-c2ccc3ccc4ccc(-c5cccc6cc(-c7cccc8nc(-c9ccccc9)c(-c9ccccc9)nc78)ccc56)nc4c3n2)cc1]

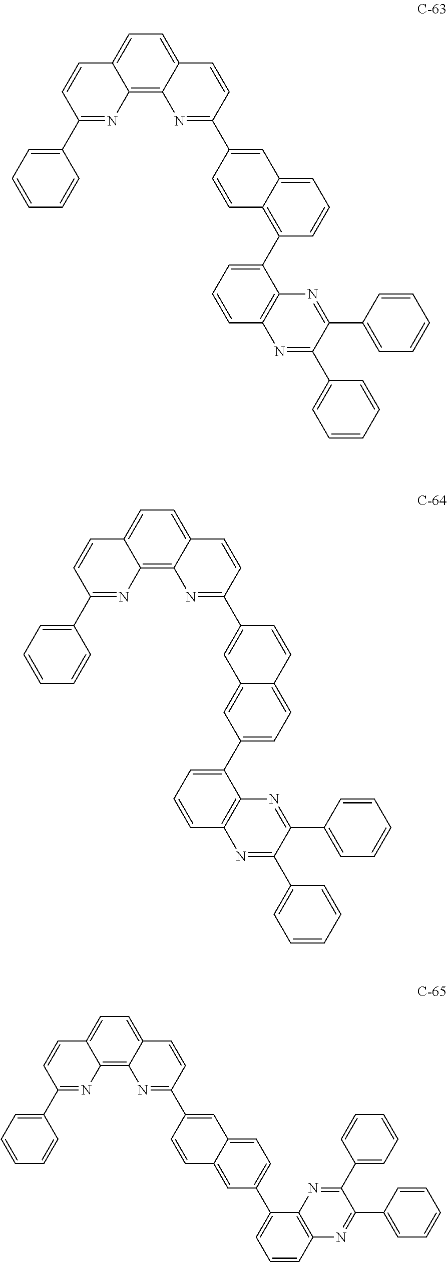 [SMILES: c1ccc(-c2ccc3ccc4ccc(-c5ccc6c(-c7cccc8nc(-c9ccccc9)c(-c9ccccc9)nc78)cccc6c5)nc4c3n2)cc1 ; c1ccc(-c2ccc3ccc4ccc(-c5ccc6ccc(-c7cccc8nc(-c9ccccc9)c(-c9ccccc9)nc78)cc6c5)nc4c3n2)cc1 ; c1ccc(-c2ccc3ccc4ccc(-c5ccc6cc(-c7cccc8nc(-c9ccccc9)c(-c9ccccc9)nc78)ccc6c5)nc4c3n2)cc1]

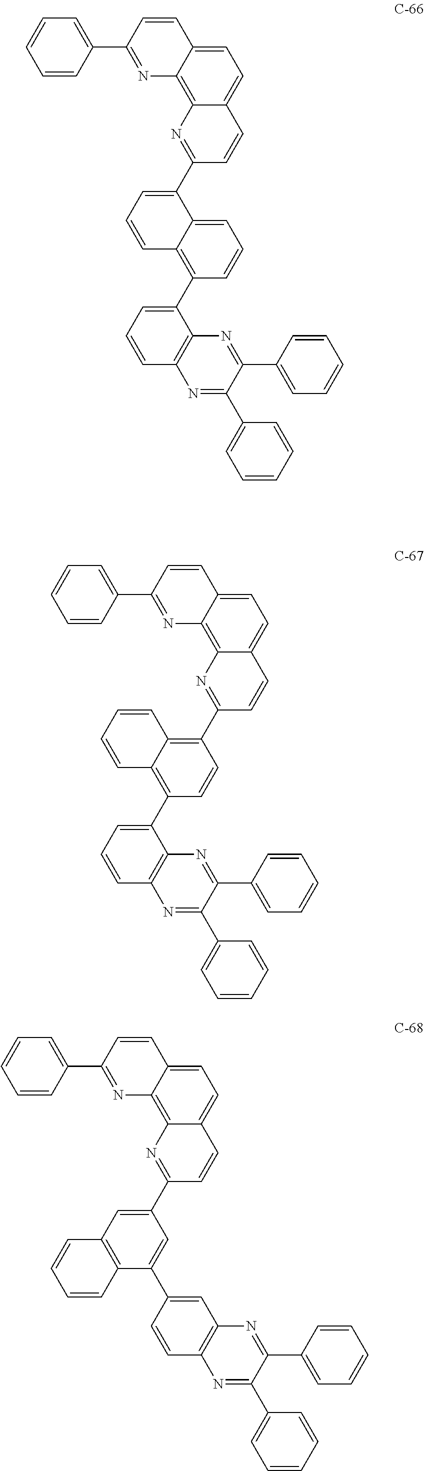 [SMILES: c1ccc(-c2ccc3ccc4ccc(-c5cccc6c(-c7cccc8nc(-c9ccccc9)c(-c9ccccc9)nc78)cccc56)nc4c3n2)cc1 ; c1ccc(-c2ccc3ccc4ccc(-c5ccc(-c6cccc7nc(-c8ccccc8)c(-c8ccccc8)nc67)c6ccccc56)nc4c3n2)cc1 ; c1ccc(-c2ccc3ccc4ccc(-c5cc(-c6ccc7nc(-c8ccccc8)c(-c8ccccc8)nc7c6)c6ccccc6c5)nc4c3n2)cc1]

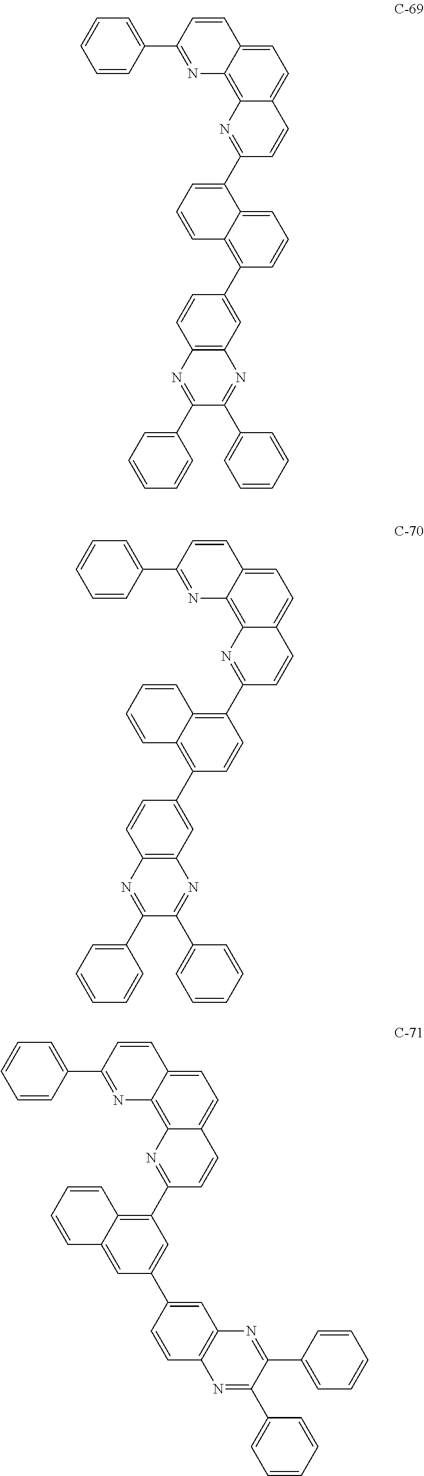

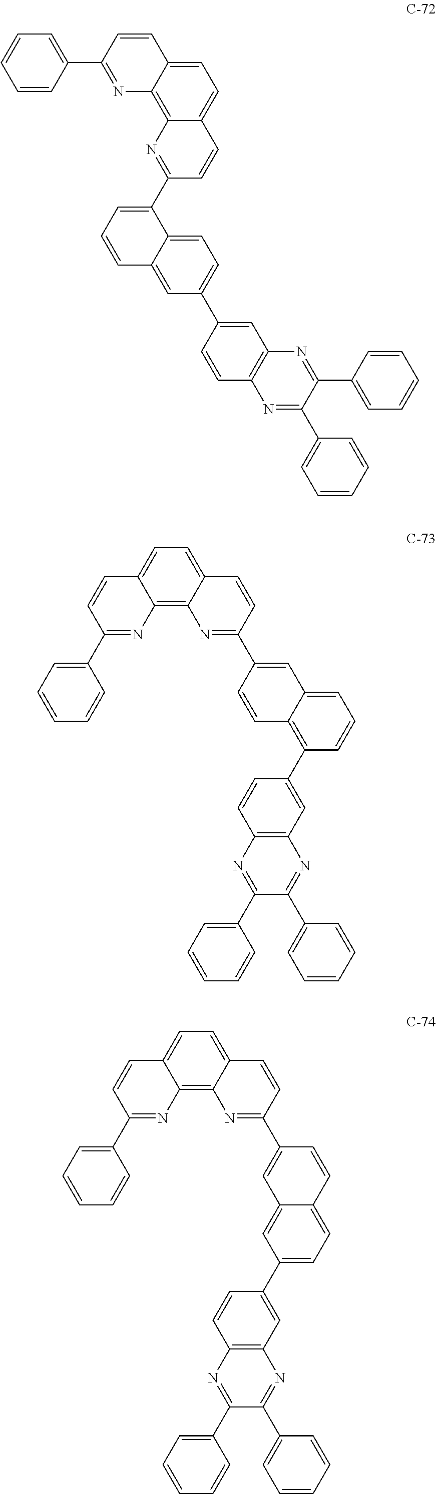 [SMILES: c1ccc(-c2ccc3ccc4ccc(-c5cccc6cc(-c7ccc8nc(-c9ccccc9)c(-c9ccccc9)nc8c7)ccc56)nc4c3n2)cc1 ; c1ccc(-c2ccc3ccc4ccc(-c5ccc6c(-c7ccc8nc(-c9ccccc9)c(-c9ccccc9)nc8c7)cccc6c5)nc4c3n2)cc1 ; c1ccc(-c2ccc3ccc4ccc(-c5ccc6ccc(-c7ccc8nc(-c9ccccc9)c(-c9ccccc9)nc8c7)cc6c5)nc4c3n2)cc1]

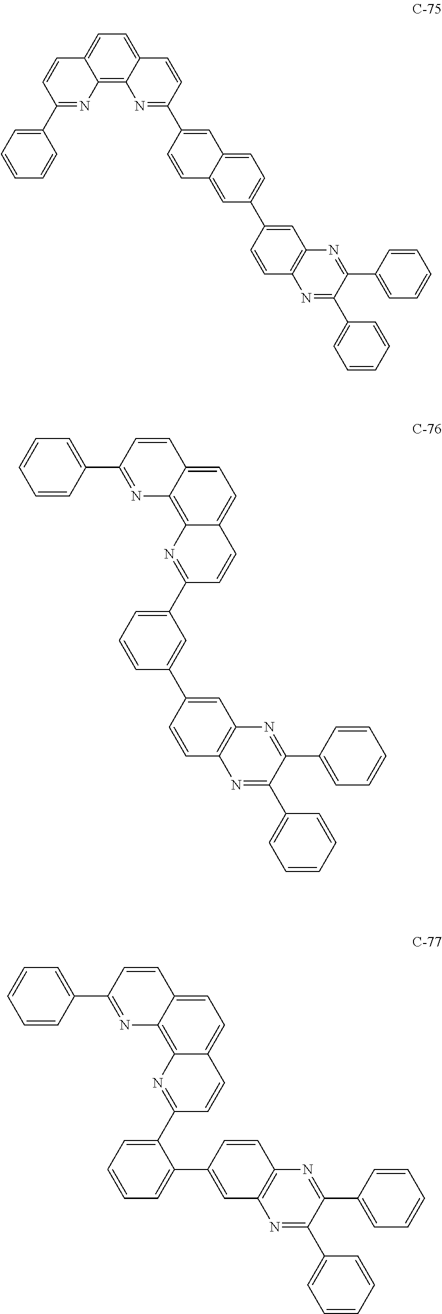 [SMILES: c1ccc(-c2ccc3ccc4ccc(-c5ccc6cc(-c7ccc8nc(-c9ccccc9)c(-c9ccccc9)nc8c7)ccc6c5)nc4c3n2)cc1 ; c1ccc(-c2ccc3ccc4ccc(-c5cccc(-c6ccc7nc(-c8ccccc8)c(-c8ccccc8)nc7c6)c5)nc4c3n2)cc1 ; c1ccc(-c2ccc3ccc4ccc(-c5ccccc5-c5ccc6nc(-c7ccccc7)c(-c7ccccc7)nc6c5)nc4c3n2)cc1]

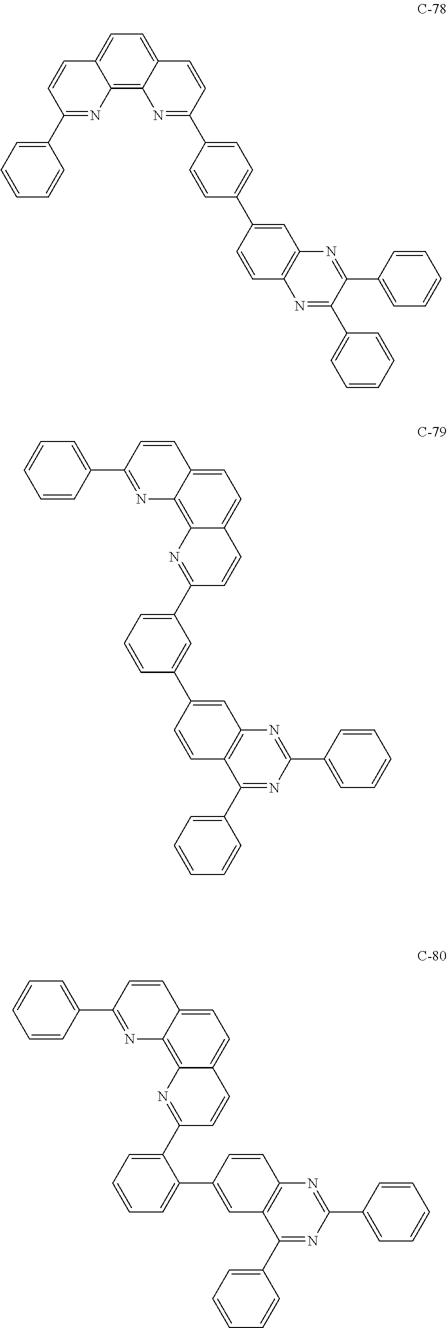 [SMILES: c1ccc(-c2ccc3ccc4ccc(-c5ccc(-c6ccc7nc(-c8ccccc8)c(-c8ccccc8)nc7c6)cc5)nc4c3n2)cc1 ; c1ccc(-c2ccc3ccc4ccc(-c5cccc(-c6ccc7c(-c8ccccc8)nc(-c8ccccc8)nc7c6)c5)nc4c3n2)cc1 ; c1ccc(-c2ccc3ccc4ccc(-c5ccccc5-c5ccc6nc(-c7ccccc7)nc(-c7ccccc7)c6c5)nc4c3n2)cc1]

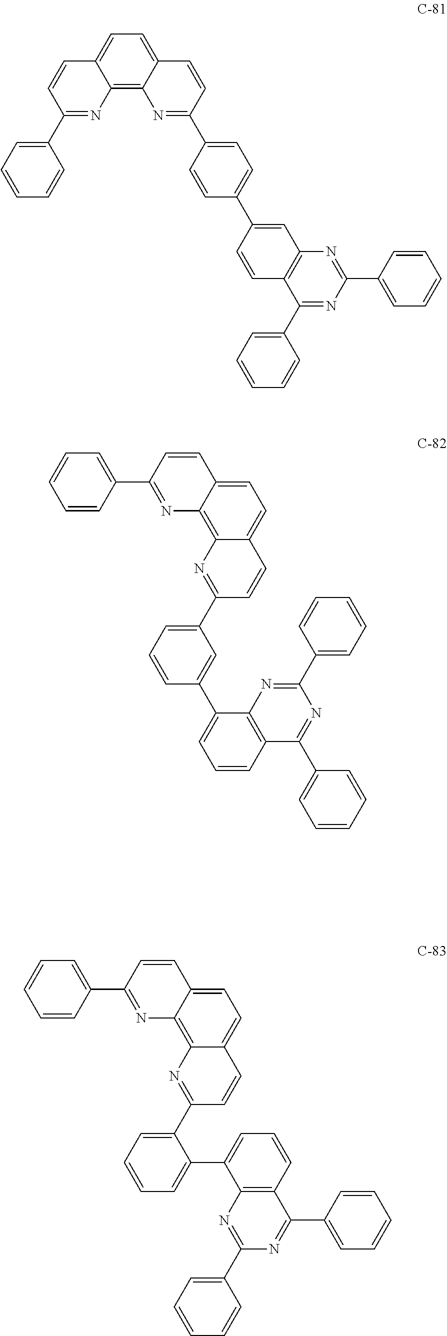 [SMILES: c1ccc(-c2ccc3ccc4ccc(-c5ccc(-c6ccc7c(-c8ccccc8)nc(-c8ccccc8)nc7c6)cc5)nc4c3n2)cc1 ; c1ccc(-c2ccc3ccc4ccc(-c5cccc(-c6cccc7c(-c8ccccc8)nc(-c8ccccc8)nc67)c5)nc4c3n2)cc1 ; c1ccc(-c2ccc3ccc4ccc(-c5ccccc5-c5cccc6c(-c7ccccc7)nc(-c7ccccc7)nc56)nc4c3n2)cc1]

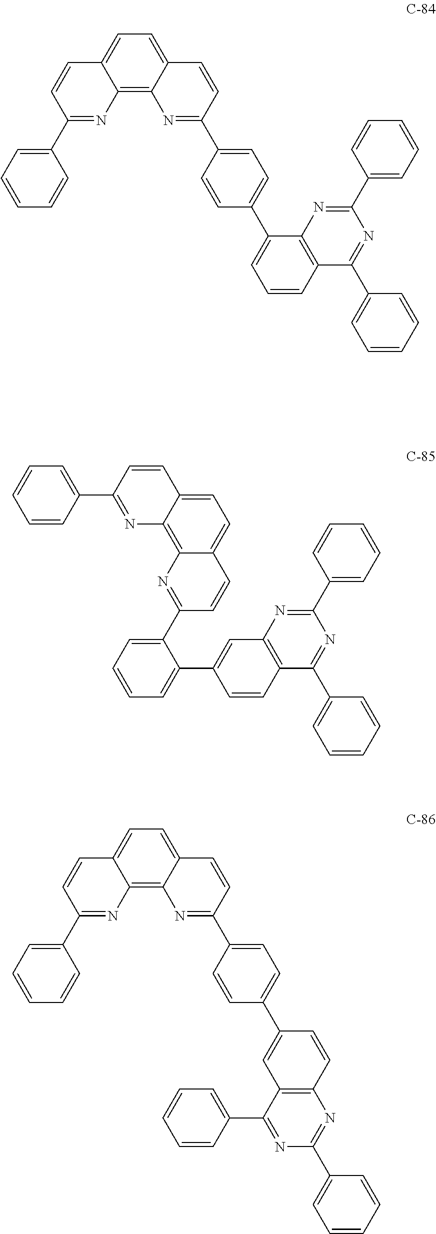 [SMILES: c1ccc(-c2ccc3ccc4ccc(-c5ccc(-c6cccc7c(-c8ccccc8)nc(-c8ccccc8)nc67)cc5)nc4c3n2)cc1 ; c1ccc(-c2ccc3ccc4ccc(-c5ccccc5-c5ccc6c(-c7ccccc7)nc(-c7ccccc7)nc6c5)nc4c3n2)cc1 ; c1ccc(-c2ccc3ccc4ccc(-c5ccc(-c6ccc7nc(-c8ccccc8)nc(-c8ccccc8)c7c6)cc5)nc4c3n2)cc1]

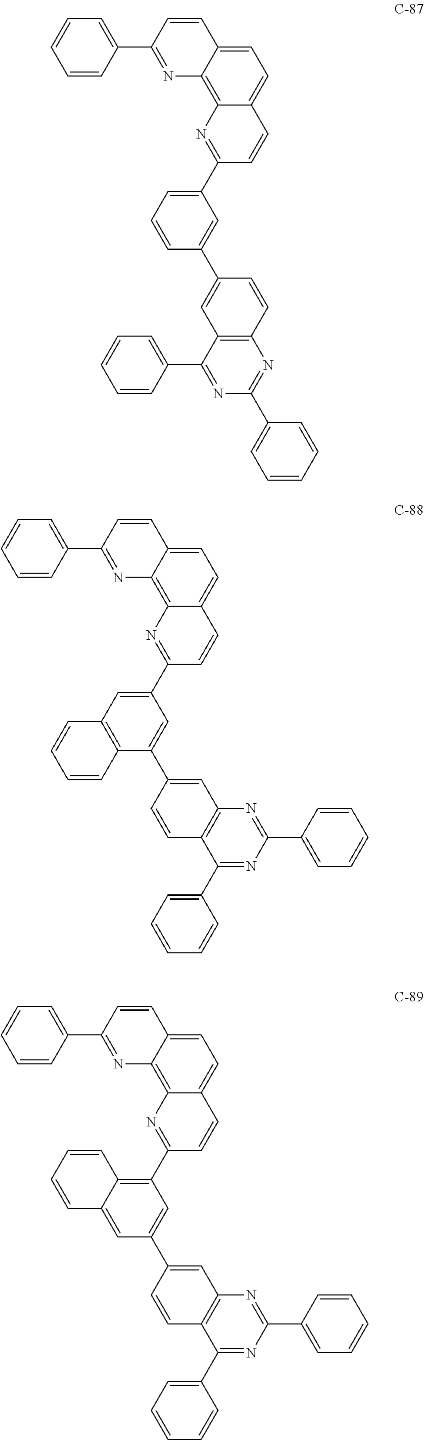 [SMILES: c1ccc(-c2ccc3ccc4ccc(-c5cccc(-c6ccc7nc(-c8ccccc8)nc(-c8ccccc8)c7c6)c5)nc4c3n2)cc1 ; c1ccc(-c2ccc3ccc4ccc(-c5cc(-c6ccc7c(-c8ccccc8)nc(-c8ccccc8)nc7c6)c6ccccc6c5)nc4c3n2)cc1 ; c1ccc(-c2ccc3ccc4ccc(-c5cc(-c6ccc7c(-c8ccccc8)nc(-c8ccccc8)nc7c6)cc6ccccc56)nc4c3n2)cc1]

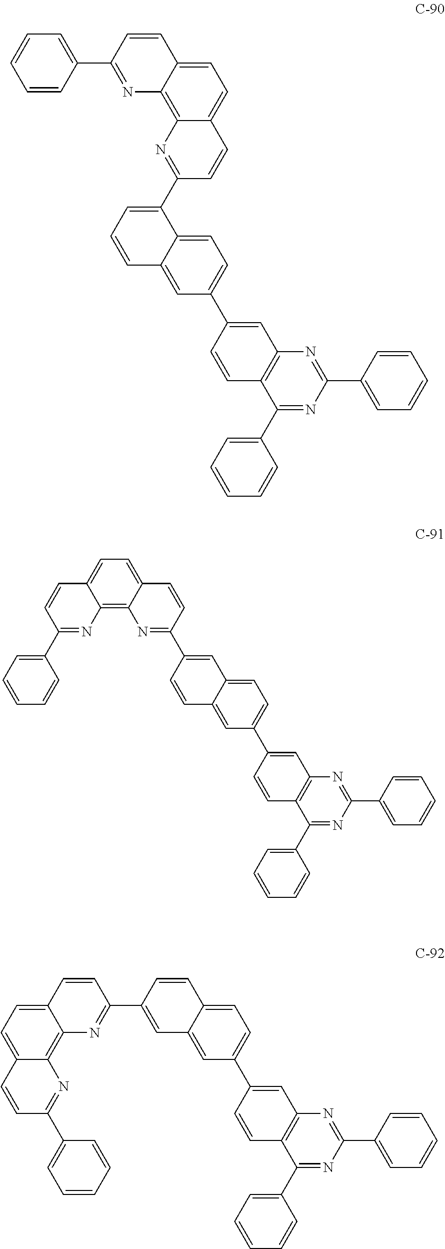 [SMILES: c1ccc(-c2ccc3ccc4ccc(-c5cccc6cc(-c7ccc8c(-c9ccccc9)nc(-c9ccccc9)nc8c7)ccc56)nc4c3n2)cc1 ; c1ccc(-c2ccc3ccc4ccc(-c5ccc6cc(-c7ccc8c(-c9ccccc9)nc(-c9ccccc9)nc8c7)ccc6c5)nc4c3n2)cc1 ; c1ccc(-c2ccc3ccc4ccc(-c5ccc6ccc(-c7ccc8c(-c9ccccc9)nc(-c9ccccc9)nc8c7)cc6c5)nc4c3n2)cc1]

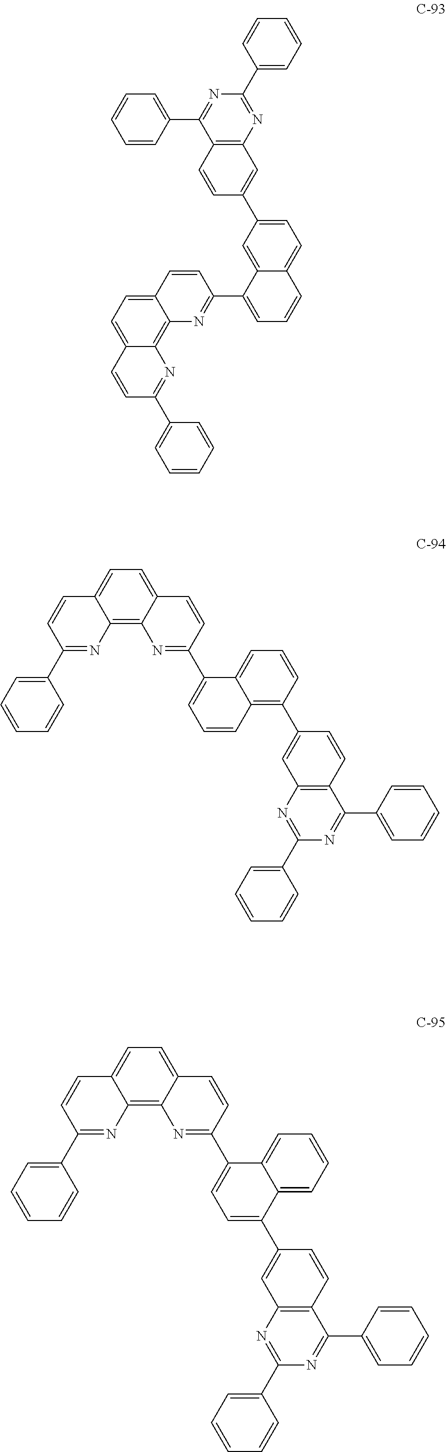 [SMILES: c1ccc(-c2ccc3ccc4ccc(-c5cccc6ccc(-c7ccc8c(-c9ccccc9)nc(-c9ccccc9)nc8c7)cc56)nc4c3n2)cc1 ; c1ccc(-c2ccc3ccc4ccc(-c5cccc6c(-c7ccc8c(-c9ccccc9)nc(-c9ccccc9)nc8c7)cccc56)nc4c3n2)cc1 ; c1ccc(-c2ccc3ccc4ccc(-c5ccc(-c6ccc7c(-c8ccccc8)nc(-c8ccccc8)nc7c6)c6ccccc56)nc4c3n2)cc1]

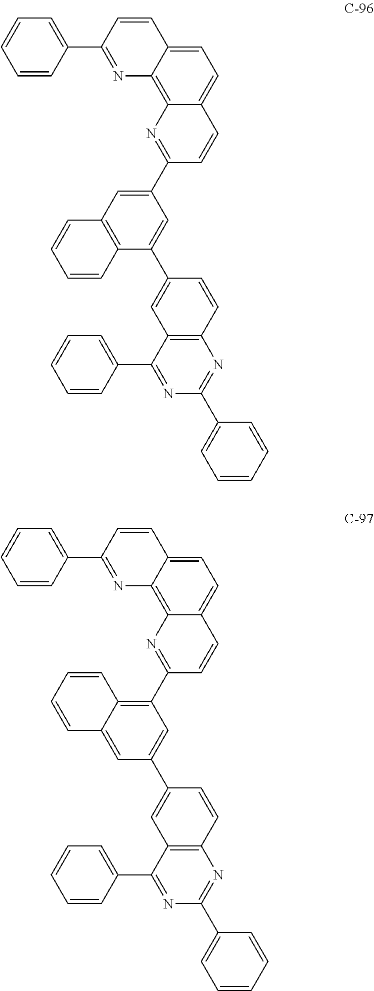 [SMILES: c1ccc(-c2ccc3ccc4ccc(-c5cc(-c6ccc7nc(-c8ccccc8)nc(-c8ccccc8)c7c6)c6ccccc6c5)nc4c3n2)cc1 ; c1ccc(-c2ccc3ccc4ccc(-c5cc(-c6ccc7nc(-c8ccccc8)nc(-c8ccccc8)c7c6)cc6ccccc56)nc4c3n2)cc1]

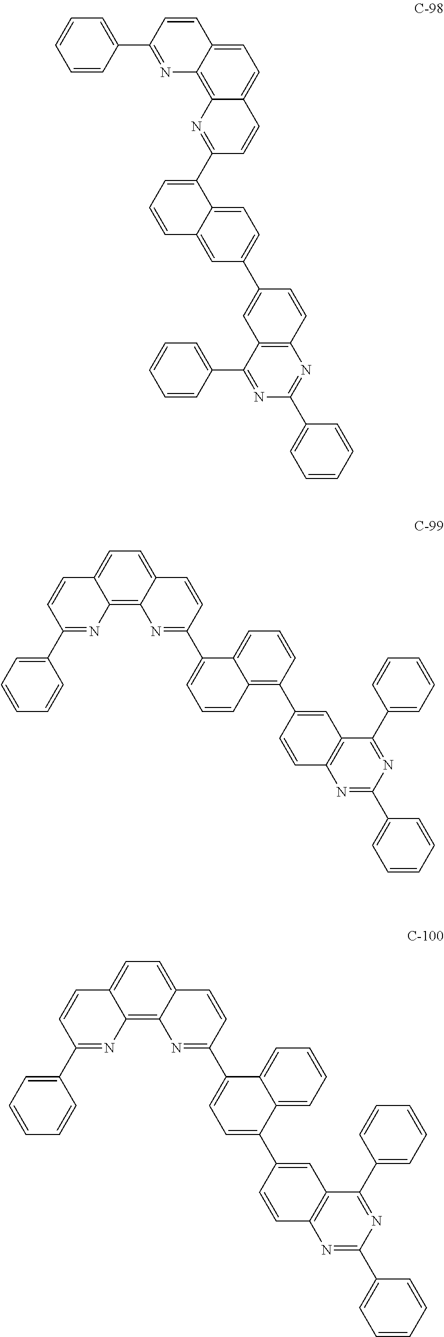 [SMILES: c1ccc(-c2ccc3ccc4ccc(-c5cccc6cc(-c7ccc8nc(-c9ccccc9)nc(-c9ccccc9)c8c7)ccc56)nc4c3n2)cc1 ; c1ccc(-c2ccc3ccc4ccc(-c5cccc6c(-c7ccc8nc(-c9ccccc9)nc(-c9ccccc9)c8c7)cccc56)nc4c3n2)cc1 ; c1ccc(-c2ccc3ccc4ccc(-c5ccc(-c6ccc7nc(-c8ccccc8)nc(-c8ccccc8)c7c6)c6ccccc56)nc4c3n2)cc1]

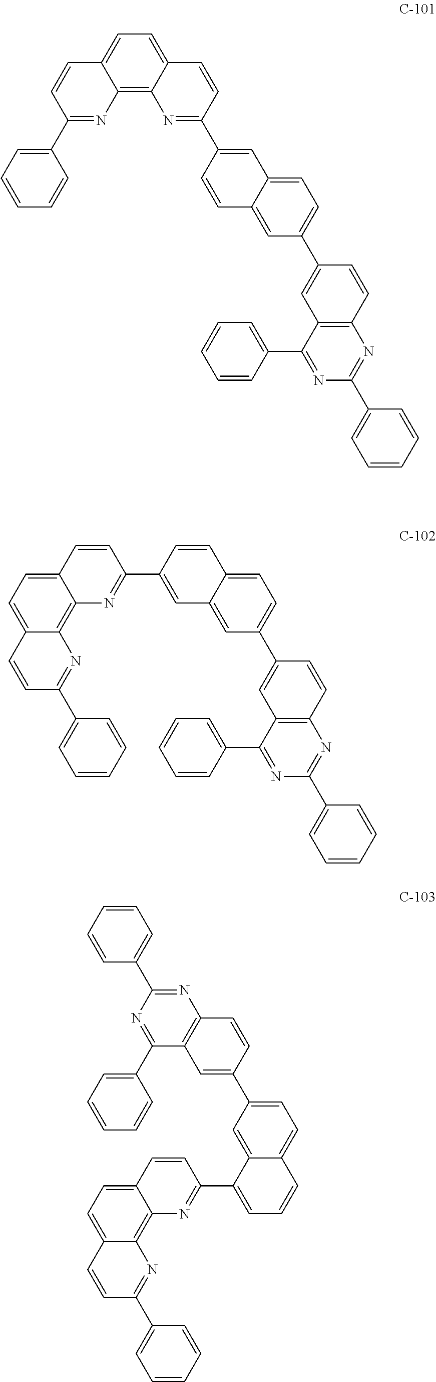 [SMILES: c1ccc(-c2ccc3ccc4ccc(-c5ccc6cc(-c7ccc8nc(-c9ccccc9)nc(-c9ccccc9)c8c7)ccc6c5)nc4c3n2)cc1 ; c1ccc(-c2ccc3ccc4ccc(-c5ccc6ccc(-c7ccc8nc(-c9ccccc9)nc(-c9ccccc9)c8c7)cc6c5)nc4c3n2)cc1 ; c1ccc(-c2ccc3ccc4ccc(-c5cccc6ccc(-c7ccc8nc(-c9ccccc9)nc(-c9ccccc9)c8c7)cc56)nc4c3n2)cc1]

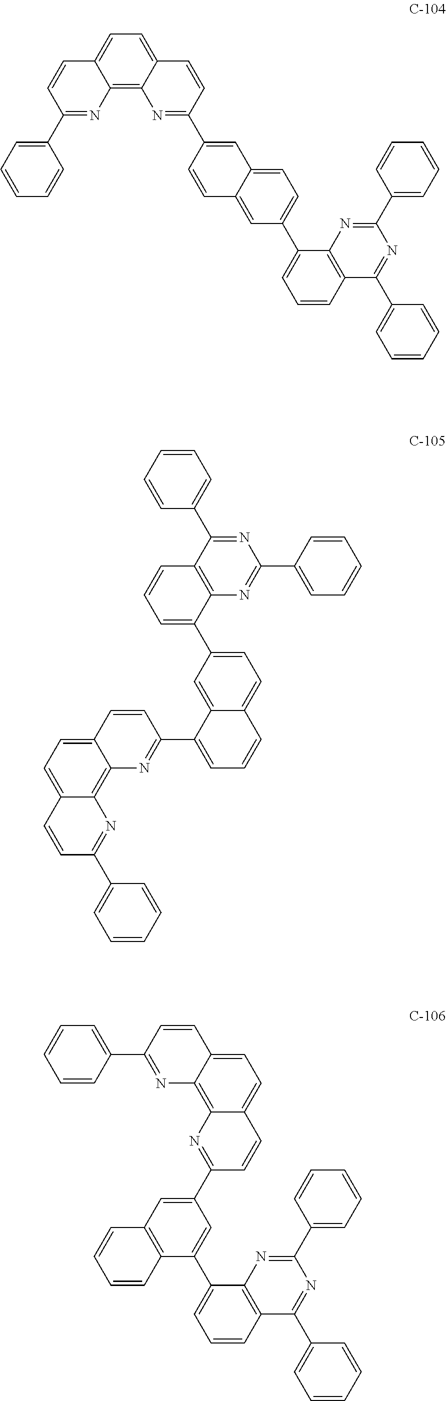 [SMILES: c1ccc(-c2ccc3ccc4ccc(-c5ccc6cc(-c7cccc8c(-c9ccccc9)nc(-c9ccccc9)nc78)ccc6c5)nc4c3n2)cc1 ; c1ccc(-c2ccc3ccc4ccc(-c5cccc6ccc(-c7cccc8c(-c9ccccc9)nc(-c9ccccc9)nc78)cc56)nc4c3n2)cc1 ; c1ccc(-c2ccc3ccc4ccc(-c5cc(-c6cccc7c(-c8ccccc8)nc(-c8ccccc8)nc67)c6ccccc6c5)nc4c3n2)cc1]

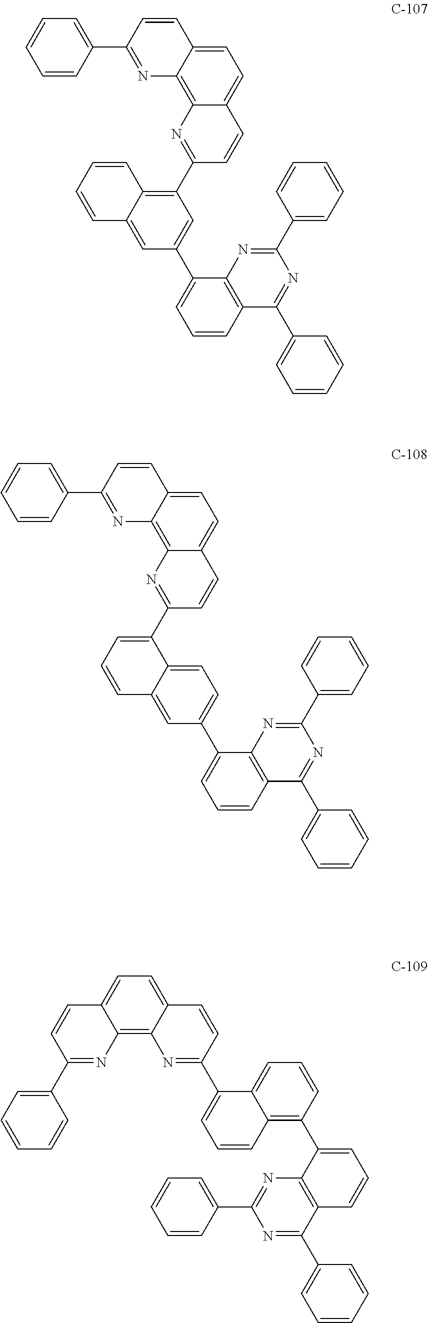 [SMILES: c1ccc(-c2ccc3ccc4ccc(-c5cc(-c6cccc7c(-c8ccccc8)nc(-c8ccccc8)nc67)cc6ccccc56)nc4c3n2)cc1 ; c1ccc(-c2ccc3ccc4ccc(-c5cccc6cc(-c7cccc8c(-c9ccccc9)nc(-c9ccccc9)nc78)ccc56)nc4c3n2)cc1 ; c1ccc(-c2ccc3ccc4ccc(-c5cccc6c(-c7cccc8c(-c9ccccc9)nc(-c9ccccc9)nc78)cccc56)nc4c3n2)cc1]

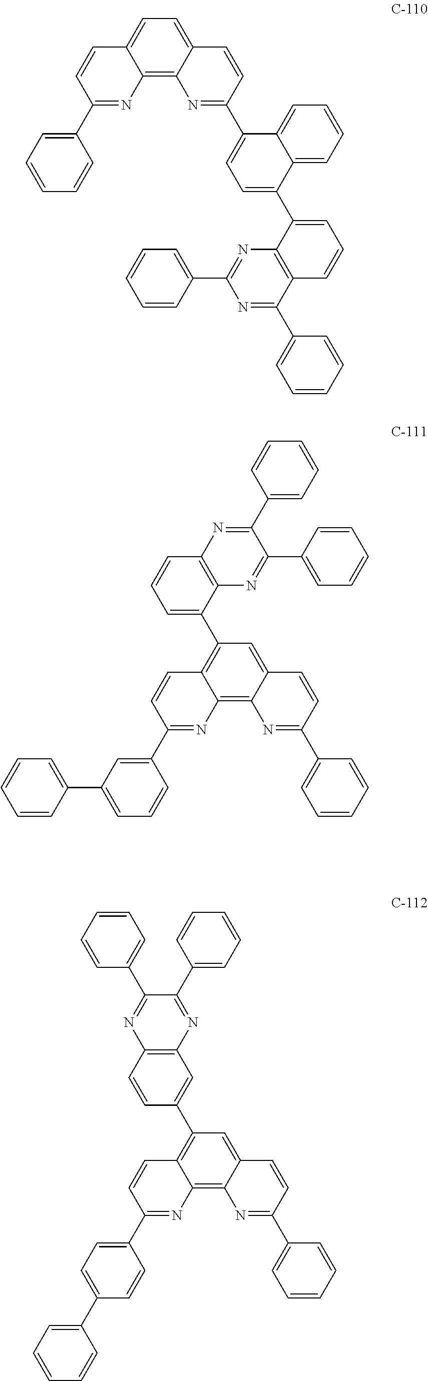 [SMILES: c1ccc(-c2ccc3ccc4ccc(-c5ccc(-c6cccc7c(-c8ccccc8)nc(-c8ccccc8)nc67)c6ccccc56)nc4c3n2)cc1 ; c1ccc(-c2cccc(-c3ccc4c(-c5cccc6nc(-c7ccccc7)c(-c7ccccc7)nc56)cc5ccc(-c6ccccc6)nc5c4n3)c2)cc1 ; c1ccc(-c2ccc(-c3ccc4c(-c5ccc6nc(-c7ccccc7)c(-c7ccccc7)nc6c5)cc5ccc(-c6ccccc6)nc5c4n3)cc2)cc1]

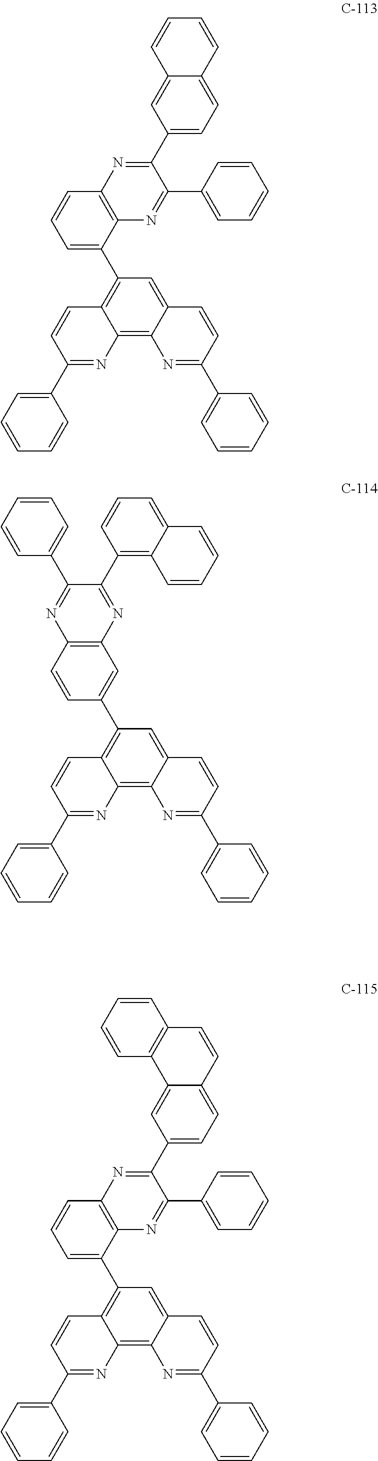 [SMILES: c1ccc(-c2ccc3cc(-c4cccc5nc(-c6ccc7ccccc7c6)c(-c6ccccc6)nc45)c4ccc(-c5ccccc5)nc4c3n2)cc1 ; c1ccc(-c2ccc3cc(-c4ccc5nc(-c6ccccc6)c(-c6cccc7ccccc67)nc5c4)c4ccc(-c5ccccc5)nc4c3n2)cc1 ; c1ccc(-c2ccc3cc(-c4cccc5nc(-c6ccc7ccc8ccccc8c7c6)c(-c6ccccc6)nc45)c4ccc(-c5ccccc5)nc4c3n2)cc1]

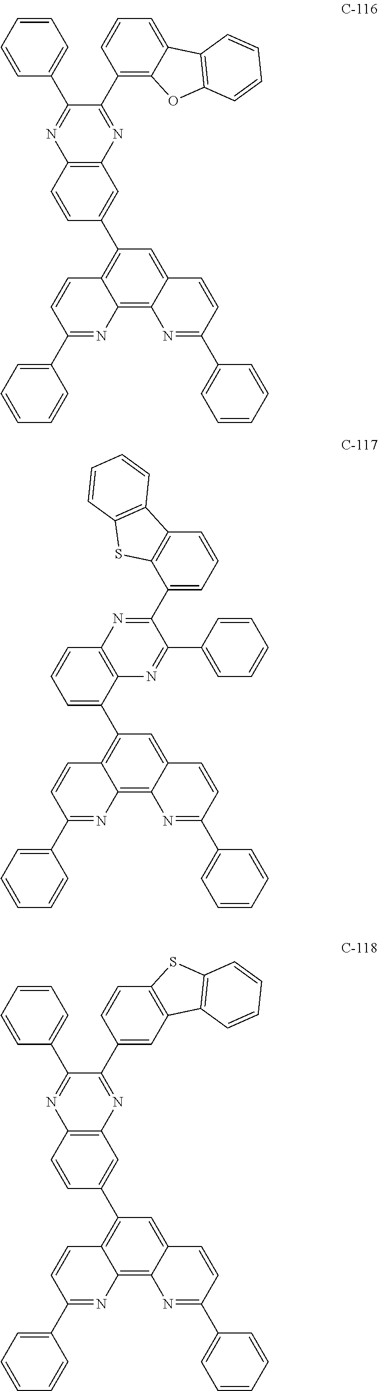 [SMILES: c1ccc(-c2ccc3cc(-c4ccc5nc(-c6ccccc6)c(-c6cccc7c6oc6ccccc67)nc5c4)c4ccc(-c5ccccc5)nc4c3n2)cc1 ; c1ccc(-c2ccc3cc(-c4cccc5nc(-c6cccc7c6sc6ccccc67)c(-c6ccccc6)nc45)c4ccc(-c5ccccc5)nc4c3n2)cc1 ; c1ccc(-c2ccc3cc(-c4ccc5nc(-c6ccccc6)c(-c6ccc7sc8ccccc8c7c6)nc5c4)c4ccc(-c5ccccc5)nc4c3n2)cc1]

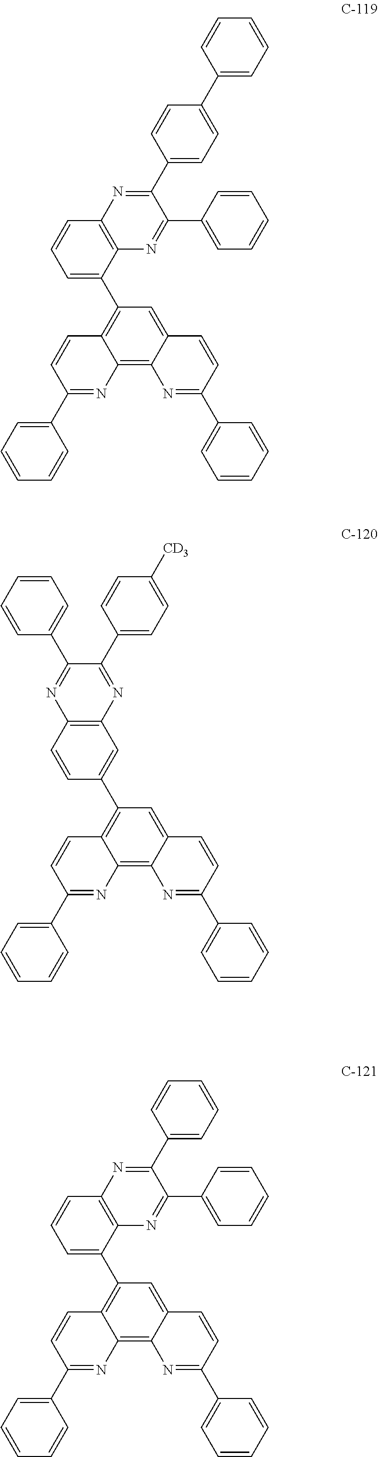 [SMILES: c1ccc(-c2ccc(-c3nc4cccc(-c5cc6ccc(-c7ccccc7)nc6c6nc(-c7ccccc7)ccc56)c4nc3-c3ccccc3)cc2)cc1 ; [2H]C([2H])([2H])c1ccc(-c2nc3cc(-c4cc5ccc(-c6ccccc6)nc5c5nc(-c6ccccc6)ccc45)ccc3nc2-c2ccccc2)cc1 ; c1ccc(-c2ccc3cc(-c4cccc5nc(-c6ccccc6)c(-c6ccccc6)nc45)c4ccc(-c5ccccc5)nc4c3n2)cc1]

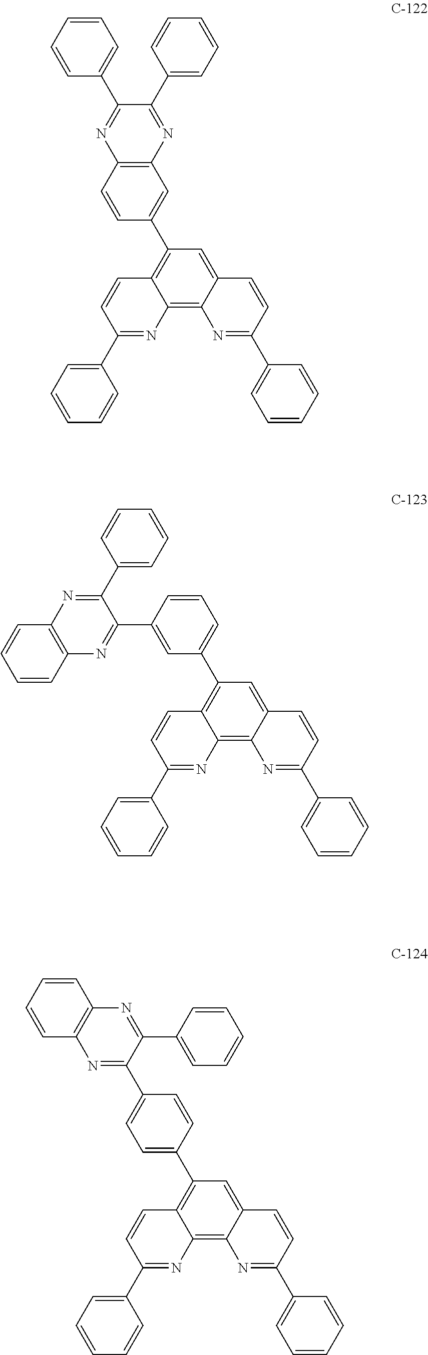 [SMILES: c1ccc(-c2ccc3cc(-c4ccc5nc(-c6ccccc6)c(-c6ccccc6)nc5c4)c4ccc(-c5ccccc5)nc4c3n2)cc1 ; c1ccc(-c2ccc3cc(-c4cccc(-c5nc6ccccc6nc5-c5ccccc5)c4)c4ccc(-c5ccccc5)nc4c3n2)cc1 ; c1ccc(-c2ccc3cc(-c4ccc(-c5nc6ccccc6nc5-c5ccccc5)cc4)c4ccc(-c5ccccc5)nc4c3n2)cc1]

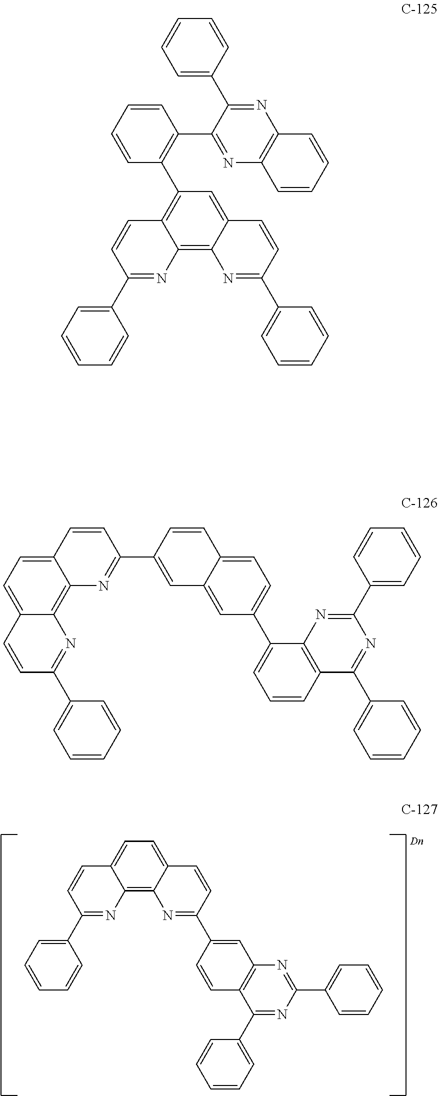

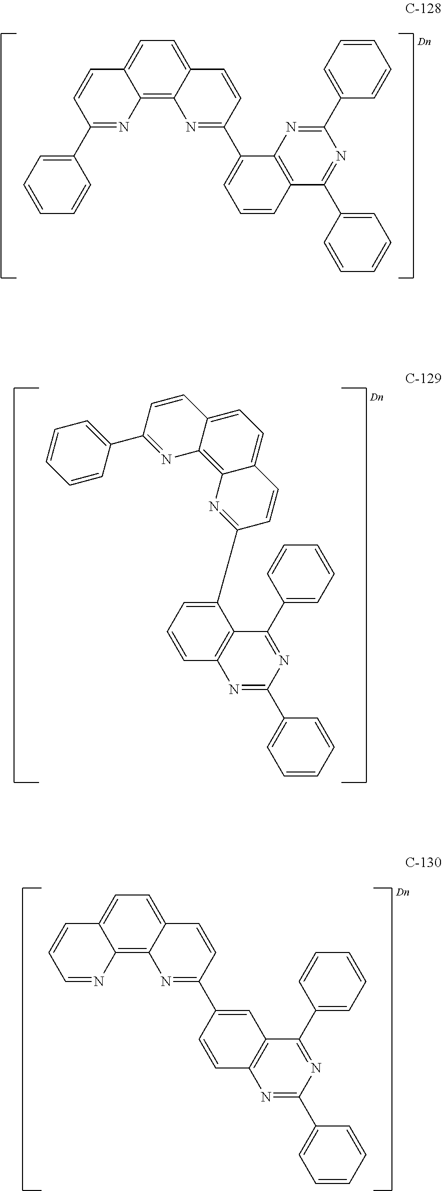

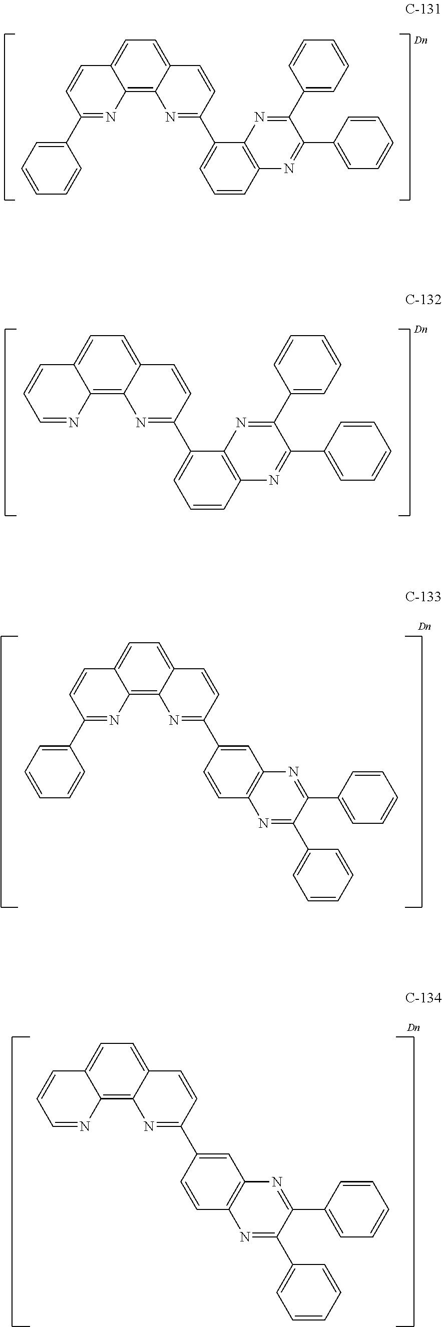

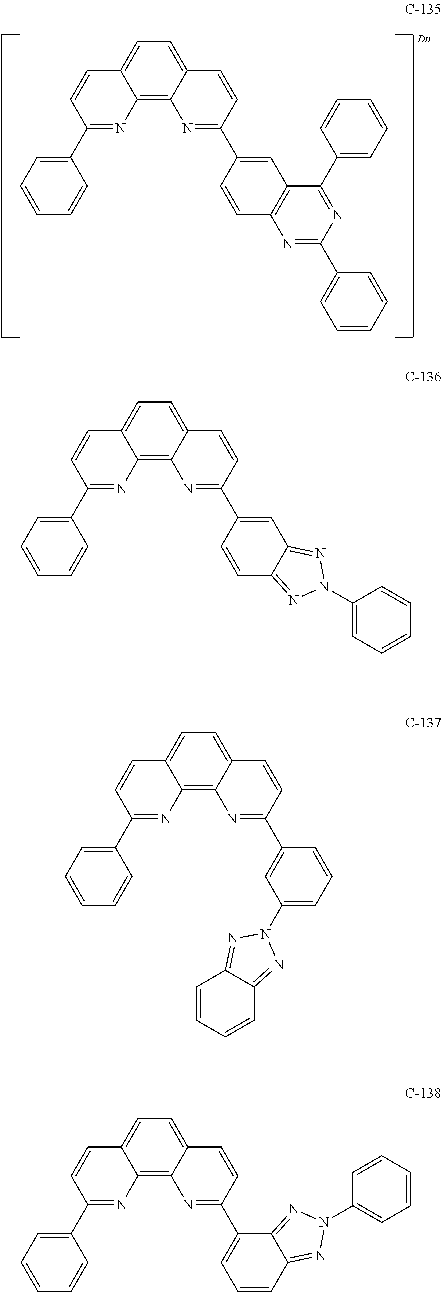

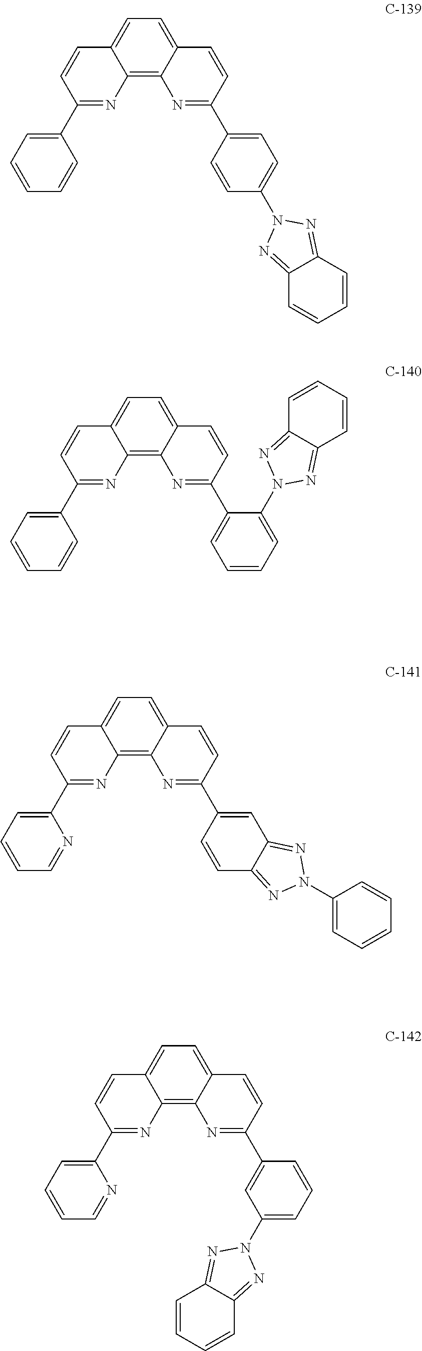 [SMILES: c1ccc(-c2ccc3ccc4ccc(-c5ccc(-n6nc7ccccc7n6)cc5)nc4c3n2)cc1 ; c1ccc(-c2ccc3ccc4ccc(-c5ccccc5-n5nc6ccccc6n5)nc4c3n2)cc1 ; c1ccc(-n2nc3ccc(-c4ccc5ccc6ccc(-c7ccccn7)nc6c5n4)cc3n2)cc1 ; c1ccc(-c2ccc3ccc4ccc(-c5cccc(-n6nc7ccccc7n6)c5)nc4c3n2)nc1]

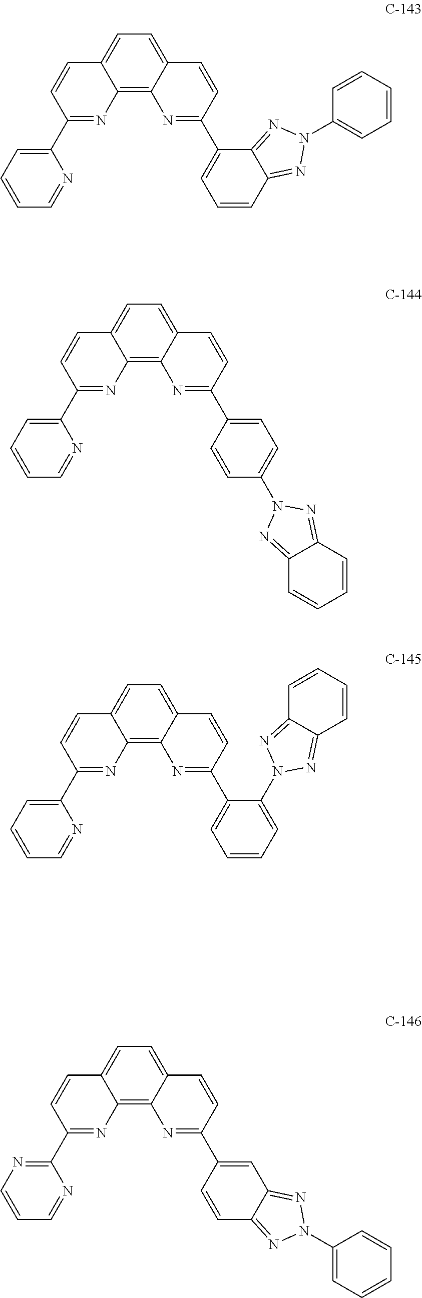 [SMILES: c1ccc(-n2nc3cccc(-c4ccc5ccc6ccc(-c7ccccn7)nc6c5n4)c3n2)cc1 ; c1ccc(-c2ccc3ccc4ccc(-c5ccc(-n6nc7ccccc7n6)cc5)nc4c3n2)nc1 ; c1ccc(-c2ccc3ccc4ccc(-c5ccccc5-n5nc6ccccc6n5)nc4c3n2)nc1 ; c1ccc(-n2nc3ccc(-c4ccc5ccc6ccc(-c7ncccn7)nc6c5n4)cc3n2)cc1]

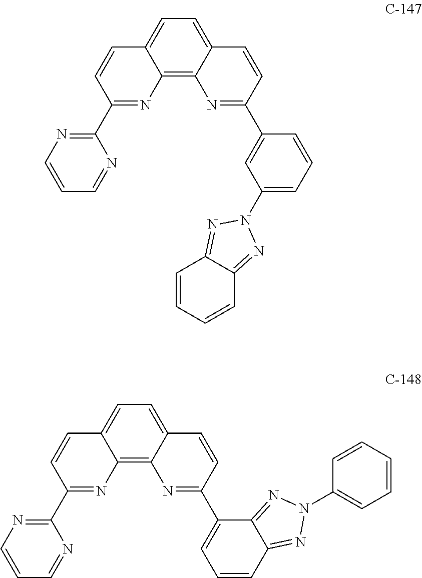 [SMILES: c1cnc(-c2ccc3ccc4ccc(-c5cccc(-n6nc7ccccc7n6)c5)nc4c3n2)nc1 ; c1ccc(-n2nc3cccc(-c4ccc5ccc6ccc(-c7ncccn7)nc6c5n4)c3n2)cc1]

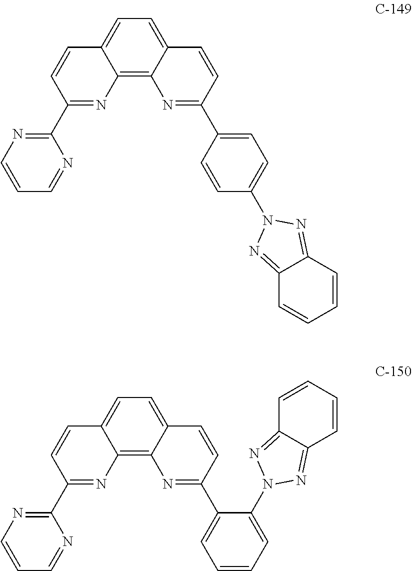 [SMILES: c1cnc(-c2ccc3ccc4ccc(-c5ccc(-n6nc7ccccc7n6)cc5)nc4c3n2)nc1 ; c1cnc(-c2ccc3ccc4ccc(-c5ccccc5-n5nc6ccccc6n5)nc4c3n2)nc1]

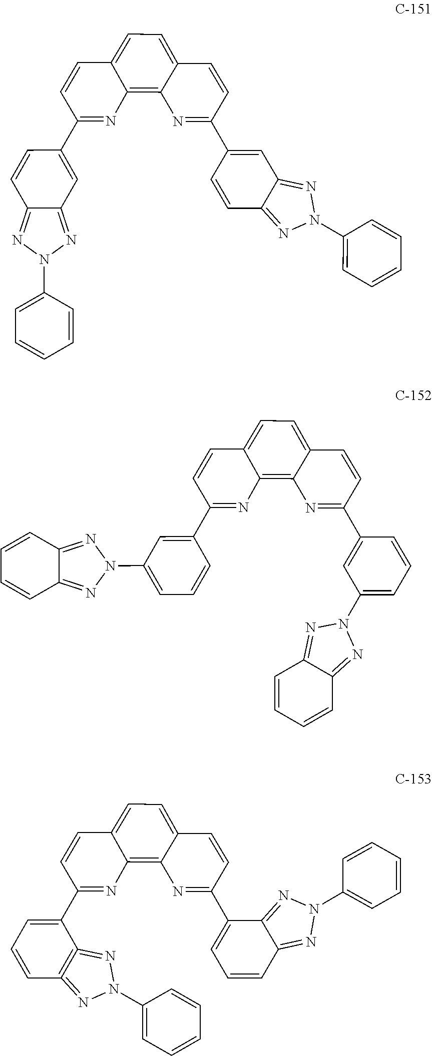

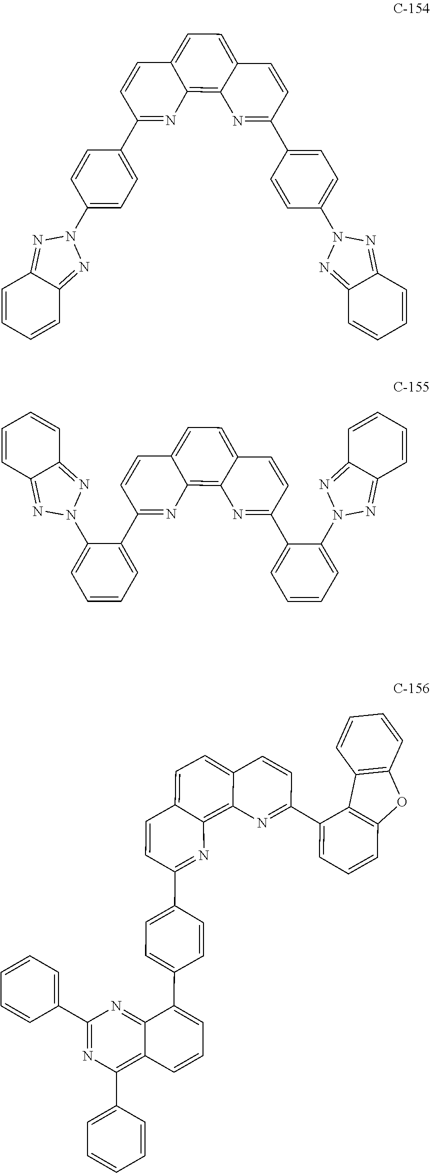 [SMILES: c1ccc2nn(-c3ccc(-c4ccc5ccc6ccc(-c7ccc(-n8nc9ccccc9n8)cc7)nc6c5n4)cc3)nc2c1 ; c1ccc(-n2nc3ccccc3n2)c(-c2ccc3ccc4ccc(-c5ccccc5-n5nc6ccccc6n5)nc4c3n2)c1 ; c1ccc(-c2nc(-c3ccccc3)c3cccc(-c4ccc(-c5ccc6ccc7ccc(-c8cccc9oc%10ccccc%10c89)nc7c6n5)cc4)c3n2)cc1]

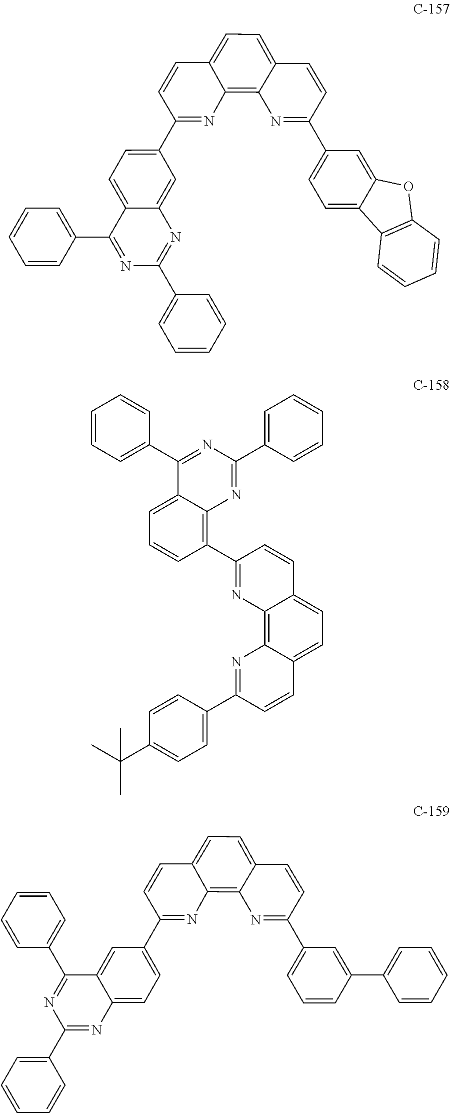 [SMILES: c1ccc(-c2nc(-c3ccccc3)c3ccc(-c4ccc5ccc6ccc(-c7ccc8c(c7)oc7ccccc78)nc6c5n4)cc3n2)cc1 ; CC(C)(C)c1ccc(-c2ccc3ccc4ccc(-c5cccc6c(-c7ccccc7)nc(-c7ccccc7)nc56)nc4c3n2)cc1 ; c1ccc(-c2cccc(-c3ccc4ccc5ccc(-c6ccc7nc(-c8ccccc8)nc(-c8ccccc8)c7c6)nc5c4n3)c2)cc1]

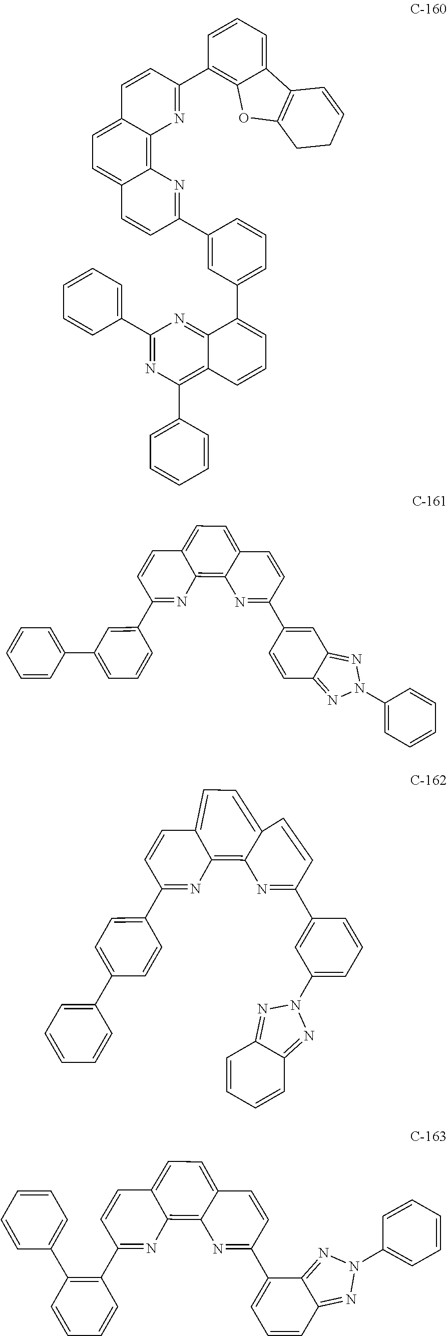

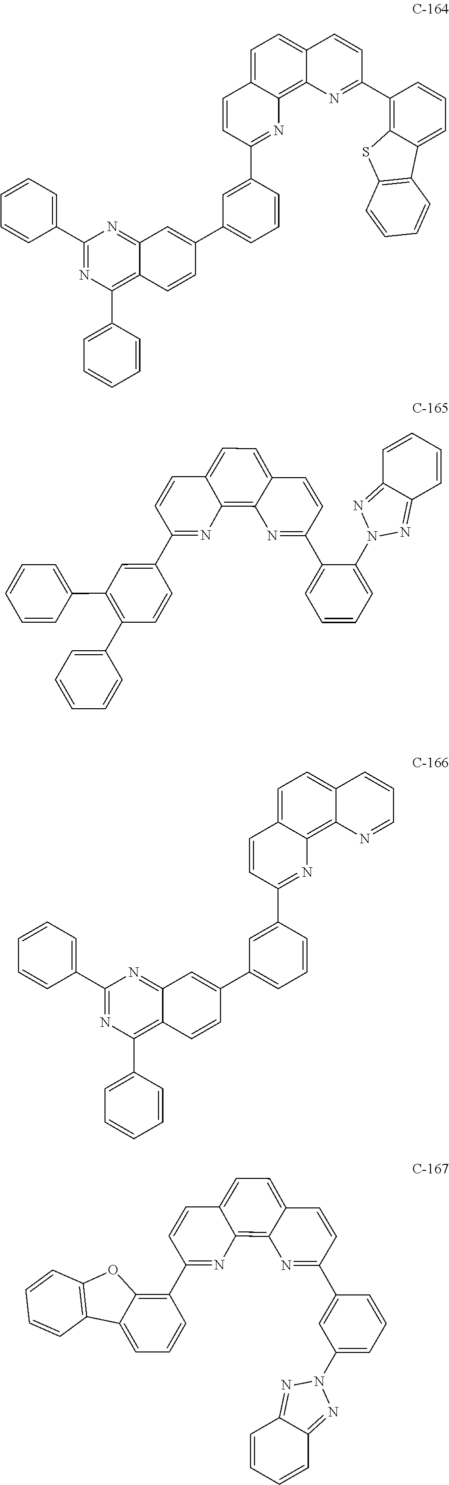 [SMILES: c1ccc(-c2nc(-c3ccccc3)c3ccc(-c4cccc(-c5ccc6ccc7ccc(-c8cccc9c8sc8ccccc89)nc7c6n5)c4)cc3n2)cc1 ; c1ccc(-c2ccc(-c3ccc4ccc5ccc(-c6ccccc6-n6nc7ccccc7n6)nc5c4n3)cc2-c2ccccc2)cc1 ; c1ccc(-c2nc(-c3ccccc3)c3ccc(-c4cccc(-c5ccc6ccc7cccnc7c6n5)c4)cc3n2)cc1 ; c1cc(-c2ccc3ccc4ccc(-c5cccc6c5oc5ccccc56)nc4c3n2)cc(-n2nc3ccccc3n2)c1]

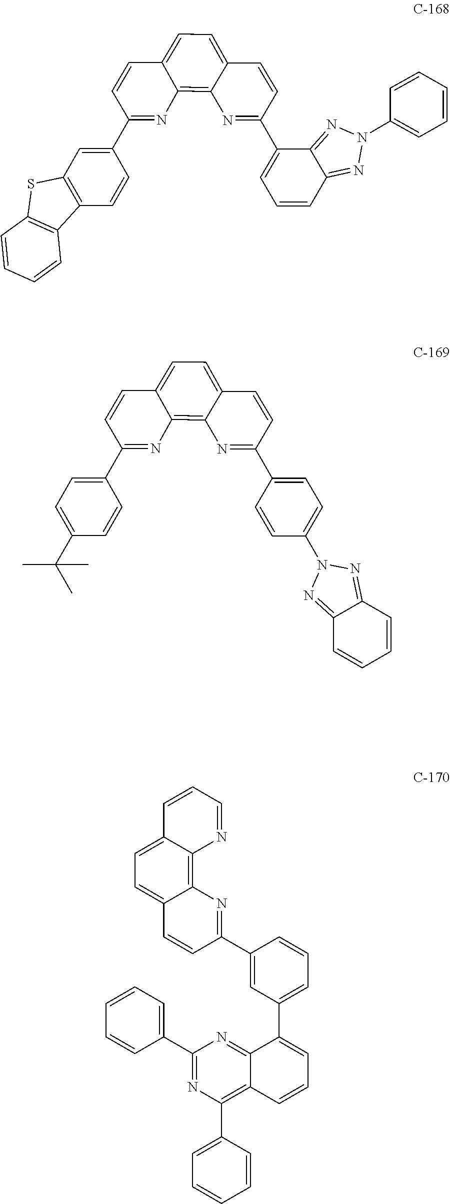 [SMILES: c1ccc(-n2nc3cccc(-c4ccc5ccc6ccc(-c7ccc8c(c7)sc7ccccc78)nc6c5n4)c3n2)cc1 ; CC(C)(C)c1ccc(-c2ccc3ccc4ccc(-c5ccc(-n6nc7ccccc7n6)cc5)nc4c3n2)cc1 ; c1ccc(-c2nc(-c3ccccc3)c3cccc(-c4cccc(-c5ccc6ccc7cccnc7c6n5)c4)c3n2)cc1]

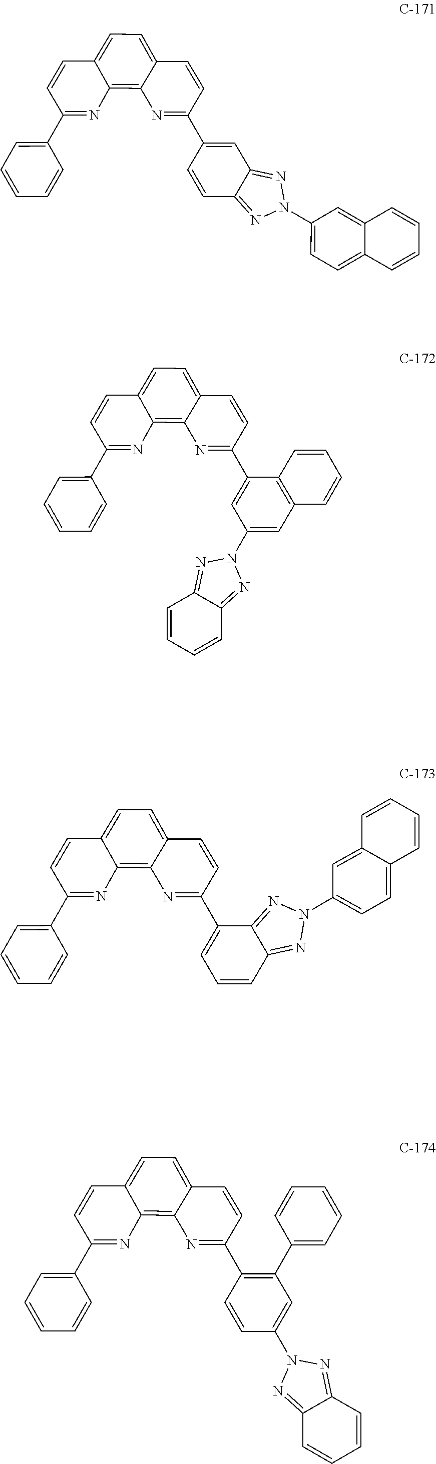 [SMILES: c1ccc(-c2ccc3ccc4ccc(-c5ccc6nn(-c7ccc8ccccc8c7)nc6c5)nc4c3n2)cc1 ; c1ccc(-c2ccc3ccc4ccc(-c5cc(-n6nc7ccccc7n6)cc6ccccc56)nc4c3n2)cc1 ; c1ccc(-c2ccc3ccc4ccc(-c5cccc6nn(-c7ccc8ccccc8c7)nc56)nc4c3n2)cc1 ; c1ccc(-c2ccc3ccc4ccc(-c5ccc(-n6nc7ccccc7n6)cc5-c5ccccc5)nc4c3n2)cc1]

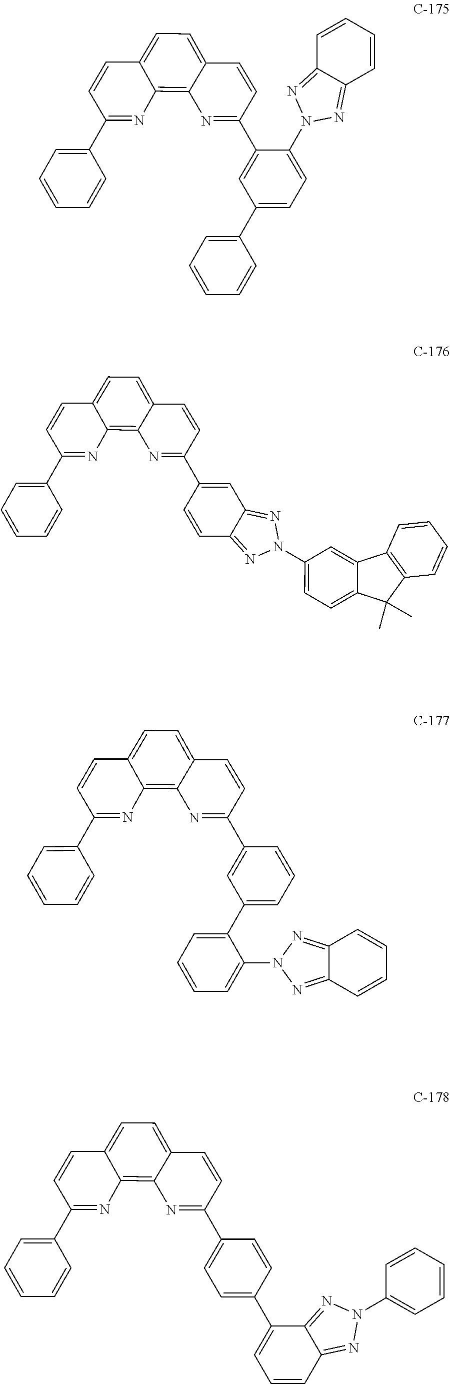 [SMILES: c1ccc(-c2ccc(-n3nc4ccccc4n3)c(-c3ccc4ccc5ccc(-c6ccccc6)nc5c4n3)c2)cc1 ; CC1(C)c2ccccc2-c2cc(-n3nc4ccc(-c5ccc6ccc7ccc(-c8ccccc8)nc7c6n5)cc4n3)ccc21 ; c1ccc(-c2ccc3ccc4ccc(-c5cccc(-c6ccccc6-n6nc7ccccc7n6)c5)nc4c3n2)cc1 ; c1ccc(-c2ccc3ccc4ccc(-c5ccc(-c6cccc7nn(-c8ccccc8)nc67)cc5)nc4c3n2)cc1]

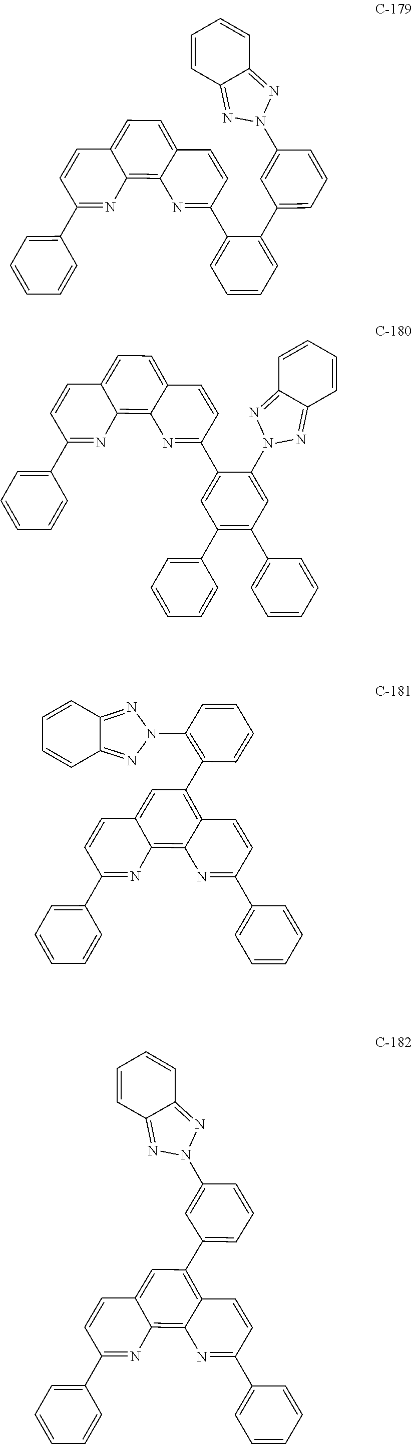 [SMILES: c1ccc(-c2ccc3ccc4ccc(-c5ccccc5-c5cccc(-n6nc7ccccc7n6)c5)nc4c3n2)cc1 ; c1ccc(-c2ccc3ccc4ccc(-c5cc(-c6ccccc6)c(-c6ccccc6)cc5-n5nc6ccccc6n5)nc4c3n2)cc1 ; c1ccc(-c2ccc3cc(-c4ccccc4-n4nc5ccccc5n4)c4ccc(-c5ccccc5)nc4c3n2)cc1 ; c1ccc(-c2ccc3cc(-c4cccc(-n5nc6ccccc6n5)c4)c4ccc(-c5ccccc5)nc4c3n2)cc1]

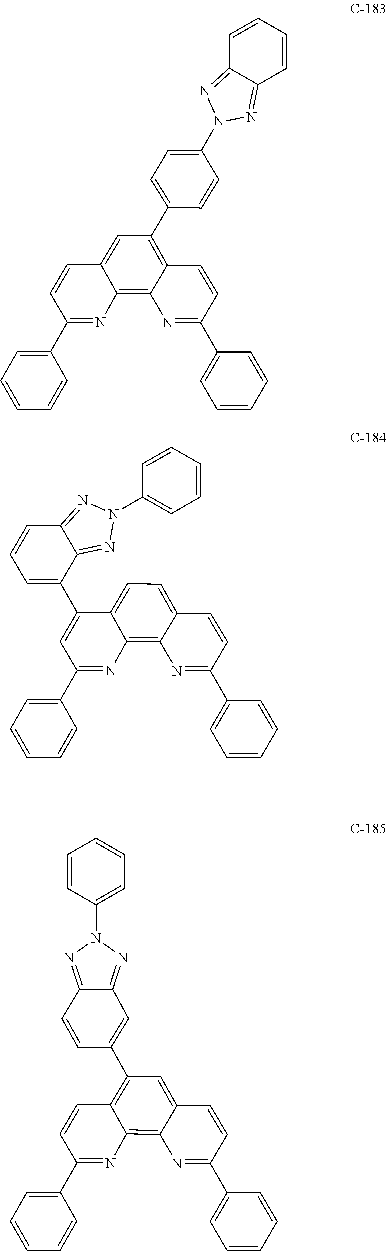 [SMILES: c1ccc(-c2ccc3cc(-c4ccc(-n5nc6ccccc6n5)cc4)c4ccc(-c5ccccc5)nc4c3n2)cc1 ; c1ccc(-c2ccc3ccc4c(-c5cccc6nn(-c7ccccc7)nc56)cc(-c5ccccc5)nc4c3n2)cc1 ; c1ccc(-c2ccc3cc(-c4ccc5nn(-c6ccccc6)nc5c4)c4ccc(-c5ccccc5)nc4c3n2)cc1]

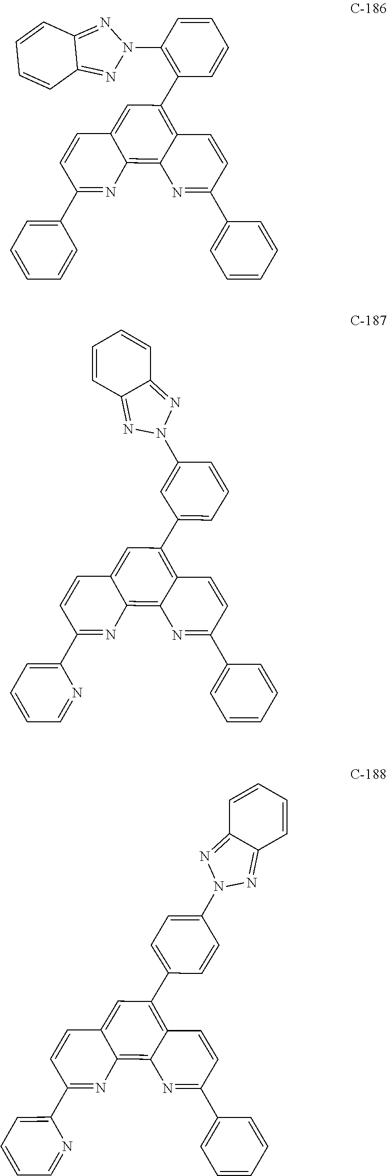 [SMILES: c1ccc(-c2ccc3cc(-c4ccccc4-n4nc5ccccc5n4)c4ccc(-c5ccccc5)nc4c3n2)cc1 ; c1ccc(-c2ccc3c(-c4cccc(-n5nc6ccccc6n5)c4)cc4ccc(-c5ccccn5)nc4c3n2)cc1 ; c1ccc(-c2ccc3c(-c4ccc(-n5nc6ccccc6n5)cc4)cc4ccc(-c5ccccn5)nc4c3n2)cc1]

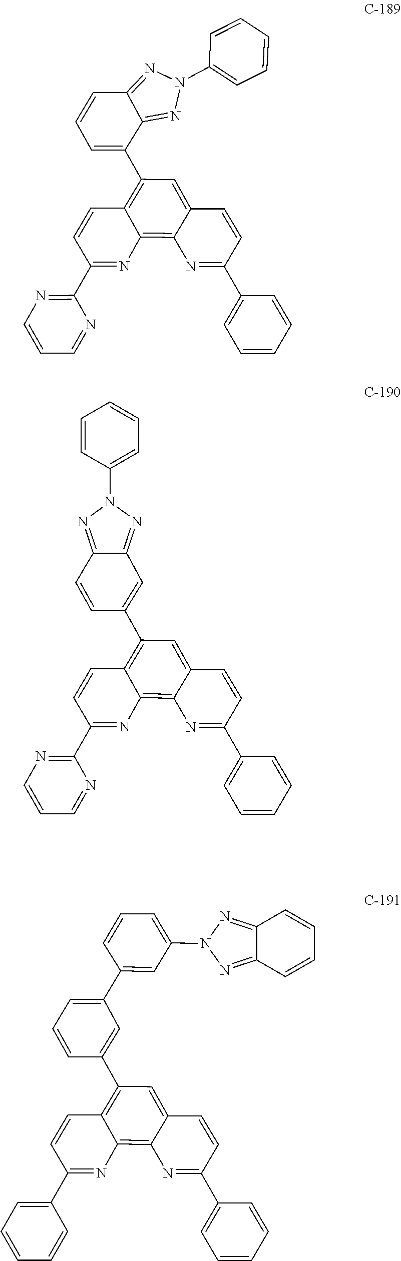 [SMILES: c1ccc(-c2ccc3cc(-c4cccc5nn(-c6ccccc6)nc45)c4ccc(-c5ncccn5)nc4c3n2)cc1 ; c1ccc(-c2ccc3cc(-c4ccc5nn(-c6ccccc6)nc5c4)c4ccc(-c5ncccn5)nc4c3n2)cc1 ; c1ccc(-c2ccc3cc(-c4cccc(-c5cccc(-n6nc7ccccc7n6)c5)c4)c4ccc(-c5ccccc5)nc4c3n2)cc1]

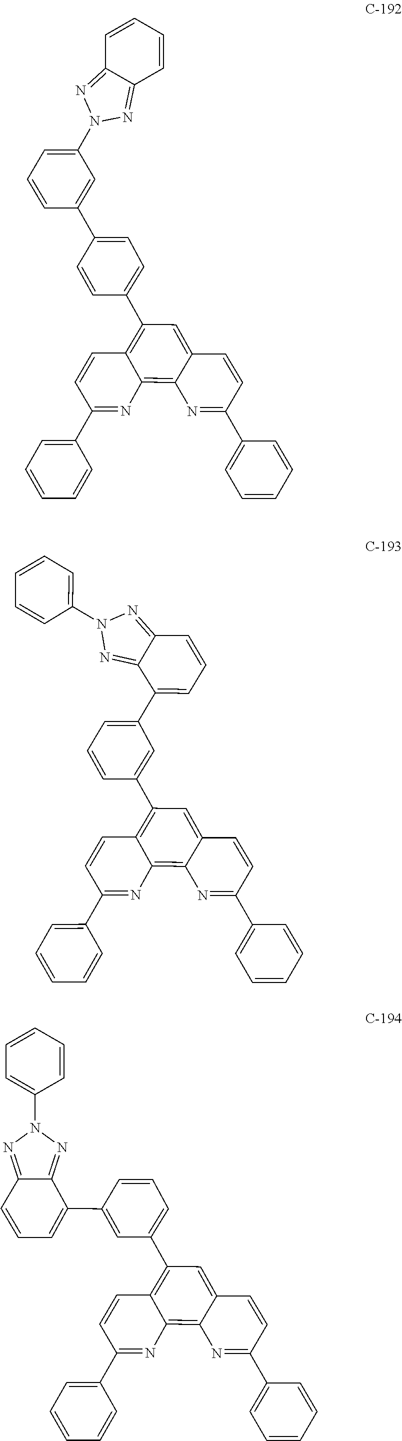 [SMILES: c1ccc(-c2ccc3cc(-c4ccc(-c5cccc(-n6nc7ccccc7n6)c5)cc4)c4ccc(-c5ccccc5)nc4c3n2)cc1 ; c1ccc(-c2ccc3cc(-c4cccc(-c5cccc6nn(-c7ccccc7)nc56)c4)c4ccc(-c5ccccc5)nc4c3n2)cc1 ; c1ccc(-c2ccc3cc(-c4cccc(-c5cccc6nn(-c7ccccc7)nc56)c4)c4ccc(-c5ccccc5)nc4c3n2)cc1]

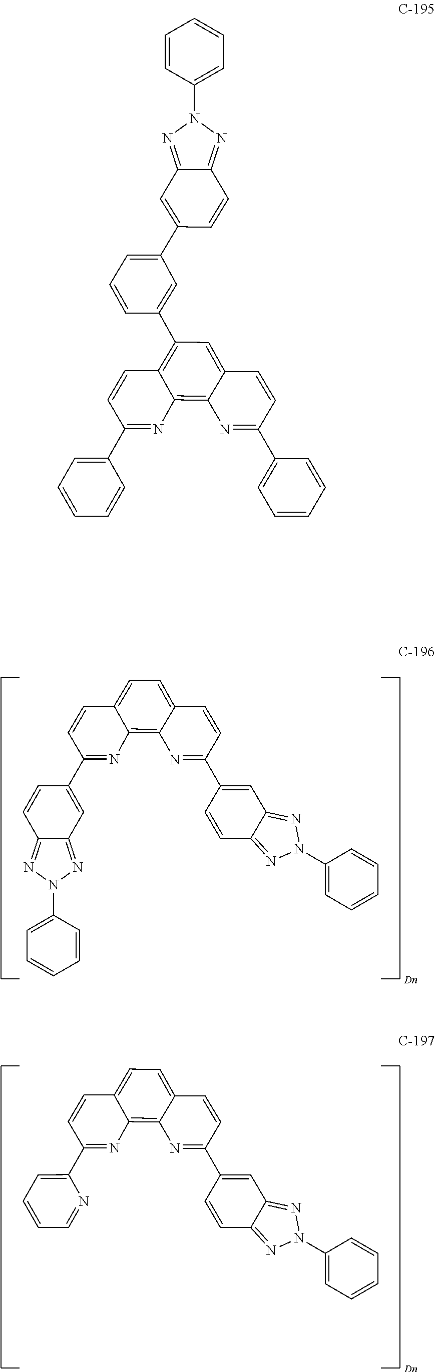

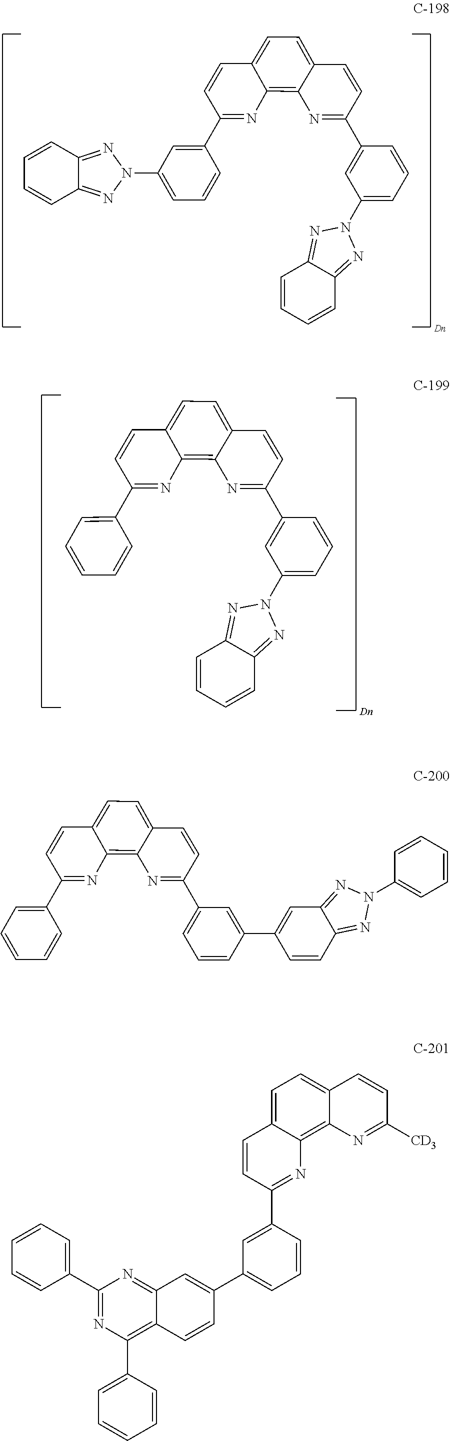

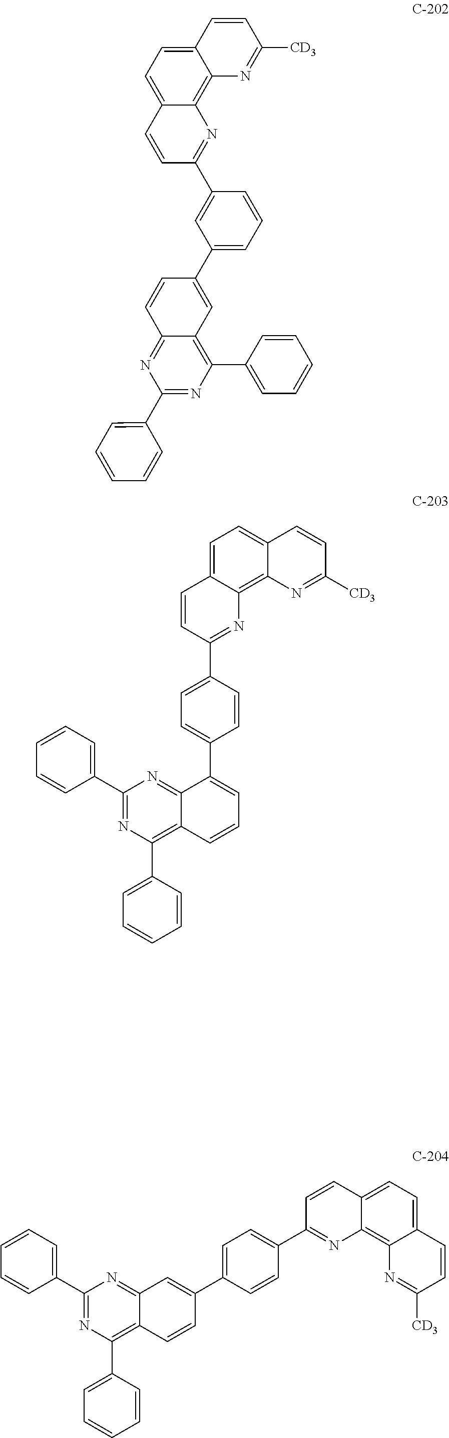 [SMILES: [2H]C([2H])([2H])c1ccc2ccc3ccc(-c4cccc(-c5ccc6nc(-c7ccccc7)nc(-c7ccccc7)c6c5)c4)nc3c2n1 ; [2H]C([2H])([2H])c1ccc2ccc3ccc(-c4ccc(-c5cccc6c(-c7ccccc7)nc(-c7ccccc7)nc56)cc4)nc3c2n1 ; [2H]C([2H])([2H])c1ccc2ccc3ccc(-c4ccc(-c5ccc6c(-c7ccccc7)nc(-c7ccccc7)nc6c5)cc4)nc3c2n1]

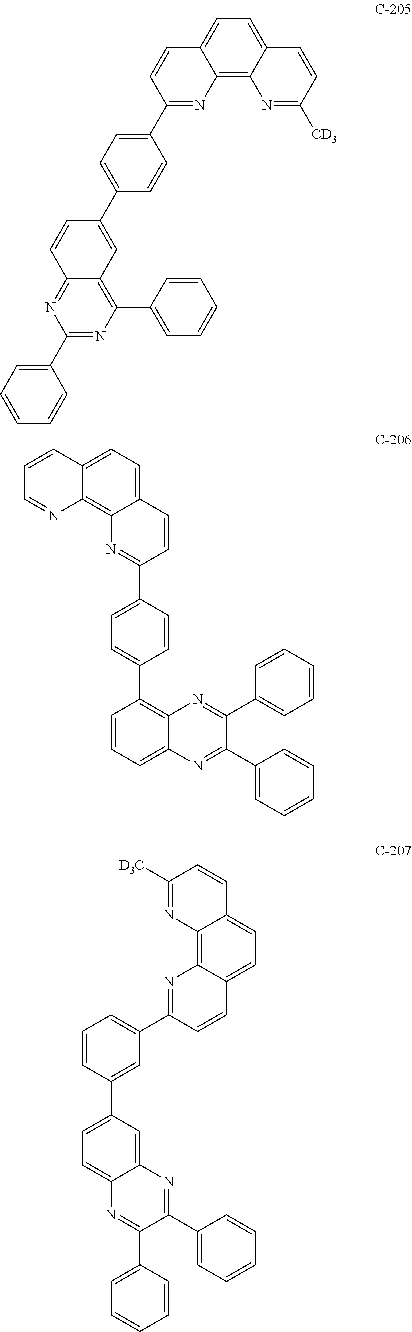

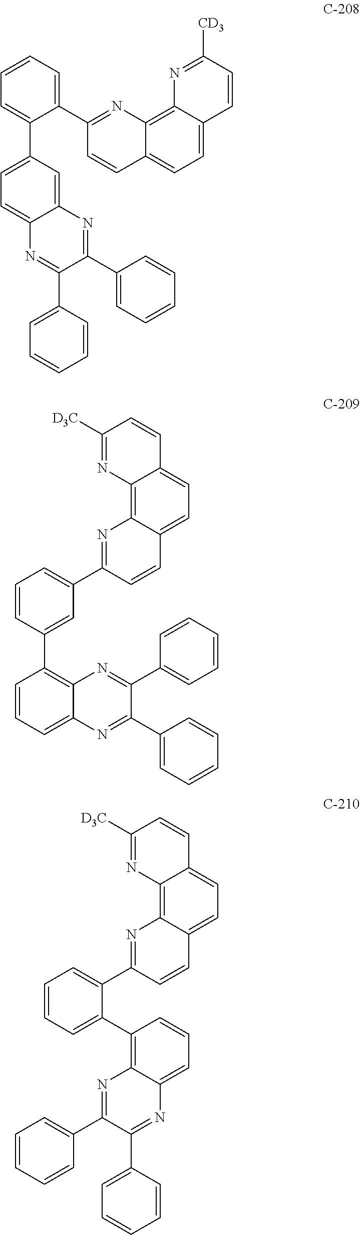

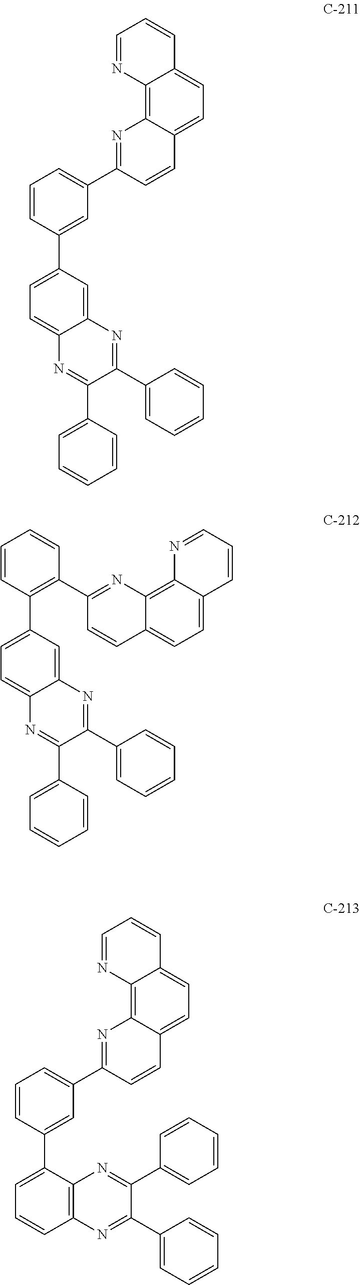 [SMILES: c1ccc(-c2nc3ccc(-c4cccc(-c5ccc6ccc7cccnc7c6n5)c4)cc3nc2-c2ccccc2)cc1 ; c1ccc(-c2nc3ccc(-c4ccccc4-c4ccc5ccc6cccnc6c5n4)cc3nc2-c2ccccc2)cc1 ; c1ccc(-c2nc3cccc(-c4cccc(-c5ccc6ccc7cccnc7c6n5)c4)c3nc2-c2ccccc2)cc1]

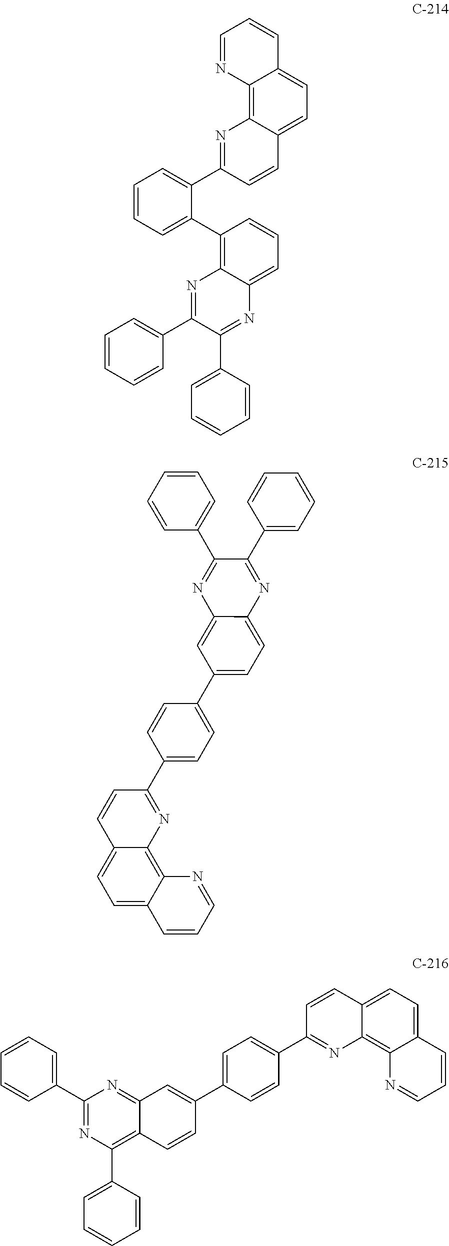

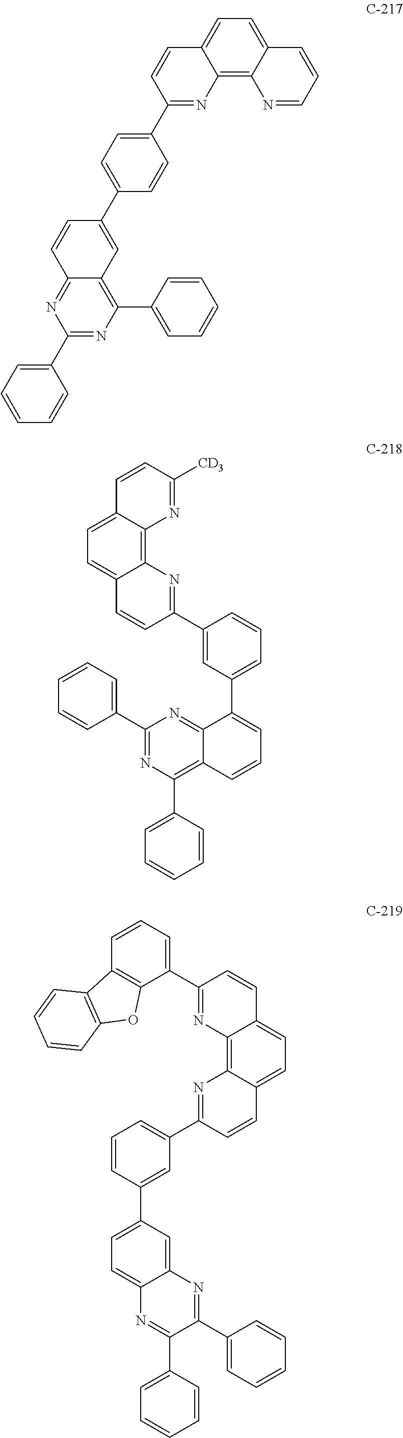 [SMILES: c1ccc(-c2nc(-c3ccccc3)c3cc(-c4ccc(-c5ccc6ccc7cccnc7c6n5)cc4)ccc3n2)cc1 ; [2H]C([2H])([2H])c1ccc2ccc3ccc(-c4cccc(-c5cccc6c(-c7ccccc7)nc(-c7ccccc7)nc56)c4)nc3c2n1 ; c1ccc(-c2nc3ccc(-c4cccc(-c5ccc6ccc7ccc(-c8cccc9c8oc8ccccc89)nc7c6n5)c4)cc3nc2-c2ccccc2)cc1]

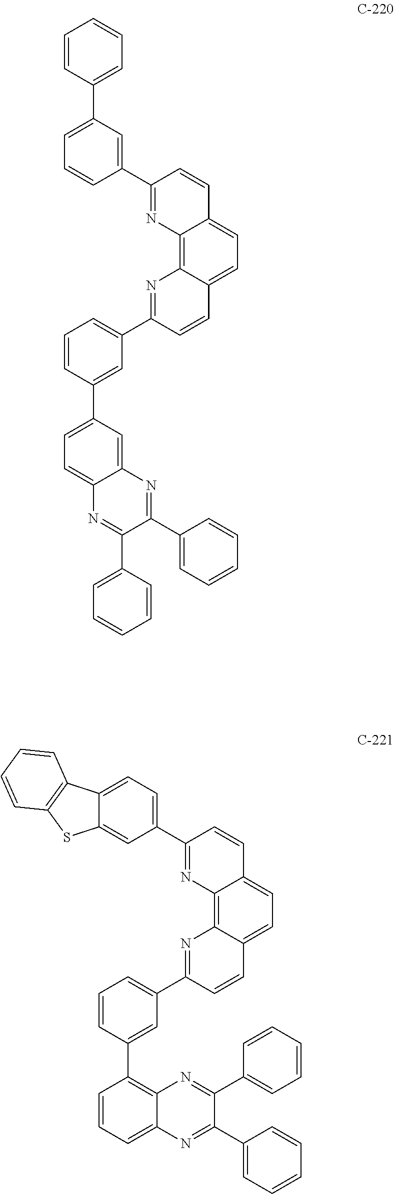 [SMILES: c1ccc(-c2cccc(-c3ccc4ccc5ccc(-c6cccc(-c7ccc8nc(-c9ccccc9)c(-c9ccccc9)nc8c7)c6)nc5c4n3)c2)cc1 ; c1ccc(-c2nc3cccc(-c4cccc(-c5ccc6ccc7ccc(-c8ccc9c(c8)sc8ccccc89)nc7c6n5)c4)c3nc2-c2ccccc2)cc1]

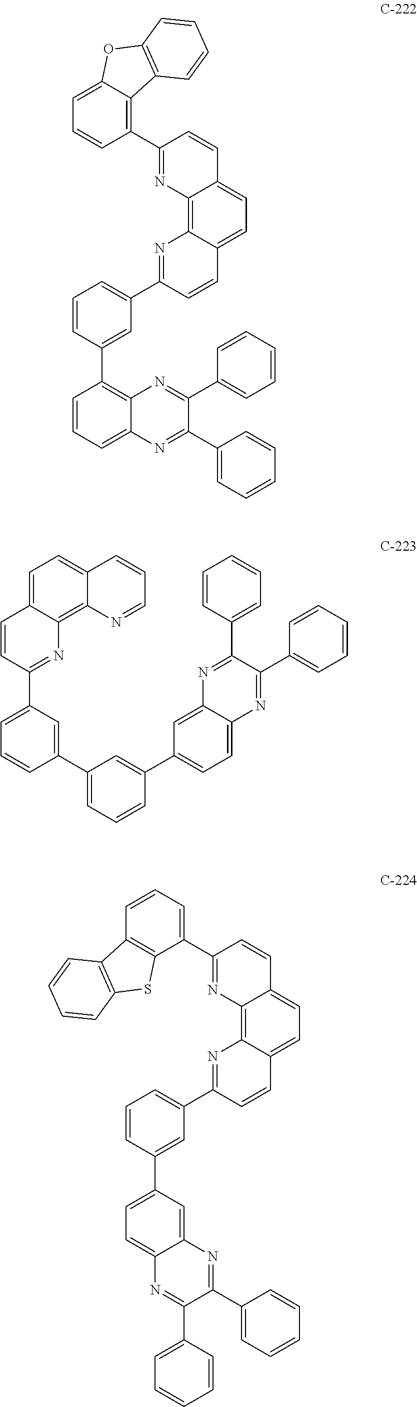 [SMILES: c1ccc(-c2nc3cccc(-c4cccc(-c5ccc6ccc7ccc(-c8cccc9oc%10ccccc%10c89)nc7c6n5)c4)c3nc2-c2ccccc2)cc1 ; c1ccc(-c2nc3ccc(-c4cccc(-c5cccc(-c6ccc7ccc8cccnc8c7n6)c5)c4)cc3nc2-c2ccccc2)cc1 ; c1ccc(-c2nc3ccc(-c4cccc(-c5ccc6ccc7ccc(-c8cccc9c8sc8ccccc89)nc7c6n5)c4)cc3nc2-c2ccccc2)cc1]

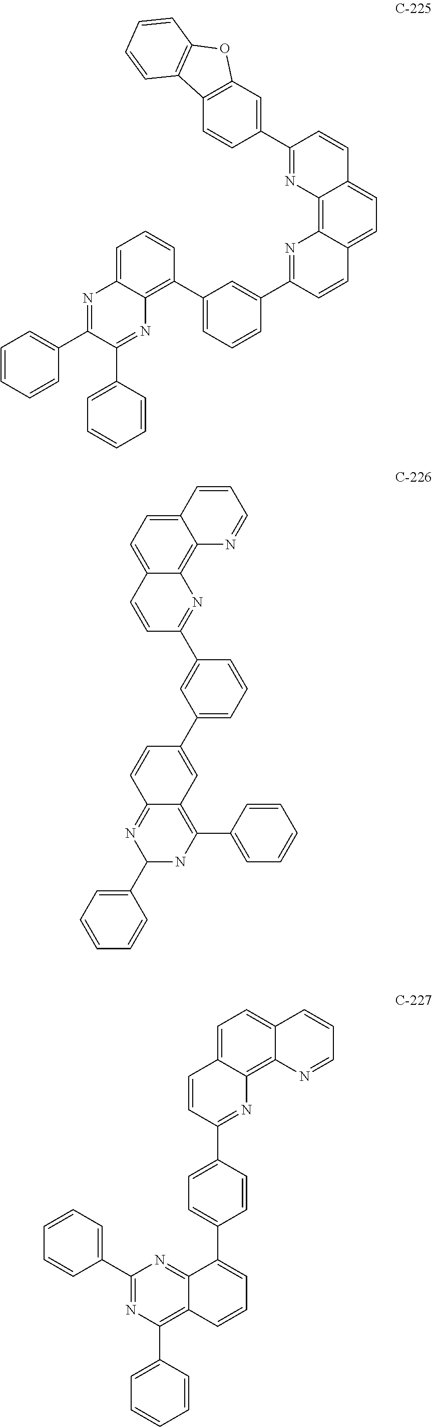 [SMILES: c1ccc(-c2nc3cccc(-c4cccc(-c5ccc6ccc7ccc(-c8ccc9c(c8)oc8ccccc89)nc7c6n5)c4)c3nc2-c2ccccc2)cc1 ; c1ccc(C2=c3cc(-c4cccc(-c5ccc6ccc7cccnc7c6n5)c4)ccc3=NC(c3ccccc3)N2)cc1 ; c1ccc(-c2nc(-c3ccccc3)c3cccc(-c4ccc(-c5ccc6ccc7cccnc7c6n5)cc4)c3n2)cc1]

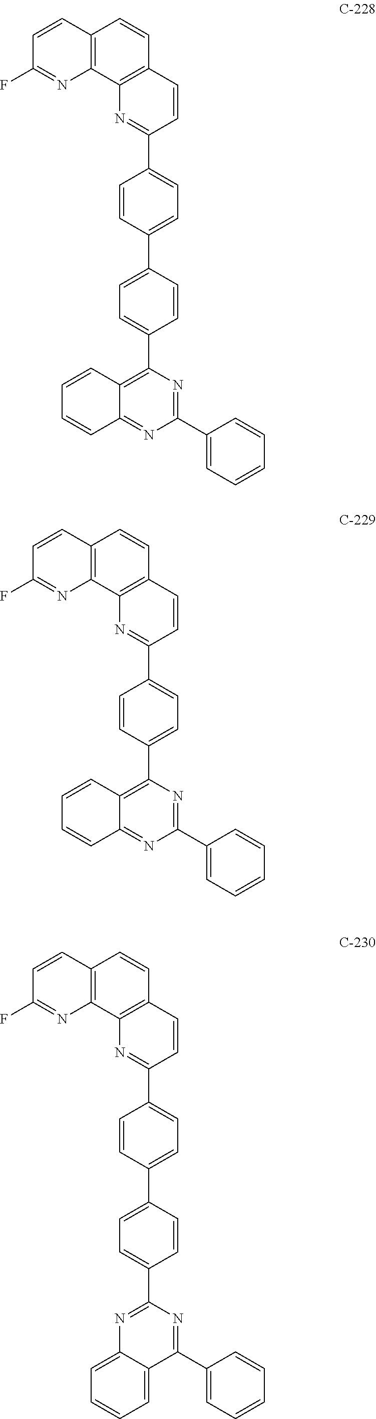 [SMILES: Fc1ccc2ccc3ccc(-c4ccc(-c5ccc(-c6nc(-c7ccccc7)nc7ccccc67)cc5)cc4)nc3c2n1 ; Fc1ccc2ccc3ccc(-c4ccc(-c5nc(-c6ccccc6)nc6ccccc56)cc4)nc3c2n1 ; Fc1ccc2ccc3ccc(-c4ccc(-c5ccc(-c6nc(-c7ccccc7)c7ccccc7n6)cc5)cc4)nc3c2n1]

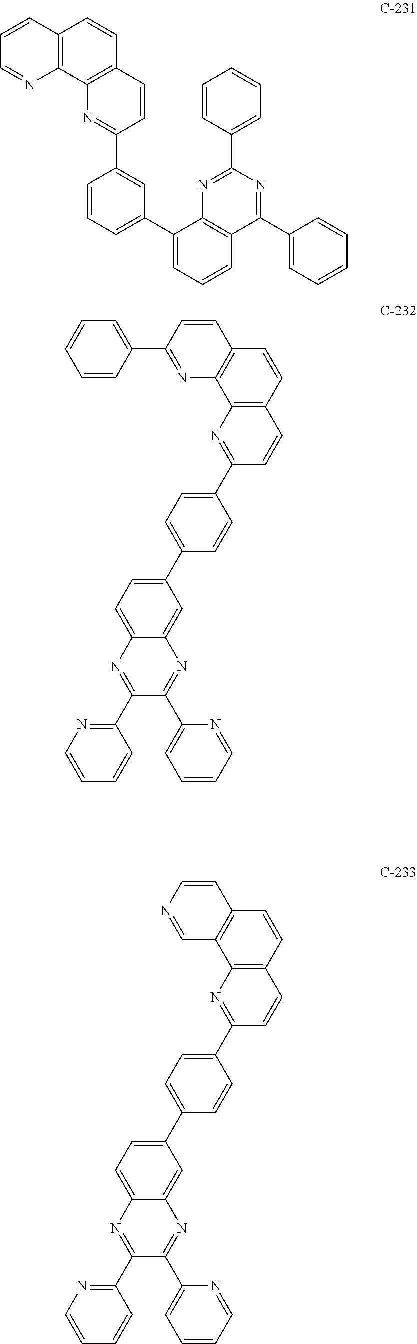

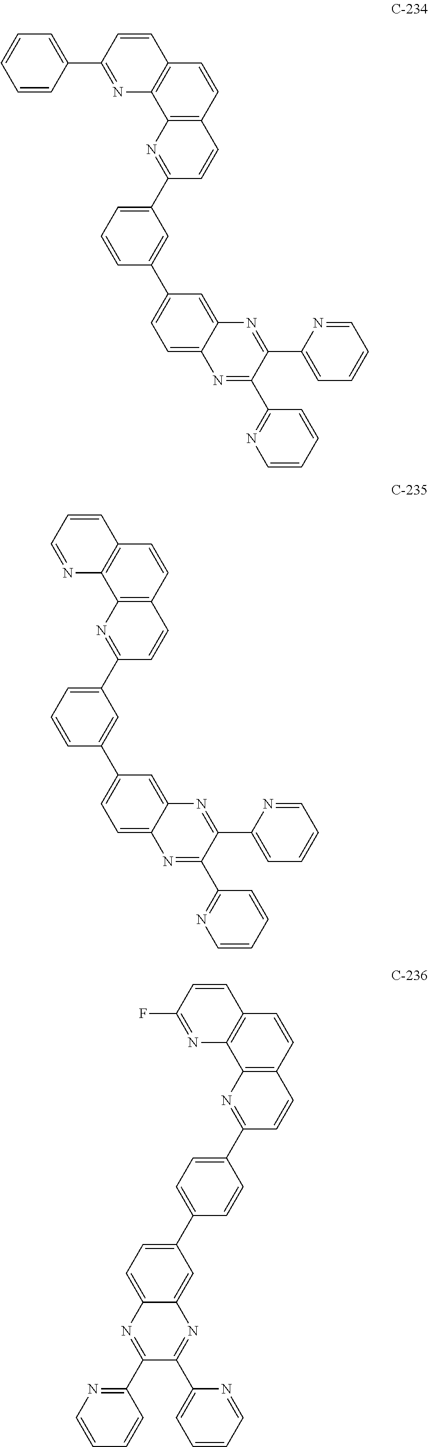

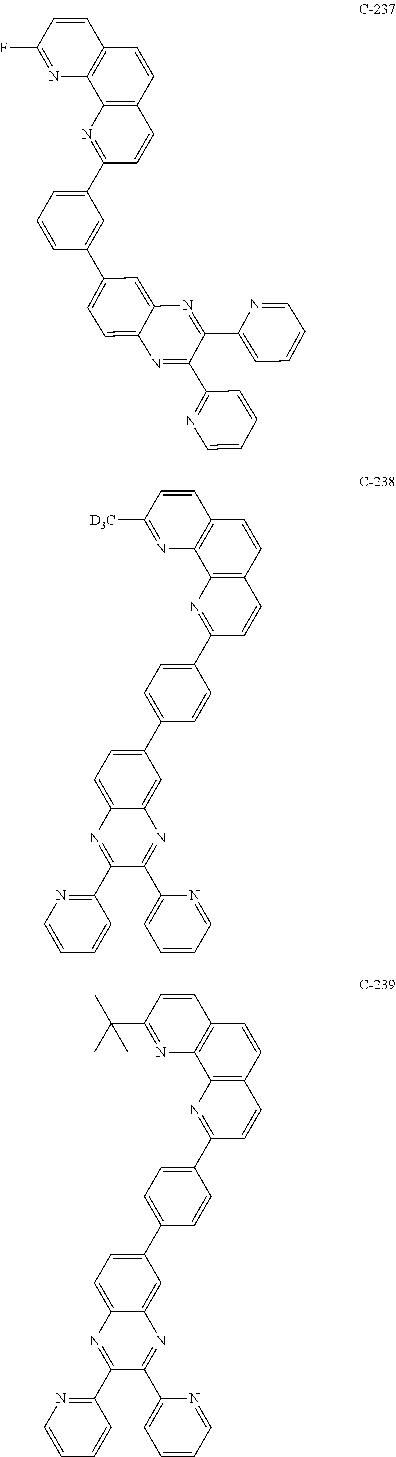

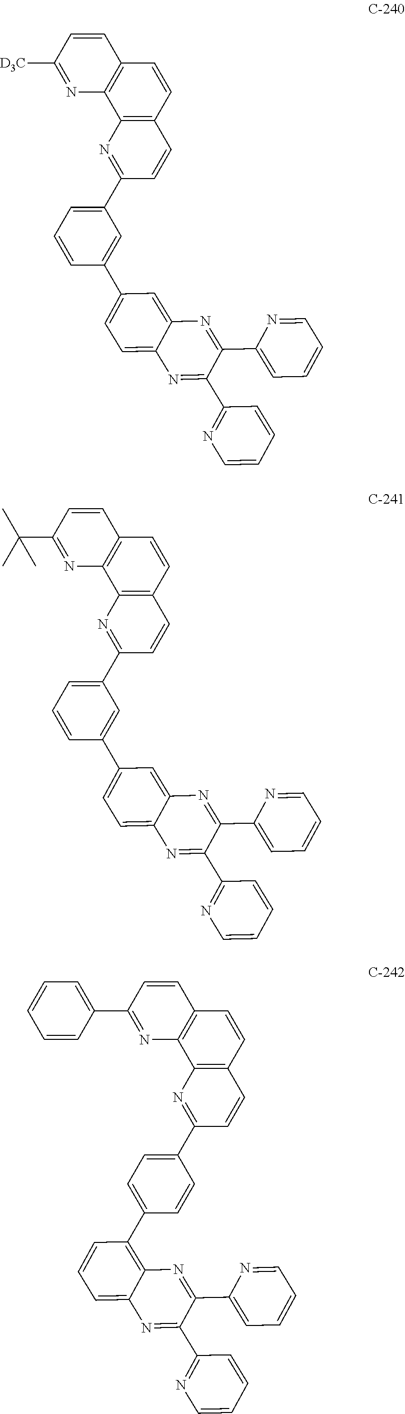 [SMILES: [2H]C([2H])([2H])c1ccc2ccc3ccc(-c4cccc(-c5ccc6nc(-c7ccccn7)c(-c7ccccn7)nc6c5)c4)nc3c2n1 ; CC(C)(C)c1ccc2ccc3ccc(-c4cccc(-c5ccc6nc(-c7ccccn7)c(-c7ccccn7)nc6c5)c4)nc3c2n1 ; c1ccc(-c2ccc3ccc4ccc(-c5ccc(-c6cccc7nc(-c8ccccn8)c(-c8ccccn8)nc67)cc5)nc4c3n2)cc1]

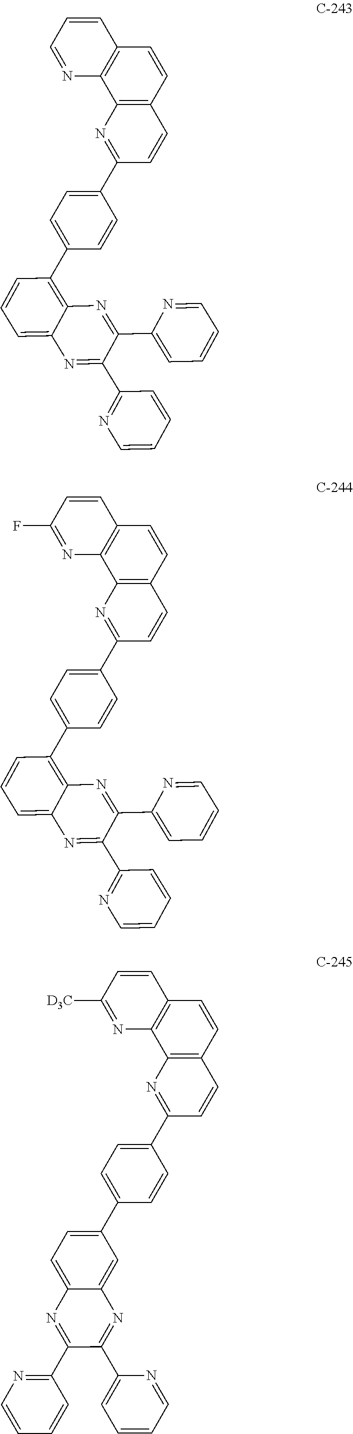

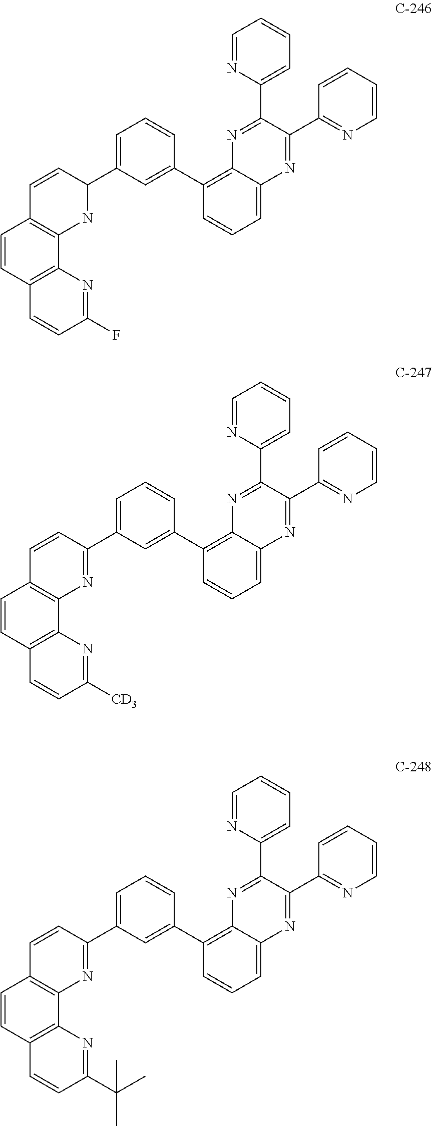 [SMILES: Fc1ccc2ccc3c(c2n1)NC(c1cccc(-c2cccc4nc(-c5ccccn5)c(-c5ccccn5)nc24)c1)C=C3 ; [2H]C([2H])([2H])c1ccc2ccc3ccc(-c4cccc(-c5cccc6nc(-c7ccccn7)c(-c7ccccn7)nc56)c4)nc3c2n1 ; CC(C)(C)c1ccc2ccc3ccc(-c4cccc(-c5cccc6nc(-c7ccccn7)c(-c7ccccn7)nc56)c4)nc3c2n1]

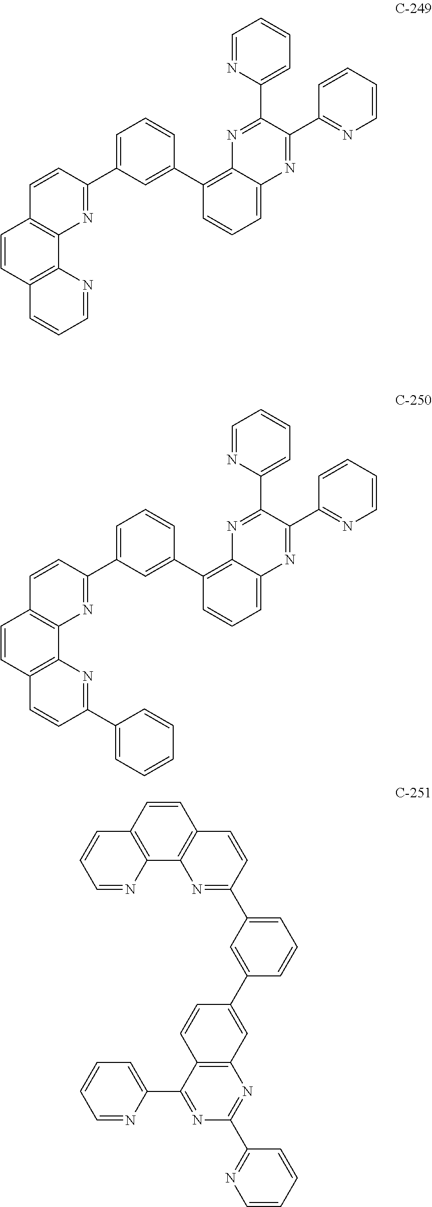

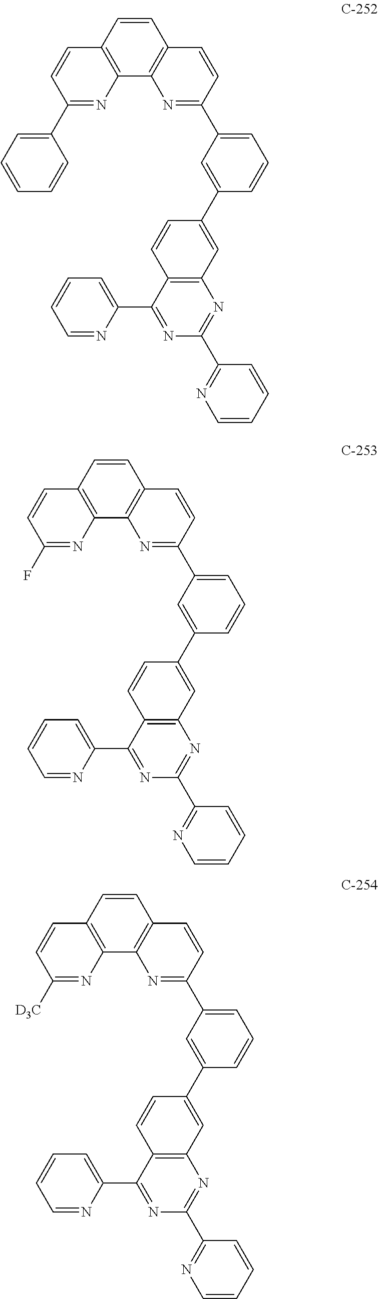 [SMILES: c1ccc(-c2ccc3ccc4ccc(-c5cccc(-c6ccc7c(-c8ccccn8)nc(-c8ccccn8)nc7c6)c5)nc4c3n2)cc1 ; Fc1ccc2ccc3ccc(-c4cccc(-c5ccc6c(-c7ccccn7)nc(-c7ccccn7)nc6c5)c4)nc3c2n1 ; [2H]C([2H])([2H])c1ccc2ccc3ccc(-c4cccc(-c5ccc6c(-c7ccccn7)nc(-c7ccccn7)nc6c5)c4)nc3c2n1]

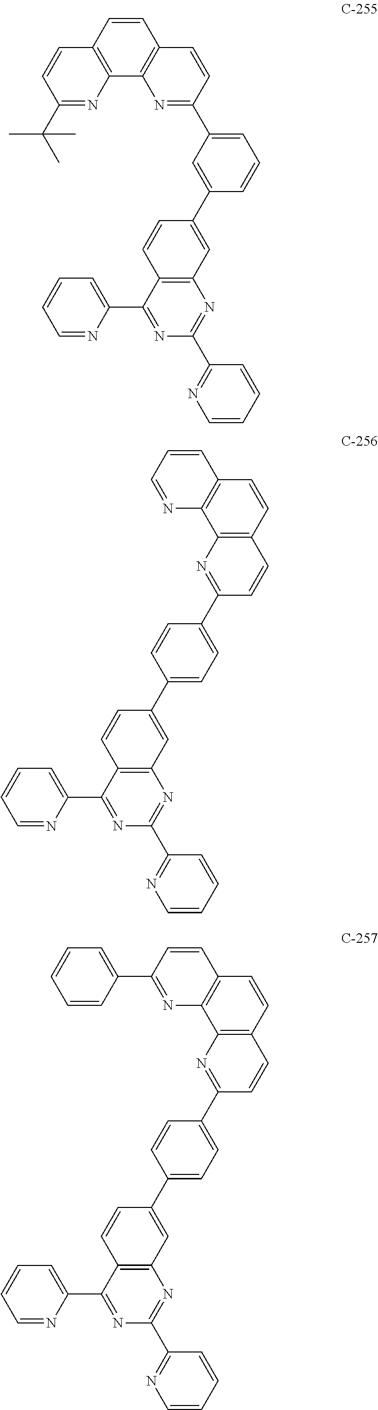

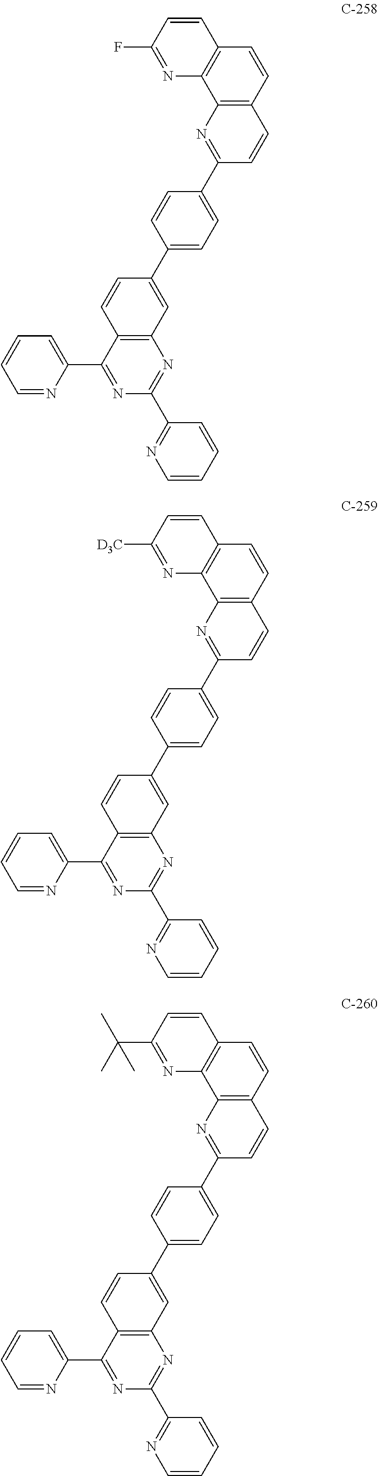

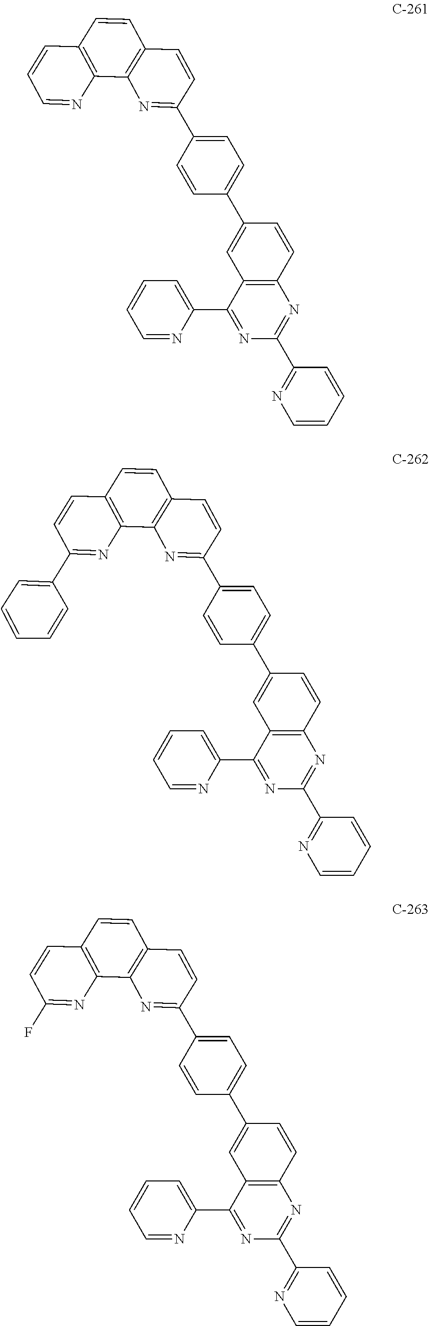 [SMILES: c1ccc(-c2nc(-c3ccccn3)c3cc(-c4ccc(-c5ccc6ccc7cccnc7c6n5)cc4)ccc3n2)nc1 ; c1ccc(-c2ccc3ccc4ccc(-c5ccc(-c6ccc7nc(-c8ccccn8)nc(-c8ccccn8)c7c6)cc5)nc4c3n2)cc1 ; Fc1ccc2ccc3ccc(-c4ccc(-c5ccc6nc(-c7ccccn7)nc(-c7ccccn7)c6c5)cc4)nc3c2n1]

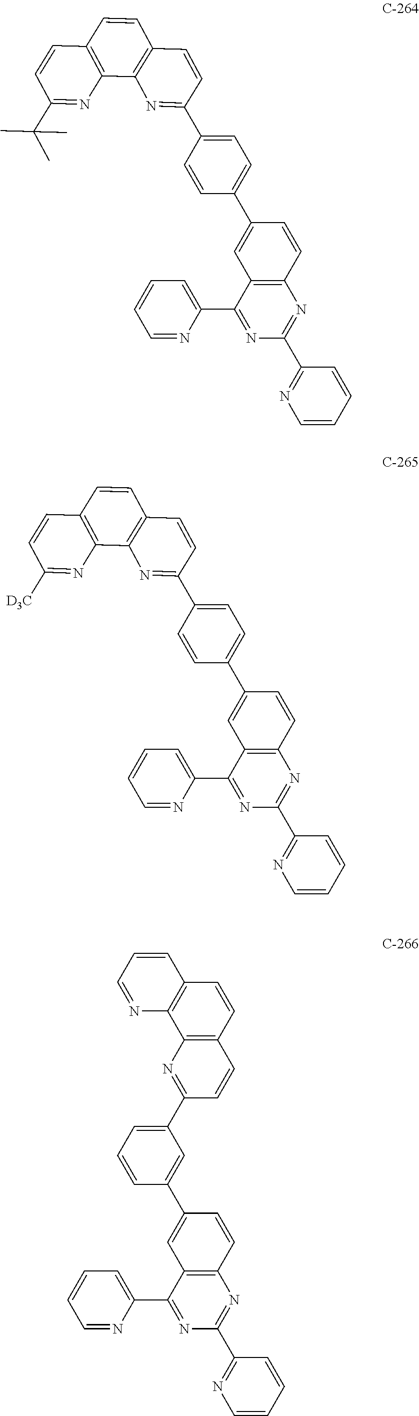 [SMILES: CC(C)(C)c1ccc2ccc3ccc(-c4ccc(-c5ccc6nc(-c7ccccn7)nc(-c7ccccn7)c6c5)cc4)nc3c2n1 ; [2H]C([2H])([2H])c1ccc2ccc3ccc(-c4ccc(-c5ccc6nc(-c7ccccn7)nc(-c7ccccn7)c6c5)cc4)nc3c2n1 ; c1ccc(-c2nc(-c3ccccn3)c3cc(-c4cccc(-c5ccc6ccc7cccnc7c6n5)c4)ccc3n2)nc1]

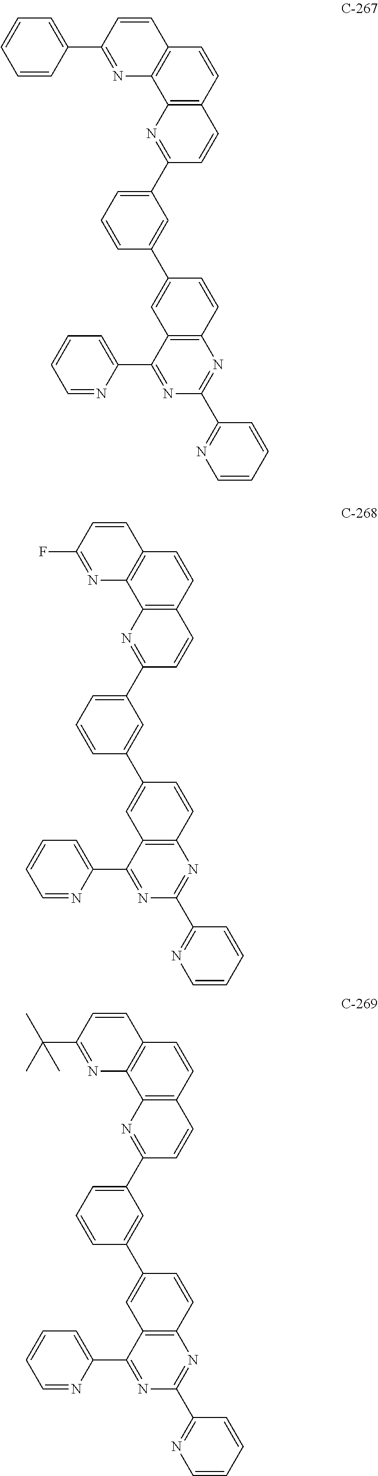 [SMILES: c1ccc(-c2ccc3ccc4ccc(-c5cccc(-c6ccc7nc(-c8ccccn8)nc(-c8ccccn8)c7c6)c5)nc4c3n2)cc1 ; Fc1ccc2ccc3ccc(-c4cccc(-c5ccc6nc(-c7ccccn7)nc(-c7ccccn7)c6c5)c4)nc3c2n1 ; CC(C)(C)c1ccc2ccc3ccc(-c4cccc(-c5ccc6nc(-c7ccccn7)nc(-c7ccccn7)c6c5)c4)nc3c2n1]

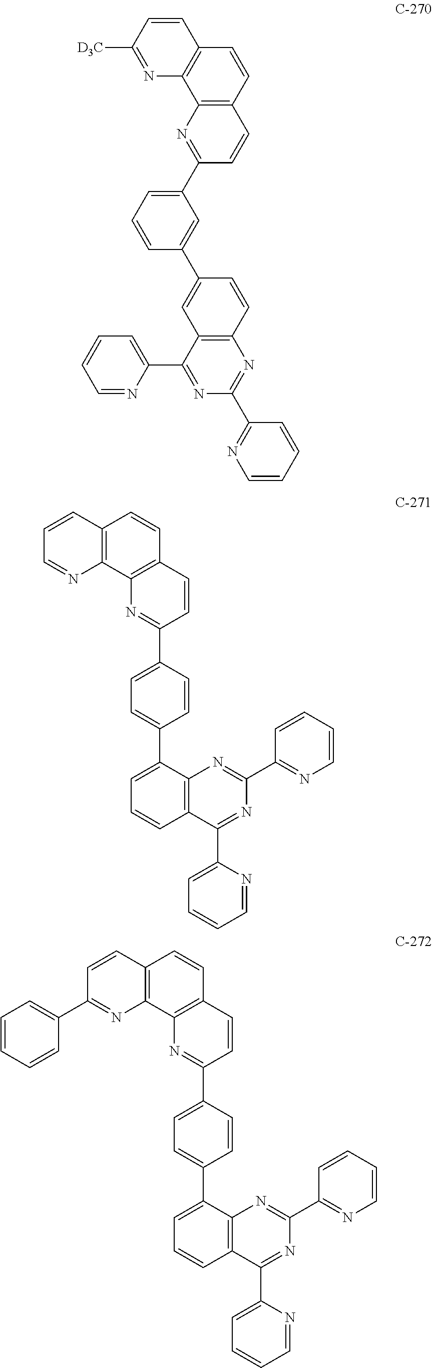

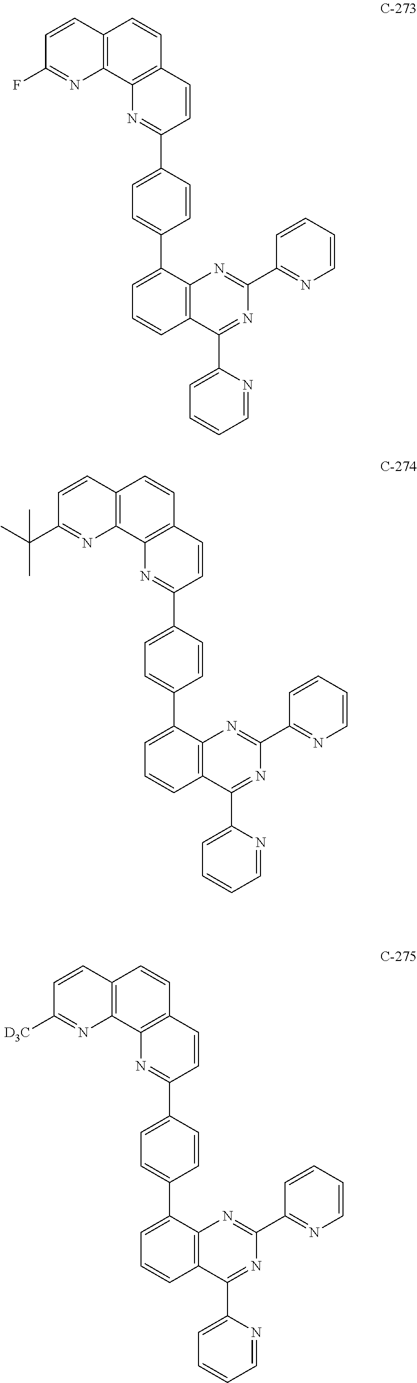 [SMILES: Fc1ccc2ccc3ccc(-c4ccc(-c5cccc6c(-c7ccccn7)nc(-c7ccccn7)nc56)cc4)nc3c2n1 ; CC(C)(C)c1ccc2ccc3ccc(-c4ccc(-c5cccc6c(-c7ccccn7)nc(-c7ccccn7)nc56)cc4)nc3c2n1 ; [2H]C([2H])([2H])c1ccc2ccc3ccc(-c4ccc(-c5cccc6c(-c7ccccn7)nc(-c7ccccn7)nc56)cc4)nc3c2n1]

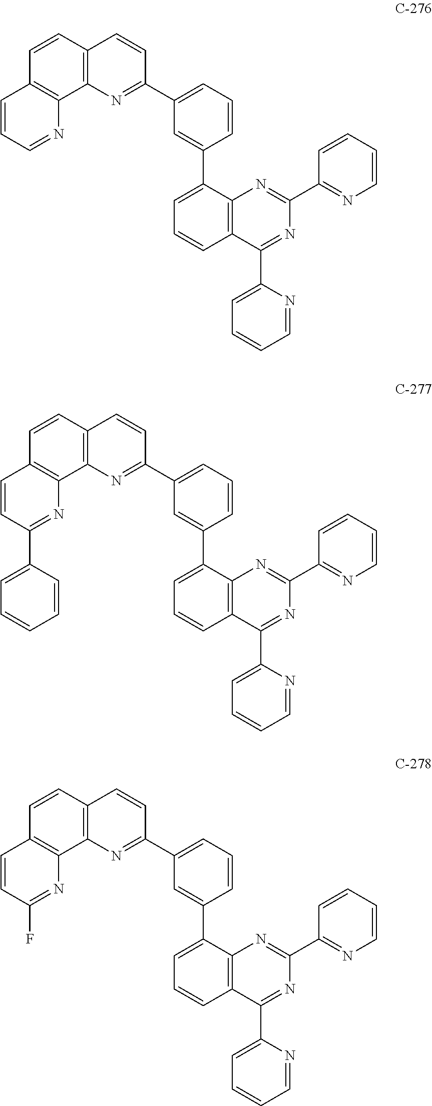 [SMILES: c1ccc(-c2nc(-c3ccccn3)c3cccc(-c4cccc(-c5ccc6ccc7cccnc7c6n5)c4)c3n2)nc1 ; c1ccc(-c2ccc3ccc4ccc(-c5cccc(-c6cccc7c(-c8ccccn8)nc(-c8ccccn8)nc67)c5)nc4c3n2)cc1 ; Fc1ccc2ccc3ccc(-c4cccc(-c5cccc6c(-c7ccccn7)nc(-c7ccccn7)nc56)c4)nc3c2n1]

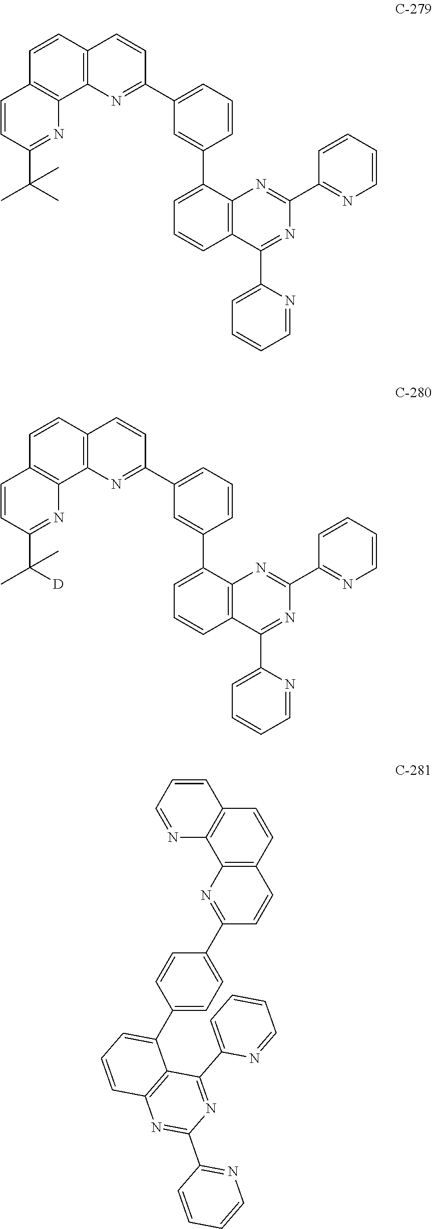 [SMILES: CC(C)(C)c1ccc2ccc3ccc(-c4cccc(-c5cccc6c(-c7ccccn7)nc(-c7ccccn7)nc56)c4)nc3c2n1 ; [2H]C(C)(C)c1ccc2ccc3ccc(-c4cccc(-c5cccc6c(-c7ccccn7)nc(-c7ccccn7)nc56)c4)nc3c2n1 ; c1ccc(-c2nc(-c3ccccn3)c3c(-c4ccc(-c5ccc6ccc7cccnc7c6n5)cc4)cccc3n2)nc1]

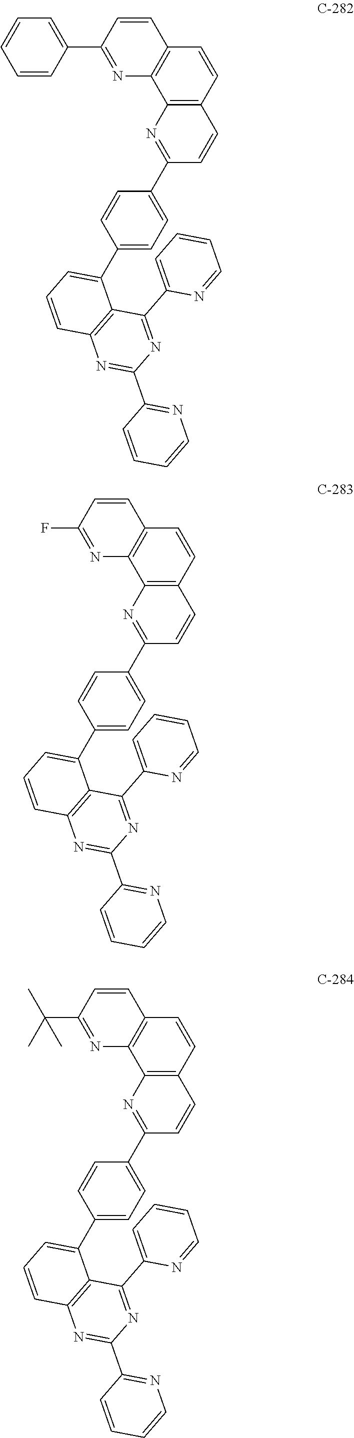

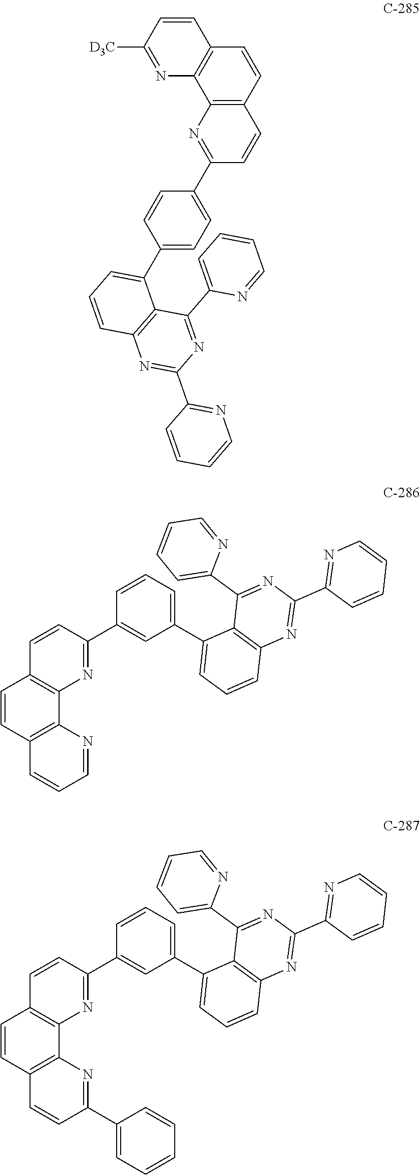 [SMILES: [2H]C([2H])([2H])c1ccc2ccc3ccc(-c4ccc(-c5cccc6nc(-c7ccccn7)nc(-c7ccccn7)c56)cc4)nc3c2n1 ; c1ccc(-c2nc(-c3ccccn3)c3c(-c4cccc(-c5ccc6ccc7cccnc7c6n5)c4)cccc3n2)nc1 ; c1ccc(-c2ccc3ccc4ccc(-c5cccc(-c6cccc7nc(-c8ccccn8)nc(-c8ccccn8)c67)c5)nc4c3n2)cc1]

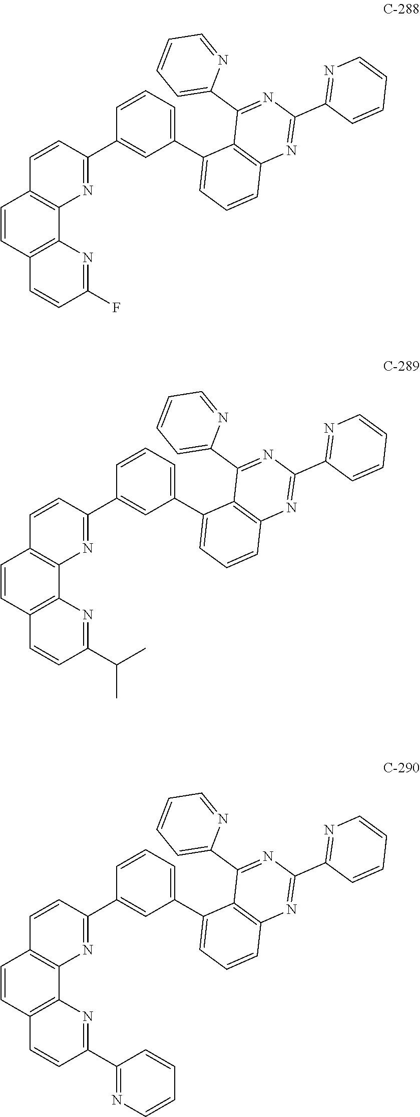 [SMILES: Fc1ccc2ccc3ccc(-c4cccc(-c5cccc6nc(-c7ccccn7)nc(-c7ccccn7)c56)c4)nc3c2n1 ; CC(C)c1ccc2ccc3ccc(-c4cccc(-c5cccc6nc(-c7ccccn7)nc(-c7ccccn7)c56)c4)nc3c2n1 ; c1ccc(-c2ccc3ccc4ccc(-c5cccc(-c6cccc7nc(-c8ccccn8)nc(-c8ccccn8)c67)c5)nc4c3n2)nc1]

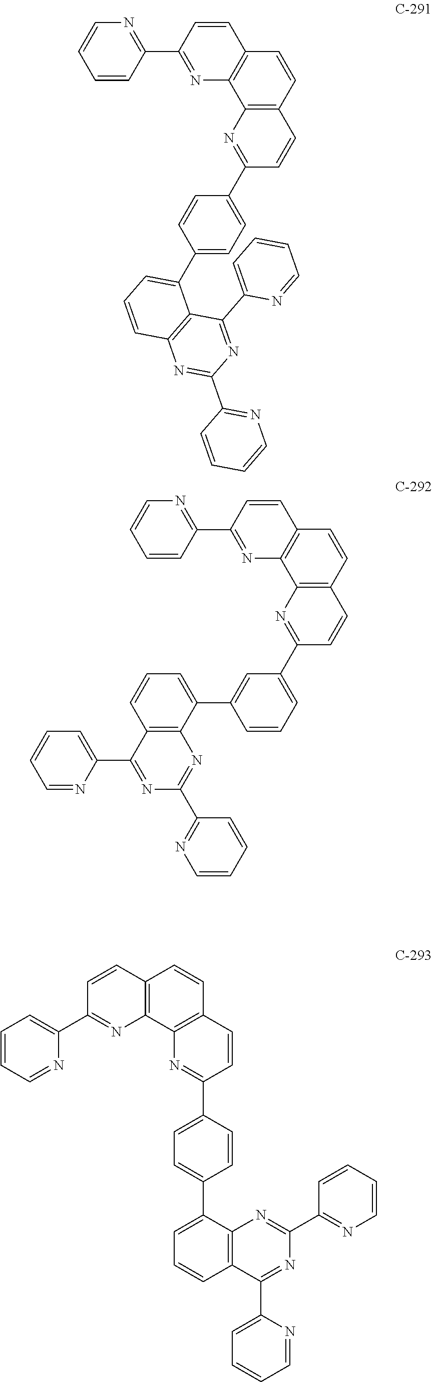

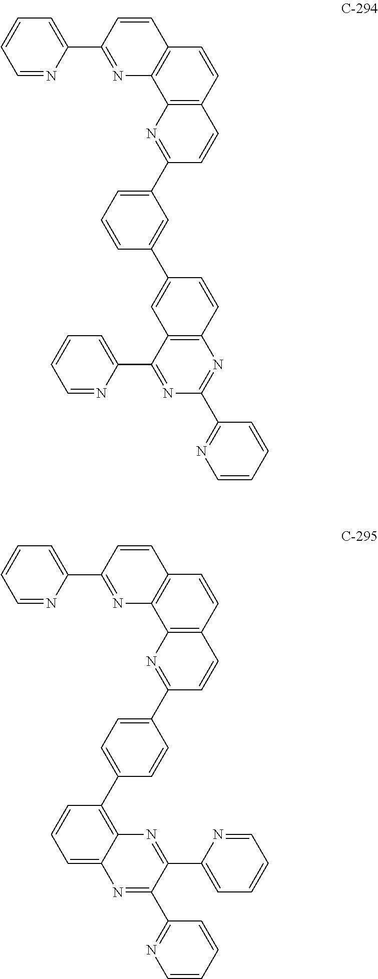 [SMILES: c1ccc(-c2ccc3ccc4ccc(-c5cccc(-c6ccc7nc(-c8ccccn8)nc(-c8ccccn8)c7c6)c5)nc4c3n2)nc1 ; c1ccc(-c2ccc3ccc4ccc(-c5ccc(-c6cccc7nc(-c8ccccn8)c(-c8ccccn8)nc67)cc5)nc4c3n2)nc1]

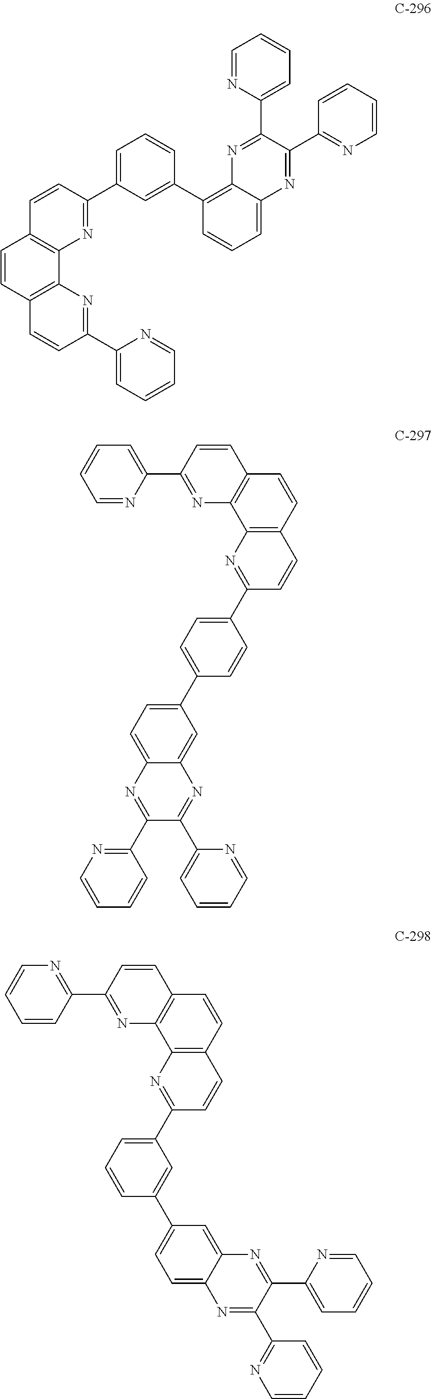 [SMILES: c1ccc(-c2ccc3ccc4ccc(-c5cccc(-c6cccc7nc(-c8ccccn8)c(-c8ccccn8)nc67)c5)nc4c3n2)nc1 ; c1ccc(-c2ccc3ccc4ccc(-c5ccc(-c6ccc7nc(-c8ccccn8)c(-c8ccccn8)nc7c6)cc5)nc4c3n2)nc1 ; c1ccc(-c2ccc3ccc4ccc(-c5cccc(-c6ccc7nc(-c8ccccn8)c(-c8ccccn8)nc7c6)c5)nc4c3n2)nc1]

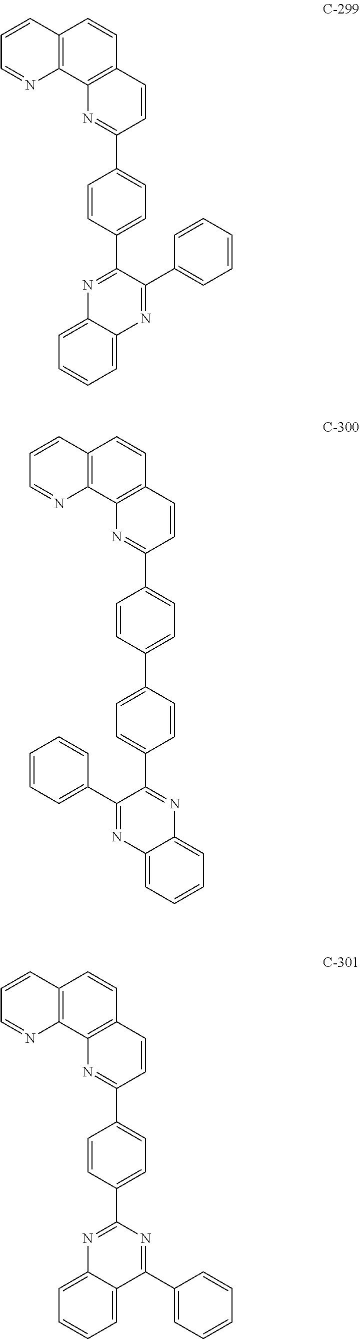 [SMILES: c1ccc(-c2nc3ccccc3nc2-c2ccc(-c3ccc4ccc5cccnc5c4n3)cc2)cc1 ; c1ccc(-c2nc3ccccc3nc2-c2ccc(-c3ccc(-c4ccc5ccc6cccnc6c5n4)cc3)cc2)cc1 ; c1ccc(-c2nc(-c3ccc(-c4ccc5ccc6cccnc6c5n4)cc3)nc3ccccc23)cc1]

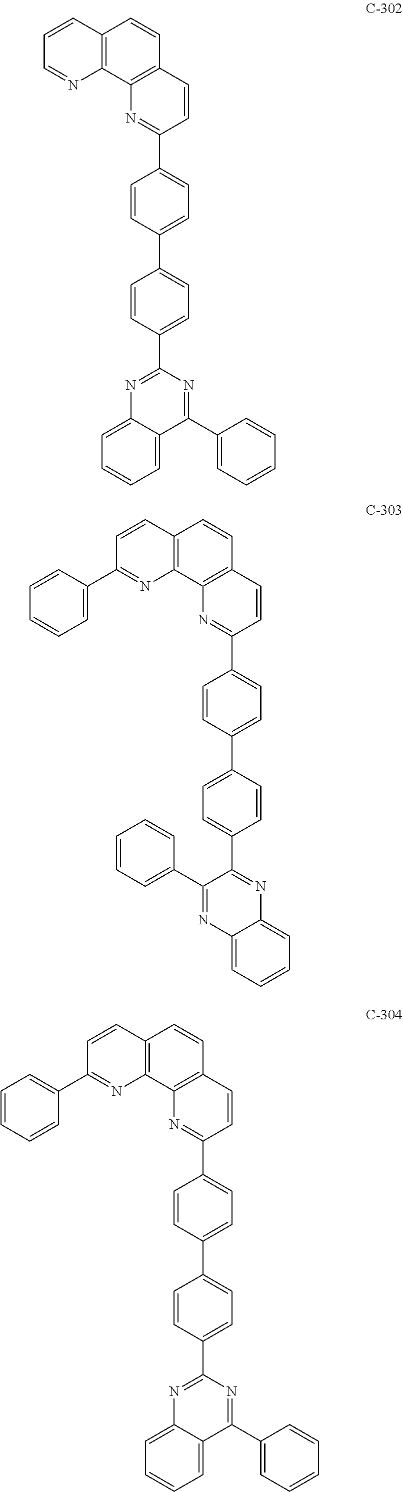

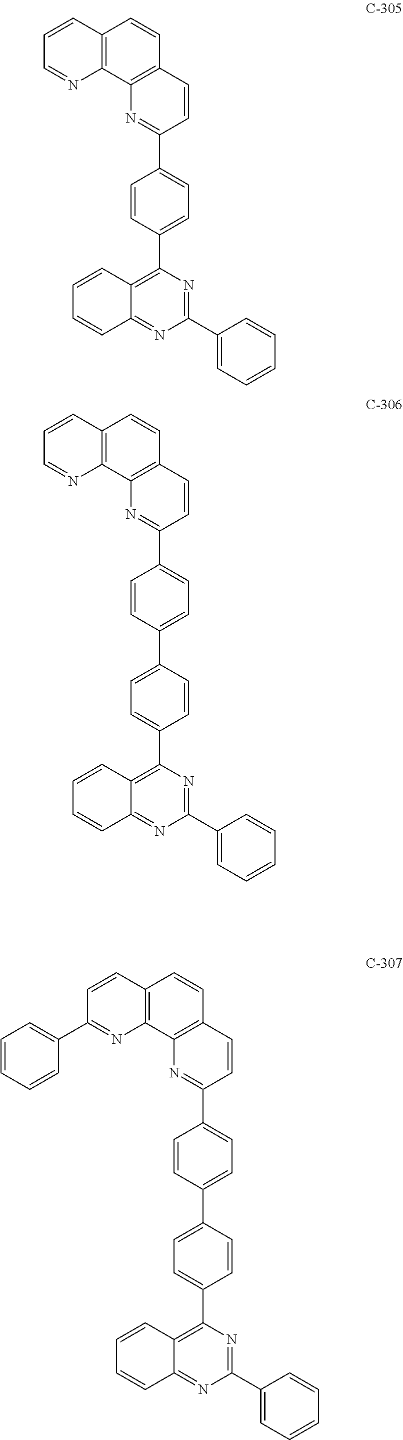 [SMILES: c1ccc(-c2nc(-c3ccc(-c4ccc5ccc6cccnc6c5n4)cc3)c3ccccc3n2)cc1 ; c1ccc(-c2nc(-c3ccc(-c4ccc(-c5ccc6ccc7cccnc7c6n5)cc4)cc3)c3ccccc3n2)cc1 ; c1ccc(-c2ccc3ccc4ccc(-c5ccc(-c6ccc(-c7nc(-c8ccccc8)nc8ccccc78)cc6)cc5)nc4c3n2)cc1]

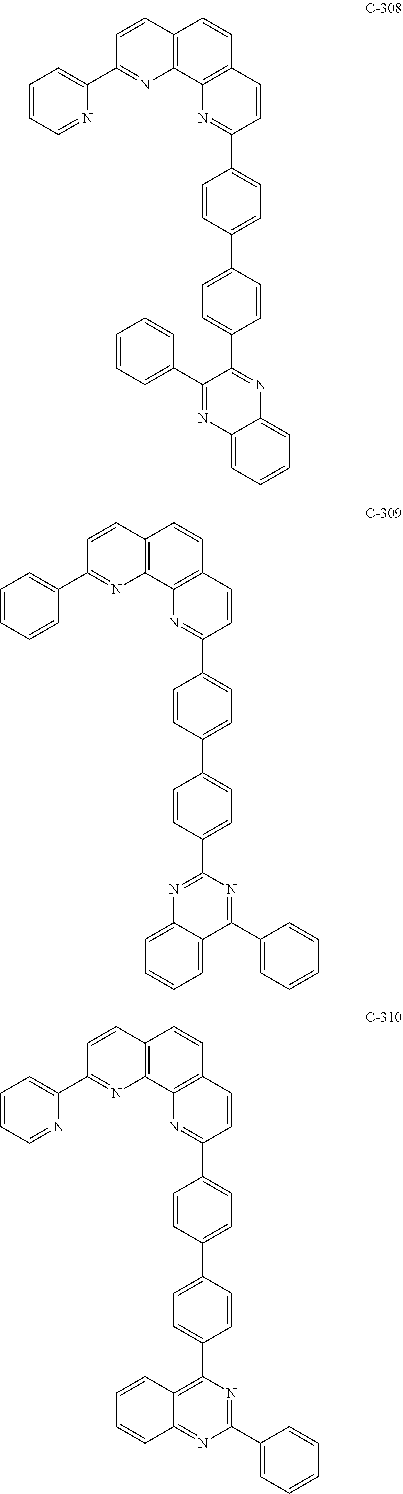

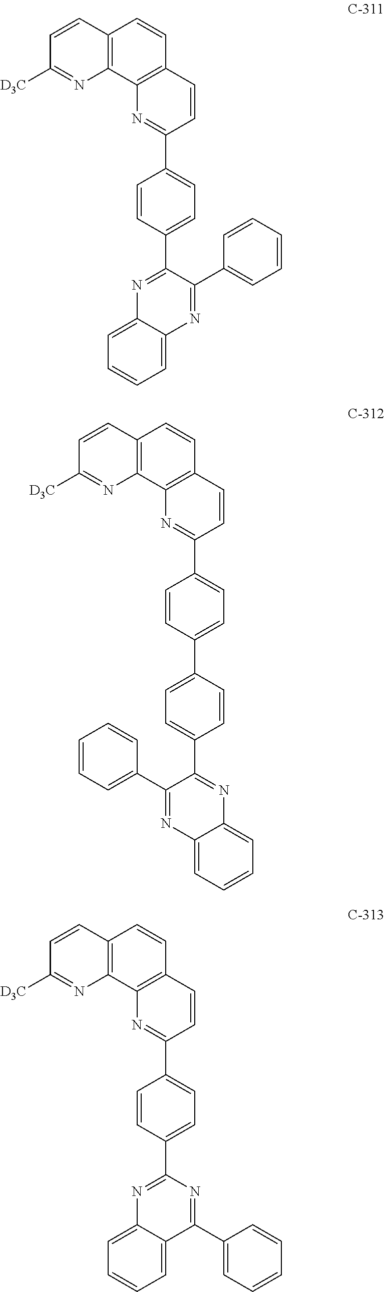 [SMILES: [2H]C([2H])([2H])c1ccc2ccc3ccc(-c4ccc(-c5nc6ccccc6nc5-c5ccccc5)cc4)nc3c2n1 ; [2H]C([2H])([2H])c1ccc2ccc3ccc(-c4ccc(-c5ccc(-c6nc7ccccc7nc6-c6ccccc6)cc5)cc4)nc3c2n1 ; [2H]C([2H])([2H])c1ccc2ccc3ccc(-c4ccc(-c5nc(-c6ccccc6)c6ccccc6n5)cc4)nc3c2n1]

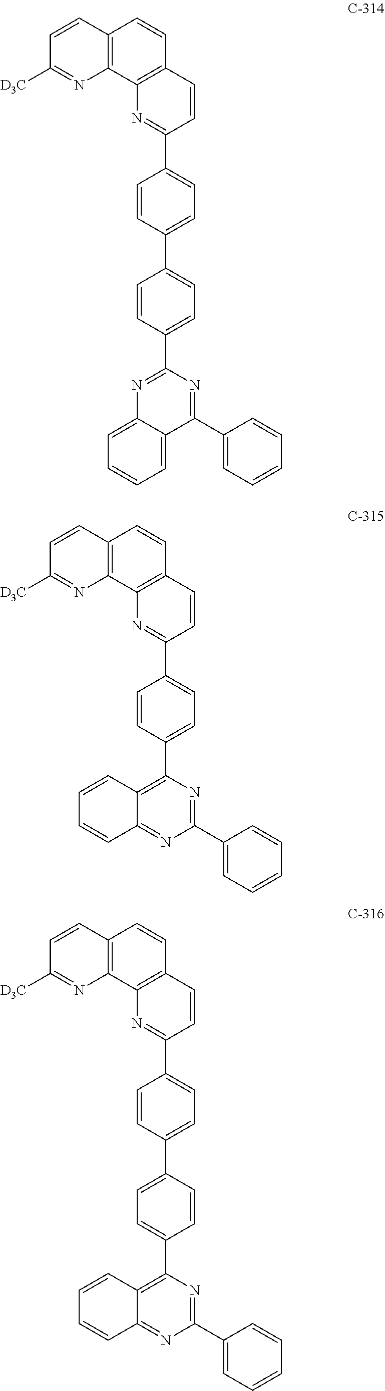

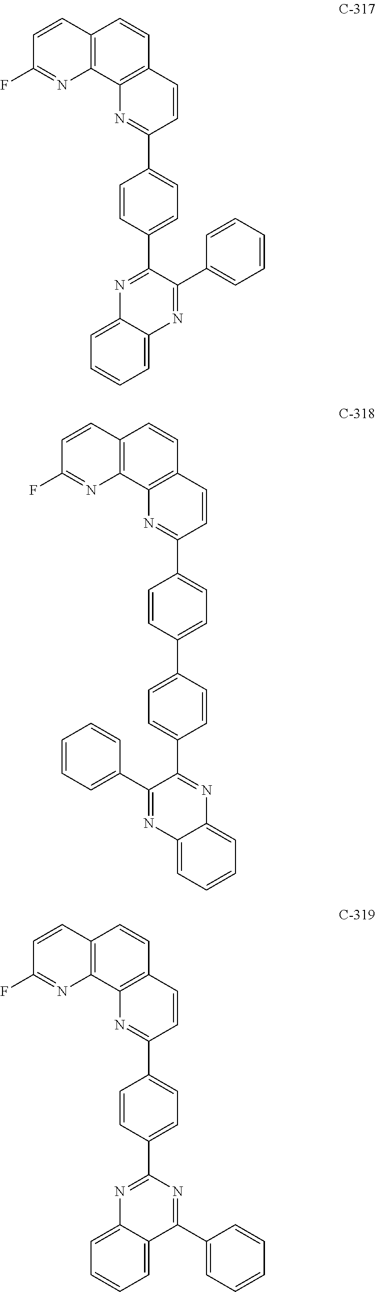 [SMILES: Fc1ccc2ccc3ccc(-c4ccc(-c5nc6ccccc6nc5-c5ccccc5)cc4)nc3c2n1 ; Fc1ccc2ccc3ccc(-c4ccc(-c5ccc(-c6nc7ccccc7nc6-c6ccccc6)cc5)cc4)nc3c2n1 ; Fc1ccc2ccc3ccc(-c4ccc(-c5nc(-c6ccccc6)c6ccccc6n5)cc4)nc3c2n1]

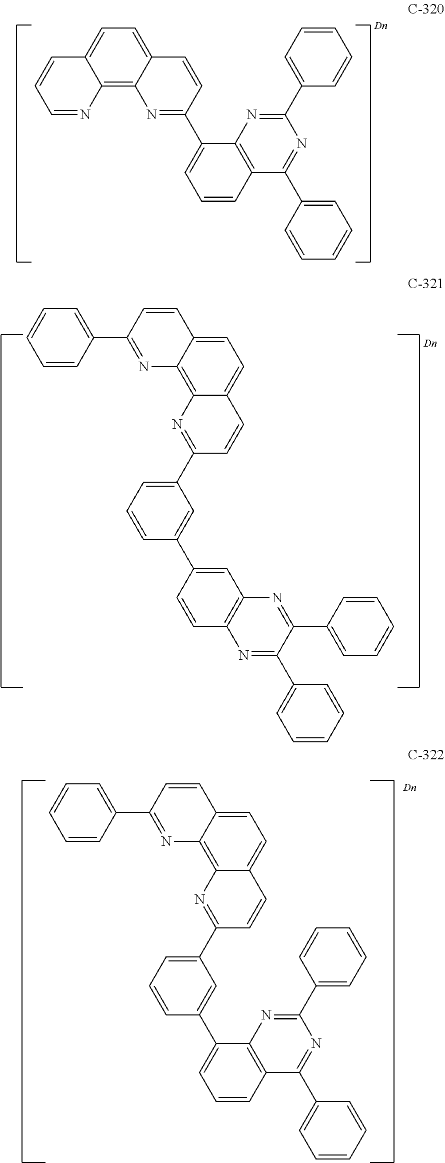

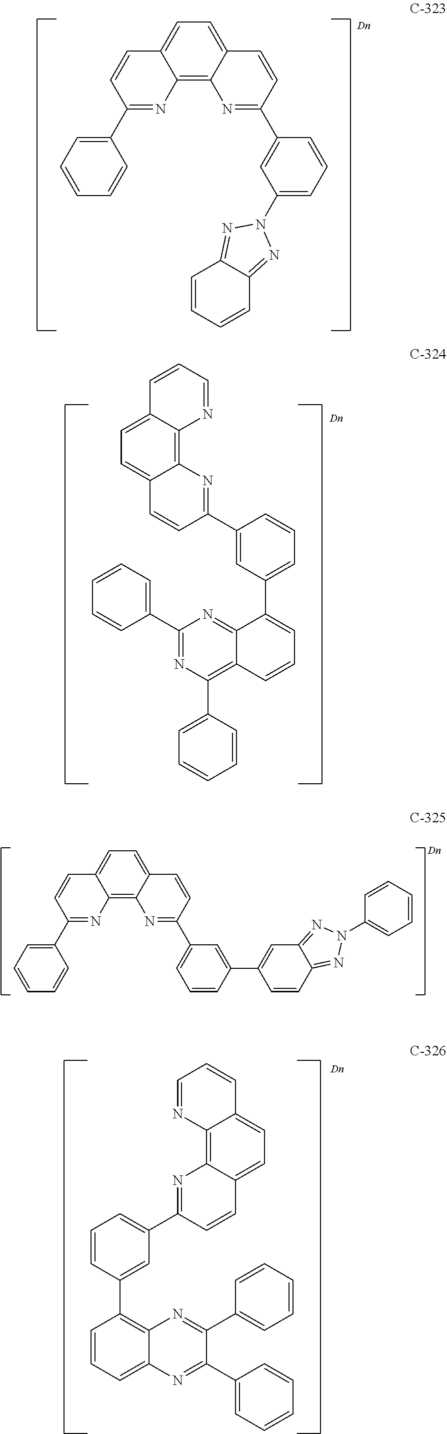

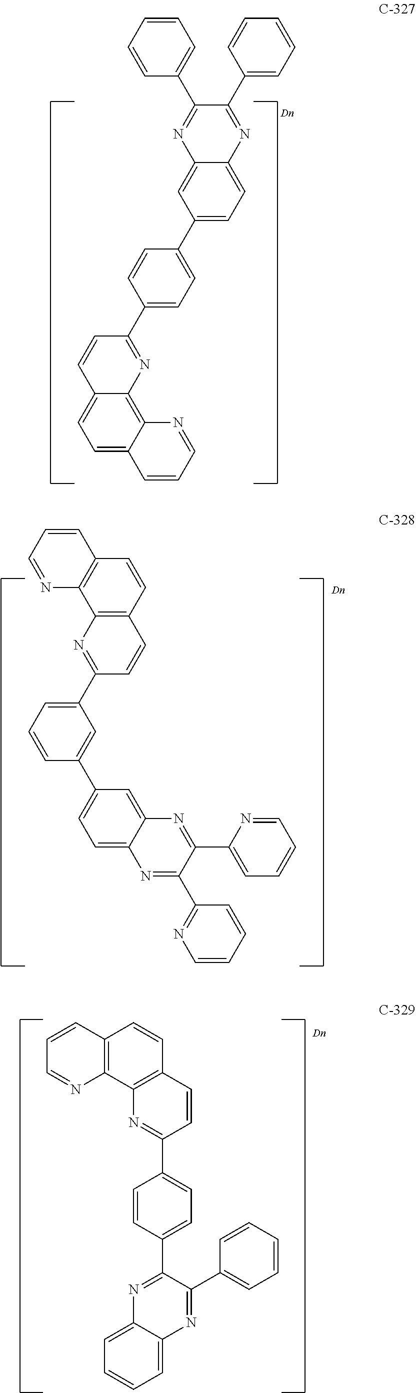

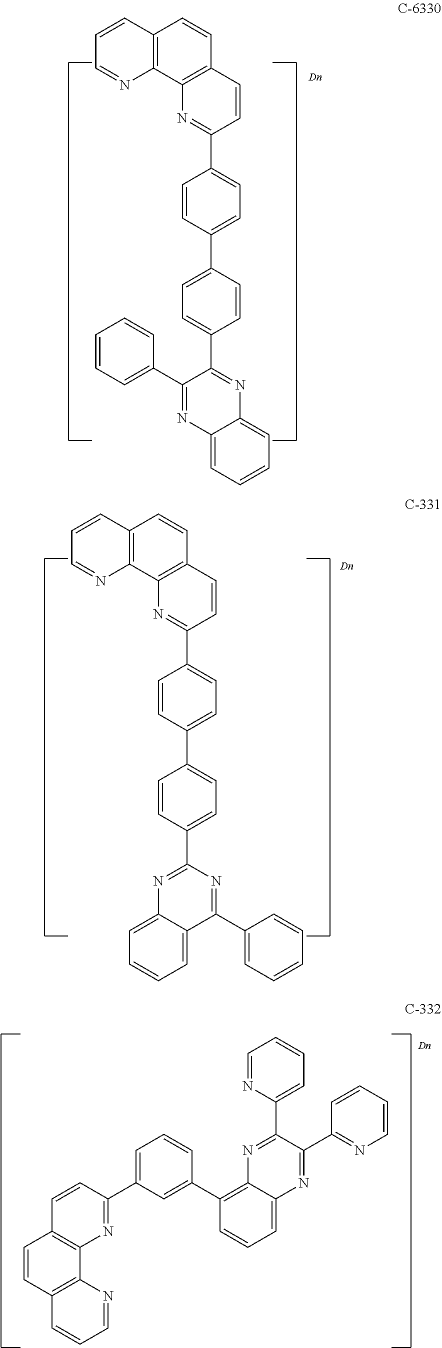

wherein Dₙ in the above compounds means that n number of hydrogens is replaced by deuterium, wherein n represents an integer of 1 or more, and the upper limit of n is determined by the number of hydrogens that can be substituted in each compound.

According to another embodiment, the present disclosure provides a compound selected from the following compounds, and an organic electroluminescent material comprising the same.

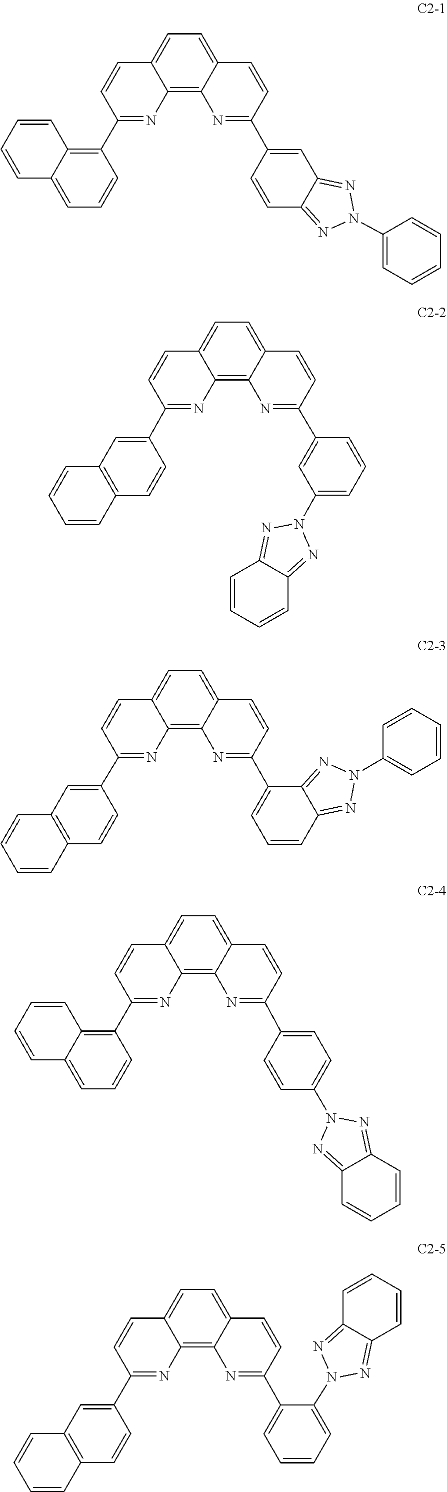 [SMILES: c1ccc(-n2nc3ccc(-c4ccc5ccc6ccc(-c7cccc8ccccc78)nc6c5n4)cc3n2)cc1 ; c1cc(-c2ccc3ccc4ccc(-c5ccc6ccccc6c5)nc4c3n2)cc(-n2nc3ccccc3n2)c1 ; c1ccc(-n2nc3cccc(-c4ccc5ccc6ccc(-c7ccc8ccccc8c7)nc6c5n4)c3n2)cc1 ; c1ccc2c(-c3ccc4ccc5ccc(-c6ccc(-n7nc8ccccc8n7)cc6)nc5c4n3)cccc2c1 ; c1ccc(-n2nc3ccccc3n2)c(-c2ccc3ccc4ccc(-c5ccc6ccccc6c5)nc4c3n2)c1]

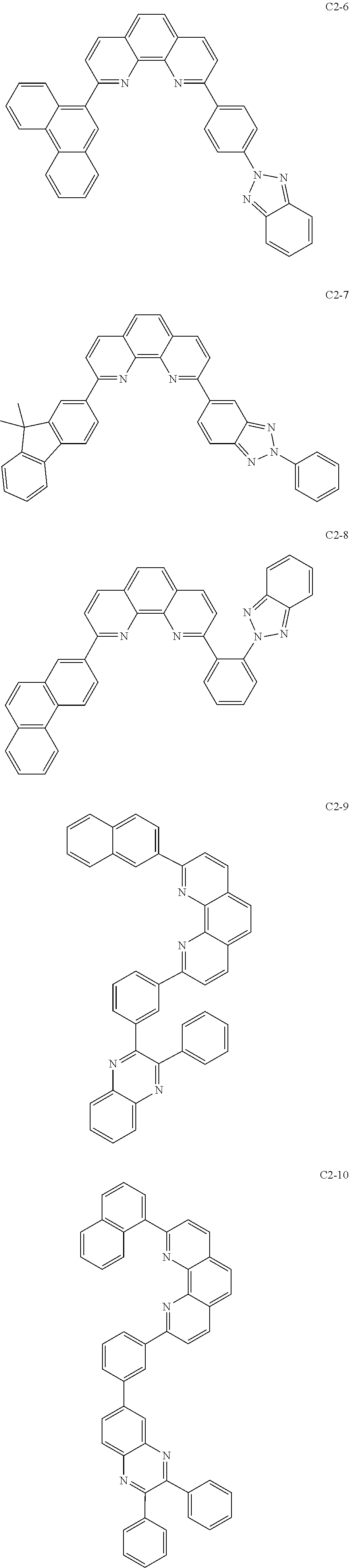 [SMILES: c1ccc2c(c1)cc(-c1ccc3ccc4ccc(-c5ccc(-n6nc7ccccc7n6)cc5)nc4c3n1)c1ccccc12 ; CC1(C)c2ccccc2-c2ccc(-c3ccc4ccc5ccc(-c6ccc7nn(-c8ccccc8)nc7c6)nc5c4n3)cc21 ; c1ccc(-n2nc3ccccc3n2)c(-c2ccc3ccc4ccc(-c5ccc6c(ccc7ccccc76)c5)nc4c3n2)c1 ; c1ccc(-c2nc3ccccc3nc2-c2cccc(-c3ccc4ccc5ccc(-c6ccc7ccccc7c6)nc5c4n3)c2)cc1 ; c1ccc(-c2nc3ccc(-c4cccc(-c5ccc6ccc7ccc(-c8cccc9ccccc89)nc7c6n5)c4)cc3nc2-c2ccccc2)cc1]

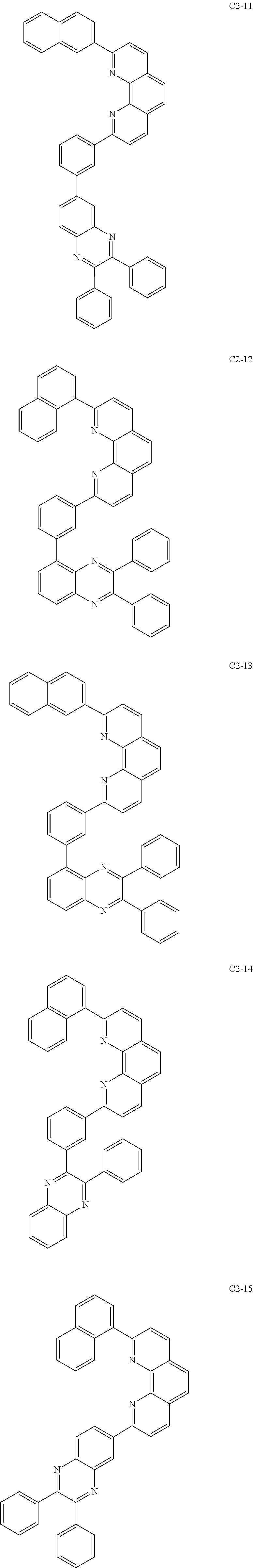 [SMILES: c1ccc(-c2nc3ccc(-c4cccc(-c5ccc6ccc7ccc(-c8ccc9ccccc9c8)nc7c6n5)c4)cc3nc2-c2ccccc2)cc1 ; c1ccc(-c2nc3cccc(-c4cccc(-c5ccc6ccc7ccc(-c8cccc9ccccc89)nc7c6n5)c4)c3nc2-c2ccccc2)cc1 ; c1ccc(-c2nc3cccc(-c4cccc(-c5ccc6ccc7ccc(-c8ccc9ccccc9c8)nc7c6n5)c4)c3nc2-c2ccccc2)cc1 ; c1ccc(-c2nc3ccccc3nc2-c2cccc(-c3ccc4ccc5ccc(-c6cccc7ccccc67)nc5c4n3)c2)cc1 ; c1ccc(-c2nc3ccc(-c4ccc5ccc6ccc(-c7cccc8ccccc78)nc6c5n4)cc3nc2-c2ccccc2)cc1]

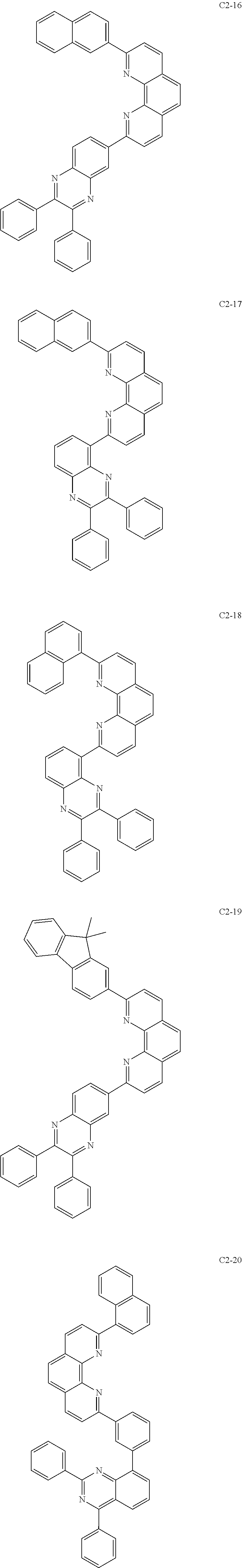 [SMILES: c1ccc(-c2nc3ccc(-c4ccc5ccc6ccc(-c7ccc8ccccc8c7)nc6c5n4)cc3nc2-c2ccccc2)cc1 ; c1ccc(-c2nc3cccc(-c4ccc5ccc6ccc(-c7ccc8ccccc8c7)nc6c5n4)c3nc2-c2ccccc2)cc1 ; c1ccc(-c2nc3cccc(-c4ccc5ccc6ccc(-c7cccc8ccccc78)nc6c5n4)c3nc2-c2ccccc2)cc1 ; CC1(C)c2ccccc2-c2ccc(-c3ccc4ccc5ccc(-c6ccc7nc(-c8ccccc8)c(-c8ccccc8)nc7c6)nc5c4n3)cc21 ; c1ccc(-c2nc(-c3ccccc3)c3cccc(-c4cccc(-c5ccc6ccc7ccc(-c8cccc9ccccc89)nc7c6n5)c4)c3n2)cc1]

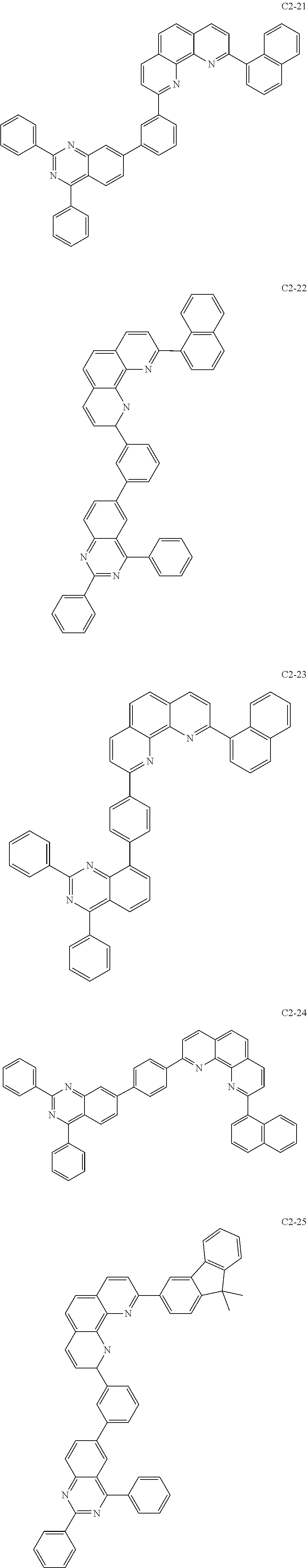

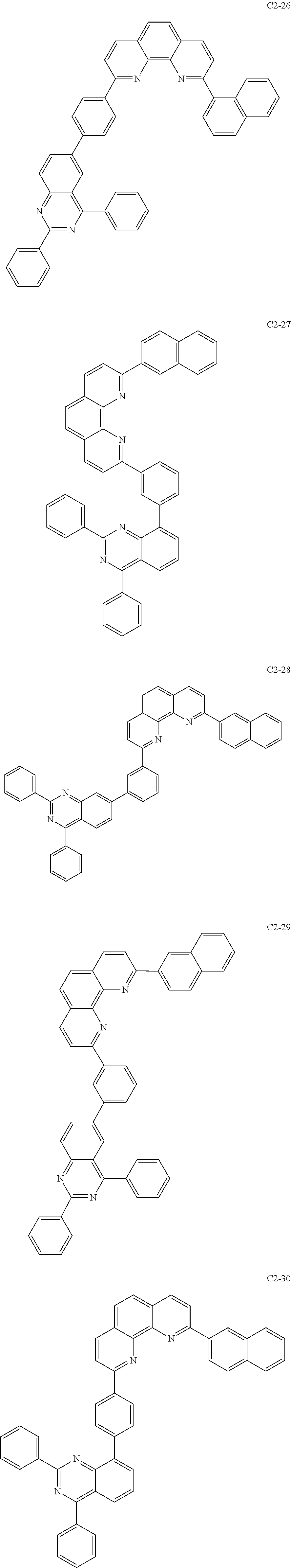 [SMILES: c1ccc(-c2nc(-c3ccccc3)c3cc(-c4ccc(-c5ccc6ccc7ccc(-c8cccc9ccccc89)nc7c6n5)cc4)ccc3n2)cc1 ; c1ccc(-c2nc(-c3ccccc3)c3cccc(-c4cccc(-c5ccc6ccc7ccc(-c8ccc9ccccc9c8)nc7c6n5)c4)c3n2)cc1 ; c1ccc(-c2nc(-c3ccccc3)c3ccc(-c4cccc(-c5ccc6ccc7ccc(-c8ccc9ccccc9c8)nc7c6n5)c4)cc3n2)cc1 ; c1ccc(-c2nc(-c3ccccc3)c3cc(-c4cccc(-c5ccc6ccc7ccc(-c8ccc9ccccc9c8)nc7c6n5)c4)ccc3n2)cc1 ; c1ccc(-c2nc(-c3ccccc3)c3cccc(-c4ccc(-c5ccc6ccc7ccc(-c8ccc9ccccc9c8)nc7c6n5)cc4)c3n2)cc1]

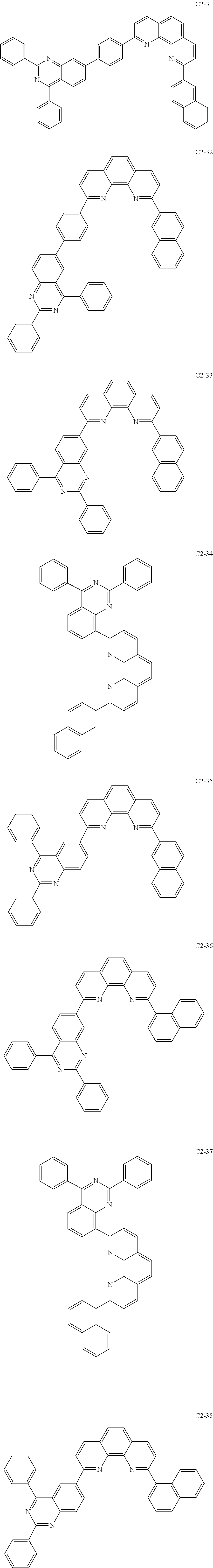

The compound represented by Formula 1 according to the present disclosure may be included in an N-type charge generation layer (CGL) of an organic electroluminescent device. In the compound of Formula 1 according to the present disclosure, the phenanthroline moiety includes an sp² hybrid orbital nitrogen that is relatively rich in electrons. In particular, the phenanthroline moiety has a structure in which two nitrogens are next to each other, and thus can covalently bond with surrounding hydrogen or coordinate with alkaline metals or alkaline earth metals such as Li and Yb. When applying the compound of Formula 1 having such a phenanthroline moiety to an N-type charge generation layer, the phenanthroline moiety can improve electron injection and transfer capabilities by trapping doped alkali metals or alkaline earth metals and increasing the electron density within the molecule. In addition, when the compound of Formula 1 according to the present disclosure is applied to an N-type charge generation layer of an organic electroluminescent device, the nitrogen of the phenanthroline moiety can form a gap state by binding with an alkali metal or alkaline earth metal, which is a dopant of the N-type charge generation layer. Accordingly, the energy level difference between the N-type charge generation layer and the P-type charge generation layer is alleviated, so that electrons can be smoothly transferred from the N-type charge generation layer to the electron transport layer.

The organic electroluminescent device according to one embodiment of the present disclosure may be an organic electroluminescent device having a tandem structure. In the case of a tandem organic electroluminescent device according to one embodiment, a single light-emitting unit (light-emitting unit) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer that is stacked between the first and second electrodes and emits light in a specific wavelength range. The plurality of light-emitting units may emit the same color or emit different colors. In addition, one light-emitting unit may include one or more light-emitting layers, and the plurality of light-emitting layers may be light-emitting layers of the same or different colors. It may include one or more charge generation layers located between each light-emitting unit. The charge generation layer refers to the layer in which holes and electrons are generated when voltage is applied. When there are three or more light-emitting units, a charge generation layer may be located between each light-emitting unit. Wherein, the plurality of charge generation layers may be the same as or different from each other. By disposing the charge generation layer between light-emitting units, current efficiency is increased in each light-emitting unit, and charges can be smoothly distributed. Specifically, the charge generation layer is provided between two adjacent stacks and can serve to drive a tandem organic electroluminescent device using only a pair of anodes and cathodes without a separate internal electrode located between the stacks.

Hereinafter, an organic electroluminescent device to which the aforementioned organic electroluminescent material according to the present disclosure is applied will be described.

An organic electroluminescent device according to one embodiment of the present invention includes a first electrode, a second electrode facing each other on the first electrode, a plurality of light-emitting units positioned between the first electrode and the second electrode, and at least one charge generation layer positioned between adjacent light-emitting units. The light-emitting units include at least one light-emitting layer(s), and the charge generation layer includes an N-type charge generation layer and a P-type charge generation layer. In this case, the N-type charge generation layer includes at least one compound selected from the compound represented by Formula 1 according to the present disclosure, for example, Compounds C-1 to C-332, or Compounds C₂-1 to C₂-38.

An organic electroluminescent device according to one embodiment comprises at least two light-emitting units, and a charge generation layer may be positioned between adjacent light-emitting units to increase the number of light-emitting units. According to one embodiment, at least one of the plurality of light-emitting units may include a first light-emitting layer and a second light-emitting layer adjacent to each other.

One of the first electrode and the second electrode may be an anode injecting a hole, and the other may be a cathode injecting an electron. Wherein, the first electrode and the second electrode may each be formed as a transmissive conductive material, a transflective conductive material, or a reflective conductive material. The organic electroluminescent device may be a top emission type, a bottom emission type, or a both-sides emission type according to the kinds of the material forming the first electrode and the second electrode.

According to one embodiment, each of light-emitting units includes each of hole transport layers, each of light-emitting layers, and each of electron transport layers.

The first light-emitting unit includes a hole injection layer positioned between the first electrode and the first light-emitting layer, a first hole transport layer positioned between the hole injection layer and the first light-emitting layer, and a first electron transport layer positioned between the first light-emitting layer and the charge generation layer.

The second light-emitting unit includes a second hole transport layer, a second light-emitting layer, a second electron transport layer, and an electron injection layer. The second hole transport layer is positioned between the charge generation layer and the second light-emitting layer, and the second light-emitting layer is positioned between the second hole transport layer and the second electrode. In addition, the second electron transport layer is positioned between the second light-emitting layer and the second electrode, and the electron injection layer is positioned between the second electron transport layer and the second electrode.

The hole injection layer may be formed of multiple layers for the purpose of lowering the hole injection barrier (or hole injection voltage) from the anode to the first hole transport layer or electron-blocking layer, and each layer may use two compounds at the same time. In addition, the hole injection layer may be doped with a p-dopant. In addition, the electron-blocking layer may be positioned between the hole transport layer (or hole injection layer) and the first light-emitting layer and the second light-emitting layer to block the overflow of electrons from the light-emitting layers, thereby confining excitons within the light-emitting layers and preventing light leakage. The first hole transport layer and the second hole transport layer or electron-blocking layers may be formed of multiple layers, and each layer may use multiple compounds. When the organic electroluminescent device includes two or more hole transport layers, the additionally included hole transport layer may be used as a hole auxiliary layer or electron-blocking layer.

The organic electroluminescent device according to one embodiment may include a light-emitting auxiliary layer placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled.

The first light-emitting layer and the second light-emitting layer are layers in which light emission occurs, including a host and a dopant, and may be a single layer or multiple layers in which two or more layers are laminated. Herein, the host mainly has the function of promoting the recombination of electrons and holes and confining excitons within the light-emitting layer, and the dopant has the function of efficiently emitting excitons obtained by the recombination. The dopant material of the first light-emitting layer and the second light-emitting layer may be doped in an amount of less than 25 wt %, preferably less than 17 wt %, and more preferably less than 10 wt %, relative to the total of the host material and the dopant material.

According to one embodiment, the first light-emitting layer and the second light-emitting layer may be an anthracene derivative compound as a host material, and, for example, the host material may be a fluorescent blue host material. In addition, the first light-emitting layer and the second light-emitting layer may further include one or more dopants. As a dopant included in the organic electroluminescent device of the present disclosure, one or more phosphorescent or fluorescent dopants may be used. For example, the dopant material may be a fluorescent blue dopant material.

According to one embodiment of the present disclosure, an organic electroluminescent device having a tandem structure has a charge generation layer positioned between a first light-emitting unit and a second light-emitting unit to increase current efficiency generated in each light-emitting layer and to smoothly distribute charges. The charge generation layer includes an N-type charge generation layer positioned adjacent to the first light-emitting unit to supply electrons to the first light-emitting unit and a P-type charge generation layer positioned adjacent to the second light-emitting unit to supply holes to the second light-emitting unit.

That is, the charge generation layer is positioned between the first light-emitting unit and the second light-emitting unit, and the first light-emitting unit and the second light-emitting unit are connected by the charge generation layer. The charge generation layer may be a PN-junction charge generation layer in which an N-type charge generation layer and a P-type charge generation layer are positioned adjacent to each other and joined.

The N-type charge generation layer supplies electrons to the first electron transport layer of the first light-emitting unit, and the first electron transport layer supplies electrons to the first light-emitting layer adjacent to the first electrode.

According to one embodiment of the present disclosure, the N-type charge generation layer includes the aforementioned compound represented by Formula 1. The compound of Formula 1 has excellent electron mobility and thus excellent electron injection and transfer capabilities. Therefore, when the compound of Formula 1 is applied to an organic electroluminescent device as an N-type charge generation layer material, an increase in the progressive driving voltage of the device and a decrease in the lifespan can be prevented.

According to another embodiment, the N-type charge generation layer includes at least one compound selected from Compounds C₂-1 to C₂-38.

In one embodiment, the N-type charge generation layer may further include an N-type dopant to improve electron injection characteristics into the N-type charge generation layer. For example, the usable N-type dopant may further include an alkali metal such as Li, Na, K, Rb, Cs, Fr, Yb, etc., an alkaline earth metal such as Be, Mg, Ca, Sr, Ba, Ra, etc., or one or more complex compounds including such metals, which are generally used in the art. In the N-type charge generation layer, the doping concentration of the dopant may be 0.5% to 10% of the compound of Formula 1.

The P-type charge generation layer supplies holes to the second hole transport layer of the second light-emitting unit, and the second hole transport layer supplies holes to the second light-emitting layer adjacent to the second electrode. That is, the P-type charge generation layer is used as a hole injection layer, and may include a hole injection layer material alone, or may include a hole injection layer material in a mixture of a hole transport material.

In one embodiment, the P-type charge generation layer may be formed of a metal or an organic material doped with a P-type dopant. For example, the metal may be formed of one or more alloys selected from the group consisting of Al, Cu, Fe, Pb, Zn, Au, Pt, W, In, Mo, Ni, and Ti. In addition, the P-type dopant and the host material used in the P-type doped organic material may use commonly used materials.

The light-emitting units according to one embodiment may further include a hole-blocking layer between the light-emitting layers and the electron transport layers. The hole-blocking layer is a layer that blocks holes from reaching the cathode, thereby improving the probability of recombination of electrons and holes in the light-emitting layer. The hole-blocking layer or the first electron transport layer and the second electron transport layer may use multiple layers, and multiple compounds may be used in each layer. In addition, the first electron injection layer and the second electron injection layer may be doped with an n-dopant.

The organic electroluminescent device includes a first electrode and a second electrode facing each other, and an organic layer positioned between the first electrode and the second electrode. The organic layer may include a first light-emitting unit, a second light-emitting unit, a third light-emitting unit, a first charge generation layer, and a second charge generation layer. In another embodiment, four or more light-emitting units and three or more charge generation layers may be positioned between the first electrode and the second electrode.

The first charge generation layer and the second charge generation layer are positioned between the first light-emitting unit and the second light-emitting unit, and the second light-emitting unit and the third light-emitting unit, respectively, and the first light-emitting unit, the first charge generation layer, the second light-emitting unit, the second charge generation layer, and the third light-emitting unit are sequentially laminated on the first electrode. That is, the first light-emitting unit is positioned between the first electrode and the first charge generation layer, the second light-emitting unit is positioned between the first charge generation layer and the second charge generation layer, and the third light-emitting unit is positioned between the second electrode and the second charge generation layer.

The first light-emitting unit may include a hole injection layer, a first hole transport layer, a first light-emitting layer, and a first electron transport layer that are sequentially laminated on the first electrode. At this time, the first electron transport layer is located between the first light-emitting layer and the first charge generation layer. The hole injection layer, the first hole transport layer, the first light-emitting layer, and the first electron transport layer are each described above, and thus a description thereof will be omitted.

The second light-emitting unit may include a second hole transport layer, a second light-emitting layer, and a second electron transport layer. The second hole transport layer is positioned between the first charge generation layer and the second light-emitting layer, and the second electron transport layer is positioned between the second light-emitting layer and the second charge generation layer. The second hole transport layer, the second light-emitting layer, and the second electron transport layer are each described above, and thus a description thereof will be omitted.

The third light-emitting unit may include a third hole transport layer, a third light-emitting layer, a third electron transport layer, and an electron injection layer. The third hole transport layer is positioned between the second charge generation layer and the third light-emitting layer, the third electron transport layer is positioned between the third light-emitting layer and the second electrode, and the electron injection layer is positioned between the third electron transport layer and the second electrode. The third hole transport layer, the third electron transport layer, and the electron injection layer may have similar characteristics to the second hole transport layer, the second electron transport layer, and the electron injection layer above, respectively, and a description thereof will be omitted. In addition, the third light-emitting layer may have similar characteristics to the first light-emitting layer or the second light-emitting layer. For example, the third light-emitting layer may include a fluorescent blue host material and a fluorescent blue dopant material.

In the second charge generation layer, the N-type charge generation layer is positioned between the second electron transport layer and the third hole transport layer, and the P-type charge generation layer is positioned between the N-type charge generation layer and the third hole transport layer. The first and second charge generation layers generate charges or separate charges into holes and electrons to supply electrons and holes to the first light-emitting unit, the second light-emitting unit, and the third light-emitting unit. The N-type charge generation layer and the P-type charge generation layer may have similar characteristics to the N-type charge generation layer and the P-type charge generation layer above, respectively, and a description thereof will be omitted.

In one embodiment according to the present disclosure, at least one of the N-type charge generation layer and the N-type charge generation layer includes the aforementioned compound represented by Formula 1.

In order to form each layer of the organic electroluminescent device of the present disclosure, dry film-forming methods such as vacuum evaporation, sputtering, plasma, ion plating methods, etc. or wet film-forming methods such as spin coating, dip coating, flow coating methods, etc. can be used. When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

When forming a layer by the compound of Formula 1 according to one embodiment, the layer can be formed by the above-listed methods, and can often be formed by co-deposition or mixture-deposition. The co-deposition is a mixed deposition method in which two or more materials are put into respective individual crucible sources, and a current is applied to both cells simultaneously to evaporate the materials and to perform mixed deposition; the mixed deposition is a mixed deposition method in which two or more materials are mixed in one crucible source before deposition, and then a current is applied to one cell to evaporate the materials.

In one embodiment, when the organic electroluminescent materials are present in the same layer or different layers within the organic electroluminescent device, they can be individually deposited.

According to one embodiment, the present disclosure can provide a display device comprising a compound represented by Formula 1. In addition, the organic electroluminescent device of the present disclosure can be used for the manufacture of display devices such as smartphones, tablets, notebooks, PCs, TVs, or display devices for vehicles, or lighting devices such as outdoor or indoor lighting.

Hereinafter, the preparation method of the compound according to the present disclosure will be explained with reference to the synthesis method of a representative compound or intermediate compound in order to understand the present disclosure in detail.

### [Example 1] Synthesis of Compound C-1

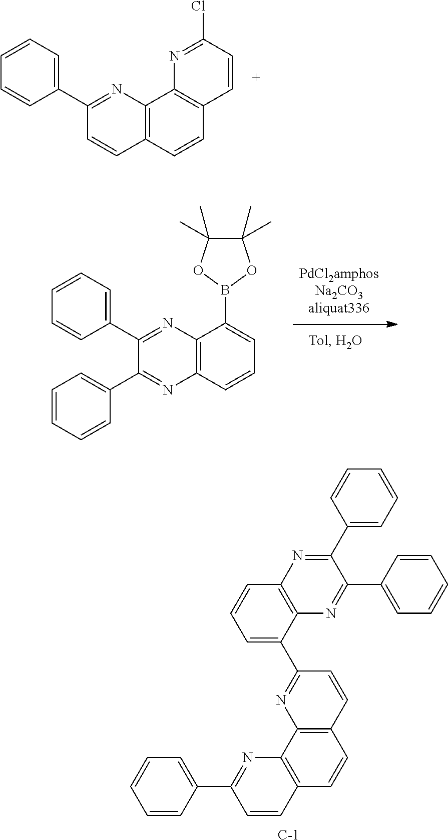

2-Chloro-9-phenyl-1,10-phenanthroline (5.3 g, 13 mmol), 2,3-diphenyl-5-(4,4,5,5-tetramethyl-1,3,2-dioxaborolan-2-yl)quinoxaline (3.8 g, 13 mmol), PdCl₂amphos (0.64 g, 0.91 mmol), Aliquat 336 (0.53 g, 1.3 mmol), Na₂CO₃ (2.8 g, 26 mmol), 50 mL of toluene, and 17 mL of distilled water were added to a flask and stirred under reflux at 140° C. After 17 hours, the mixture was cooled to room temperature, distilled water was added, and the organic layer was extracted with ethyl acetate. Next, the remaining moisture was removed using magnesium sulfate, and the resultant was distilled under reduced pressure and separated using column chromatography to obtain Compound C-1 (2.4 g, yield: 34%).

| | MW | M.P. |
|---|---|---|
| C-1 | 536.2 | 270° C. |

### [Example 2] Synthesis of Compound C-3

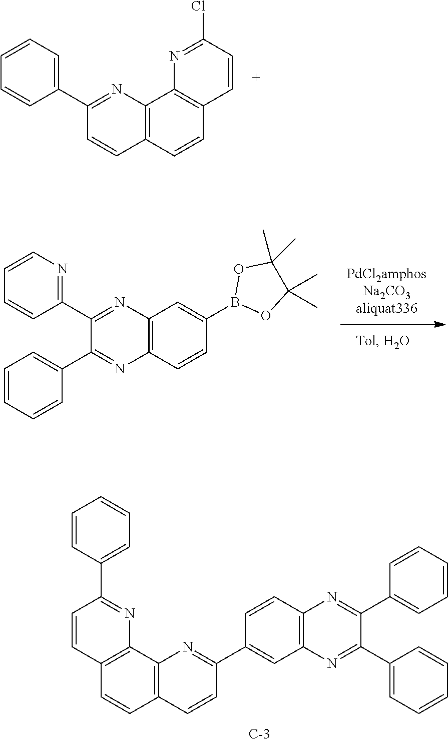

2-Chloro-9-phenyl-1,10-phenanthroline (6.8 g, 23 mmol), 2,3-diphenyl-6-(4,4,5,5-tetramethyl-1,3,2-dioxaborolan-2-yl)quinoxaline (9.5 g, 23 mmol), PdCl₂amphos (1.2 g, 1.6 mmol), Aliquat 336 (0.89 g, 2.3 mmol), Na₂CO₃ (2.8 g, 27 mmol), 90 mL of toluene, and 30 mL of distilled water were added to a flask and stirred under reflux at 140° C. After 16 hours, the mixture was cooled to room temperature, distilled water was added, and the organic layer was extracted with ethyl acetate. Next, the remaining moisture was removed using magnesium sulfate, and the resultant was distilled under reduced pressure and separated using column chromatography to obtain Compound C-3 (8.8 g, yield: 70%).

| | MW | M.P. |
|---|---|---|
| C-3 | 536.2 | 227° C. |

### [Example 3] Synthesis of Compound C-5

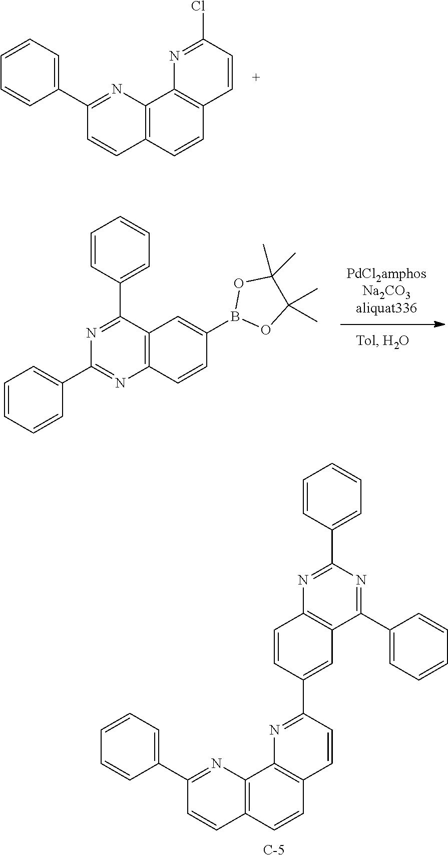

2-Chloro-9-phenyl-1,10-phenanthroline (7.1 g, 24 mmol), 2,4-diphenyl-6-(4,4,5,5-tetramethyl-1,3,2-dioxaborolan-2-yl)quinazoline (10 g, 24 mmol), PdCl₂amphos (1.2 g, 1.7 mmol), Aliquat 336 (1.0 g, 2.5 mmol), Na₂CO₃ (5.2 g, 49 mmol), 122 mL of toluene, and 41 mL of distilled water were added to a flask and stirred under reflux at 140° C. After 3 hours, the mixture was cooled to room temperature, and the resulting solid was filtered and then separated using column chromatography to obtain Compound C-5 (8.3 g, yield: 64%).

| | MW | M.P. |
|---|---|---|
| C-5 | 536.2 | 292° C. |

### [Example 4] Synthesis of Compound C-57

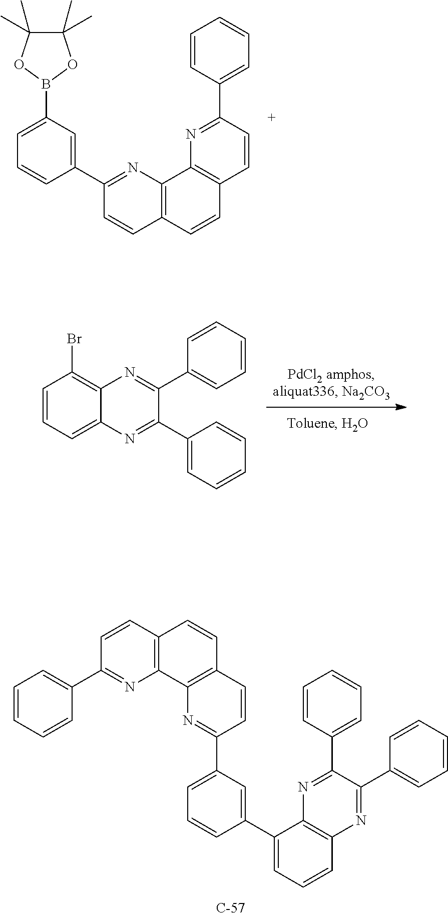

2-Phenyl-9-(3-(4,4,5,5-tetramethyl-1,3,2-dioxaborolan-2-yl)phenyl)-1,10-phenanthroline (7.0 g, 15.27 mmol), 5-bromo-2,3-diphenylquinoxaline (5.5 g, 15.27 mmol), Pd(amphos)Cl₂ (0.8 g, 1.07 mmol), Aliquat 336 (0.6 g, 1.53 mmol), Na₂CO₃ (3.2 g, 30.54 mmol), 76 mL of toluene, and 25 mL of distilled water were added to a flask and stirred under reflux at 130° C. After 2 hours, the mixture was cooled to room temperature, and the layers were separated. Next, this was filtered using silica, and recrystallized to obtain Compound C-57 (6.8 g, yield: 72.72%).

| | MW | M.P. |
|---|---|---|
| C-57 | 612.74 | 276° C. |

### [Example 5] Synthesis of Compound C-4

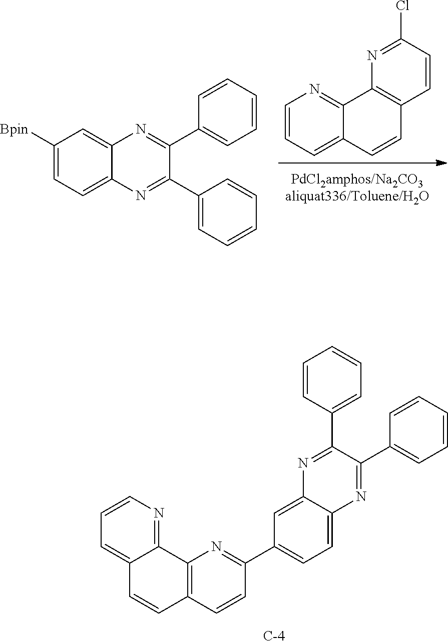

2-Chloro-1,10-phenanthroline (5.0 g, 23.3 mmol), 2,3-diphenyl-6-(4,4,5,5-tetramethyl-1,3,2-dioxaborolan-2-yl)quinoxaline (10 g, 24.5 mmol), Pd(amphos)Cl₂ (1.15 g, 1.63 mmol), Na₂CO₃ (4.94 g, 46.6 mmol), Aliquat 336 (0.188 g, 0.466 mmol), 50 mL of toluene, and 50 mL of distilled water were added to a flask and stirred under reflux at 140° C. After 12 hours, the mixture was cooled to room temperature, and the layers were separated. Next, this was filtered using silica, and recrystallized to obtain Compound C-4 (4.7 g, yield: 44%).

| | MW | M.P. |
|---|---|---|
| C-4 | 460.54 | 288° C. |

### [Example 6] Synthesis of Compound C-137

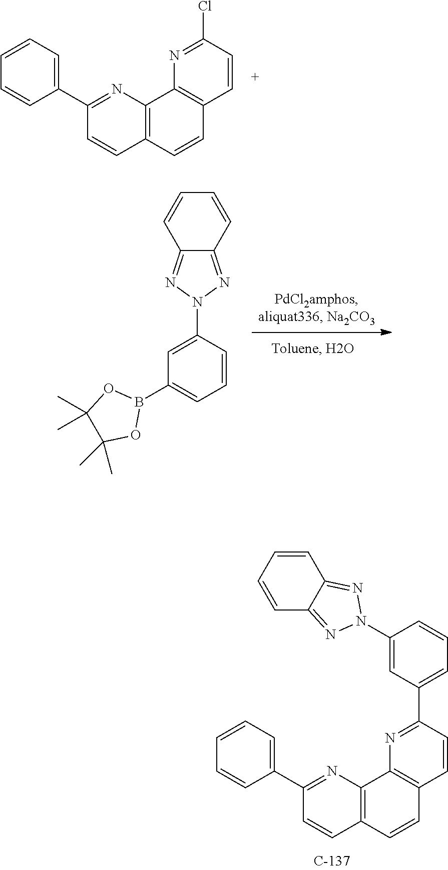

2-Chloro-9-phenyl-1,10-phenanthroline (8.6 g, 29.6 mmol), 2-(3-(4,4,5,5-tetramethyl-1,3,2-dioxaborolan-2-yl)phenyl)-2H-benzo[d][1,2,3]triazole (11.4 g, 35.5 mmol), Pd(amphos)Cl₂ (1.8 g, 2.48 mmol), Aliquat 336 (1.4 g, 3.55 mmol), Na₂CO₃ (7.5 g, 71.0 mmol), 177 mL of toluene, and 59 mL of distilled water were added to a flask and stirred under reflux at 130° C. After 3 hours, the mixture was cooled to room temperature, and the layers were separated. Next, this was filtered using silica, and recrystallized to obtain Compound C-137 (3.5 g, yield: 26.35%).

| | MW | M.P. |
|---|---|---|
| C-137 | 449.52 | 194° C. |

### [Example 7] Synthesis of Compound C-6

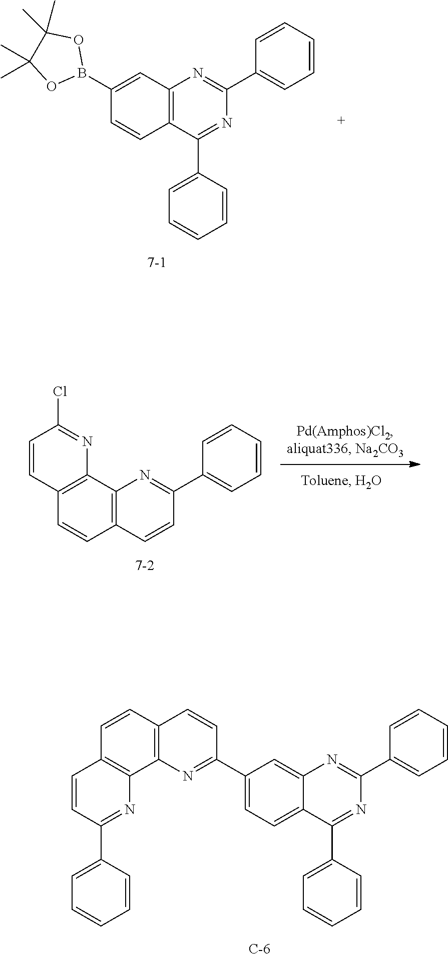

Compound 7-1 (10.0 g, 24.49 mmol), Compound 7-2 (7.8 g, 26.94 mmol), Pd(amphos)Cl₂ (1.2 g, 1.71 mmol), Aliquat 336 (1.0 g, 2.45 mmol), and Na₂CO₃ (5.2 g, 48.98 mmol) in a flask were dissolved in 122 mL of toluene and 41 mL of distilled water, and stirred under reflux at 130° C. After 2 hours and 30 minutes, the mixture was cooled to room temperature, and the layers in the resulting solid reaction product were separated. Next, this was filtered using silica and recrystallized. Thereafter, it was separated by column chromatography to obtain Compound C-6 (3.5 g, yield: 26.63%).

| | MW | M.P. |
|---|---|---|
| C-6 | 536.62 | 283° C. |

### [Example 8] Synthesis of Compound C-200

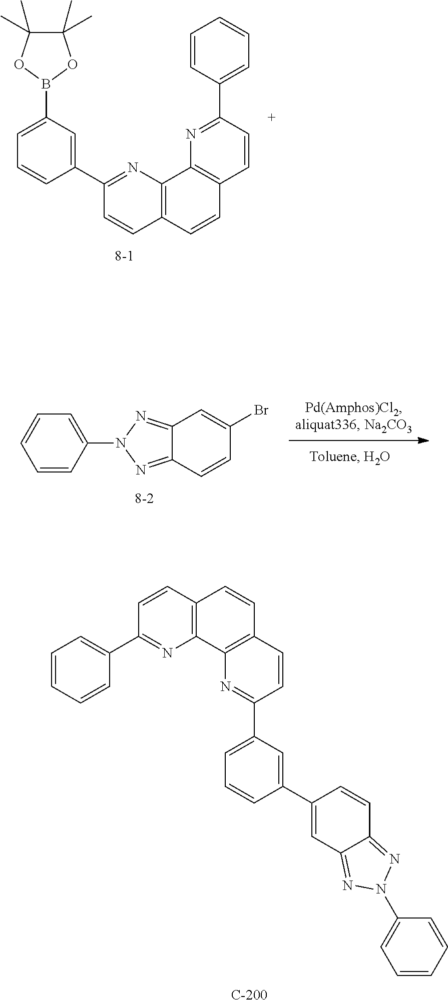 [SMILES: CC1(C)OB(c2cccc(-c3ccc4ccc5ccc(-c6ccccc6)nc5c4n3)c2)OC1(C)C ; Brc1ccc2nn(-c3ccccc3)nc2c1 ; c1ccc(-c2ccc3ccc4ccc(-c5cccc(-c6ccc7nn(-c8ccccc8)nc7c6)c5)nc4c3n2)cc1 ; "C-200"]

Compound 8-1 (8.0 g, 17.45 mmol), Compound 8-2 (5.3 g, 19.19 mmol), Pd(amphos)Cl₂ (0.9 g, 1.22 mmol), Aliquat 336 (0.7 g, 1.74 mmol), and Na₂CO₃ (3.7 g, 34.90 mmol) in a flask were dissolved in 87 mL of toluene and 29 mL of distilled water, and stirred under reflux at 130° C. After 2 hours, the mixture was cooled to room temperature, and the layers were separated. Next, this was filtered using silica, and recrystallized to obtain Compound C-200 (3.3 g, yield: 35.98%).

| | MW | M.P. |
|---|---|---|
| C-200 | 525.60 | 204° C. |

### [Example 9] Synthesis of Compound C-12

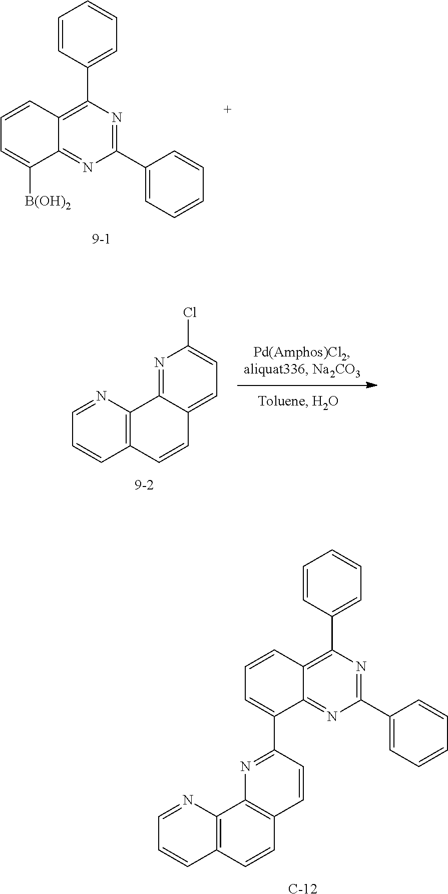

Compound 9-1 (7.8 g, 23.91 mmol), Compound 9-2 (7.7 g, 35.87 mmol), Pd(amphos)Cl₂ (1.2 g, 1.67 mmol), Aliquat 336 (1.0 g, 2.39 mmol), and Na₂CO₃ (5.1 g, 47.82 mmol) in a flask were dissolved in 120 mL of toluene and 40 mL of distilled water, and stirred under reflux at 130° C. After 2 hours, the mixture was cooled to room temperature, and the layers were separated. Next, this was filtered using silica, and recrystallized to obtain Compound C-12 (8.1 g, yield: 73.56%).

| | MW | M.P. |
|---|---|---|
| C-12 | 460.53 | 284° C. |

### [Example 10] Synthesis of Compound C-82

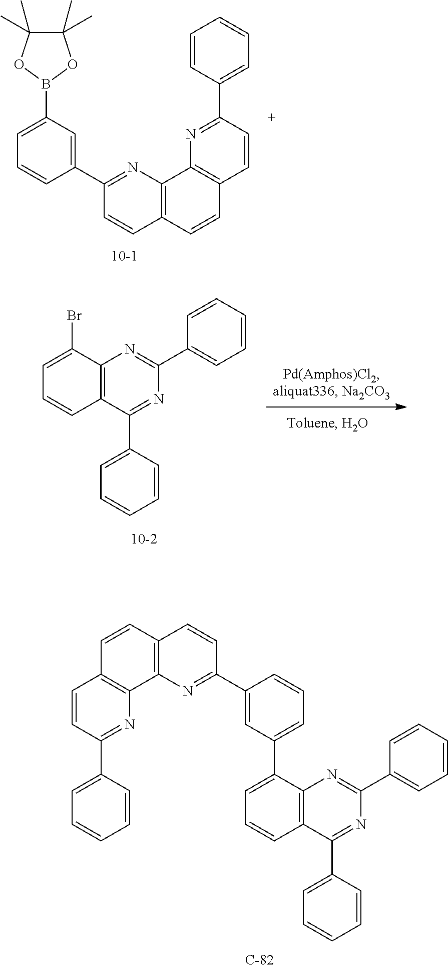

Compound 10-1 (15.0 g, 32.72 mmol), Compound 10-2 (13.0 g, 35.99 mmol), Pd(amphos)Cl₂ (1.6 g, 2.29 mmol), Aliquat 336 (1.3 g, 3.27 mmol), and Na₂CO₃ (6.9 g, 65.44 mmol) in a flask were dissolved in 165 mL of toluene and 55 mL of distilled water, and stirred under reflux at 130° C. After 3 hours, the mixture was cooled to room temperature, and the layers were separated. Next, this was filtered using silica to obtain Compound C-82 (13.0 g, yield: 64.83%).

| | MW | M.P. |
|---|---|---|
| C-82 | 612.72 | 251° C. |

### [Example 11] Synthesis of Compound C-76

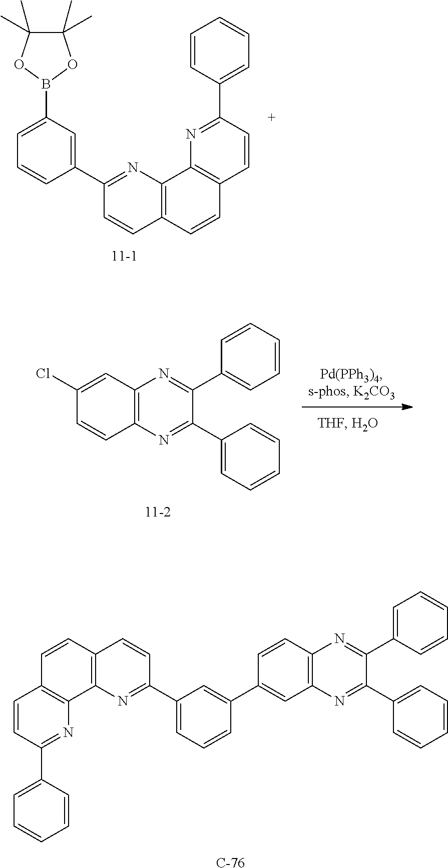

Compound 11-1 (17.0 g, 37.1 mmol), Compound 11-2 (12.0 g, 37.9 mmol), Pd(PPh₃)₄ (0.416 g, 1.85 mmol), SPhos (1.52 g, 3.71 mmol), and K2CO₃ (15.4 g, 111 mmol) in a flask were dissolved in 340 mL of THE and 34 mL of distilled water and stirred under reflux at 70° C. After 3 hours, the mixture was cooled to room temperature and filtered with silica to obtain Compound C-76 (15.3 g, yield: 67.4%).

| | MW | M.P. |
|---|---|---|
| C-76 | 612.74 | 284° C. |

### [Example 12] Synthesis of Compound C-151

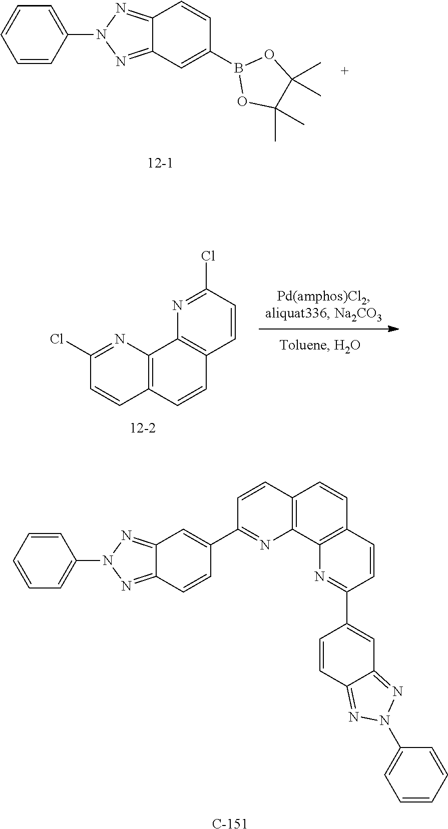 [SMILES: CC1(C)OB(c2ccc3nn(-c4ccccc4)nc3c2)OC1(C)C ; Clc1ccc2ccc3ccc(Cl)nc3c2n1 ; c1ccc(-n2nc3ccc(-c4ccc5ccc6ccc(-c7ccc8nn(-c9ccccc9)nc8c7)nc6c5n4)cc3n2)cc1 ; "C-151"]

Compound 12-1 (15.7 g, 49.0 mmol), Compound 12-2 (5.5 g, 22.1 mmol), Pd(amphos)Cl₂ (2.42 g, 3.43 mmol), Aliquat 336 (1.98 g, 4.9 mmol), and Na₂CO₃ (10.4 g, 98.0 mmol) in a flask were dissolved in 200 mL of toluene and 50 mL of distilled water and stirred under reflux at 100° C. After 18 hours, methanol was added, the resulting solid reaction product was cooled to room temperature, and the layers were separated. Next, this was separated through a silica filter to obtain Compound C-151 (7.5 g, yield: 59.9%).

| | MW | M.P. |
|---|---|---|
| C-151 | 566.63 | 327° C. |

### [Example 13] Synthesis of Compound C-213

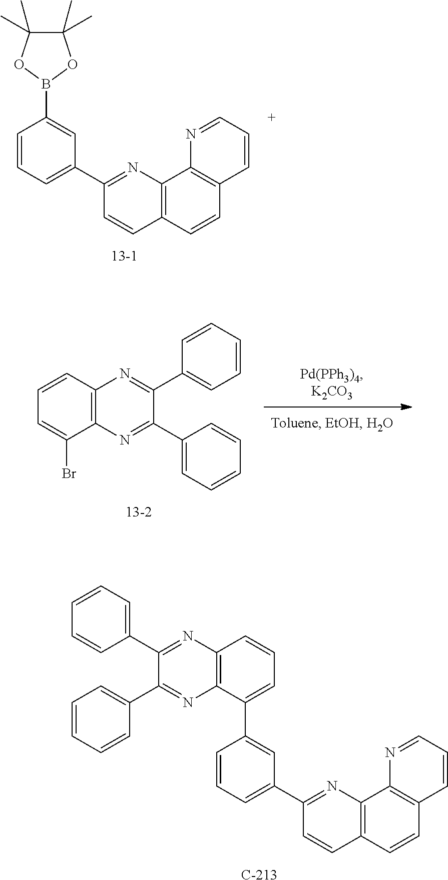 [SMILES: CC1(C)OB(c2cccc(-c3ccc4ccc5cccnc5c4n3)c2)OC1(C)C ; Brc1cccc2nc(-c3ccccc3)c(-c3ccccc3)nc12 ; c1ccc(-c2nc3cccc(-c4cccc(-c5ccc6ccc7cccnc7c6n5)c4)c3nc2-c2ccccc2)cc1 ; "C-213"]

Compound 13-1 (8.8 g, 23 mmol), Compound 13-2 (8.5 g, 24 mmol), Pd(PPh₃)₄ (1.3 g, 1.1 mmol), and K2CO₃ (6.3 g, 46 mmol) in a flask were dissolved in 125 mL of toluene, 25 mL of ethanol, and 25 mL of distilled water and stirred under reflux at 100° C. for 4 hours. After the reaction was completed, the reaction product was cooled to room temperature, the organic matter was extracted with methylene chloride and then distilled under reduced pressure. Thereafter, this was separated by column chromatography to obtain Compound C-213 (9.0 g, yield: 73%).

| | MW | M.P. |
|---|---|---|
| C-213 | 536.64 | 180° C. |

### [Example 14] Synthesis of Compound C-170

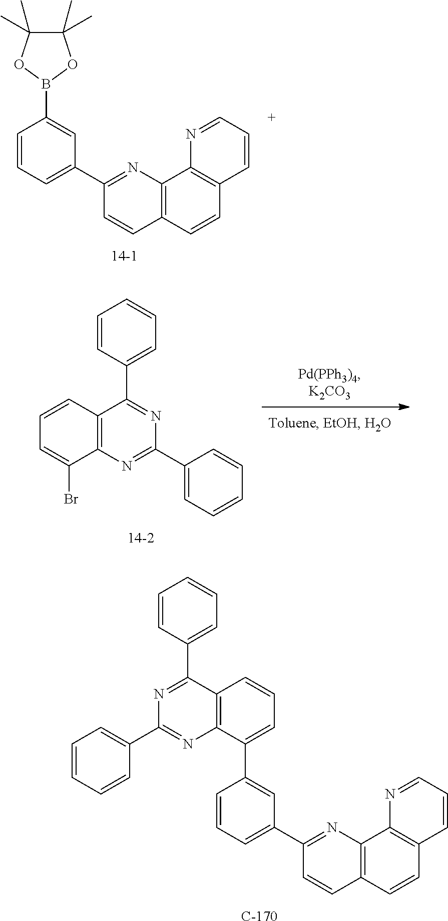 [SMILES: CC1(C)OB(c2cccc(-c3ccc4ccc5cccnc5c4n3)c2)OC1(C)C ; Brc1cccc2c(-c3ccccc3)nc(-c3ccccc3)nc12 ; c1ccc(-c2nc(-c3ccccc3)c3cccc(-c4cccc(-c5ccc6ccc7cccnc7c6n5)c4)c3n2)cc1 ; "C-170"]

Compound 14-1 (8.8 g, 23 mmol), Compound 14-2 (8.5 g, 24 mmol), Pd(PPh₃)₄ (1.3 g, 1.1 mmol), and K2CO₃ (6.3 g, 46 mmol) in a flask were dissolved in 125 mL of toluene, 25 mL of ethanol, and 25 mL of distilled water and stirred under reflux at 100° C. for 4 hours. After the reaction was completed, the reaction product was cooled to room temperature, the organic matter was extracted with methylene chloride, and then distilled under reduced pressure. After solidifying with ethyl acetate, this was separated by column chromatography to obtain Compound C-170 (7.5 g, yield: 61%).

| | MW | M.P. |
|---|---|---|
| C-170 | 536.64 | 230° C. |

### [Example 15] Synthesis of Compound C-300

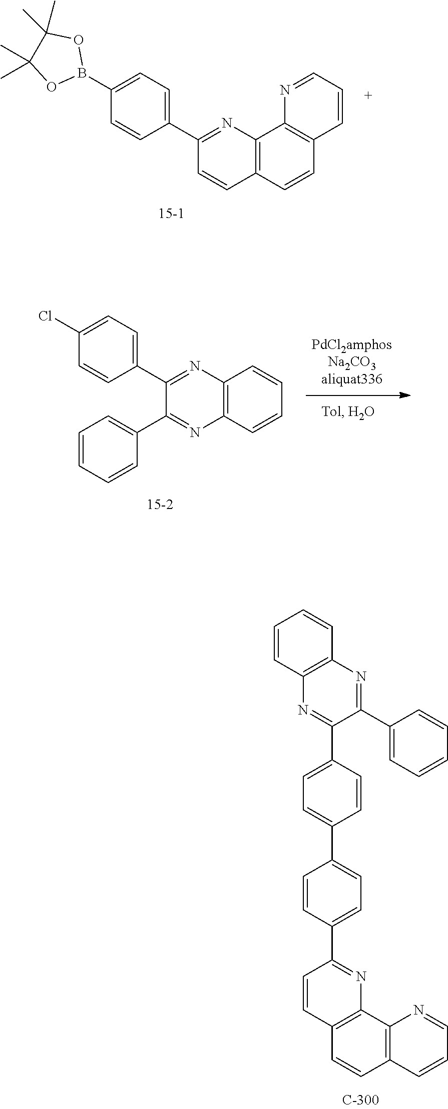 [SMILES: CC1(C)OB(c2ccc(-c3ccc4ccc5cccnc5c4n3)cc2)OC1(C)C ; Clc1ccc(-c2nc3ccccc3nc2-c2ccccc2)cc1 ; c1ccc(-c2nc3ccccc3nc2-c2ccc(-c3ccc(-c4ccc5ccc6cccnc6c5n4)cc3)cc2)cc1]

Compound 15-1 (5.0 g, 13.08 mmol), Compound 15-2 (4.6 g, 14.39 mmol), Pd(amphos)Cl₂ (0.6 g, 0.91 mmol), Aliquat 336 (1.1 g, 2.61 mmol), and Na₂CO₃ (2.8 g, 26.16 mmol) in a flask were dissolved in 65 mL of toluene and 22 mL of distilled water and stirred under reflux at 130° C. for 5 hours. After the reaction was completed, the mixture was cooled to room temperature, its layers were separated, and the resultant was then filtered with Celite. Afterwards, this was filtered with silica and recrystallized to obtain Compound C-300 (4.8 g, yield: 69%).

| | MW | M.P. |
|---|---|---|
| C-300 | 536.64 | 270° C. |

### [Example 16] Synthesis of Compound C-302

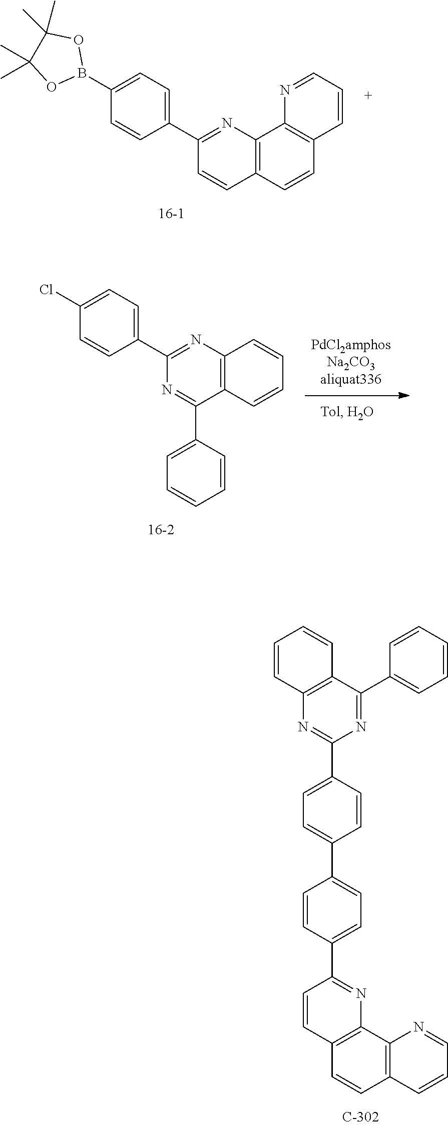 [SMILES: CC1(C)OB(c2ccc(-c3ccc4ccc5cccnc5c4n3)cc2)OC1(C)C ; Clc1ccc(-c2nc(-c3ccccc3)c3ccccc3n2)cc1 ; c1ccc(-c2nc(-c3ccc(-c4ccc(-c5ccc6ccc7cccnc7c6n5)cc4)cc3)nc3ccccc23)cc1]

Compound 16-1 (5.0 g, 13.08 mmol), Compound 16-2 (4.6 g, 14.39 mmol), Pd(amphos)Cl₂ (0.6 g, 0.91 mmol), Aliquat 336 (1.1 g, 2.61 mmol), and Na₂CO₃ (2.8 g, 26.16 mmol) in a flask were dissolved in 65 mL of toluene and 22 mL of distilled water and stirred under reflux at 130° C. for 5 hours. After the reaction was completed, the mixture was cooled to room temperature, the layers were separated, and the resultant was then filtered with Celite. Next, this was filtered with silica and recrystallized to obtain Compound C-302 (3.5 g, yield: 49%).

| | MW | M.P. |
|---|---|---|
| C-302 | 536.64 | 268° C. |

### [Example 17] Synthesis of Compound C-233

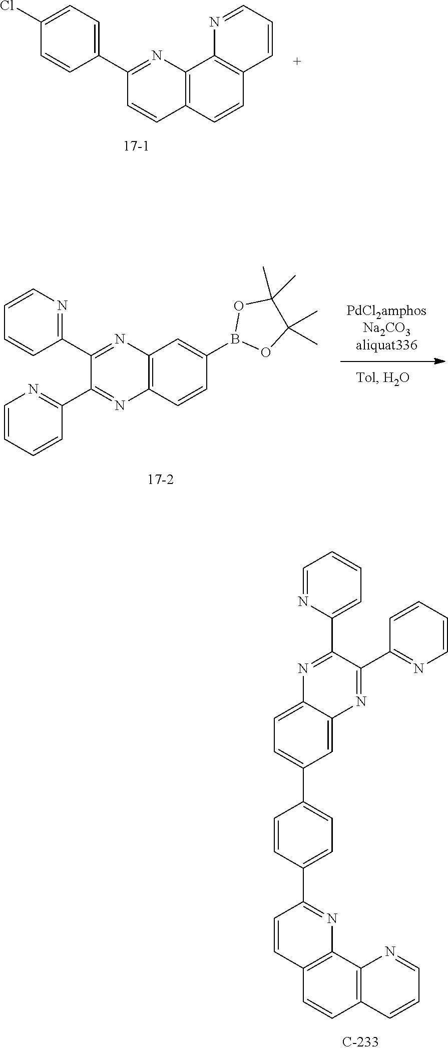 [SMILES: Clc1ccc(-c2ccc3ccc4cccnc4c3n2)cc1 ; CC1(C)OB(c2ccc3nc(-c4ccccn4)c(-c4ccccn4)nc3c2)OC1(C)C ; c1ccc(-c2nc3ccc(-c4ccc(-c5ccc6ccc7cccnc7c6n5)cc4)cc3nc2-c2ccccn2)nc1]

Compound 17-1 (5.0 g, 12.2 mmol), Compound 17-2 (3.9 g, 13.4 mmol), Pd(amphos)Cl₂ (0.6 g, 0.91 mmol), Aliquat 336 (1.0 g, 2.44 mmol), and Na₂CO₃ (2.6 g, 24.4 mmol) in a flask were dissolved in 61 mL of toluene and 20 mL of distilled water and stirred under reflux at 130° C. for 5 hours. After the reaction was completed, the mixture was cooled to room temperature, the layers were separated, and the resultant was then filtered with Celite. Next, this was filtered with silica and recrystallized to obtain Compound C-233 (2.7 g, yield: 41%).

| | MW | M.P. |
|---|---|---|
| C-233 | 538.61 | 315° C. |

### [Example 18] Synthesis of Compound C-235

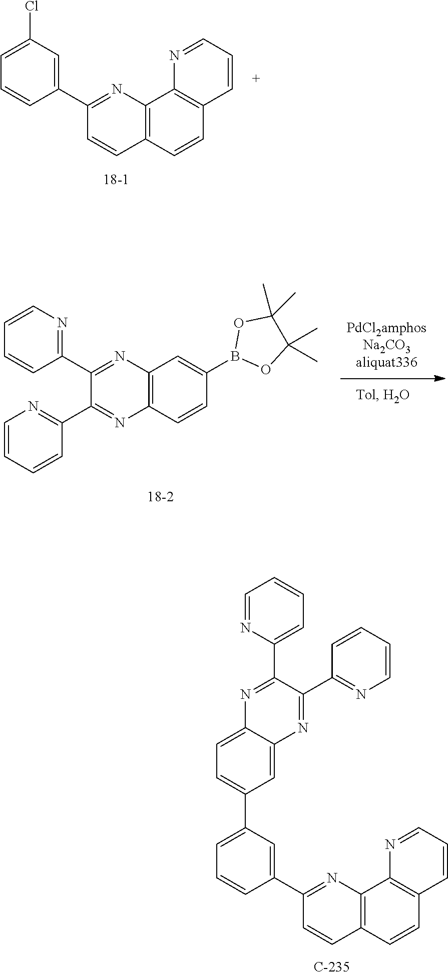 [SMILES: Clc1cccc(-c2ccc3ccc4cccnc4c3n2)c1 ; CC1(C)OB(c2ccc3nc(-c4ccccn4)c(-c4ccccn4)nc3c2)OC1(C)C ; c1ccc(-c2nc3ccc(-c4cccc(-c5ccc6ccc7cccnc7c6n5)c4)cc3nc2-c2ccccn2)nc1]

Compound 18-1 (13.7 g, 33.4 mmol), Compound 18-2 (10.7 g, 36.7 mmol), Pd(amphos)Cl₂ (1.6 g, 2.3 mmol), Aliquat 336 (2.7 g, 6.7 mmol), and Na₂CO₃ (7.1 g, 66.8 mmol) in a flask were dissolved in 167 mL of toluene and 55 mL of distilled water and stirred under reflux at 130° C. for 3 hours. After the reaction was completed, the mixture was cooled to room temperature, the layers were separated, and the resultant was then filtered with Celite. Next, this was filtered with silica and recrystallized to obtain Compound C-235 (4.2 g, yield: 23%).

| | MW | M.P. |
|---|---|---|
| C-235 | 538.61 | 224° C. |

### [Example 19] Synthesis of Compound C-299

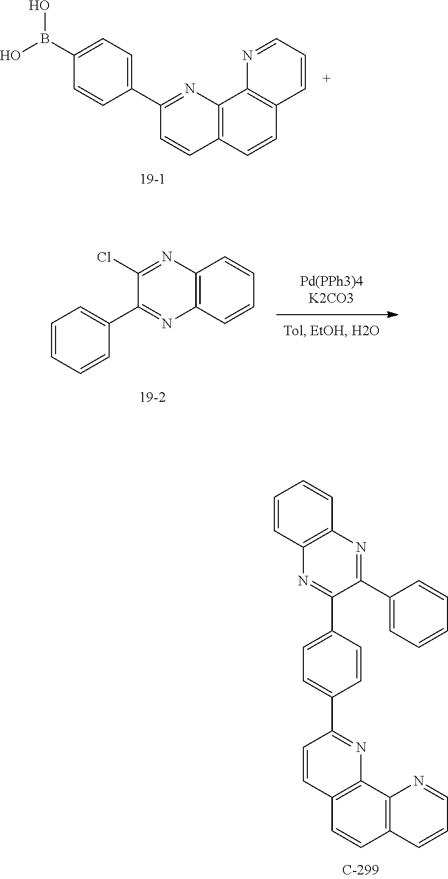 [SMILES: OB(O)c1ccc(-c2ccc3ccc4cccnc4c3n2)cc1 ; Clc1nc2ccccc2nc1-c1ccccc1 ; c1ccc(-c2nc3ccccc3nc2-c2ccc(-c3ccc4ccc5cccnc5c4n3)cc2)cc1]

Compound 19-1 (9.1 g, 30.2 mmol), Compound 19-2 (5.6 g, 23.3 mmol), Pd(PPh₃)₄ (1.3 g, 1.2 mmol), and K2CO₃ (6.4 g, 46.5 mmol) in a flask were dissolved in 110 mL of toluene, 22 mL of ethanol, and 22 mL of distilled water and stirred under reflux at 100° C. for 3 hours. After the reaction was completed, the mixture was cooled to room temperature, the layers were separated, and the resultant was then filtered with Celite. Next, this was filtered with silica and recrystallized to obtain Compound C-299 (9.4 g, yield: 88%).

| | MW | M.P. |
|---|---|---|
| C-299 | 460.5 | 253° C. |

### [Example 20] Synthesis of Compound C-215

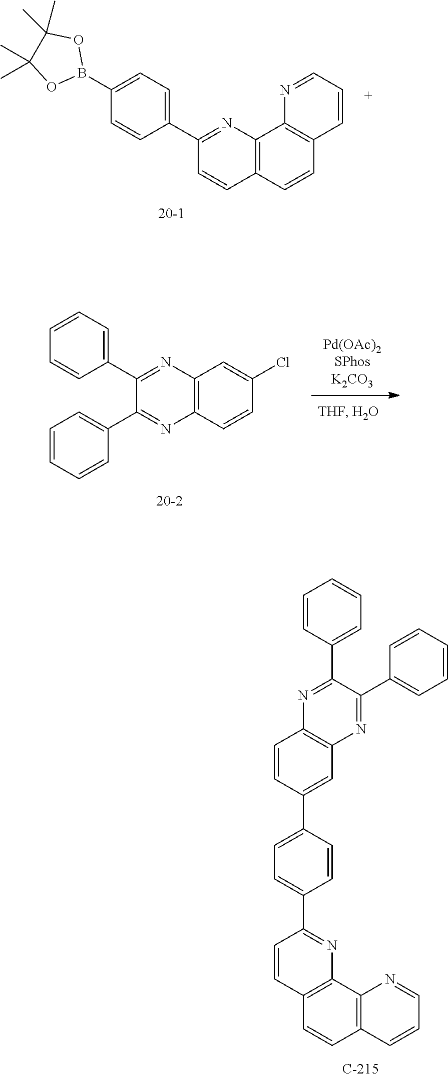 [SMILES: CC1(C)OB(c2ccc(-c3ccc4ccc5cccnc5c4n3)cc2)OC1(C)C ; Clc1ccc2nc(-c3ccccc3)c(-c3ccccc3)nc2c1 ; c1ccc(-c2nc3ccc(-c4ccc(-c5ccc6ccc7cccnc7c6n5)cc4)cc3nc2-c2ccccc2)cc1]

Compound 20-1 (16.3 g, 42.6 mmol), Compound 20-2 (7.5 g, 23.7 mmol), Pd(OAc)₂ (0.3 g, 1.2 mmol), SPhos (0.93 g, 2.4 mmol), and K2CO₃ (6.5 g, 47.3 mmol) in a flask were dissolved in 150 mL of THE and 15 mL of distilled water and stirred under reflux at 65° C. for 15 hours. After the reaction was completed, the mixture was cooled to room temperature, the layers were separated, and the resultant was then filtered with Celite. Next, this was filtered with silica and recrystallized to obtain Compound C-215 (8.5 g, yield: 67%).

| | MW | M.P. |
|---|---|---|
| C-215 | 536.6 | 270° C. |

Hereinafter, the preparation method of an organic electroluminescent device comprising the compound according to the present disclosure and the device properties thereof will be explained for detailed understanding of the present disclosure.

### [Device Examples 1 to 7] Preparation of OLEDs Deposited with a Compound According to the Present Disclosure as an N-Type Charge Generation Layer Material

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and thereafter was stored in isopropyl alcohol and then used. Thereafter, the ITO substrate was mounted on a substrate holder of a vacuum vapor deposition apparatus. Then, Compound HI-1 was introduced into a cell of the vacuum vapor deposition apparatus, and Compound HT-1 was introduced into another cell. The two materials were evaporated at different rates, and Compound HI-1 was deposited in a doping amount of 3 wt % based on the total amount of Compounds HI-1 and HT-1 to form a hole injection layer having a thickness of 5 nm. Next, Compound HT-1 was deposited as a first hole transport layer having a thickness of 30 nm on the hole injection layer. Compound HT-2 was then introduced into another cell of the vacuum vapor deposition apparatus and was evaporated by applying an electric current to the cell, thereby forming a second hole transport layer having a thickness of 5 nm on the first hole transport layer. After forming the hole injection layer and the hole transport layers, a light-emitting layer was formed thereon as follows: Compound H-1 was introduced into a cell of the vacuum vapor deposition apparatus as a host and Compound D-1 was introduced into another cell as a dopant. The two materials were evaporated at different rates, and the dopant was deposited in a doping amount of 2 wt % based on the total amount of the host and dopant to form a first light-emitting layer having a thickness of 20 nm on the second hole transport layer. Next, on the first light-emitting layer, Compound ET-1 was deposited to a thickness of 5 nm as a first hole-blocking layer material. Subsequently, Compound ET-2 was doped to a thickness of 10 nm as an electron transport layer material, thereby forming a first electron transport layer. Thereafter, Li was deposited in an amount of 0.5 wt % in the compound of Table 1 below to form an N-type charge generation layer having a thickness of 4 nm. Subsequently, Compound HI-1 was doped in an amount of 6 wt % based on the total amount of Compound HI-1 and Compound HT-1, thereby forming a P-type charge generation layer having a thickness of 10 nm. Subsequently, Compound HT-1 was deposited to a thickness of 30 nm to form a third hole transport layer, and then Compound HT-2 was deposited to a thickness of 5 nm to form a fourth hole transport layer. Then, a second light-emitting layer was deposited thereon as follows: After placing Compound H-1 as a host was introduced into a cell in vacuum deposition equipment, and Compound D-1 as a dopant was introduced into another cell. The two materials were evaporated at different rates, and the dopant was deposited in an amount of 2 wt % based on the total amount of the host and the dopant, thereby forming a second light-emitting layer having a thickness of 20 nm on the fourth hole transport layer. Compound ET-1, as a second hole-blocking layer material having a thickness of 5 nm, was deposited on the second light-emitting layer, and Compounds ET-3 and EI-1, as second electron transport layer materials, were respectively placed in two cells in a vacuum deposition equipment, and the two materials were deposited at a weight ratio of 2:1 to a thickness of 25 nm. Next, Yb was deposited on the second electron transport layer as an electron injection layer having a thickness of 1 nm, and then an Al cathode having a thickness of 80 nm was deposited on the electron injection layer using further vacuum deposition equipment. Thus, OLEDs were produced. Each of the compounds used for all of the materials were purified by vacuum sublimation at 10⁻⁶ Torr.

### [Device Comparative Example 1] Preparation of an OLED Comprising the Conventional Compound as an N-Type Charge Generation Layer

An OLED was manufactured in the same manner as in Device Example 1, except that the compound described in Table 1 below was used as the N-type charge generation layer material.

The driving voltage and the power efficiency at a luminance of 1,000 nit and the time taken for luminance to decrease from 100% to 95% (lifespan: T₉₅) of the OLEDs of Device Examples 1 to 7 and Device Comparative Example 1 produced as described above were measured, and the results thereof are shown in Table 1 below.

TABLE 1

| | N-type | | | |
|---|---|---|---|---|
| | charge | Driving | Power | |
| | generation | Voltage | Efficiency | Lifespan |
| | layer | [V] | [cd/A] | [T₉₅, hr] |
| Device Example 1 | C-57 | 6.4 | 7.5 | 175.0 |
| Device Example 2 | C-137 | 6.4 | 7.5 | 172.0 |
| Device Example 3 | C-82 | 6.4 | 7.4 | 158.1 |
| Device Example 4 | C-151 | 6.4 | 7.4 | 172.0 |
| Device Example 5 | C-200 | 6.5 | 7.4 | 177.7 |
| Device Example 6 | C-76 | 6.4 | 7.4 | 171.0 |
| Device Example 7 | C-235 | 6.4 | 7.5 | 164.1 |
| Device | CG-0 | 6.6 | 7.4 | 152.0 |
| Comparative | | | | |
| Example 1 | | | | |

### [Device Examples 8 to 13] Preparation of OLEDs Deposited with a Compound According to the Present Disclosure as an N-Type Charge Generation Layer Material

OLEDs were manufactured in the same manner as Device Example 1, except that Yb was deposited in an amount of 2 wt % in the N-type charge generation layer material as in Table 2 below to form an N-type charge generation layer having a thickness of 8 nm, and the thickness of the P-type charge generation layer was changed to 5 nm.

### [Device Comparative Example 2] Preparation of an OLED Containing the Conventional Compound as an N-Type Charge Generation Layer

An OLED was manufactured in the same manner as Device Example 8, except that the compound described in Table 2 below was used as N-type charge generation layer materials.

The driving voltage and the power efficiency at a luminance of 1,000 nit and the time taken for luminance to decrease from 100% to 95% at a luminance of 1,000 nit (lifespan: T₉₅) of the OLEDs of Device Examples 8 to 13 and Device Comparative Example 2 produced as described above were measured, and the results thereof are shown in Table 2 below.

TABLE 2

| | N-type charge | Driving | Power | |
|---|---|---|---|---|
| | generation | Voltage | Efficiency | Lifespan |
| | layer | [V] | [cd/A] | [T₉₅, hr] |
| Device Example 8 | C-3 | 7.0 | 7.5 | 473.1 |
| Device Example 9 | C-5 | 6.7 | 7.7 | 361.6 |
| Device Example 10 | C-57 | 6.6 | 7.7 | 415.8 |
| Device Example 11 | C-1 | 7.0 | 7.5 | 404.4 |
| Device Example 12 | C-4 | 6.6 | 7.3 | 408.3 |
| Device Example 13 | C-6 | 7.0 | 7.5 | 349.6 |
| Device Comparative | CG-0 | 8.1 | 7.1 | 348.1 |
| Example 2 | | | | |

From the results in Tables 1 and 2 above, it can be confirmed that an organic electroluminescent device comprising the compound according to the present disclosure in an N-type charge generation layer exhibits low driving voltage and/or high power efficiency and/or significantly improved long-lifespan characteristics.

The compounds used in the Device Examples and Device Comparative Examples are specifically shown in Table 3 below.

TABLE 3

| | |
|---|---|
| Hole Injection Layer/Hole Transport Layer | 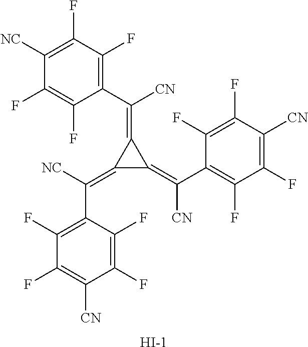 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 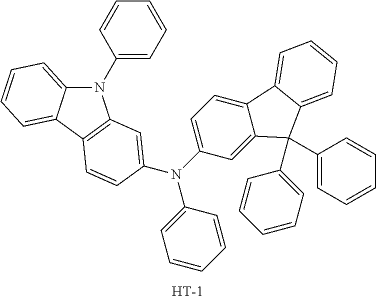 |
| | 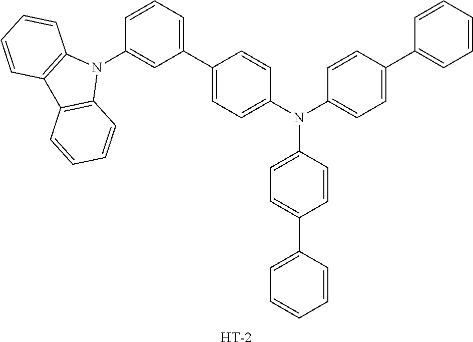 |
| Light- Emitting Layer | 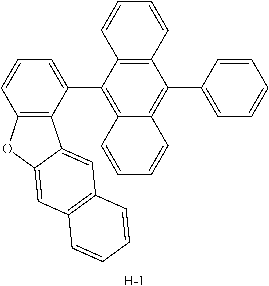 |
| | 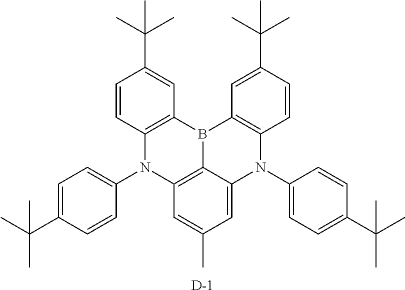 |
| Electron Transport Layer/ Electron Injection Layer | 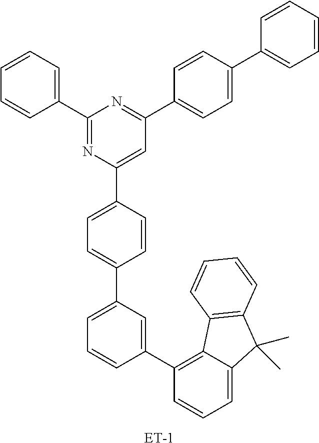 |
| | 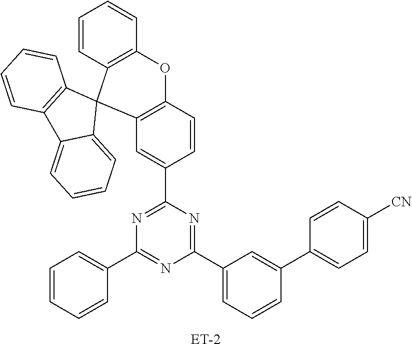 |
| | 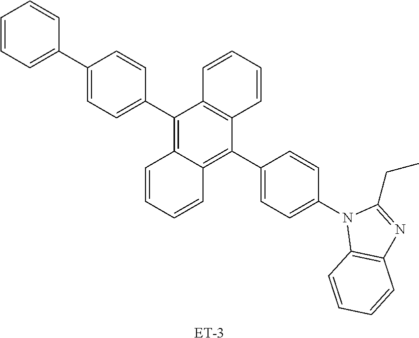 [SMILES: CCc1nc2ccccc2n1-c1ccc(-c2c3ccccc3c(-c3ccc(-c4ccccc4)cc3)c3ccccc23)cc1 ; "ET-3"] |
| | 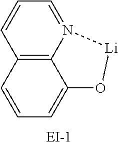 |
| N-type Charge Generation Layer | 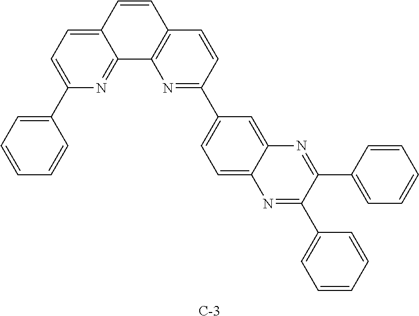 |
| | 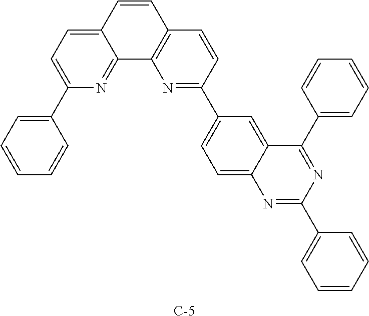 |
| | 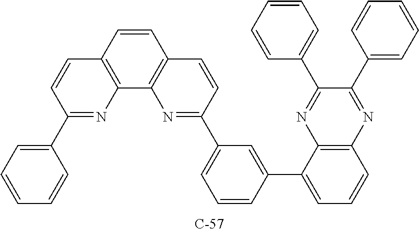 |
| | 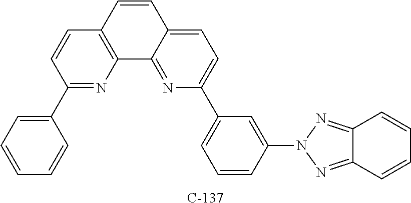 |
| | 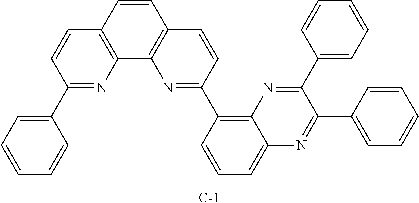 |
| | 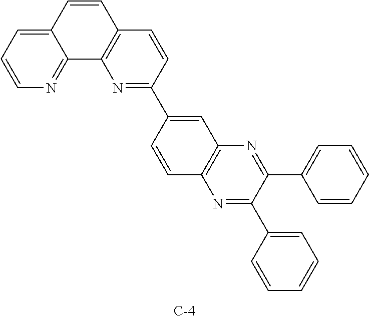 |
| | 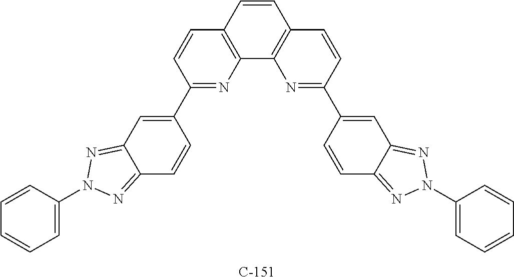 [SMILES: c1ccc(-n2nc3ccc(-c4ccc5ccc6ccc(-c7ccc8nn(-c9ccccc9)nc8c7)nc6c5n4)cc3n2)cc1 ; "C-151"] |
| | 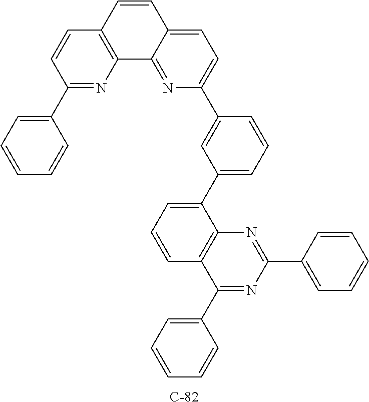 |
| | 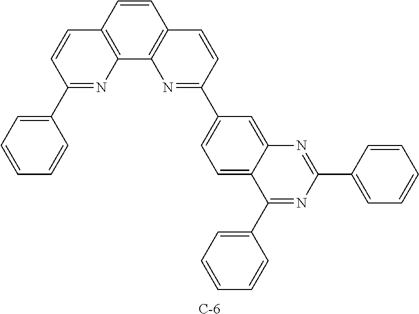 |
| | 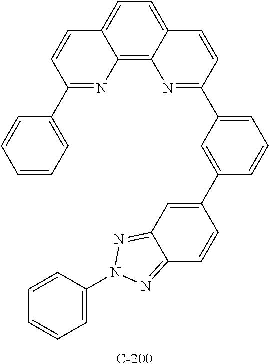 [SMILES: c1ccc(-c2ccc3ccc4ccc(-c5cccc(-c6ccc7nn(-c8ccccc8)nc7c6)c5)nc4c3n2)cc1 ; "C-200"] |
| | 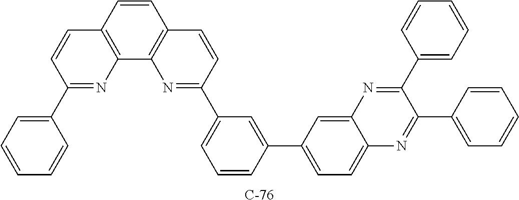 |
| | 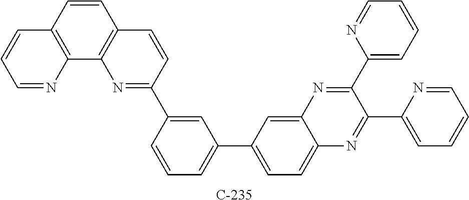 |
| | 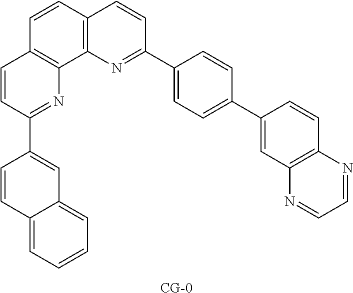 [SMILES: c1ccc2cc(-c3ccc4ccc5ccc(-c6ccc(-c7ccc8nccnc8c7)cc6)nc5c4n3)ccc2c1 ; "CG-0"] |

### [Device Examples 14 to 21] Preparation of OLEDs Deposited with a Compound According to the Present Disclosure as an N-Type Charge Generation Layer

OLEDs were manufactured in the same manner as in Device Example 1, except that the thickness of the first electron transport layer was changed to 12 nm, Yb was deposited in an amount of 2 wt % as the N-type charge generation layer material in the compound shown in Table 4 below to form an N-type charge generation layer having a thickness of 9 nm, and the thickness of the P-type charge generation layer was changed to 6 nm.

### [Device Comparative Example 3] Preparation of an OLED Containing the Conventional Compound as an N-Type Charge Generation Layer

An OLED was manufactured in the same manner as Device Example 14, except that the compound described in Table 4 below was used as the N-type charge generation layer material.

The driving voltage, power efficiency, and progressive driving voltage change (ΔV) at a luminance of 1,000 nit of the OLEDs of Device Examples 14 to 21 and Device Comparative Example 3 produced as described above were measured, and the results thereof are shown in Table 4 below.

TABLE 4

| | | | | Progressive |
|---|---|---|---|---|
| | N-type charge | Driving | Power | Driving Voltage |
| | generation | Voltage | Efficiency | ΔV |
| | layer | [V] | [cd/A] | [%] |
| Device | C-12 | 7.0 | 7.6 | 102.4 |
| Example 14 | | | | |
| Device | C-213 | 6.5 | 7.9 | 102.0 |
| Example 15 | | | | |
| Device | C-170 | 6.5 | 7.9 | 102.5 |
| Example 16 | | | | |
| Device | C-249 | 7.9 | 7.7 | 101.6 |
| Example 17 | | | | |
| Device | C-299 | 6.8 | 7.5 | 102.3 |
| Example 18 | | | | |
| Device | C-215 | 6.8 | 7.5 | 102.6 |
| Example 19 | | | | |
| Device | C-300 | 6.8 | 7.5 | 102.0 |
| Example 20 | | | | |
| Device | C-302 | 6.7 | 7.6 | 102.6 |
| Example21 | | | | |
| Device | CG-0 | 8.0 | 7.4 | 102.6 |
| Comparative | | | | |
| Example 3 | | | | |

From the results in Table 4 above, it can be confirmed that an organic electroluminescent device including a compound according to the present disclosure in an N-type charge generation layer exhibits low driving voltage and/or high power efficiency and/or low driving voltage change.

The compounds used in the Device Examples and Device Comparative Examples are specifically shown in Table 5 below.

TABLE 5

| | |
|---|---|
| Hole Injection Layer/Hole Transport Layer | 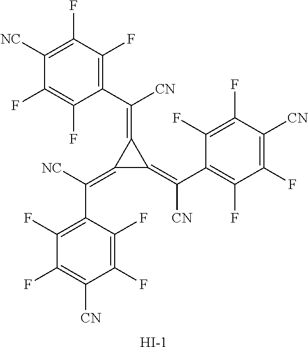 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 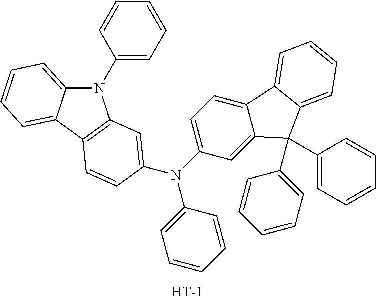 |
| | 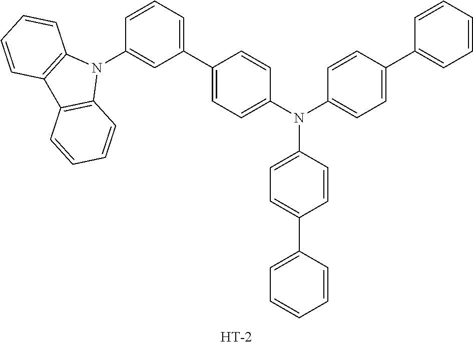 |
| Light- Emitting Layer | 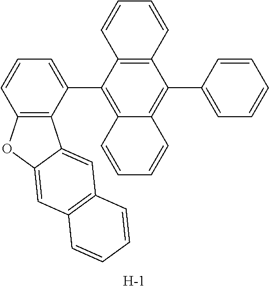 |
| | 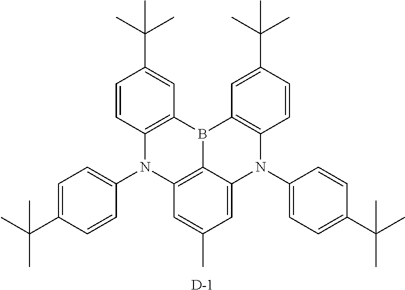 |
| Electron Transport Layer/Hole- Blocking Layer/ Electron Injection Layer | 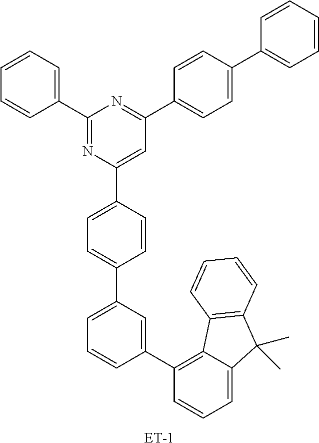 |
| | 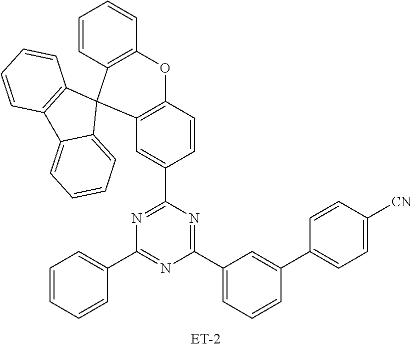 |
| | 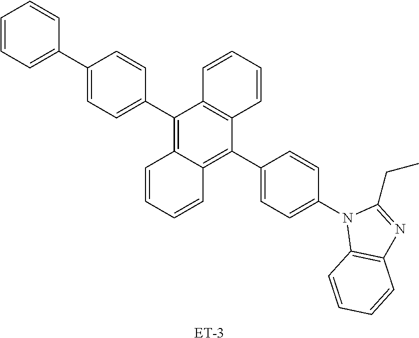 [SMILES: CCc1nc2ccccc2n1-c1ccc(-c2c3ccccc3c(-c3ccc(-c4ccccc4)cc3)c3ccccc23)cc1 ; "ET-3"] |
| | 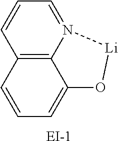 |
| N-type Charge Generation Layer/ | 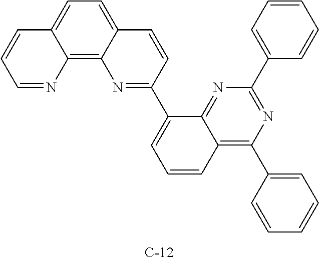 |
| | 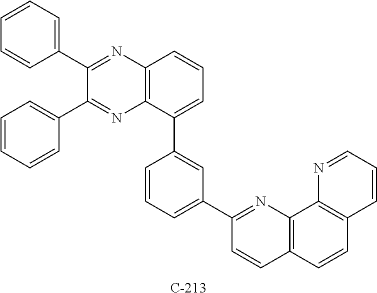 [SMILES: c1ccc(-c2nc3cccc(-c4cccc(-c5ccc6ccc7cccnc7c6n5)c4)c3nc2-c2ccccc2)cc1 ; "C-213"] |
| | 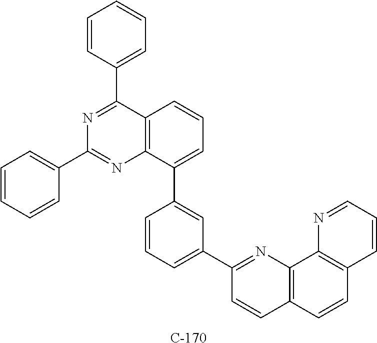 [SMILES: c1ccc(-c2nc(-c3ccccc3)c3cccc(-c4cccc(-c5ccc6ccc7cccnc7c6n5)c4)c3n2)cc1 ; "C-170"] |
| | 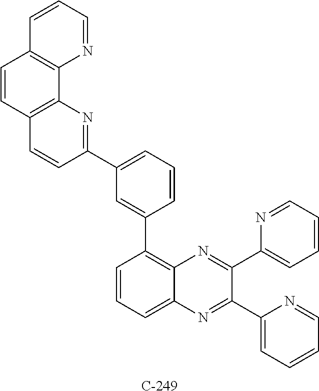 [SMILES: c1ccc(-c2nc3cccc(-c4cccc(-c5ccc6ccc7cccnc7c6n5)c4)c3nc2-c2ccccn2)nc1 ; "C-249"] |
| | 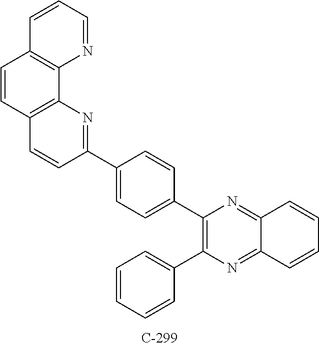 |
| | 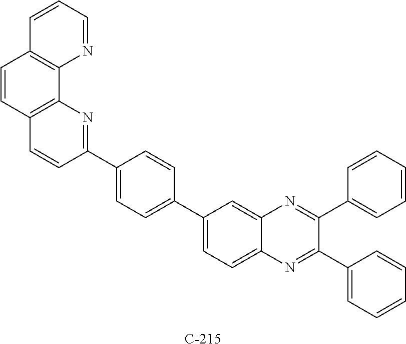 [SMILES: c1ccc(-c2nc3ccc(-c4ccc(-c5ccc6ccc7cccnc7c6n5)cc4)cc3nc2-c2ccccc2)cc1 ; "C-215"] |
| | 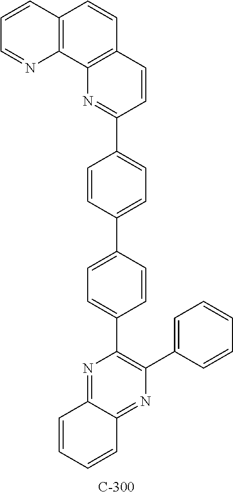 |
| | 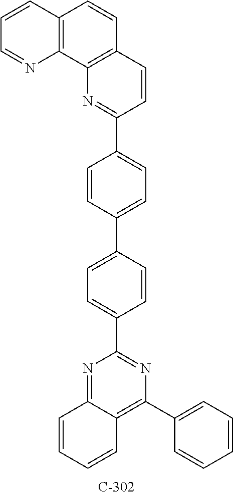 |
| | 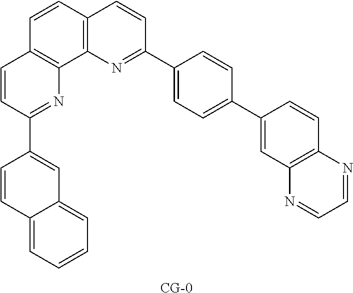 [SMILES: c1ccc2cc(-c3ccc4ccc5ccc(-c6ccc(-c7ccc8nccnc8c7)cc6)nc5c4n3)ccc2c1 ; "CG-0"] |

1. A compound represented by the following Formula 1: 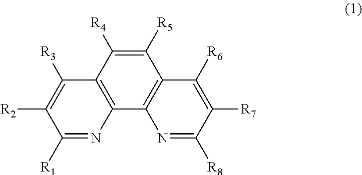
wherein,
R₁ represents hydrogen, deuterium, halogen, a substituted or unsubstituted phenyl, a substituted or unsubstituted biphenyl, a substituted or unsubstituted terphenyl, a substituted or unsubstituted (C₁-C₃₀)alkyl, or a substituted or unsubstituted (3- to 30-membered)heteroaryl;
R₂ to R₈ each independently represent hydrogen, deuterium, halogen, cyano, a substituted or unsubstituted (C₁-C₃₀)alkyl, a substituted or unsubstituted (C₂-C₃₀)alkenyl, a substituted or unsubstituted (C₆-C₃₀)aryl, a substituted or unsubstituted (3- to 30-membered)heteroaryl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, a substituted or unsubstituted (C₁-C₃₀)alkoxy, a substituted or unsubstituted tri(C₁-C₃₀)alkylsilyl, a substituted or unsubstituted di(C₁-C₃₀)alkyl(C₆-C₃₀)arylsilyl, a substituted or unsubstituted (C₁-C₃₀)alkyldi(C₆-C₃₀)arylsilyl, a substituted or unsubstituted tri(C₆-C₃₀)arylsilyl, a substituted or unsubstituted fused ring of (C₃-C₃₀)aliphatic ring and (C₆-C₃₀)aromatic ring or -L-HAr; or may be linked to the adjacent substituents to form a ring(s);
provided that at least one of R₂ to R₈ is -L-HAr;
L represents a single bond, a substituted or unsubstituted (C₆-C₃₀)arylene, or a substituted or unsubstituted (3- to 30-membered)heteroarylene; and
HAr represents a substituted or unsubstituted (3- to 30-membered)heteroaryl containing at least one nitrogen atom;
provided that when R₄ or R₅ is -L-HAr, the compounds in which HAr is quinazoline are excluded.

2. The compound according to claim 1, wherein the substituted alkyl, the substituted alkenyl, the substituted aryl(ene), the substituted heteroaryl(ene), the substituted cycloalkyl, the substituted cycloalkenyl, the substituted heterocycloalkyl, the substituted alkoxy, the substituted trialkylsilyl, the substituted dialkylarylsilyl, the substituted alkyldiarylsilyl, the substituted triarylsilyl, and the substituted fused ring of (C₃-C₃₀) aliphatic ring and (C₆-C₃₀) aromatic ring each independently are substituted with at least one selected from the group consisting of deuterium, halogen, cyano, carboxyl, nitro, hydroxyl, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₃-C₃₀)aryloxy, (C₆-C₃₀)arylthio, (5- to 30-membered)heteroaryl unsubstituted or substituted with (C₆-C₃₀)aryl, (C₆-C₃₀)aryl unsubstituted or substituted with (5- to 30-membered)heteroaryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, a fused ring of (C₃-C₃₀)aliphatic ring and (C₃-C₃₀)aromatic ring, amino, mono- or di(C₁-C₃₀)alkylamino, mono- or di(C₆-C₃₀)arylamino, (C₁-C₃₀)alkyl(C₆-C₃₀)arylamino, mono- or di(3- to 30-membered)heteroarylamino, (C₁-C₃₀)alkyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₃-C₃₀)arylcarbonyl, (C₃-C₃₀)arylphosphinyl, di(C₆-C₃₀)arylboronyl, di(C₁-C₃₀)alkylboronyl, (C₁-C₃₀)alkyl(C₆-C₃₀)arylboronyl, (C₆-C₃₀)ar(C₁-C₃₀)alkyl, and (C₆-C₃₀)alkyl(C₆-C₃₀)aryl.

3. The compound according to claim 1, wherein Formula 1 is represented by any one of the following Formulas 1-1 to 1-7: 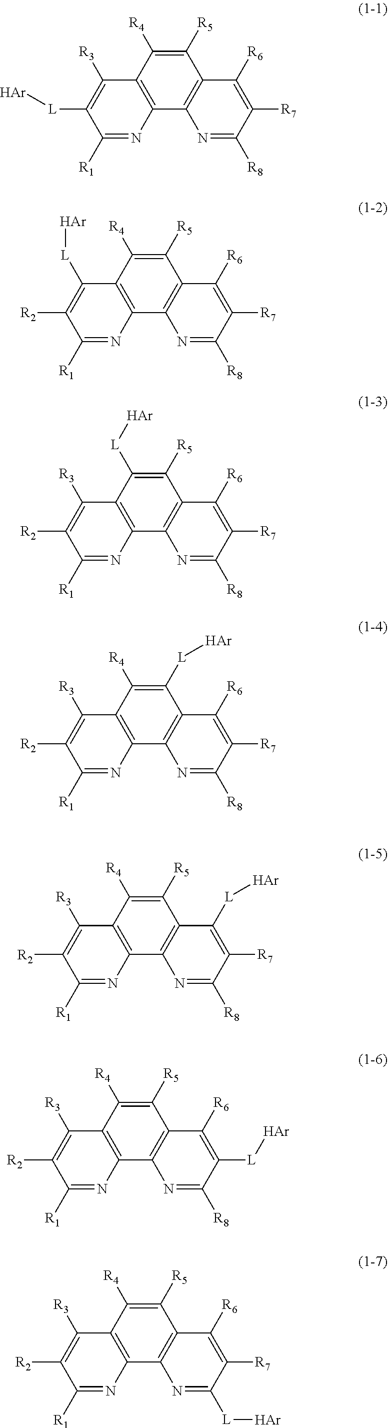
wherein,
R₁, L, and HAr are as defined in claim 1; and
R₂ to R₈ each independently represent hydrogen, deuterium, a substituted or unsubstituted (C₆-C₃₀)aryl, or a substituted or unsubstituted (3- to 30-membered)heteroaryl.

4. The compound according to claim 1, wherein HAr is represented by the following Formula 1-a or 1-b: 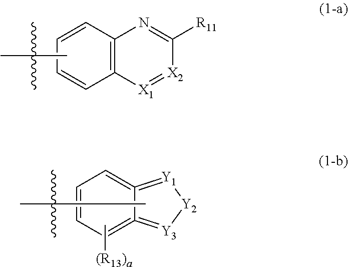
wherein, 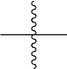
represents a site connected to L;
X₁ and X₂ each independently represent N or CR₁₂;
Y₁ to Y₃ each independently represent N or NR₁₄;
R₁₁ and R₁₂ each independently represent hydrogen, deuterium, a substituted or unsubstituted (C₆-C₃₀)aryl, or a substituted or unsubstituted (3- to 30-membered)heteroaryl;
R₁₃ and R₁₄ each independently represent hydrogen, deuterium, a substituted or unsubstituted (C₆-C₃₀)aryl, a substituted or unsubstituted (3- to 30-membered)heteroaryl, or a site directly connected to L; and
a represents an integer of 1 to 4, wherein when a is 2 or more, each of R₁₃ may be the same as or different from each other.

5. The compound according to claim 1, wherein the compound represented by Formula 1 is selected from the following compounds: 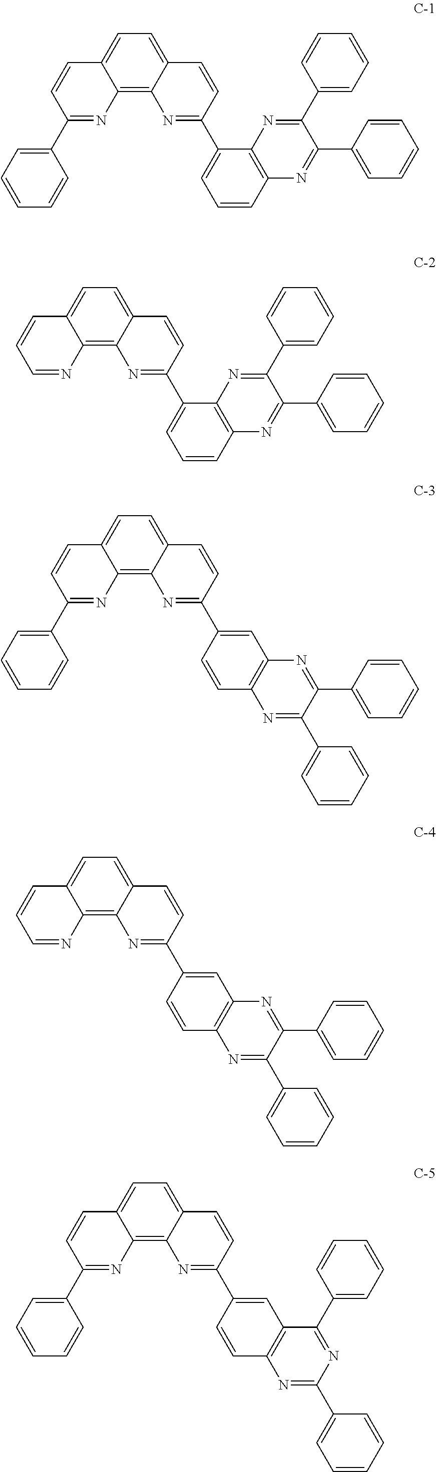 [SMILES: c1ccc(-c2ccc3ccc4ccc(-c5cccc6nc(-c7ccccc7)c(-c7ccccc7)nc56)nc4c3n2)cc1 ; c1ccc(-c2nc3cccc(-c4ccc5ccc6cccnc6c5n4)c3nc2-c2ccccc2)cc1 ; c1ccc(-c2ccc3ccc4ccc(-c5ccc6nc(-c7ccccc7)c(-c7ccccc7)nc6c5)nc4c3n2)cc1 ; c1ccc(-c2nc3ccc(-c4ccc5ccc6cccnc6c5n4)cc3nc2-c2ccccc2)cc1 ; c1ccc(-c2ccc3ccc4ccc(-c5ccc6nc(-c7ccccc7)nc(-c7ccccc7)c6c5)nc4c3n2)cc1] 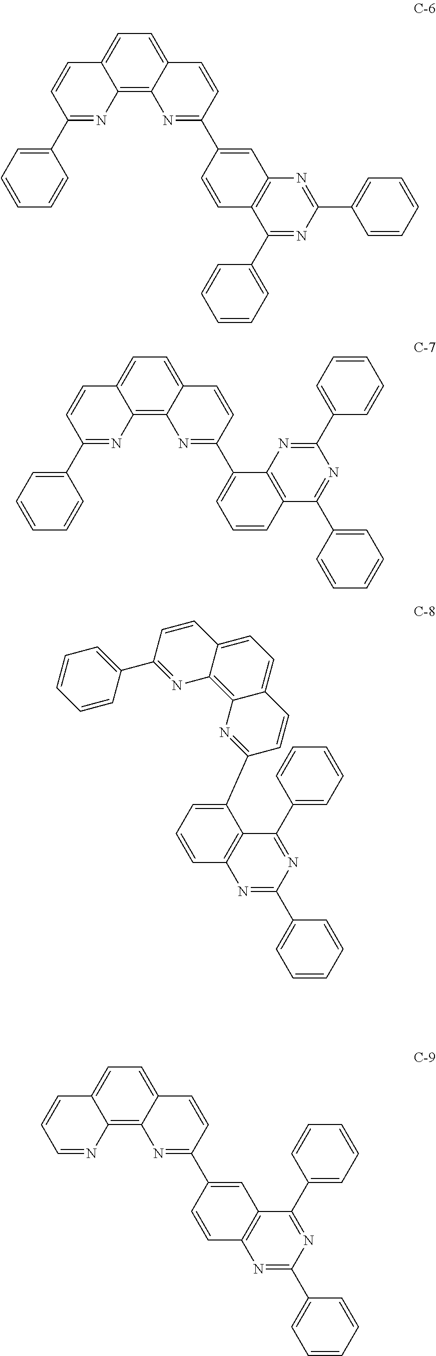 [SMILES: c1ccc(-c2ccc3ccc4ccc(-c5ccc6c(-c7ccccc7)nc(-c7ccccc7)nc6c5)nc4c3n2)cc1 ; c1ccc(-c2ccc3ccc4ccc(-c5cccc6c(-c7ccccc7)nc(-c7ccccc7)nc56)nc4c3n2)cc1 ; c1ccc(-c2ccc3ccc4ccc(-c5cccc6nc(-c7ccccc7)nc(-c7ccccc7)c56)nc4c3n2)cc1 ; c1ccc(-c2nc(-c3ccccc3)c3cc(-c4ccc5ccc6cccnc6c5n4)ccc3n2)cc1] 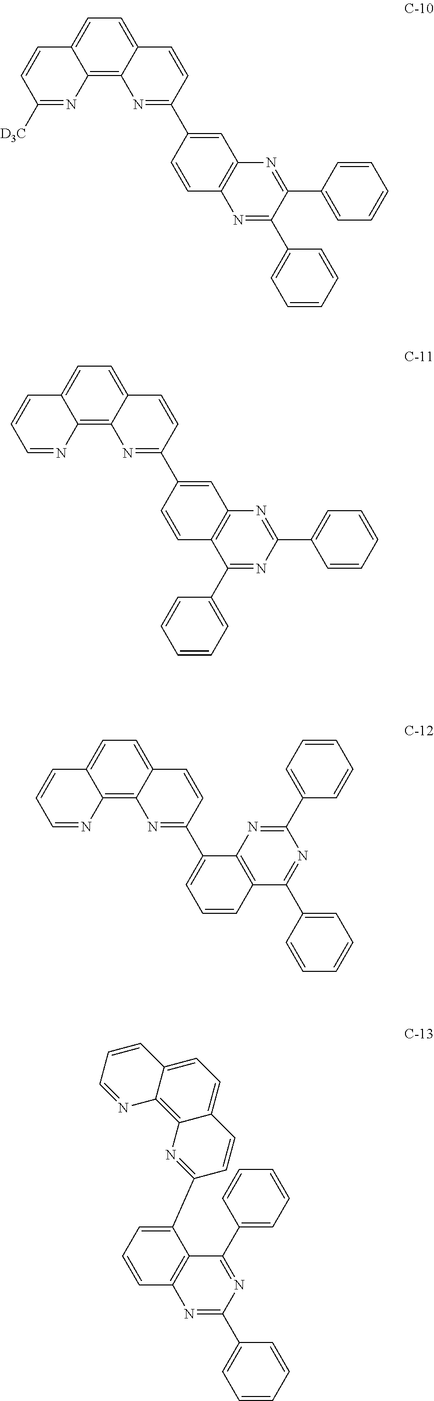 [SMILES: [2H]C([2H])([2H])c1ccc2ccc3ccc(-c4ccc5nc(-c6ccccc6)c(-c6ccccc6)nc5c4)nc3c2n1 ; c1ccc(-c2nc(-c3ccccc3)c3ccc(-c4ccc5ccc6cccnc6c5n4)cc3n2)cc1 ; c1ccc(-c2nc(-c3ccccc3)c3cccc(-c4ccc5ccc6cccnc6c5n4)c3n2)cc1 ; c1ccc(-c2nc(-c3ccccc3)c3c(-c4ccc5ccc6cccnc6c5n4)cccc3n2)cc1] 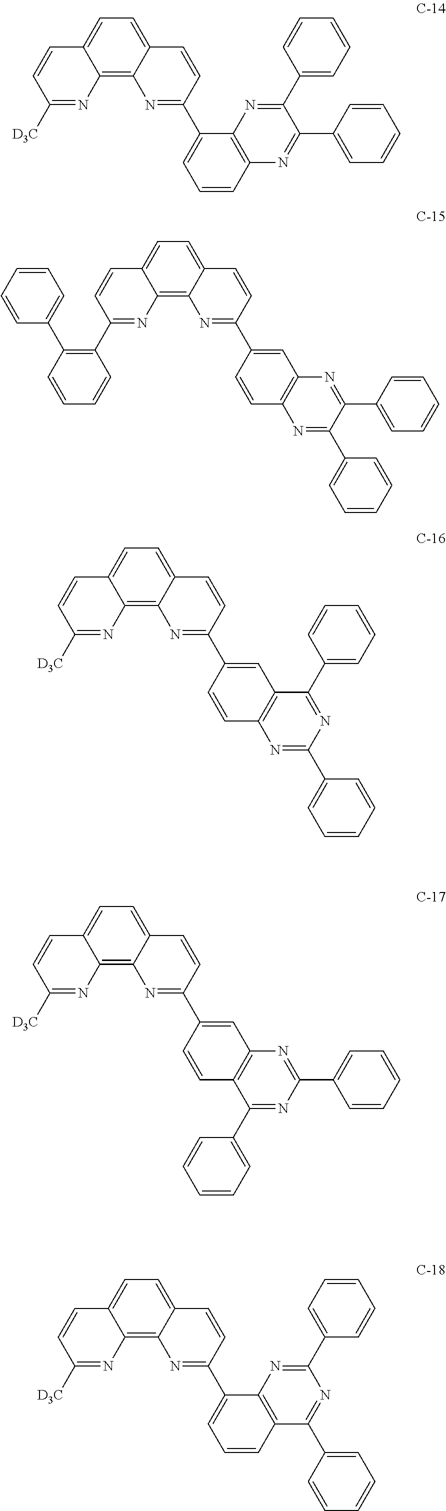 [SMILES: [2H]C([2H])([2H])c1ccc2ccc3ccc(-c4cccc5nc(-c6ccccc6)c(-c6ccccc6)nc45)nc3c2n1 ; c1ccc(-c2ccccc2-c2ccc3ccc4ccc(-c5ccc6nc(-c7ccccc7)c(-c7ccccc7)nc6c5)nc4c3n2)cc1 ; [2H]C([2H])([2H])c1ccc2ccc3ccc(-c4ccc5nc(-c6ccccc6)nc(-c6ccccc6)c5c4)nc3c2n1 ; [2H]C([2H])([2H])c1ccc2ccc3ccc(-c4ccc5c(-c6ccccc6)nc(-c6ccccc6)nc5c4)nc3c2n1 ; [2H]C([2H])([2H])c1ccc2ccc3ccc(-c4cccc5c(-c6ccccc6)nc(-c6ccccc6)nc45)nc3c2n1] 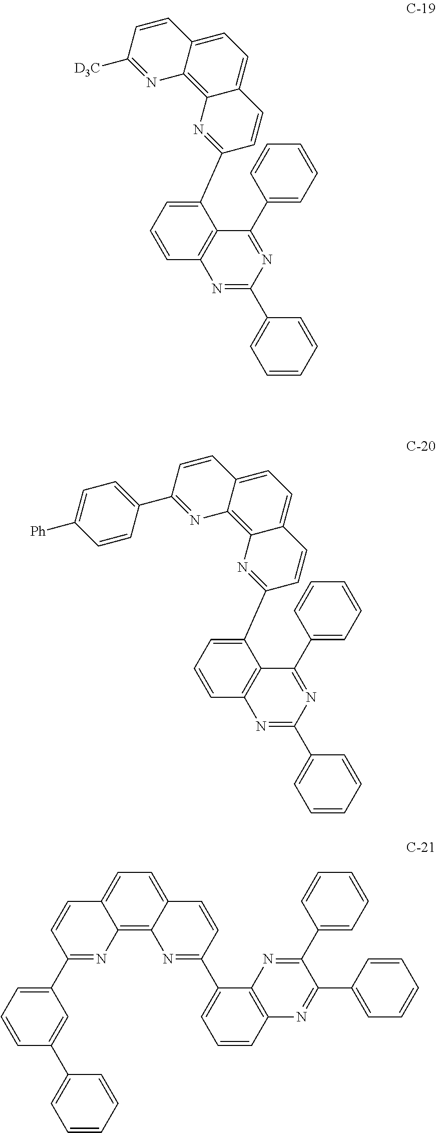 [SMILES: [2H]C([2H])([2H])c1ccc2ccc3ccc(-c4cccc5nc(-c6ccccc6)nc(-c6ccccc6)c45)nc3c2n1 ; c1ccc(-c2ccc(-c3ccc4ccc5ccc(-c6cccc7nc(-c8ccccc8)nc(-c8ccccc8)c67)nc5c4n3)cc2)cc1 ; c1ccc(-c2cccc(-c3ccc4ccc5ccc(-c6cccc7nc(-c8ccccc8)c(-c8ccccc8)nc67)nc5c4n3)c2)cc1] 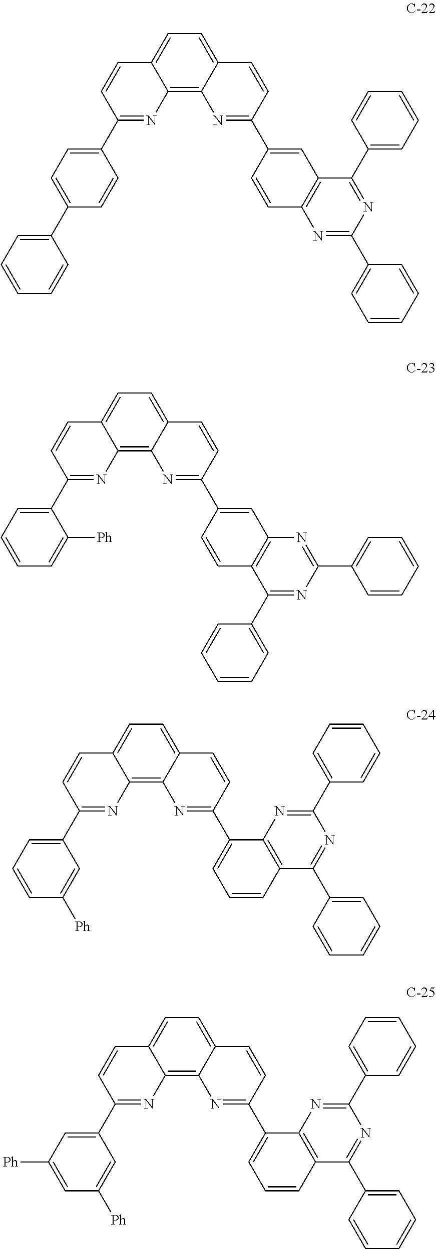 [SMILES: c1ccc(-c2ccc(-c3ccc4ccc5ccc(-c6ccc7nc(-c8ccccc8)nc(-c8ccccc8)c7c6)nc5c4n3)cc2)cc1 ; c1ccc(-c2nc(-c3ccccc3)c3ccc(-c4ccc5ccc6ccc(-c7ccccc7-c7ccccc7)nc6c5n4)cc3n2)cc1 ; c1ccc(-c2cccc(-c3ccc4ccc5ccc(-c6cccc7c(-c8ccccc8)nc(-c8ccccc8)nc67)nc5c4n3)c2)cc1 ; c1ccc(-c2cc(-c3ccccc3)cc(-c3ccc4ccc5ccc(-c6cccc7c(-c8ccccc8)nc(-c8ccccc8)nc67)nc5c4n3)c2)cc1] 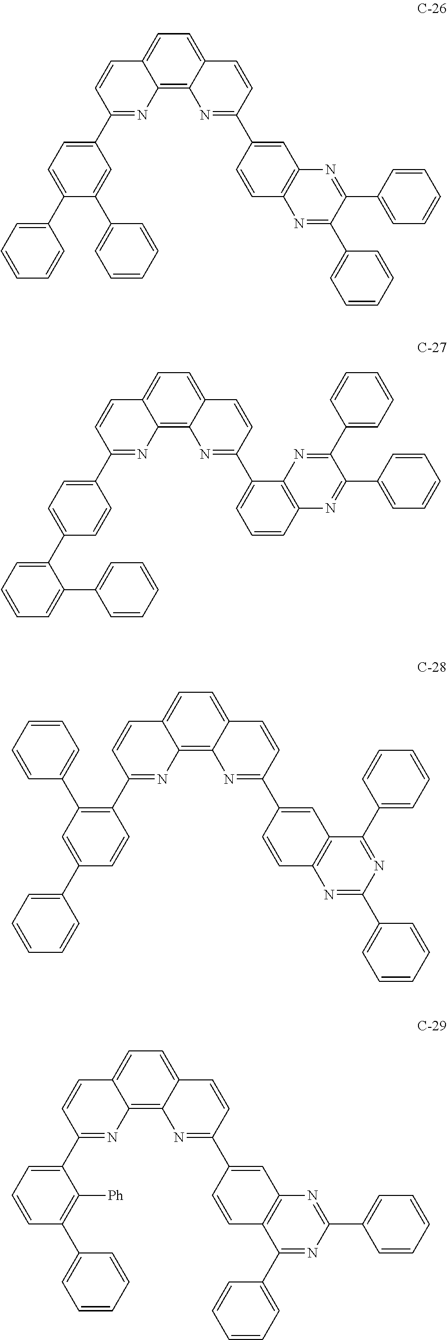 [SMILES: c1ccc(-c2ccc(-c3ccc4ccc5ccc(-c6ccc7nc(-c8ccccc8)c(-c8ccccc8)nc7c6)nc5c4n3)cc2-c2ccccc2)cc1 ; c1ccc(-c2ccccc2-c2ccc(-c3ccc4ccc5ccc(-c6cccc7nc(-c8ccccc8)c(-c8ccccc8)nc67)nc5c4n3)cc2)cc1 ; c1ccc(-c2ccc(-c3ccc4ccc5ccc(-c6ccc7nc(-c8ccccc8)nc(-c8ccccc8)c7c6)nc5c4n3)c(-c3ccccc3)c2)cc1 ; c1ccc(-c2nc(-c3ccccc3)c3ccc(-c4ccc5ccc6ccc(-c7cccc(-c8ccccc8)c7-c7ccccc7)nc6c5n4)cc3n2)cc1] 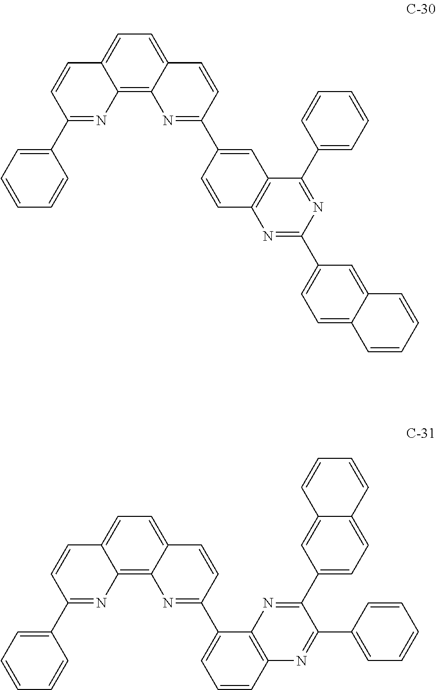 [SMILES: c1ccc(-c2ccc3ccc4ccc(-c5ccc6nc(-c7ccc8ccccc8c7)nc(-c7ccccc7)c6c5)nc4c3n2)cc1 ; c1ccc(-c2ccc3ccc4ccc(-c5cccc6nc(-c7ccccc7)c(-c7ccc8ccccc8c7)nc56)nc4c3n2)cc1] 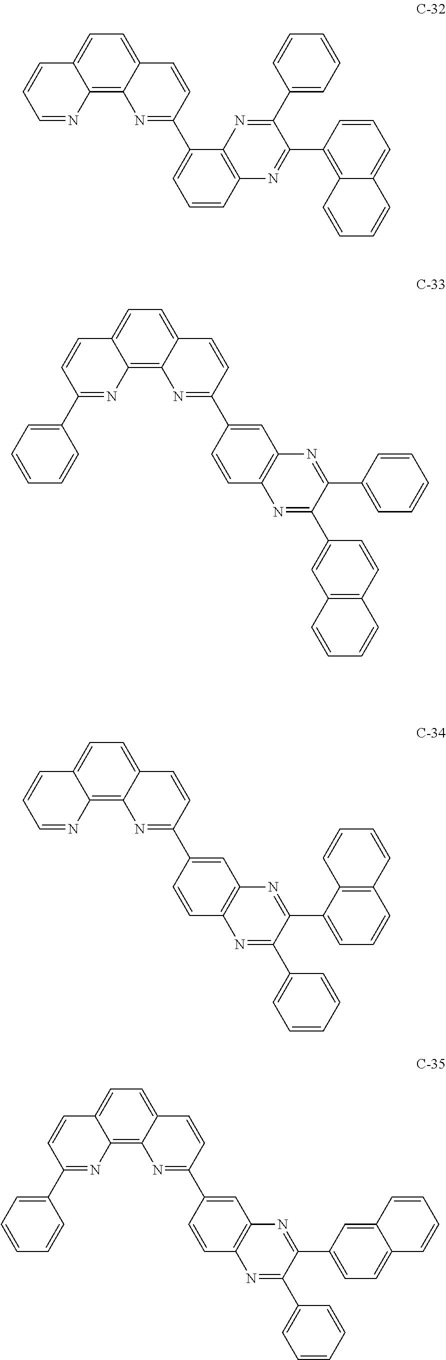 [SMILES: c1ccc(-c2nc3c(-c4ccc5ccc6cccnc6c5n4)cccc3nc2-c2cccc3ccccc23)cc1 ; c1ccc(-c2ccc3ccc4ccc(-c5ccc6nc(-c7ccc8ccccc8c7)c(-c7ccccc7)nc6c5)nc4c3n2)cc1 ; c1ccc(-c2nc3ccc(-c4ccc5ccc6cccnc6c5n4)cc3nc2-c2cccc3ccccc23)cc1 ; c1ccc(-c2ccc3ccc4ccc(-c5ccc6nc(-c7ccccc7)c(-c7ccc8ccccc8c7)nc6c5)nc4c3n2)cc1] 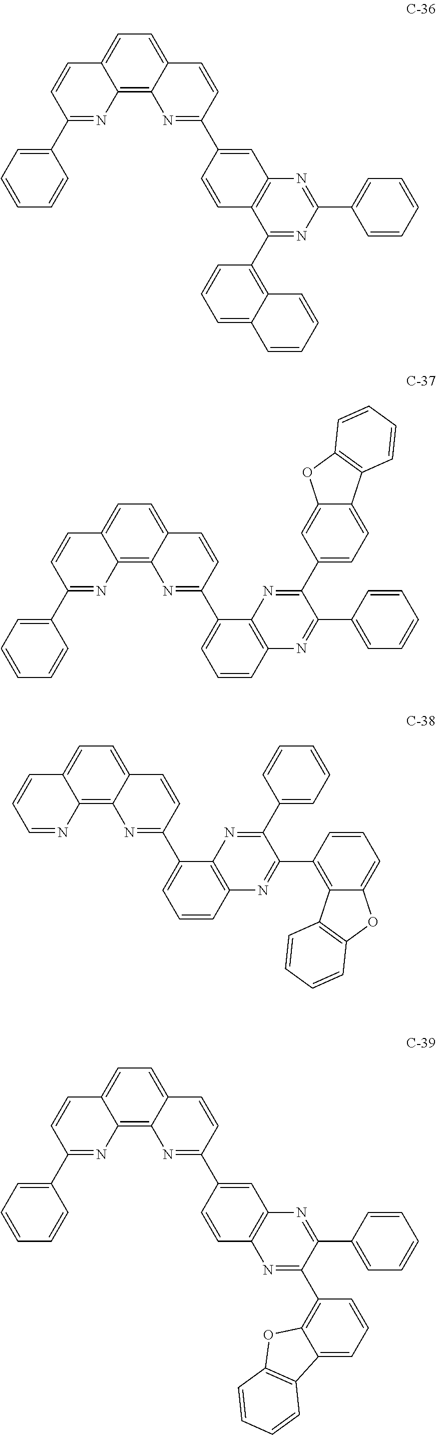 [SMILES: c1ccc(-c2ccc3ccc4ccc(-c5ccc6c(-c7cccc8ccccc78)nc(-c7ccccc7)nc6c5)nc4c3n2)cc1 ; c1ccc(-c2ccc3ccc4ccc(-c5cccc6nc(-c7ccccc7)c(-c7ccc8c(c7)oc7ccccc78)nc56)nc4c3n2)cc1 ; c1ccc(-c2nc3c(-c4ccc5ccc6cccnc6c5n4)cccc3nc2-c2cccc3oc4ccccc4c23)cc1 ; c1ccc(-c2ccc3ccc4ccc(-c5ccc6nc(-c7cccc8c7oc7ccccc78)c(-c7ccccc7)nc6c5)nc4c3n2)cc1] 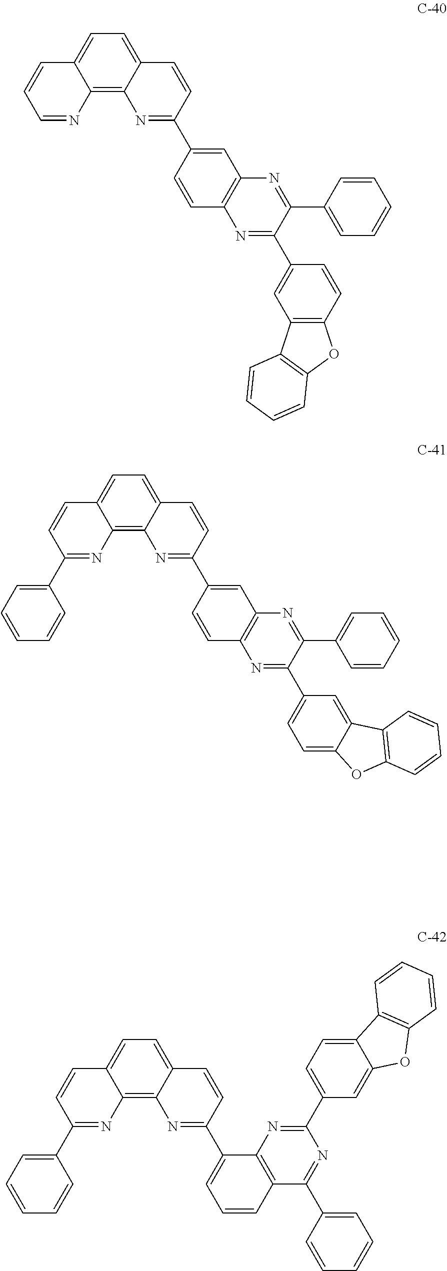 [SMILES: c1ccc(-c2nc3cc(-c4ccc5ccc6cccnc6c5n4)ccc3nc2-c2ccc3oc4ccccc4c3c2)cc1 ; c1ccc(-c2ccc3ccc4ccc(-c5ccc6nc(-c7ccc8oc9ccccc9c8c7)c(-c7ccccc7)nc6c5)nc4c3n2)cc1 ; c1ccc(-c2ccc3ccc4ccc(-c5cccc6c(-c7ccccc7)nc(-c7ccc8c(c7)oc7ccccc78)nc56)nc4c3n2)cc1] 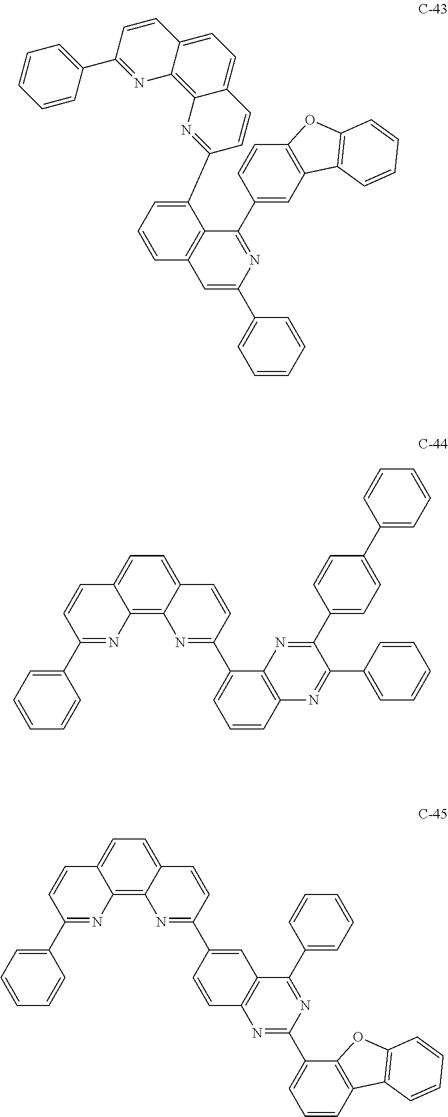 [SMILES: c1ccc(-c2cc3cccc(-c4ccc5ccc6ccc(-c7ccccc7)nc6c5n4)c3c(-c3ccc4oc5ccccc5c4c3)n2)cc1 ; c1ccc(-c2ccc(-c3nc4c(-c5ccc6ccc7ccc(-c8ccccc8)nc7c6n5)cccc4nc3-c3ccccc3)cc2)cc1 ; c1ccc(-c2ccc3ccc4ccc(-c5ccc6nc(-c7cccc8c7oc7ccccc78)nc(-c7ccccc7)c6c5)nc4c3n2)cc1] 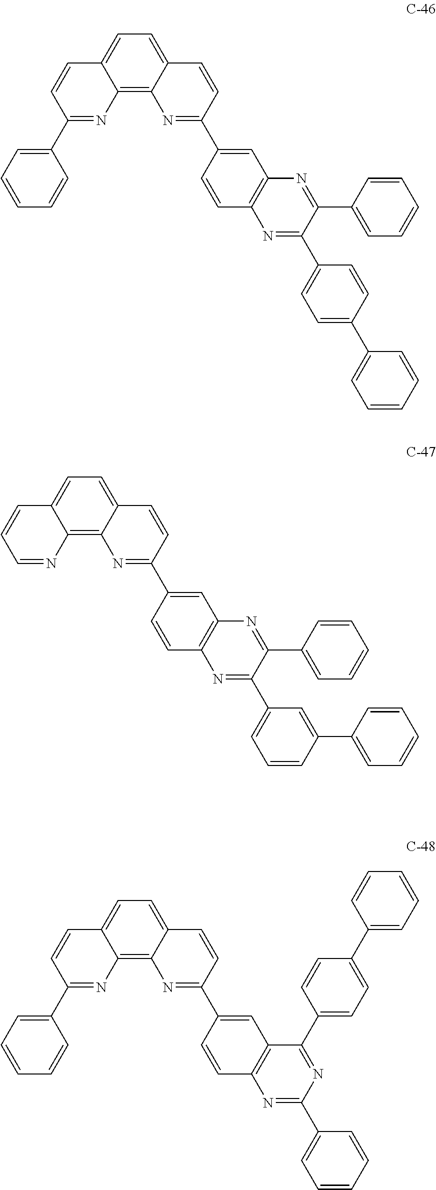 [SMILES: c1ccc(-c2ccc(-c3nc4ccc(-c5ccc6ccc7ccc(-c8ccccc8)nc7c6n5)cc4nc3-c3ccccc3)cc2)cc1 ; c1ccc(-c2cccc(-c3nc4ccc(-c5ccc6ccc7cccnc7c6n5)cc4nc3-c3ccccc3)c2)cc1 ; c1ccc(-c2ccc(-c3nc(-c4ccccc4)nc4ccc(-c5ccc6ccc7ccc(-c8ccccc8)nc7c6n5)cc34)cc2)cc1] 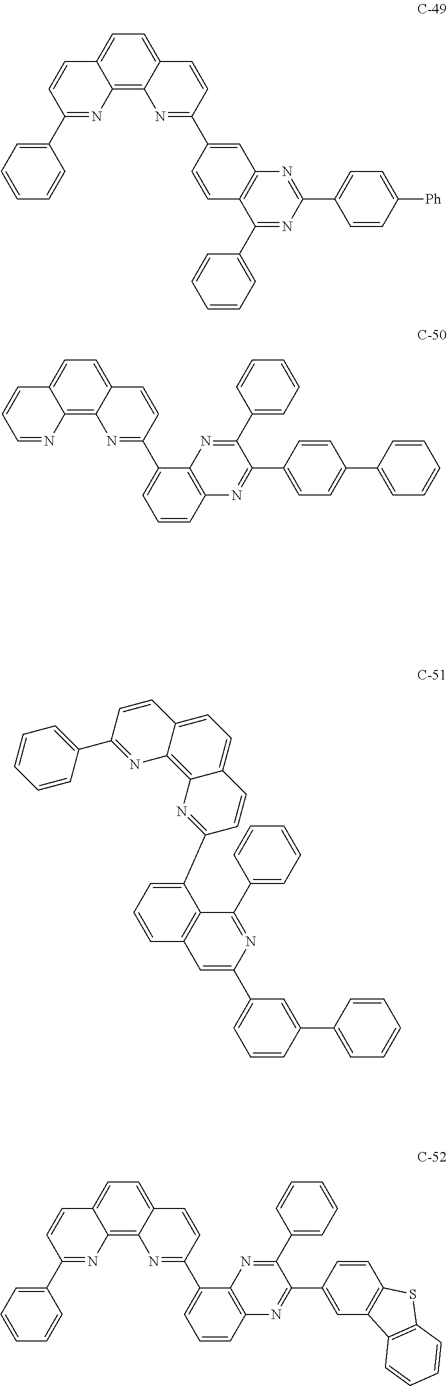 [SMILES: c1ccc(-c2ccc(-c3nc(-c4ccccc4)c4ccc(-c5ccc6ccc7ccc(-c8ccccc8)nc7c6n5)cc4n3)cc2)cc1 ; c1ccc(-c2ccc(-c3nc4cccc(-c5ccc6ccc7cccnc7c6n5)c4nc3-c3ccccc3)cc2)cc1 ; c1ccc(-c2cccc(-c3cc4cccc(-c5ccc6ccc7ccc(-c8ccccc8)nc7c6n5)c4c(-c4ccccc4)n3)c2)cc1 ; c1ccc(-c2ccc3ccc4ccc(-c5cccc6nc(-c7ccc8sc9ccccc9c8c7)c(-c7ccccc7)nc56)nc4c3n2)cc1] 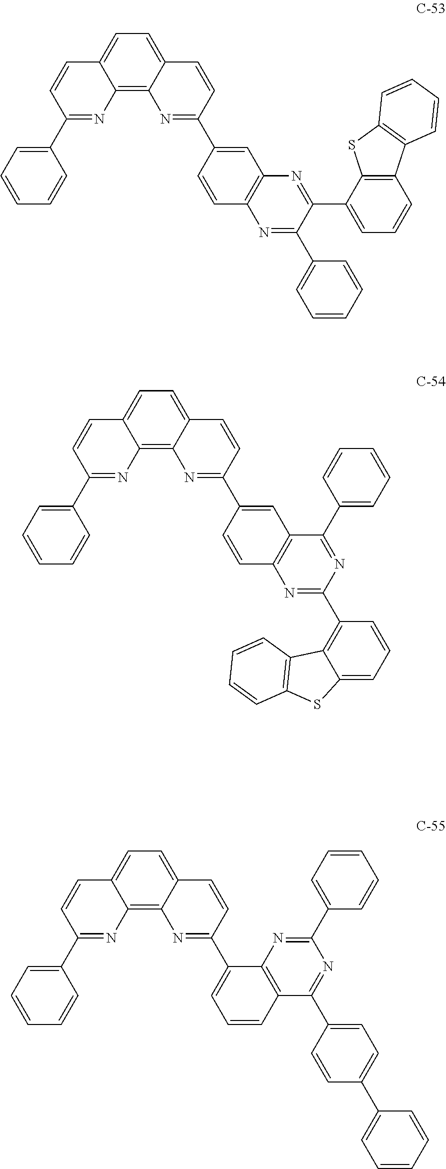 [SMILES: c1ccc(-c2ccc3ccc4ccc(-c5ccc6nc(-c7ccccc7)c(-c7cccc8c7sc7ccccc78)nc6c5)nc4c3n2)cc1 ; c1ccc(-c2ccc3ccc4ccc(-c5ccc6nc(-c7cccc8sc9ccccc9c78)nc(-c7ccccc7)c6c5)nc4c3n2)cc1 ; c1ccc(-c2ccc(-c3nc(-c4ccccc4)nc4c(-c5ccc6ccc7ccc(-c8ccccc8)nc7c6n5)cccc34)cc2)cc1] 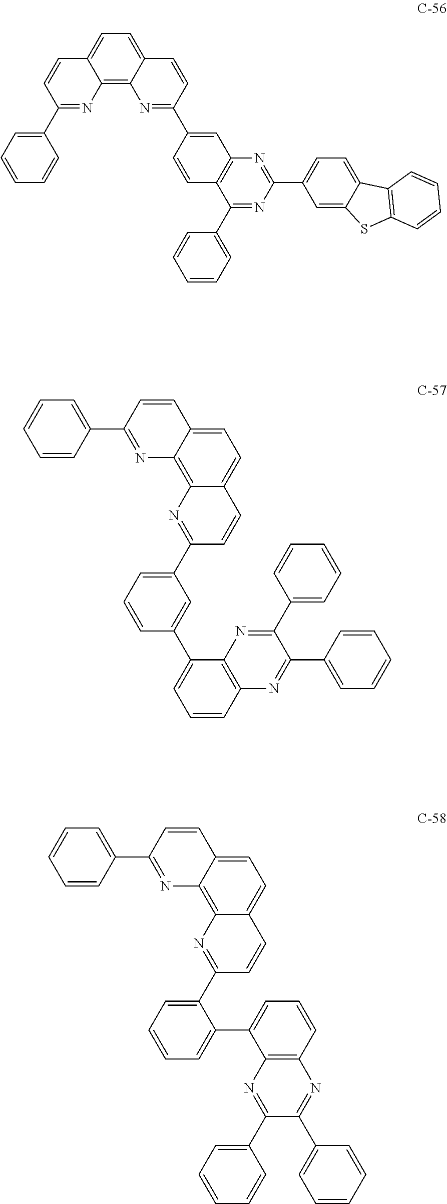 [SMILES: c1ccc(-c2ccc3ccc4ccc(-c5ccc6c(-c7ccccc7)nc(-c7ccc8c(c7)sc7ccccc78)nc6c5)nc4c3n2)cc1 ; c1ccc(-c2ccc3ccc4ccc(-c5cccc(-c6cccc7nc(-c8ccccc8)c(-c8ccccc8)nc67)c5)nc4c3n2)cc1 ; c1ccc(-c2ccc3ccc4ccc(-c5ccccc5-c5cccc6nc(-c7ccccc7)c(-c7ccccc7)nc56)nc4c3n2)cc1] 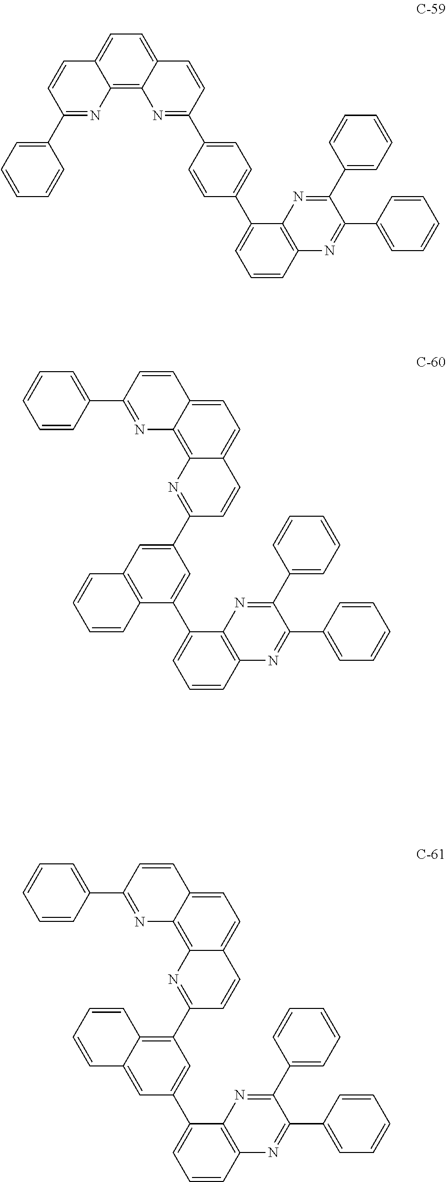 [SMILES: c1ccc(-c2ccc3ccc4ccc(-c5ccc(-c6cccc7nc(-c8ccccc8)c(-c8ccccc8)nc67)cc5)nc4c3n2)cc1 ; c1ccc(-c2ccc3ccc4ccc(-c5cc(-c6cccc7nc(-c8ccccc8)c(-c8ccccc8)nc67)c6ccccc6c5)nc4c3n2)cc1 ; c1ccc(-c2ccc3ccc4ccc(-c5cc(-c6cccc7nc(-c8ccccc8)c(-c8ccccc8)nc67)cc6ccccc56)nc4c3n2)cc1] 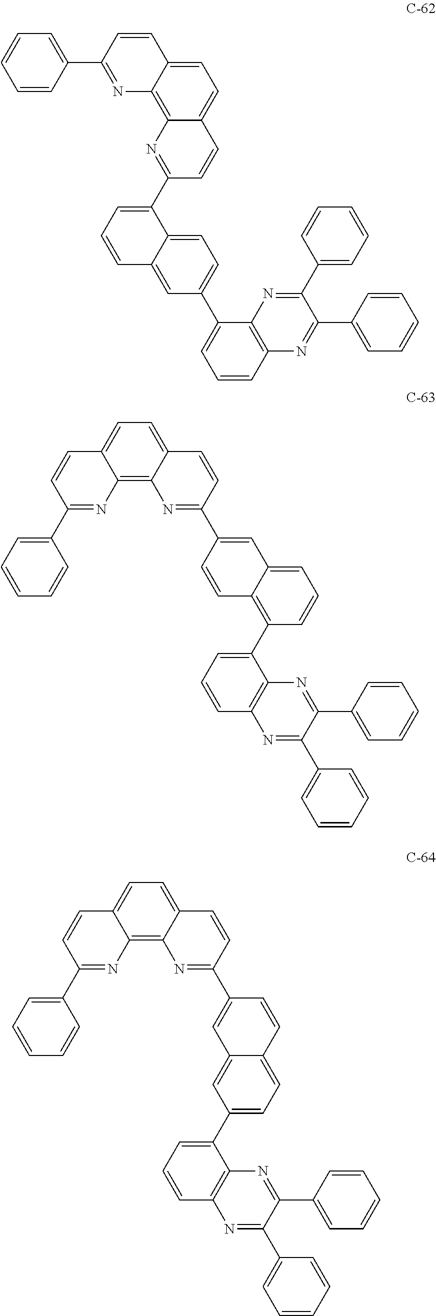 [SMILES: c1ccc(-c2ccc3ccc4ccc(-c5cccc6cc(-c7cccc8nc(-c9ccccc9)c(-c9ccccc9)nc78)ccc56)nc4c3n2)cc1 ; c1ccc(-c2ccc3ccc4ccc(-c5ccc6c(-c7cccc8nc(-c9ccccc9)c(-c9ccccc9)nc78)cccc6c5)nc4c3n2)cc1 ; c1ccc(-c2ccc3ccc4ccc(-c5ccc6ccc(-c7cccc8nc(-c9ccccc9)c(-c9ccccc9)nc78)cc6c5)nc4c3n2)cc1] 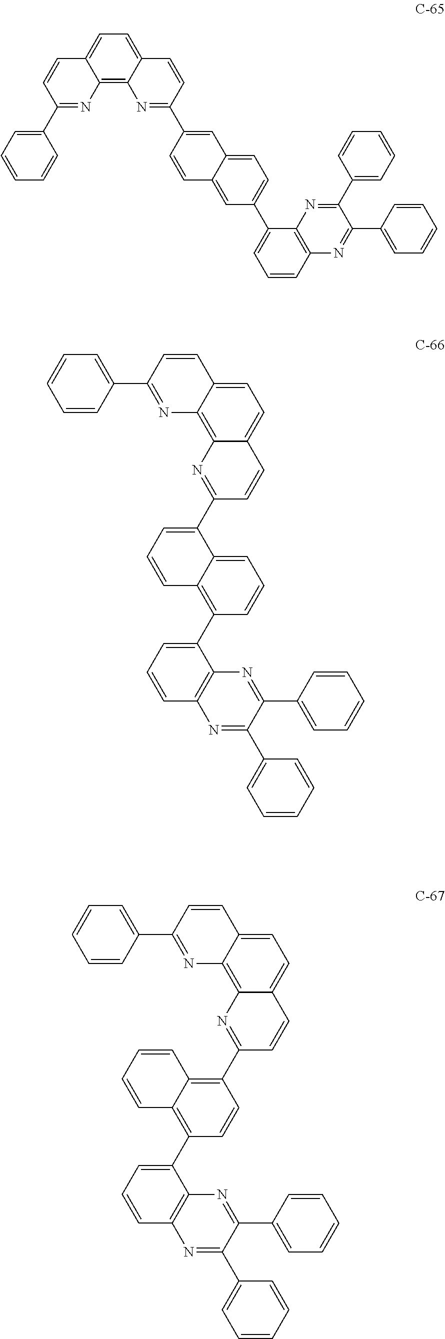 [SMILES: c1ccc(-c2ccc3ccc4ccc(-c5ccc6cc(-c7cccc8nc(-c9ccccc9)c(-c9ccccc9)nc78)ccc6c5)nc4c3n2)cc1 ; c1ccc(-c2ccc3ccc4ccc(-c5cccc6c(-c7cccc8nc(-c9ccccc9)c(-c9ccccc9)nc78)cccc56)nc4c3n2)cc1 ; c1ccc(-c2ccc3ccc4ccc(-c5ccc(-c6cccc7nc(-c8ccccc8)c(-c8ccccc8)nc67)c6ccccc56)nc4c3n2)cc1] 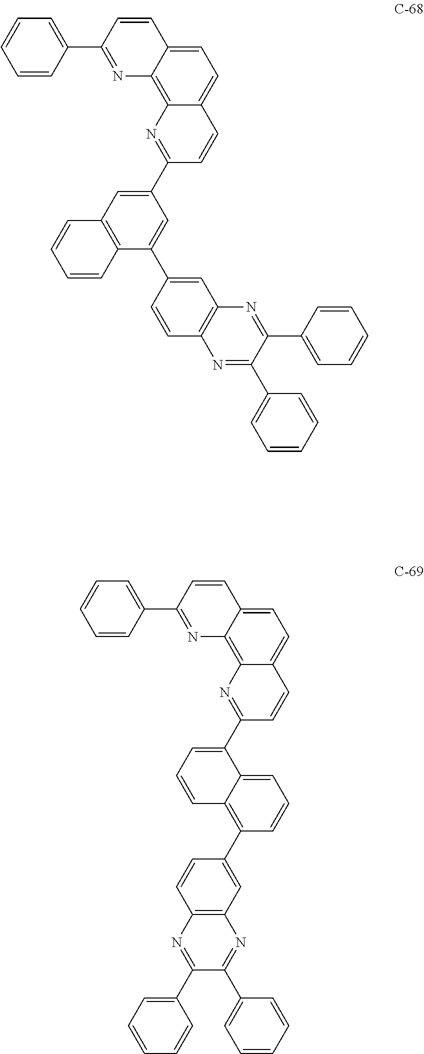 [SMILES: c1ccc(-c2ccc3ccc4ccc(-c5cc(-c6ccc7nc(-c8ccccc8)c(-c8ccccc8)nc7c6)c6ccccc6c5)nc4c3n2)cc1 ; c1ccc(-c2ccc3ccc4ccc(-c5cccc6c(-c7ccc8nc(-c9ccccc9)c(-c9ccccc9)nc8c7)cccc56)nc4c3n2)cc1] 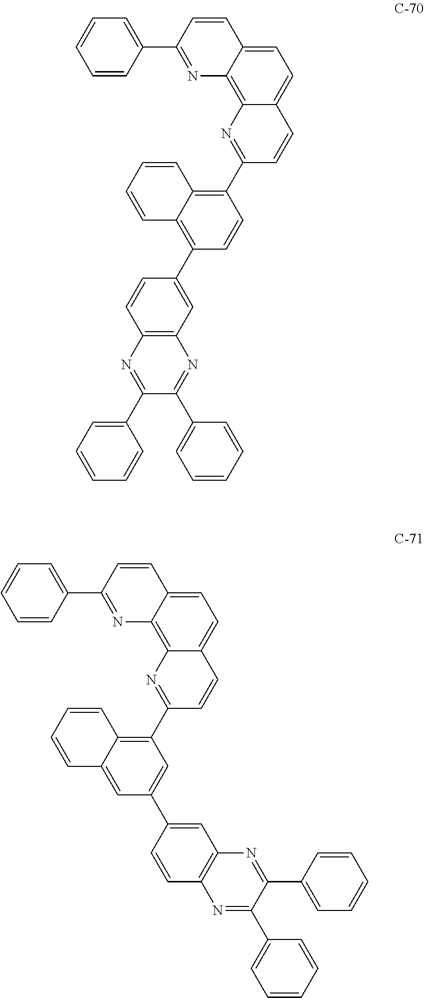 [SMILES: c1ccc(-c2ccc3ccc4ccc(-c5ccc(-c6ccc7nc(-c8ccccc8)c(-c8ccccc8)nc7c6)c6ccccc56)nc4c3n2)cc1 ; c1ccc(-c2ccc3ccc4ccc(-c5cc(-c6ccc7nc(-c8ccccc8)c(-c8ccccc8)nc7c6)cc6ccccc56)nc4c3n2)cc1] 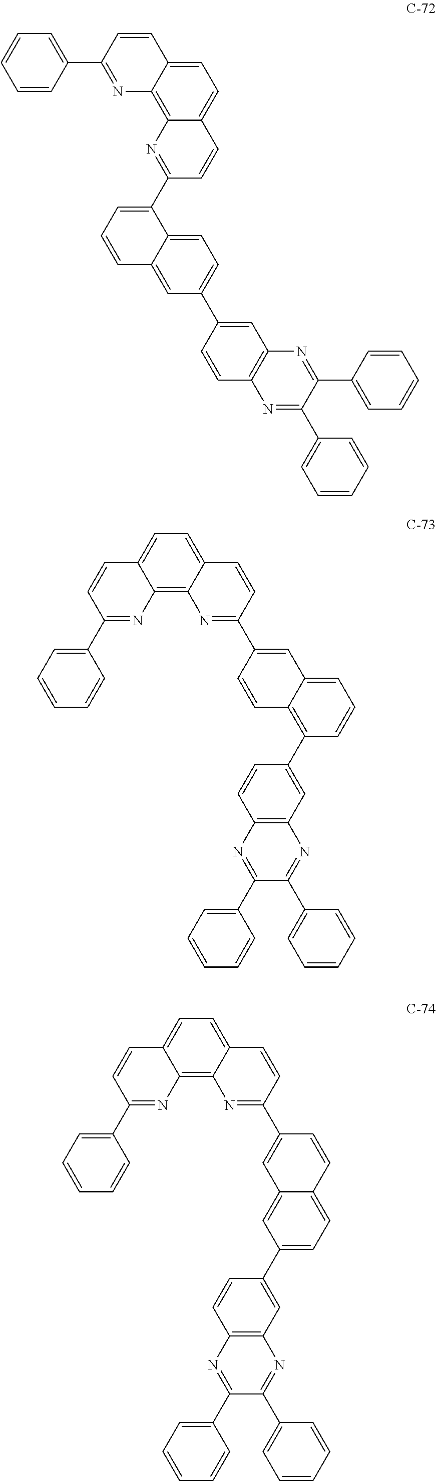 [SMILES: c1ccc(-c2ccc3ccc4ccc(-c5cccc6cc(-c7ccc8nc(-c9ccccc9)c(-c9ccccc9)nc8c7)ccc56)nc4c3n2)cc1 ; c1ccc(-c2ccc3ccc4ccc(-c5ccc6c(-c7ccc8nc(-c9ccccc9)c(-c9ccccc9)nc8c7)cccc6c5)nc4c3n2)cc1 ; c1ccc(-c2ccc3ccc4ccc(-c5ccc6ccc(-c7ccc8nc(-c9ccccc9)c(-c9ccccc9)nc8c7)cc6c5)nc4c3n2)cc1] 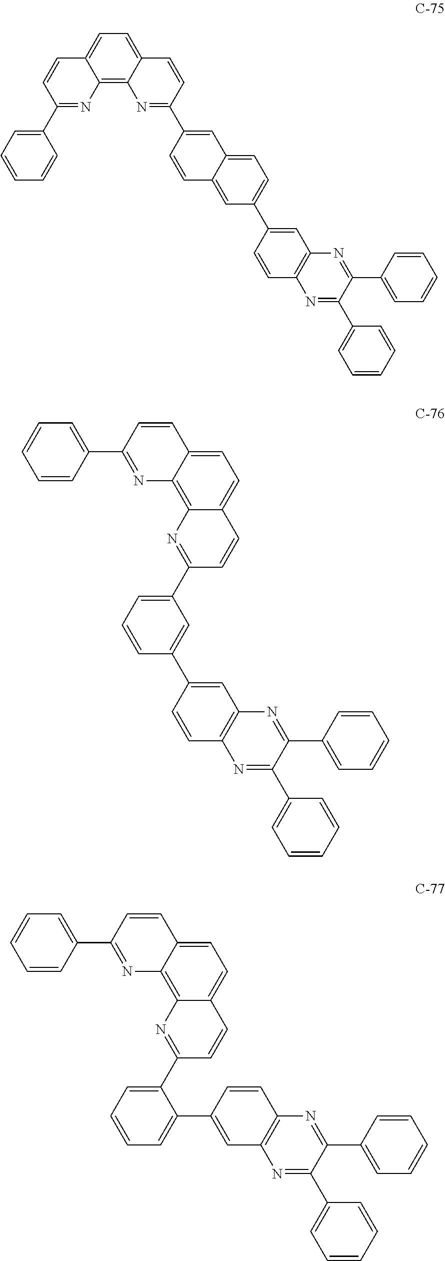 [SMILES: c1ccc(-c2ccc3ccc4ccc(-c5ccc6cc(-c7ccc8nc(-c9ccccc9)c(-c9ccccc9)nc8c7)ccc6c5)nc4c3n2)cc1 ; c1ccc(-c2ccc3ccc4ccc(-c5cccc(-c6ccc7nc(-c8ccccc8)c(-c8ccccc8)nc7c6)c5)nc4c3n2)cc1 ; c1ccc(-c2ccc3ccc4ccc(-c5ccccc5-c5ccc6nc(-c7ccccc7)c(-c7ccccc7)nc6c5)nc4c3n2)cc1] 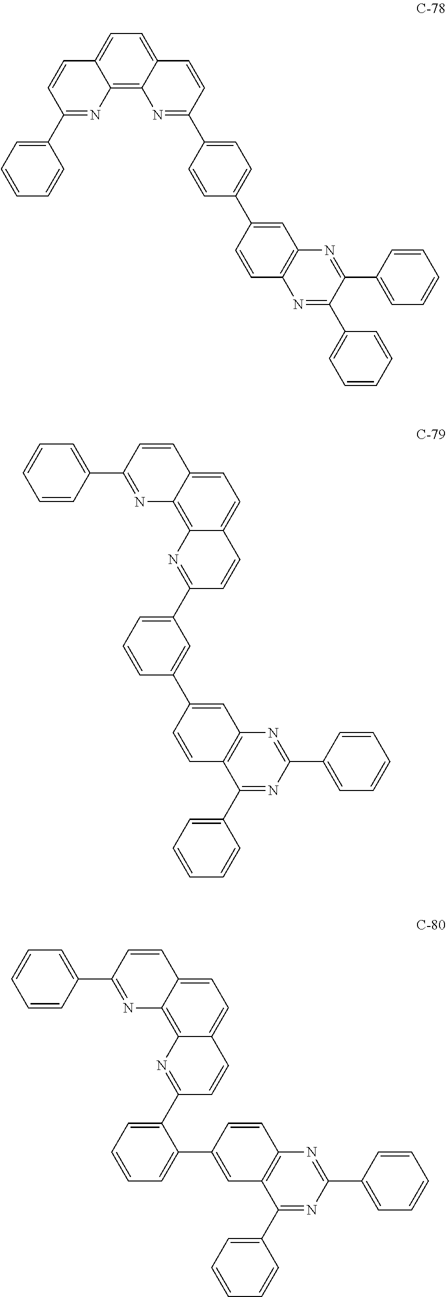 [SMILES: c1ccc(-c2ccc3ccc4ccc(-c5ccc(-c6ccc7nc(-c8ccccc8)c(-c8ccccc8)nc7c6)cc5)nc4c3n2)cc1 ; c1ccc(-c2ccc3ccc4ccc(-c5cccc(-c6ccc7c(-c8ccccc8)nc(-c8ccccc8)nc7c6)c5)nc4c3n2)cc1 ; c1ccc(-c2ccc3ccc4ccc(-c5ccccc5-c5ccc6nc(-c7ccccc7)nc(-c7ccccc7)c6c5)nc4c3n2)cc1] 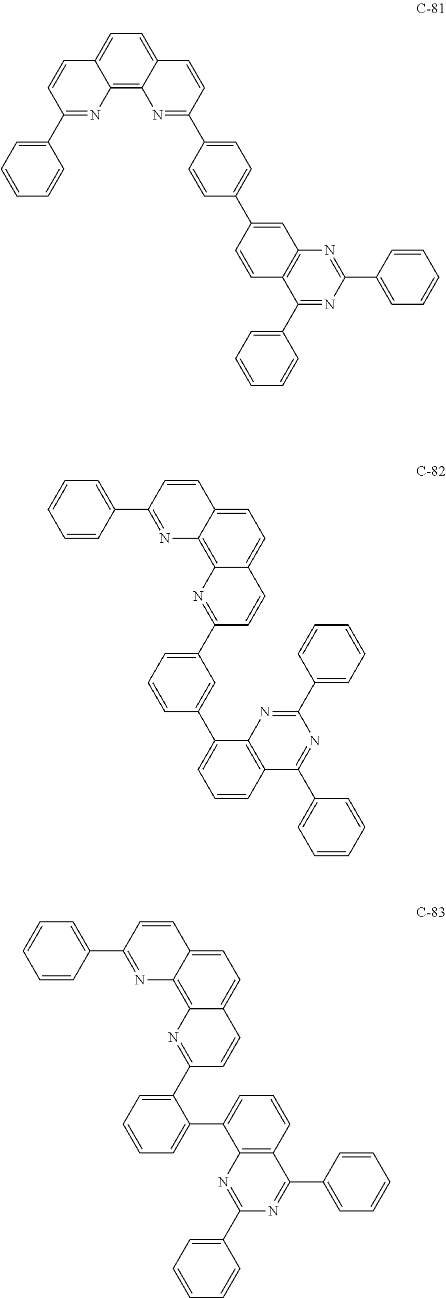 [SMILES: c1ccc(-c2ccc3ccc4ccc(-c5ccc(-c6ccc7c(-c8ccccc8)nc(-c8ccccc8)nc7c6)cc5)nc4c3n2)cc1 ; c1ccc(-c2ccc3ccc4ccc(-c5cccc(-c6cccc7c(-c8ccccc8)nc(-c8ccccc8)nc67)c5)nc4c3n2)cc1 ; c1ccc(-c2ccc3ccc4ccc(-c5ccccc5-c5cccc6c(-c7ccccc7)nc(-c7ccccc7)nc56)nc4c3n2)cc1] 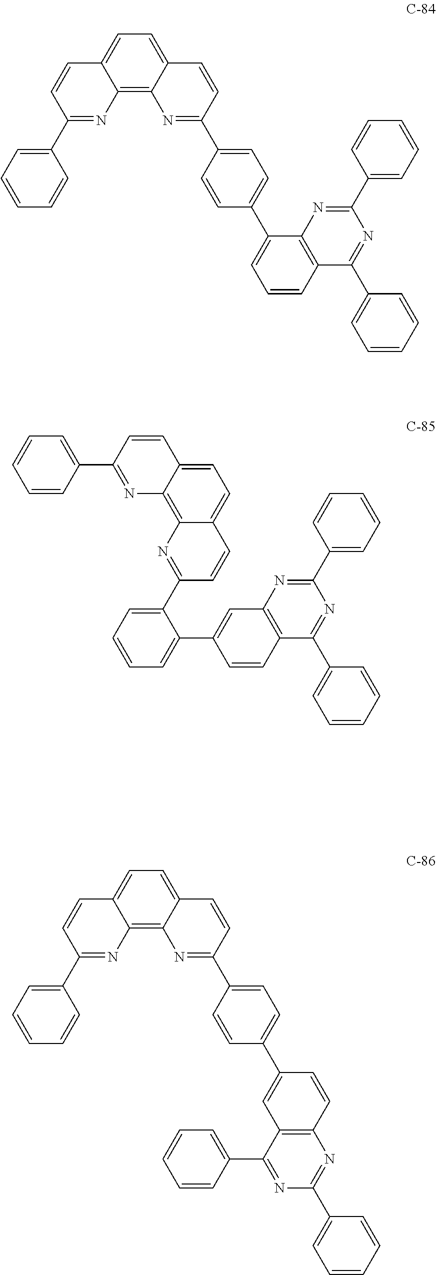 [SMILES: c1ccc(-c2ccc3ccc4ccc(-c5ccc(-c6cccc7c(-c8ccccc8)nc(-c8ccccc8)nc67)cc5)nc4c3n2)cc1 ; c1ccc(-c2ccc3ccc4ccc(-c5ccccc5-c5ccc6c(-c7ccccc7)nc(-c7ccccc7)nc6c5)nc4c3n2)cc1 ; c1ccc(-c2ccc3ccc4ccc(-c5ccc(-c6ccc7nc(-c8ccccc8)nc(-c8ccccc8)c7c6)cc5)nc4c3n2)cc1] 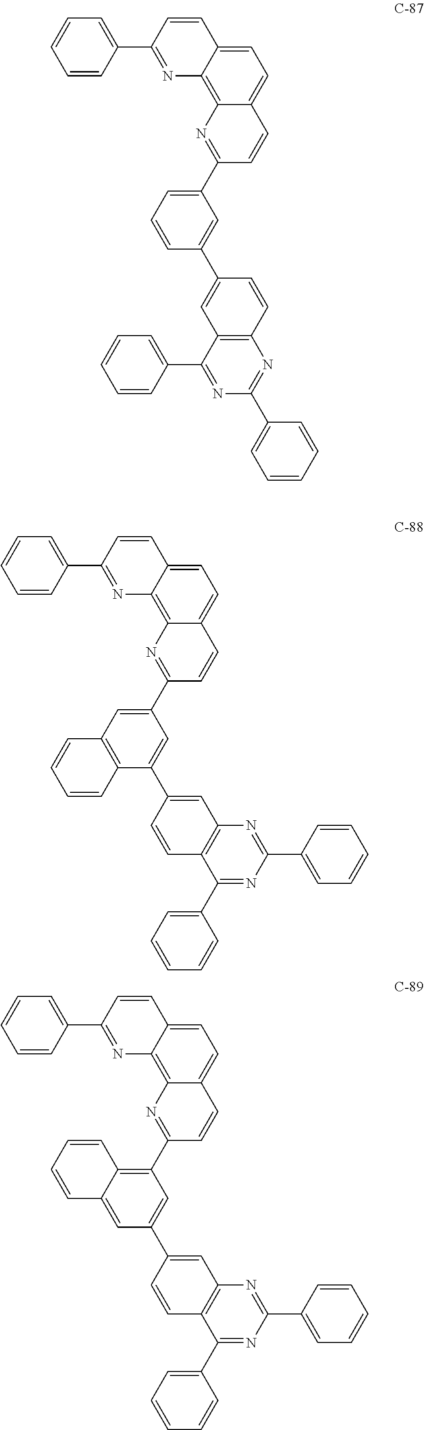 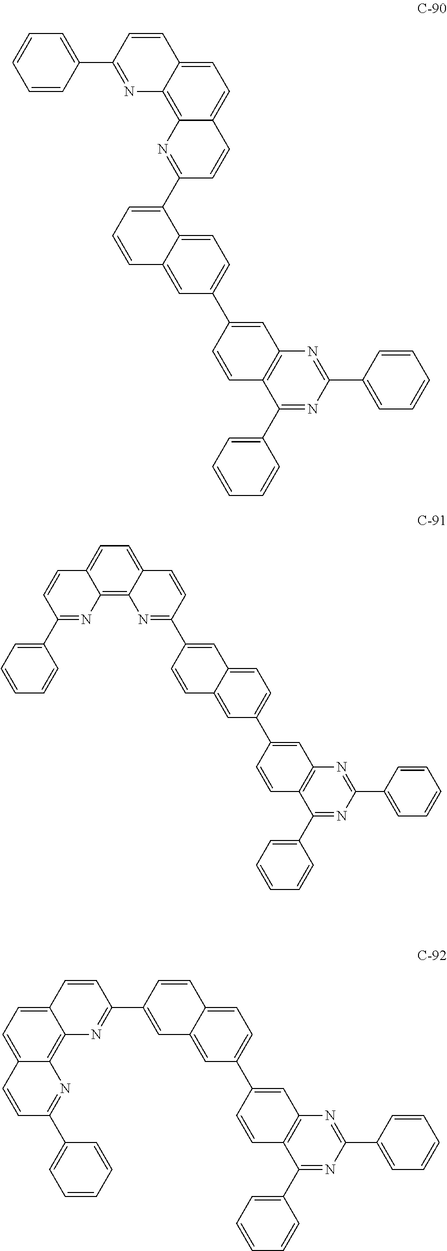 [SMILES: c1ccc(-c2ccc3ccc4ccc(-c5cccc6cc(-c7ccc8c(-c9ccccc9)nc(-c9ccccc9)nc8c7)ccc56)nc4c3n2)cc1 ; c1ccc(-c2ccc3ccc4ccc(-c5ccc6cc(-c7ccc8c(-c9ccccc9)nc(-c9ccccc9)nc8c7)ccc6c5)nc4c3n2)cc1 ; c1ccc(-c2ccc3ccc4ccc(-c5ccc6ccc(-c7ccc8c(-c9ccccc9)nc(-c9ccccc9)nc8c7)cc6c5)nc4c3n2)cc1] 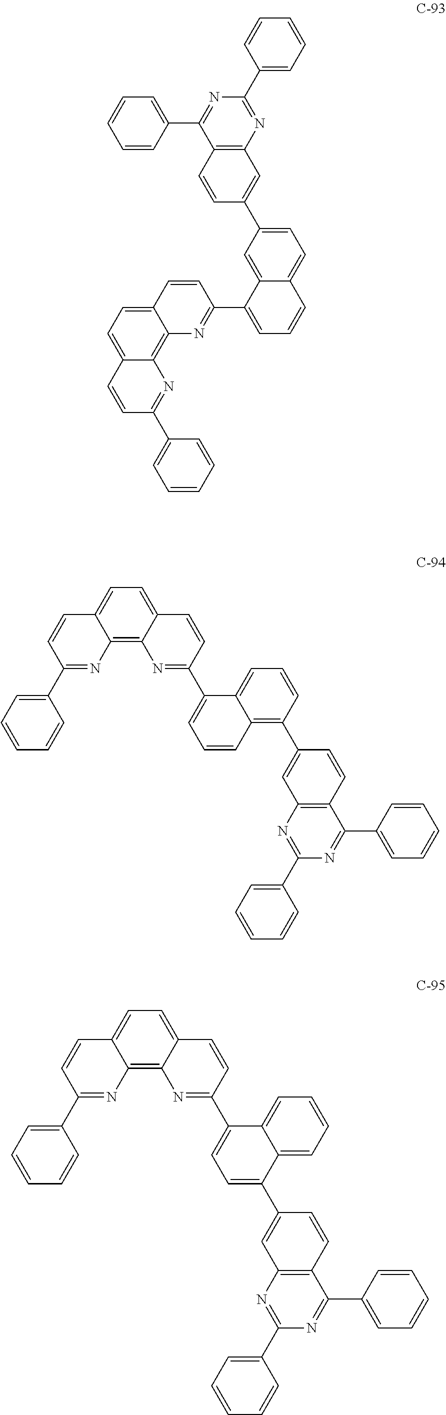 [SMILES: c1ccc(-c2ccc3ccc4ccc(-c5cccc6ccc(-c7ccc8c(-c9ccccc9)nc(-c9ccccc9)nc8c7)cc56)nc4c3n2)cc1 ; c1ccc(-c2ccc3ccc4ccc(-c5cccc6c(-c7ccc8c(-c9ccccc9)nc(-c9ccccc9)nc8c7)cccc56)nc4c3n2)cc1 ; c1ccc(-c2ccc3ccc4ccc(-c5ccc(-c6ccc7c(-c8ccccc8)nc(-c8ccccc8)nc7c6)c6ccccc56)nc4c3n2)cc1] 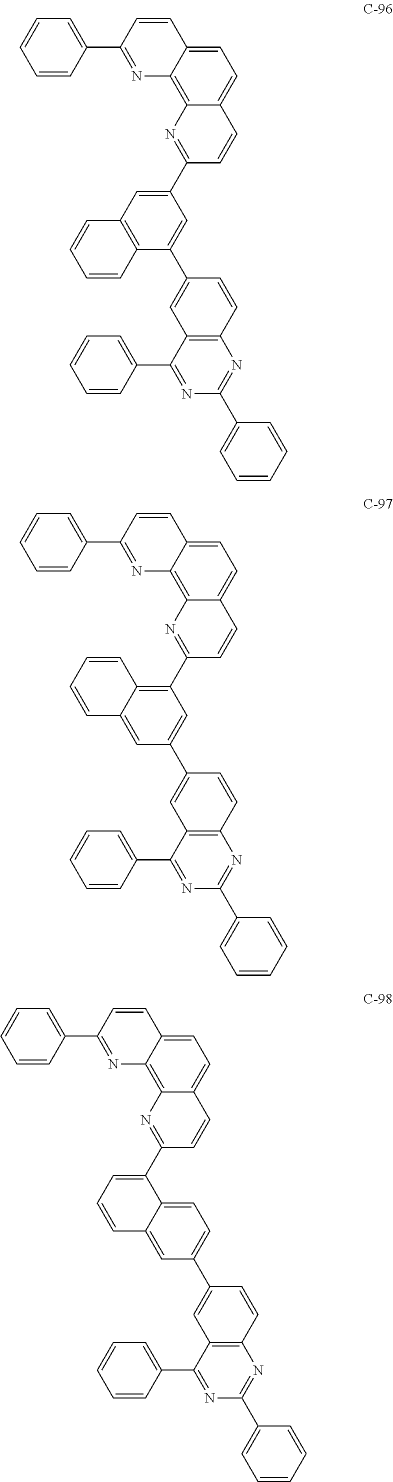 [SMILES: c1ccc(-c2ccc3ccc4ccc(-c5cc(-c6ccc7nc(-c8ccccc8)nc(-c8ccccc8)c7c6)c6ccccc6c5)nc4c3n2)cc1 ; c1ccc(-c2ccc3ccc4ccc(-c5cc(-c6ccc7nc(-c8ccccc8)nc(-c8ccccc8)c7c6)cc6ccccc56)nc4c3n2)cc1 ; c1ccc(-c2ccc3ccc4ccc(-c5cccc6cc(-c7ccc8nc(-c9ccccc9)nc(-c9ccccc9)c8c7)ccc56)nc4c3n2)cc1] 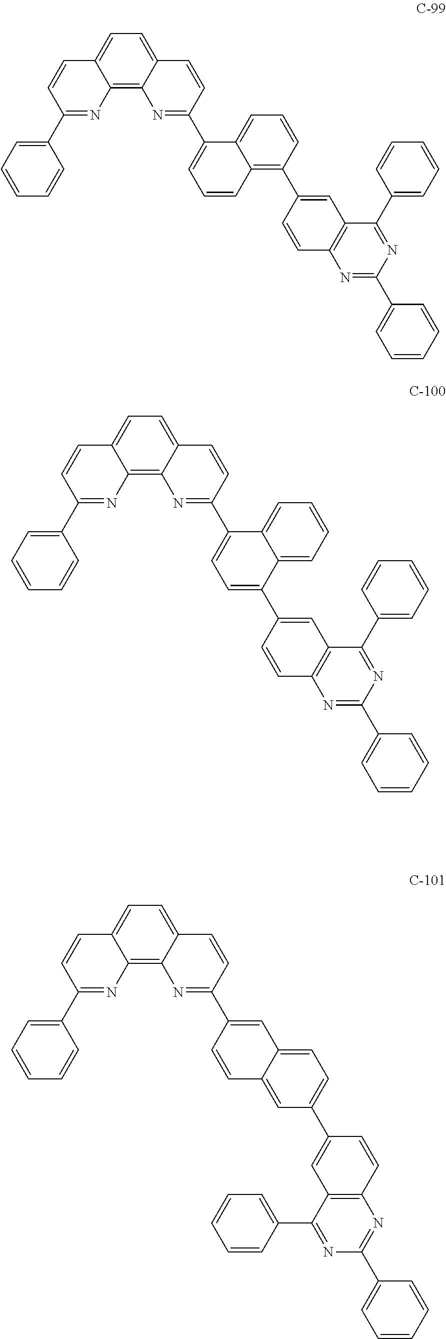 [SMILES: c1ccc(-c2ccc3ccc4ccc(-c5cccc6c(-c7ccc8nc(-c9ccccc9)nc(-c9ccccc9)c8c7)cccc56)nc4c3n2)cc1 ; c1ccc(-c2ccc3ccc4ccc(-c5ccc(-c6ccc7nc(-c8ccccc8)nc(-c8ccccc8)c7c6)c6ccccc56)nc4c3n2)cc1 ; c1ccc(-c2ccc3ccc4ccc(-c5ccc6cc(-c7ccc8nc(-c9ccccc9)nc(-c9ccccc9)c8c7)ccc6c5)nc4c3n2)cc1] 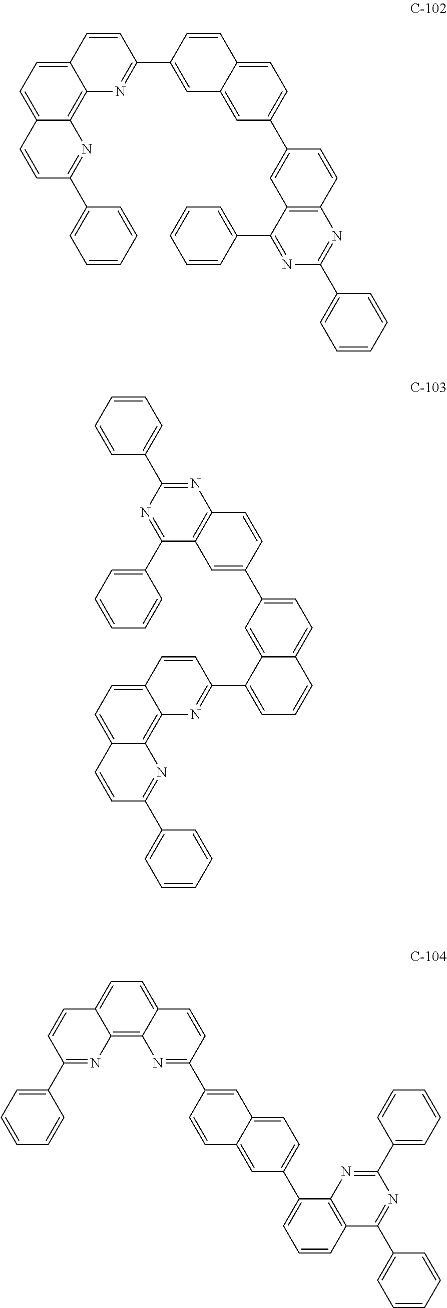 [SMILES: c1ccc(-c2ccc3ccc4ccc(-c5ccc6ccc(-c7ccc8nc(-c9ccccc9)nc(-c9ccccc9)c8c7)cc6c5)nc4c3n2)cc1 ; c1ccc(-c2ccc3ccc4ccc(-c5cccc6ccc(-c7ccc8nc(-c9ccccc9)nc(-c9ccccc9)c8c7)cc56)nc4c3n2)cc1 ; c1ccc(-c2ccc3ccc4ccc(-c5ccc6cc(-c7cccc8c(-c9ccccc9)nc(-c9ccccc9)nc78)ccc6c5)nc4c3n2)cc1] 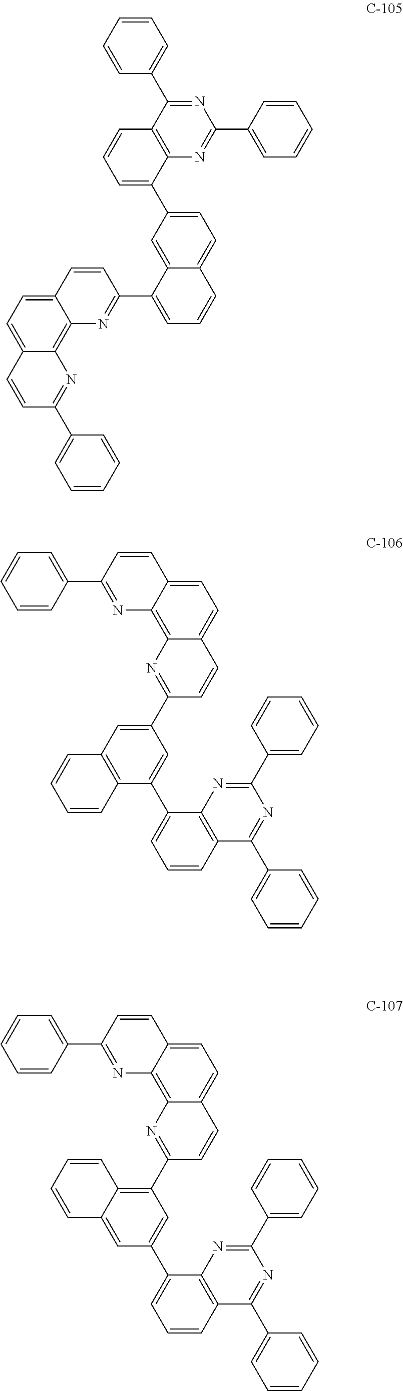 [SMILES: c1ccc(-c2ccc3ccc4ccc(-c5cccc6ccc(-c7cccc8c(-c9ccccc9)nc(-c9ccccc9)nc78)cc56)nc4c3n2)cc1 ; c1ccc(-c2ccc3ccc4ccc(-c5cc(-c6cccc7c(-c8ccccc8)nc(-c8ccccc8)nc67)c6ccccc6c5)nc4c3n2)cc1 ; c1ccc(-c2ccc3ccc4ccc(-c5cc(-c6cccc7c(-c8ccccc8)nc(-c8ccccc8)nc67)cc6ccccc56)nc4c3n2)cc1] 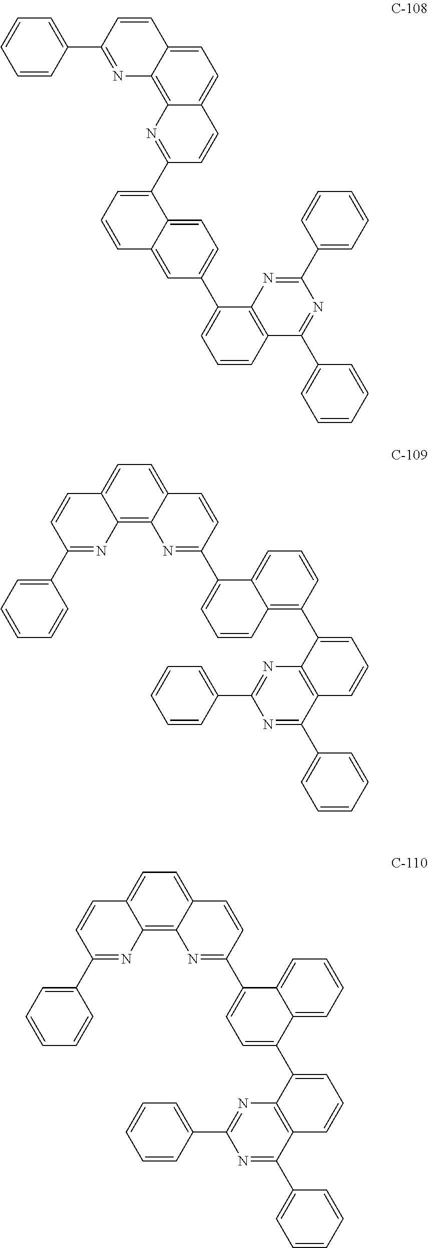 [SMILES: c1ccc(-c2ccc3ccc4ccc(-c5cccc6cc(-c7cccc8c(-c9ccccc9)nc(-c9ccccc9)nc78)ccc56)nc4c3n2)cc1 ; c1ccc(-c2ccc3ccc4ccc(-c5cccc6c(-c7cccc8c(-c9ccccc9)nc(-c9ccccc9)nc78)cccc56)nc4c3n2)cc1 ; c1ccc(-c2ccc3ccc4ccc(-c5ccc(-c6cccc7c(-c8ccccc8)nc(-c8ccccc8)nc67)c6ccccc56)nc4c3n2)cc1] 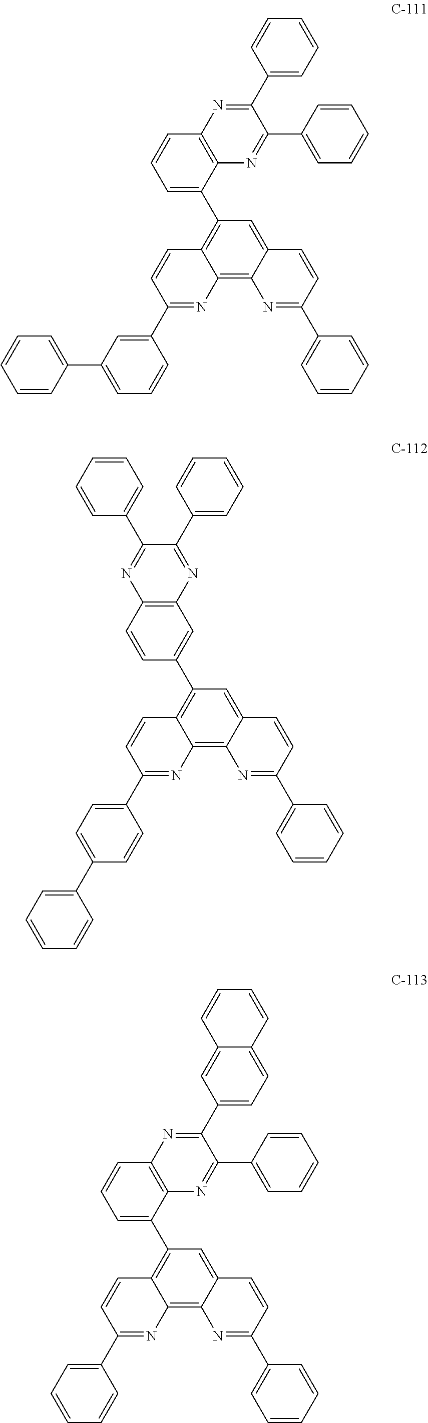 [SMILES: c1ccc(-c2cccc(-c3ccc4c(-c5cccc6nc(-c7ccccc7)c(-c7ccccc7)nc56)cc5ccc(-c6ccccc6)nc5c4n3)c2)cc1 ; c1ccc(-c2ccc(-c3ccc4c(-c5ccc6nc(-c7ccccc7)c(-c7ccccc7)nc6c5)cc5ccc(-c6ccccc6)nc5c4n3)cc2)cc1 ; c1ccc(-c2ccc3cc(-c4cccc5nc(-c6ccc7ccccc7c6)c(-c6ccccc6)nc45)c4ccc(-c5ccccc5)nc4c3n2)cc1] 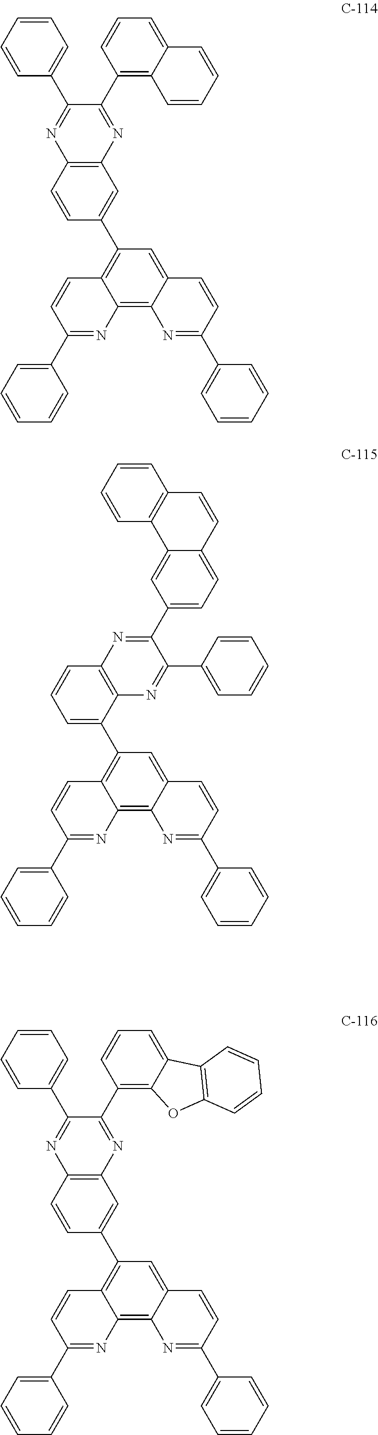 [SMILES: c1ccc(-c2ccc3cc(-c4ccc5nc(-c6ccccc6)c(-c6cccc7ccccc67)nc5c4)c4ccc(-c5ccccc5)nc4c3n2)cc1 ; c1ccc(-c2ccc3cc(-c4cccc5nc(-c6ccc7ccc8ccccc8c7c6)c(-c6ccccc6)nc45)c4ccc(-c5ccccc5)nc4c3n2)cc1 ; c1ccc(-c2ccc3cc(-c4ccc5nc(-c6ccccc6)c(-c6cccc7c6oc6ccccc67)nc5c4)c4ccc(-c5ccccc5)nc4c3n2)cc1] 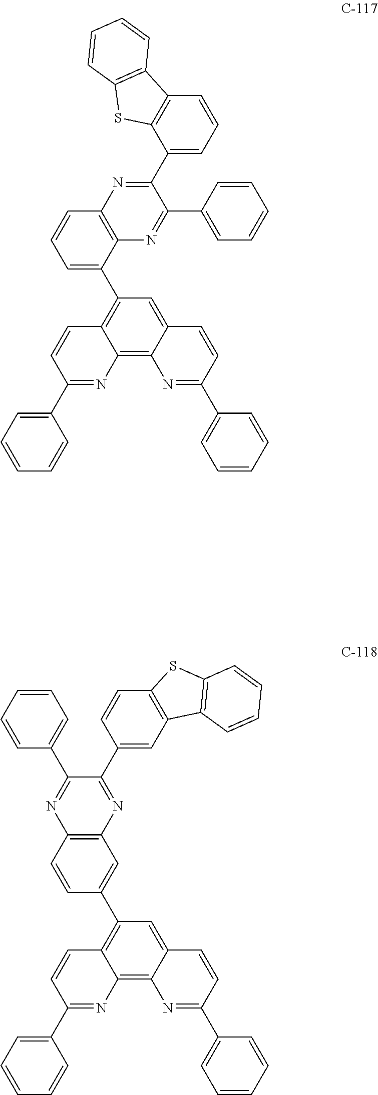 [SMILES: c1ccc(-c2ccc3cc(-c4cccc5nc(-c6cccc7c6sc6ccccc67)c(-c6ccccc6)nc45)c4ccc(-c5ccccc5)nc4c3n2)cc1 ; c1ccc(-c2ccc3cc(-c4ccc5nc(-c6ccccc6)c(-c6ccc7sc8ccccc8c7c6)nc5c4)c4ccc(-c5ccccc5)nc4c3n2)cc1] 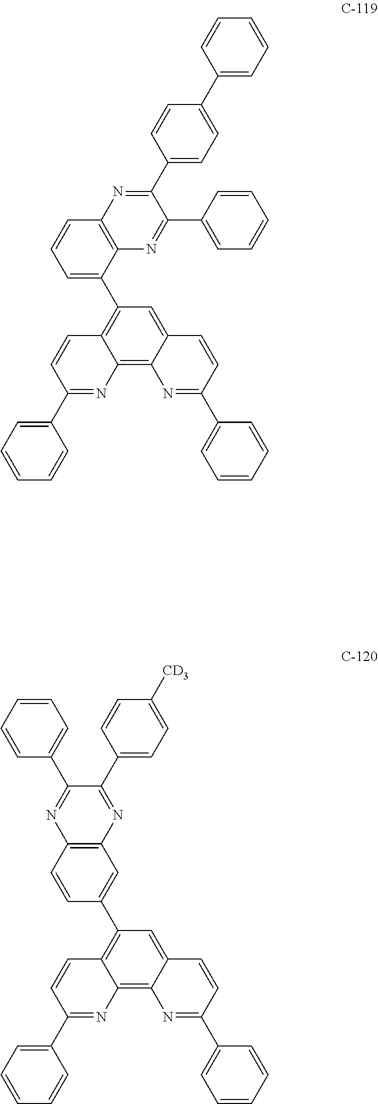 [SMILES: c1ccc(-c2ccc(-c3nc4cccc(-c5cc6ccc(-c7ccccc7)nc6c6nc(-c7ccccc7)ccc56)c4nc3-c3ccccc3)cc2)cc1 ; [2H]C([2H])([2H])c1ccc(-c2nc3cc(-c4cc5ccc(-c6ccccc6)nc5c5nc(-c6ccccc6)ccc45)ccc3nc2-c2ccccc2)cc1] 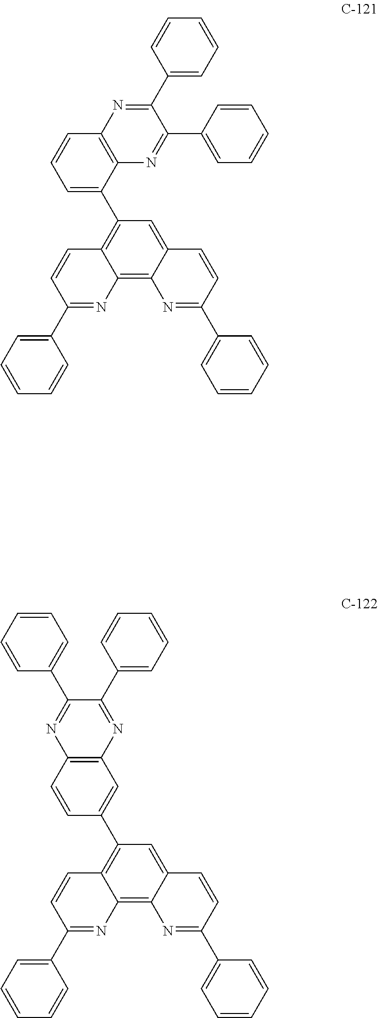 [SMILES: c1ccc(-c2ccc3cc(-c4cccc5nc(-c6ccccc6)c(-c6ccccc6)nc45)c4ccc(-c5ccccc5)nc4c3n2)cc1 ; c1ccc(-c2ccc3cc(-c4ccc5nc(-c6ccccc6)c(-c6ccccc6)nc5c4)c4ccc(-c5ccccc5)nc4c3n2)cc1] 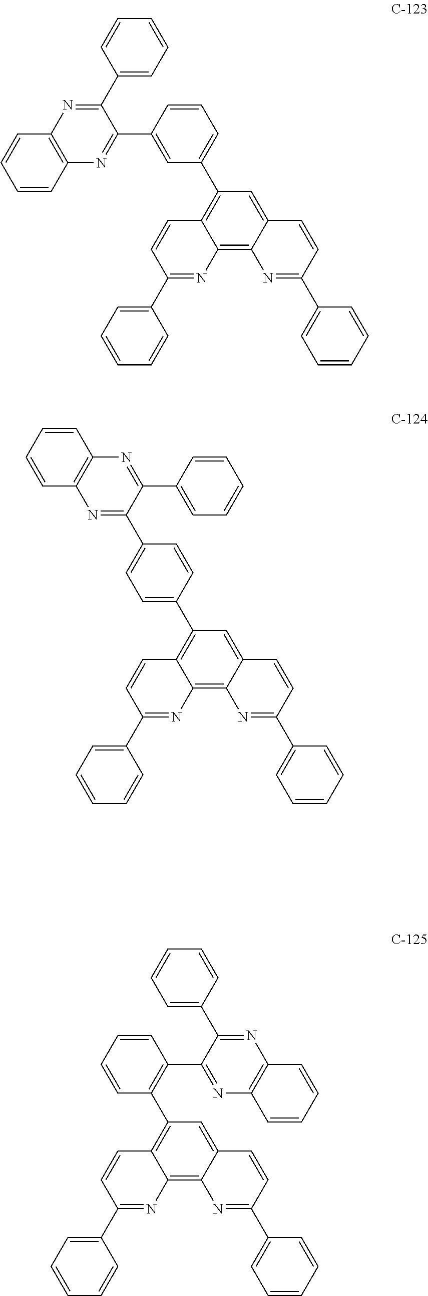 [SMILES: c1ccc(-c2ccc3cc(-c4cccc(-c5nc6ccccc6nc5-c5ccccc5)c4)c4ccc(-c5ccccc5)nc4c3n2)cc1 ; c1ccc(-c2ccc3cc(-c4ccc(-c5nc6ccccc6nc5-c5ccccc5)cc4)c4ccc(-c5ccccc5)nc4c3n2)cc1 ; c1ccc(-c2ccc3cc(-c4ccccc4-c4nc5ccccc5nc4-c4ccccc4)c4ccc(-c5ccccc5)nc4c3n2)cc1] 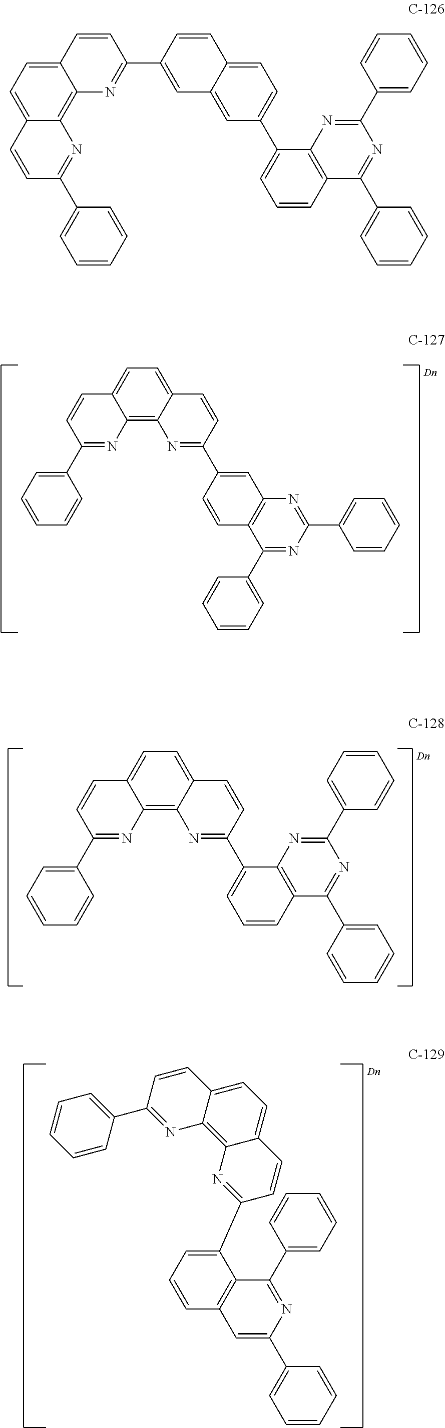 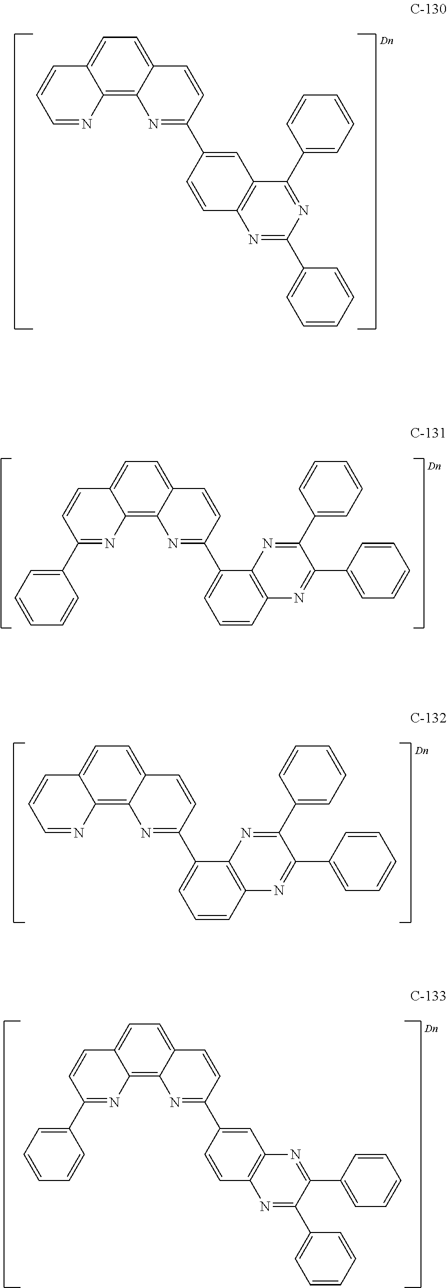 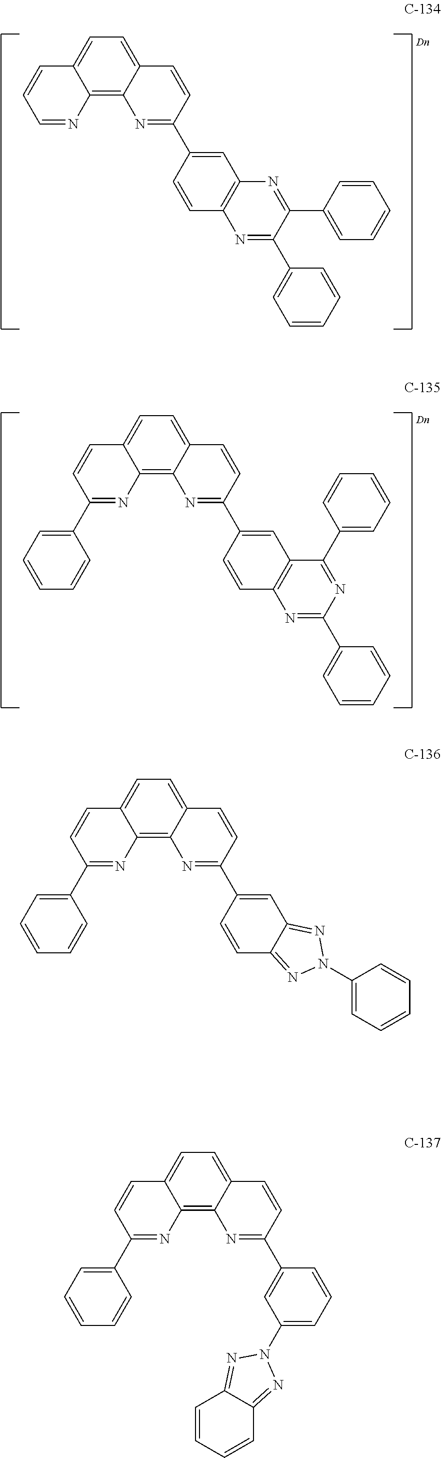 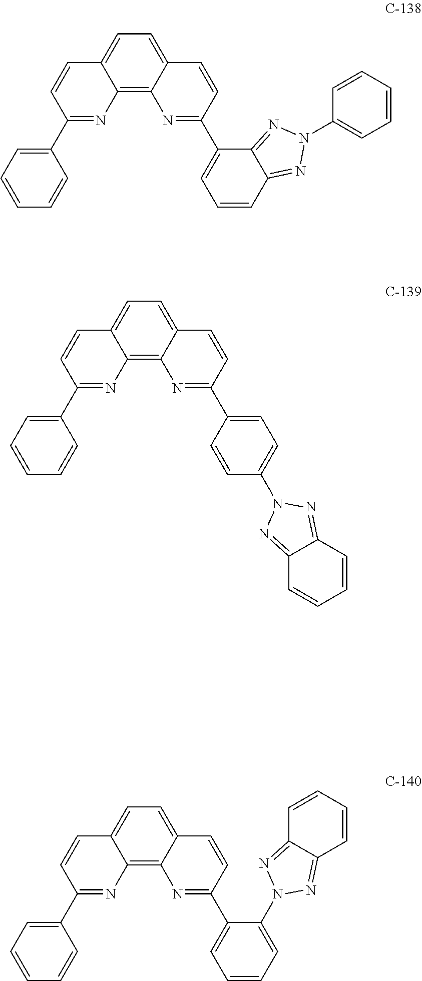 [SMILES: c1ccc(-c2ccc3ccc4ccc(-c5cccc6nn(-c7ccccc7)nc56)nc4c3n2)cc1 ; c1ccc(-c2ccc3ccc4ccc(-c5ccc(-n6nc7ccccc7n6)cc5)nc4c3n2)cc1 ; c1ccc(-c2ccc3ccc4ccc(-c5ccccc5-n5nc6ccccc6n5)nc4c3n2)cc1] 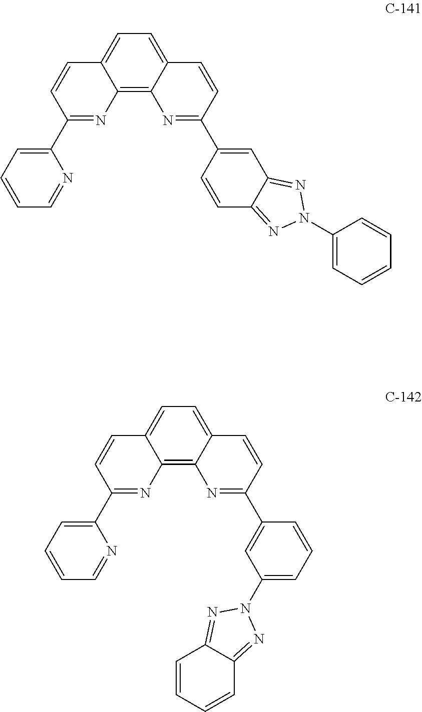 [SMILES: c1ccc(-n2nc3ccc(-c4ccc5ccc6ccc(-c7ccccn7)nc6c5n4)cc3n2)cc1 ; c1ccc(-c2ccc3ccc4ccc(-c5cccc(-n6nc7ccccc7n6)c5)nc4c3n2)nc1] 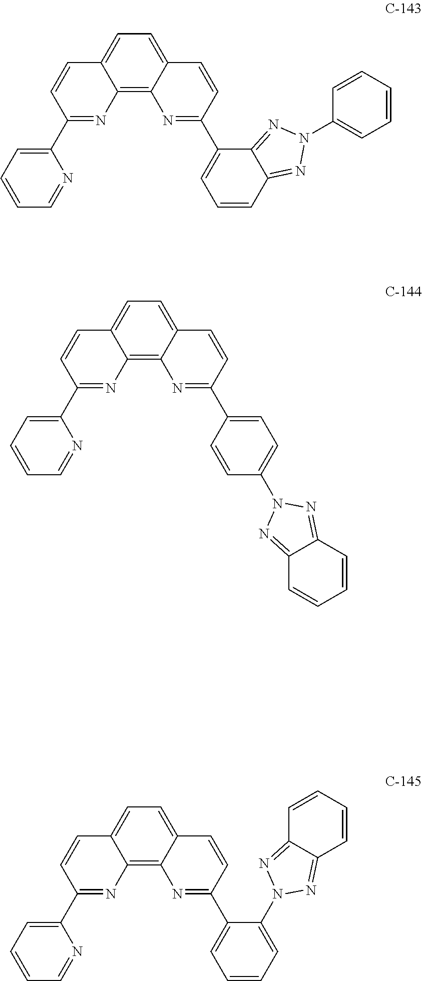 [SMILES: c1ccc(-n2nc3cccc(-c4ccc5ccc6ccc(-c7ccccn7)nc6c5n4)c3n2)cc1 ; c1ccc(-c2ccc3ccc4ccc(-c5ccc(-n6nc7ccccc7n6)cc5)nc4c3n2)nc1 ; c1ccc(-c2ccc3ccc4ccc(-c5ccccc5-n5nc6ccccc6n5)nc4c3n2)nc1] 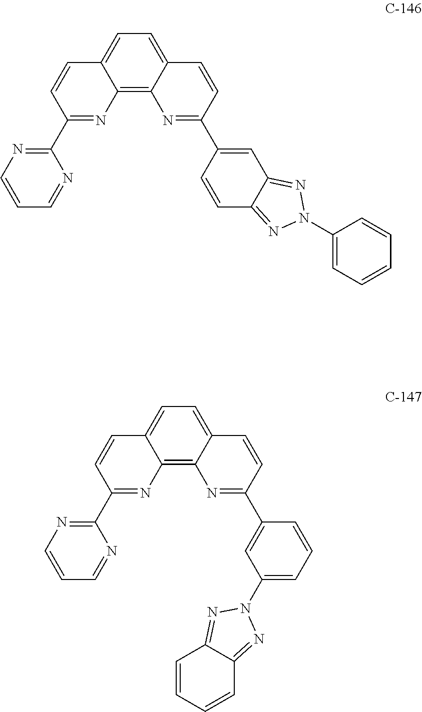 [SMILES: c1ccc(-n2nc3ccc(-c4ccc5ccc6ccc(-c7ncccn7)nc6c5n4)cc3n2)cc1 ; c1cnc(-c2ccc3ccc4ccc(-c5cccc(-n6nc7ccccc7n6)c5)nc4c3n2)nc1] 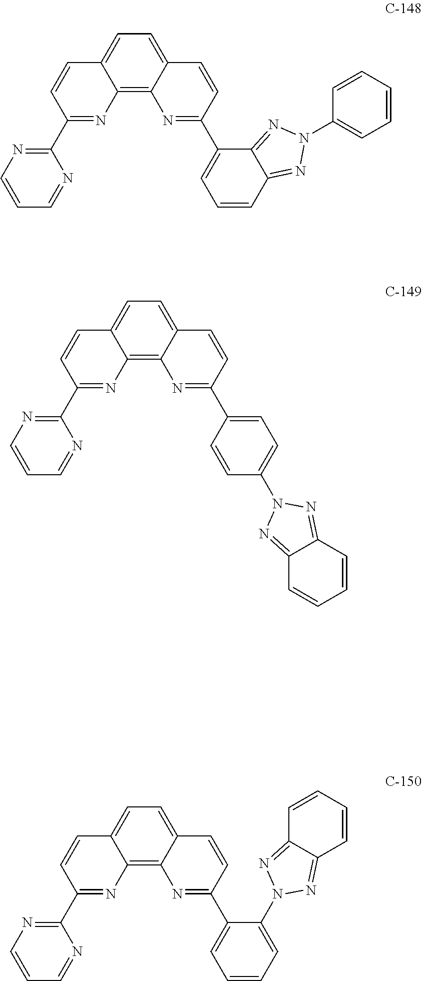 [SMILES: c1ccc(-n2nc3cccc(-c4ccc5ccc6ccc(-c7ncccn7)nc6c5n4)c3n2)cc1 ; c1cnc(-c2ccc3ccc4ccc(-c5ccc(-n6nc7ccccc7n6)cc5)nc4c3n2)nc1 ; c1cnc(-c2ccc3ccc4ccc(-c5ccccc5-n5nc6ccccc6n5)nc4c3n2)nc1] 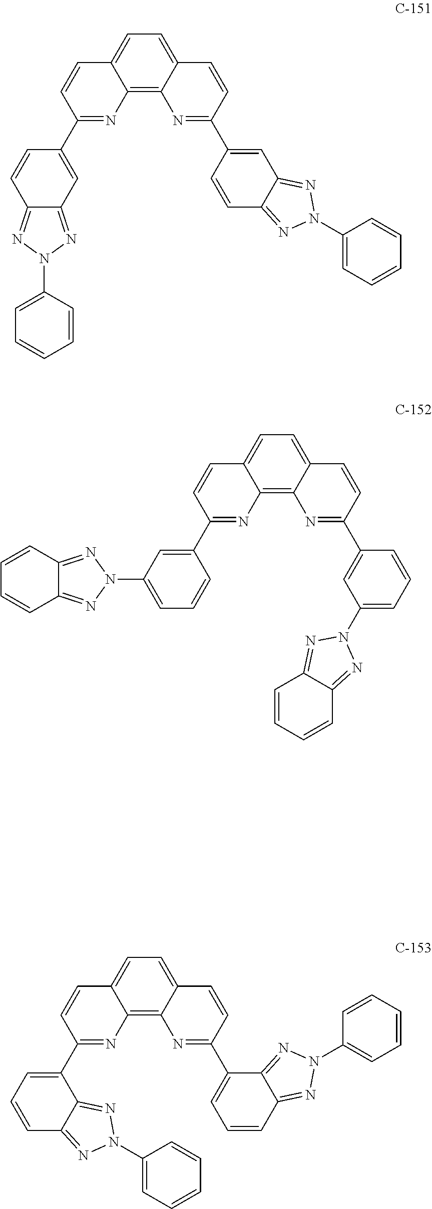 [SMILES: c1ccc(-n2nc3ccc(-c4ccc5ccc6ccc(-c7ccc8nn(-c9ccccc9)nc8c7)nc6c5n4)cc3n2)cc1 ; c1cc(-c2ccc3ccc4ccc(-c5cccc(-n6nc7ccccc7n6)c5)nc4c3n2)cc(-n2nc3ccccc3n2)c1 ; c1ccc(-n2nc3cccc(-c4ccc5ccc6ccc(-c7cccc8nn(-c9ccccc9)nc78)nc6c5n4)c3n2)cc1] 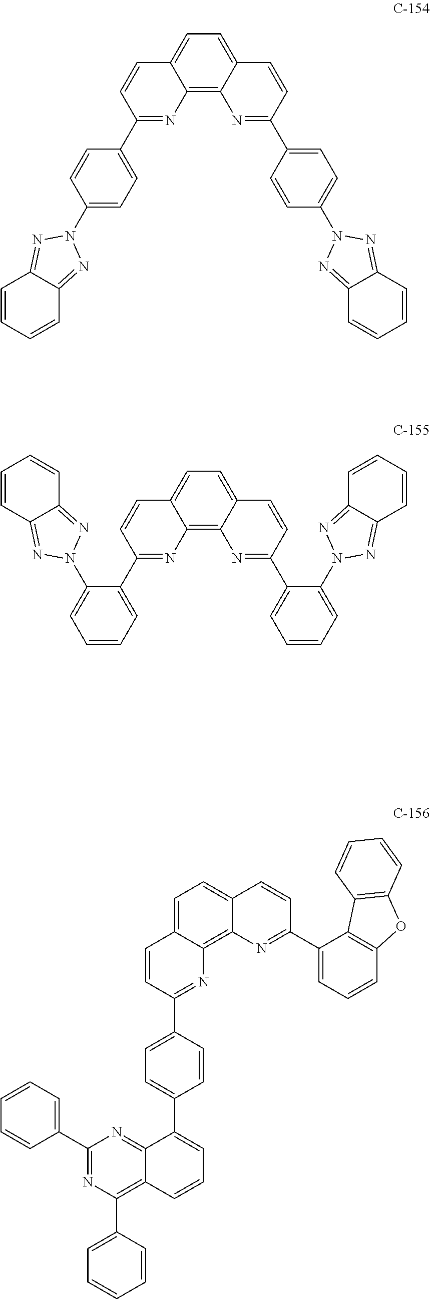 [SMILES: c1ccc2nn(-c3ccc(-c4ccc5ccc6ccc(-c7ccc(-n8nc9ccccc9n8)cc7)nc6c5n4)cc3)nc2c1 ; c1ccc(-n2nc3ccccc3n2)c(-c2ccc3ccc4ccc(-c5ccccc5-n5nc6ccccc6n5)nc4c3n2)c1 ; c1ccc(-c2nc(-c3ccccc3)c3cccc(-c4ccc(-c5ccc6ccc7ccc(-c8cccc9oc%10ccccc%10c89)nc7c6n5)cc4)c3n2)cc1] 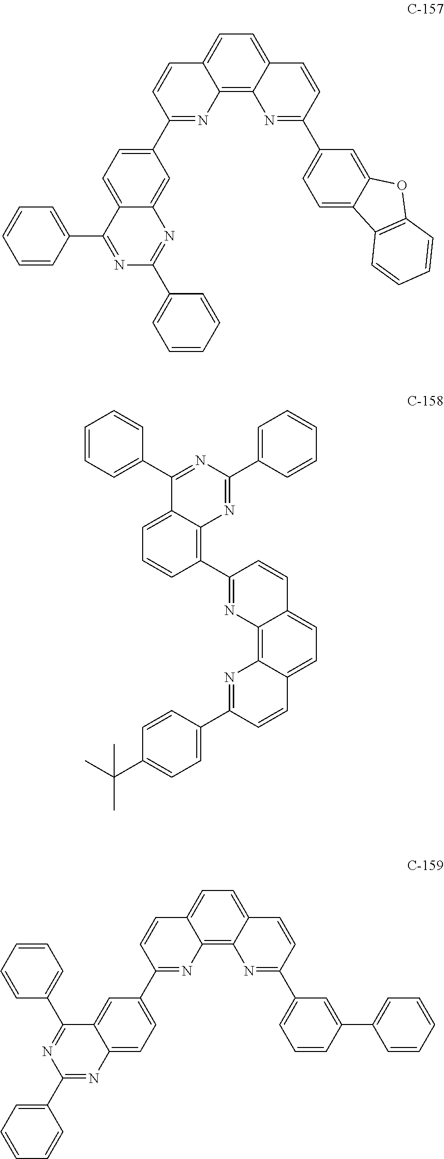 [SMILES: c1ccc(-c2nc(-c3ccccc3)c3ccc(-c4ccc5ccc6ccc(-c7ccc8c(c7)oc7ccccc78)nc6c5n4)cc3n2)cc1 ; CC(C)(C)c1ccc(-c2ccc3ccc4ccc(-c5cccc6c(-c7ccccc7)nc(-c7ccccc7)nc56)nc4c3n2)cc1 ; c1ccc(-c2cccc(-c3ccc4ccc5ccc(-c6ccc7nc(-c8ccccc8)nc(-c8ccccc8)c7c6)nc5c4n3)c2)cc1] 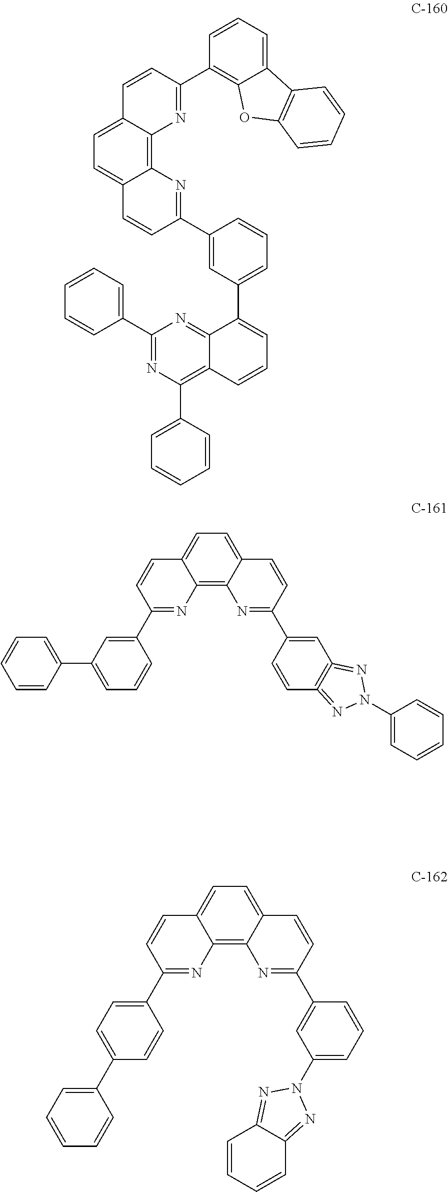 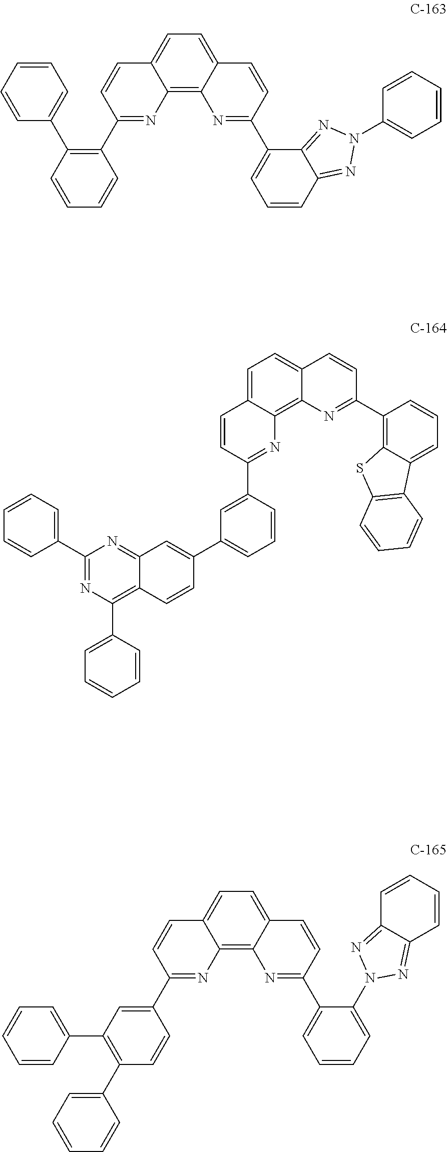 [SMILES: c1ccc(-c2ccccc2-c2ccc3ccc4ccc(-c5cccc6nn(-c7ccccc7)nc56)nc4c3n2)cc1 ; c1ccc(-c2nc(-c3ccccc3)c3ccc(-c4cccc(-c5ccc6ccc7ccc(-c8cccc9c8sc8ccccc89)nc7c6n5)c4)cc3n2)cc1 ; c1ccc(-c2ccc(-c3ccc4ccc5ccc(-c6ccccc6-n6nc7ccccc7n6)nc5c4n3)cc2-c2ccccc2)cc1] 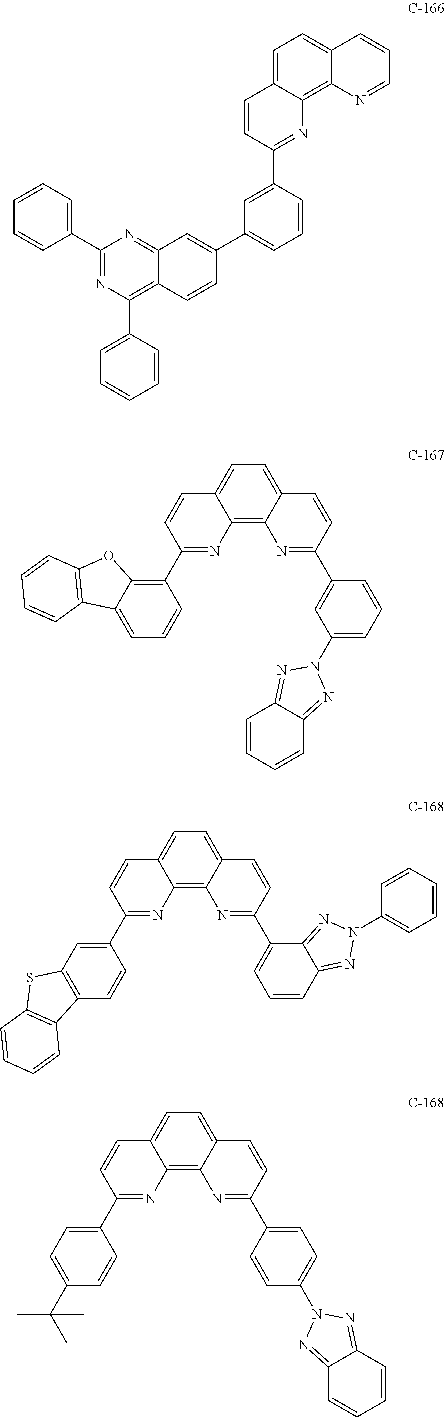 [SMILES: c1ccc(-c2nc(-c3ccccc3)c3ccc(-c4cccc(-c5ccc6ccc7cccnc7c6n5)c4)cc3n2)cc1 ; c1cc(-c2ccc3ccc4ccc(-c5cccc6c5oc5ccccc56)nc4c3n2)cc(-n2nc3ccccc3n2)c1 ; c1ccc(-n2nc3cccc(-c4ccc5ccc6ccc(-c7ccc8c(c7)sc7ccccc78)nc6c5n4)c3n2)cc1 ; CC(C)(C)c1ccc(-c2ccc3ccc4ccc(-c5ccc(-n6nc7ccccc7n6)cc5)nc4c3n2)cc1] 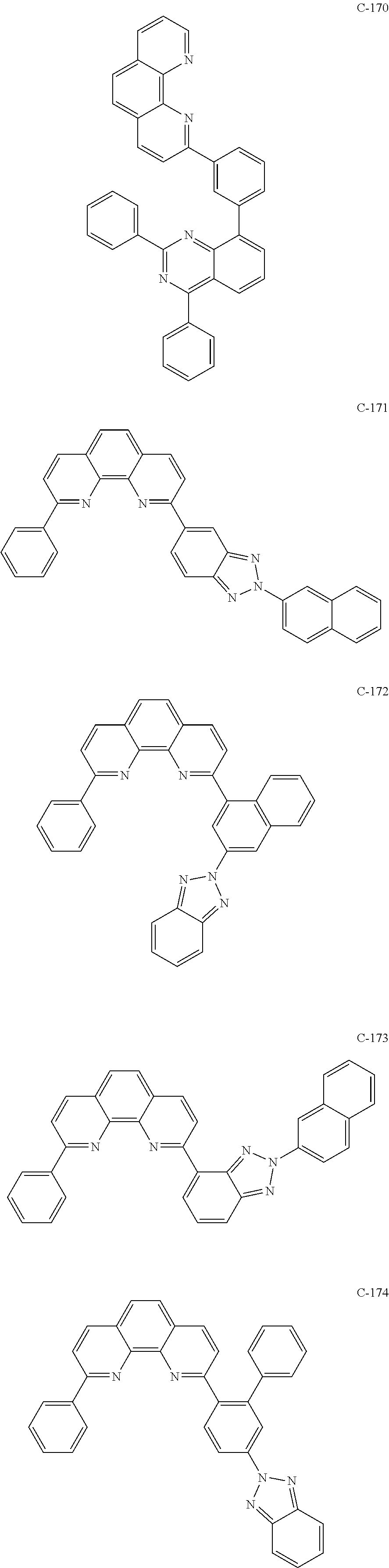 [SMILES: c1ccc(-c2nc(-c3ccccc3)c3cccc(-c4cccc(-c5ccc6ccc7cccnc7c6n5)c4)c3n2)cc1 ; c1ccc(-c2ccc3ccc4ccc(-c5ccc6nn(-c7ccc8ccccc8c7)nc6c5)nc4c3n2)cc1 ; c1ccc(-c2ccc3ccc4ccc(-c5cc(-n6nc7ccccc7n6)cc6ccccc56)nc4c3n2)cc1 ; c1ccc(-c2ccc3ccc4ccc(-c5cccc6nn(-c7ccc8ccccc8c7)nc56)nc4c3n2)cc1 ; c1ccc(-c2ccc3ccc4ccc(-c5ccc(-n6nc7ccccc7n6)cc5-c5ccccc5)nc4c3n2)cc1] 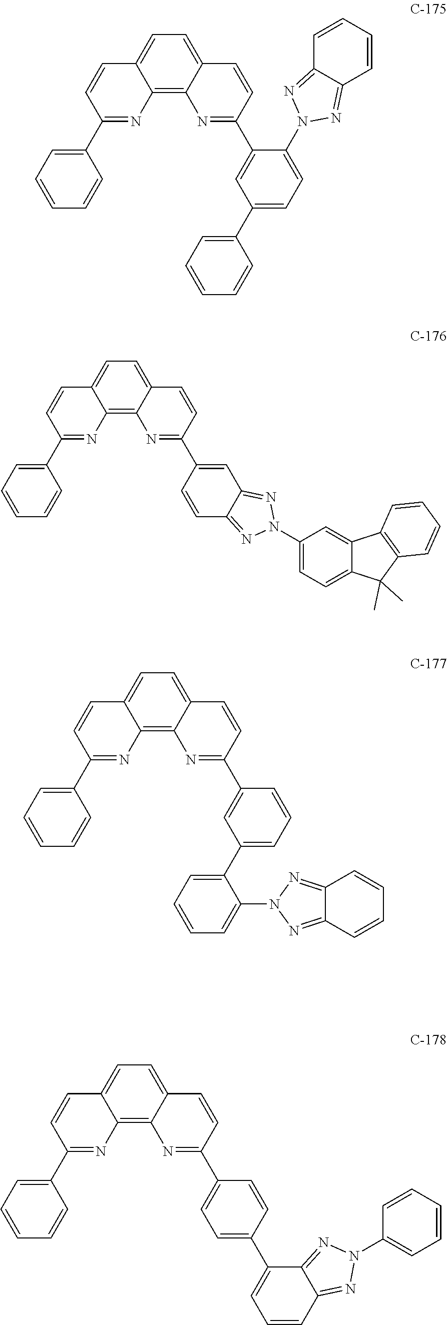 [SMILES: c1ccc(-c2ccc(-n3nc4ccccc4n3)c(-c3ccc4ccc5ccc(-c6ccccc6)nc5c4n3)c2)cc1 ; CC1(C)c2ccccc2-c2cc(-n3nc4ccc(-c5ccc6ccc7ccc(-c8ccccc8)nc7c6n5)cc4n3)ccc21 ; c1ccc(-c2ccc3ccc4ccc(-c5cccc(-c6ccccc6-n6nc7ccccc7n6)c5)nc4c3n2)cc1 ; c1ccc(-c2ccc3ccc4ccc(-c5ccc(-c6cccc7nn(-c8ccccc8)nc67)cc5)nc4c3n2)cc1] 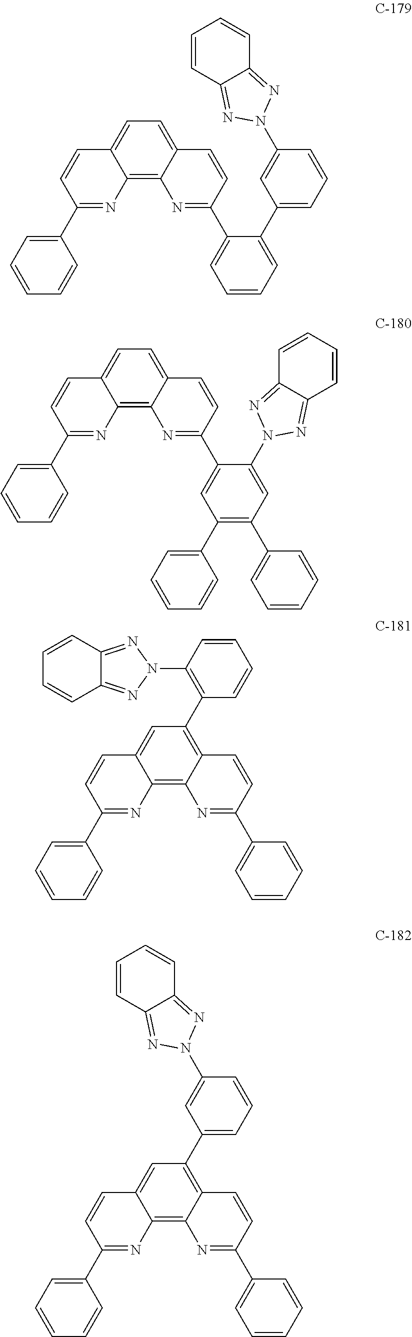 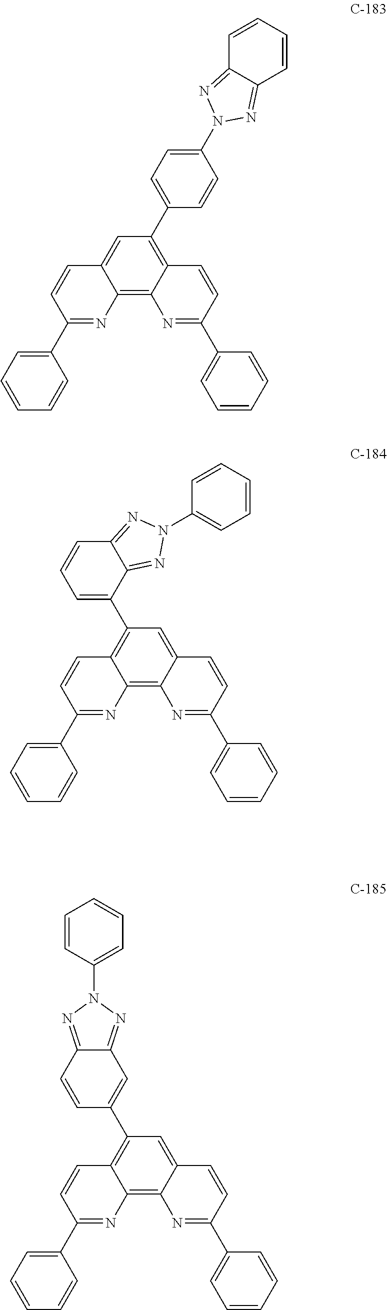 [SMILES: c1ccc(-c2ccc3cc(-c4ccc(-n5nc6ccccc6n5)cc4)c4ccc(-c5ccccc5)nc4c3n2)cc1 ; c1ccc(-c2ccc3cc(-c4cccc5nn(-c6ccccc6)nc45)c4ccc(-c5ccccc5)nc4c3n2)cc1 ; c1ccc(-c2ccc3cc(-c4ccc5nn(-c6ccccc6)nc5c4)c4ccc(-c5ccccc5)nc4c3n2)cc1] 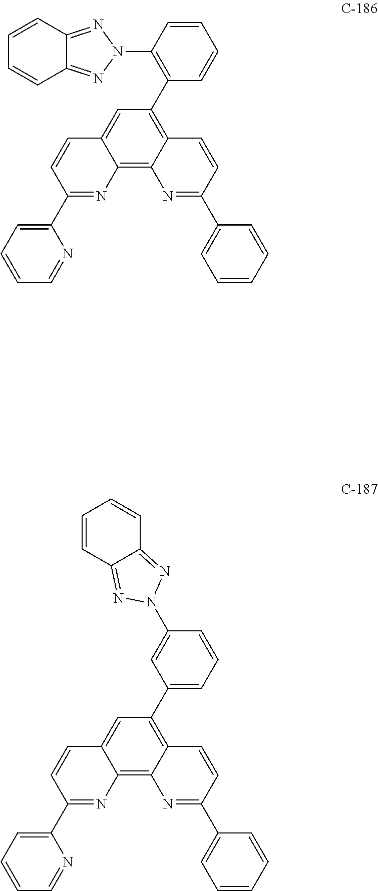 [SMILES: c1ccc(-c2ccc3c(-c4ccccc4-n4nc5ccccc5n4)cc4ccc(-c5ccccn5)nc4c3n2)cc1 ; c1ccc(-c2ccc3c(-c4cccc(-n5nc6ccccc6n5)c4)cc4ccc(-c5ccccn5)nc4c3n2)cc1] 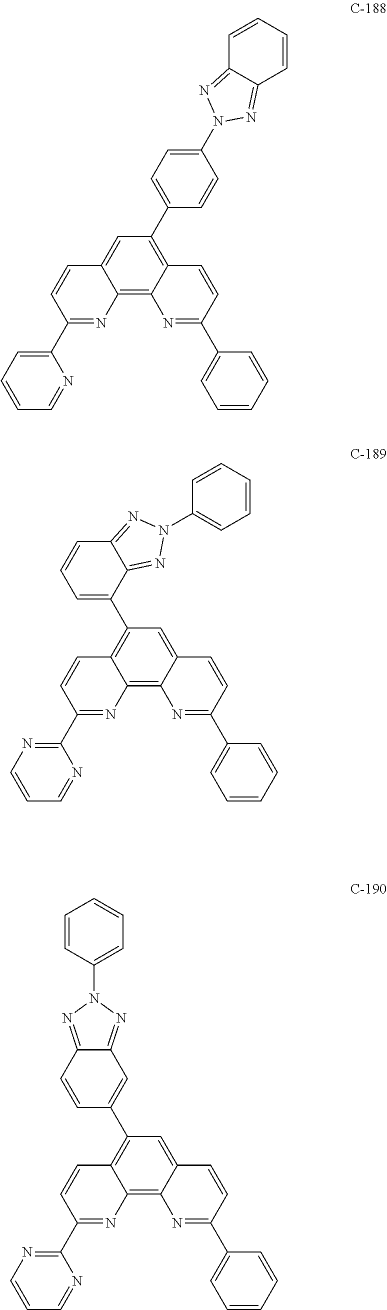 [SMILES: c1ccc(-c2ccc3c(-c4ccc(-n5nc6ccccc6n5)cc4)cc4ccc(-c5ccccn5)nc4c3n2)cc1 ; c1ccc(-c2ccc3cc(-c4cccc5nn(-c6ccccc6)nc45)c4ccc(-c5ncccn5)nc4c3n2)cc1 ; c1ccc(-c2ccc3cc(-c4ccc5nn(-c6ccccc6)nc5c4)c4ccc(-c5ncccn5)nc4c3n2)cc1] 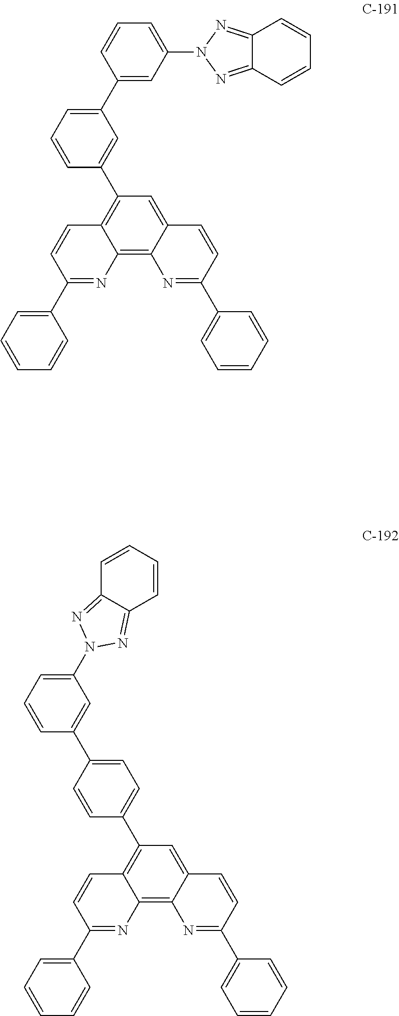 [SMILES: c1ccc(-c2ccc3cc(-c4cccc(-c5cccc(-n6nc7ccccc7n6)c5)c4)c4ccc(-c5ccccc5)nc4c3n2)cc1 ; c1ccc(-c2ccc3cc(-c4ccc(-c5cccc(-n6nc7ccccc7n6)c5)cc4)c4ccc(-c5ccccc5)nc4c3n2)cc1] 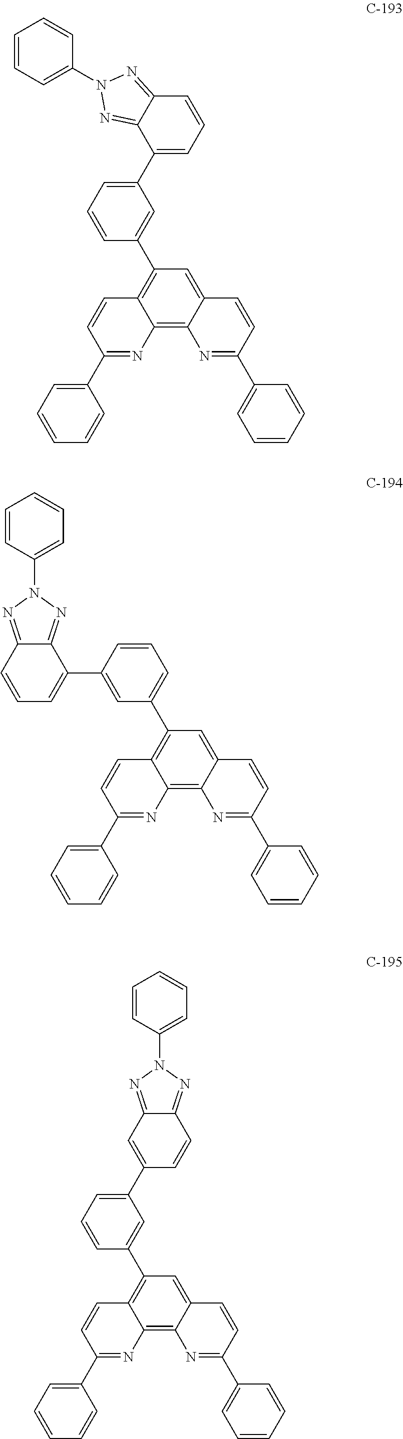 [SMILES: c1ccc(-c2ccc3cc(-c4cccc(-c5cccc6nn(-c7ccccc7)nc56)c4)c4ccc(-c5ccccc5)nc4c3n2)cc1 ; c1ccc(-c2ccc3cc(-c4cccc(-c5cccc6nn(-c7ccccc7)nc56)c4)c4ccc(-c5ccccc5)nc4c3n2)cc1 ; c1ccc(-c2ccc3cc(-c4cccc(-c5ccc6nn(-c7ccccc7)nc6c5)c4)c4ccc(-c5ccccc5)nc4c3n2)cc1] 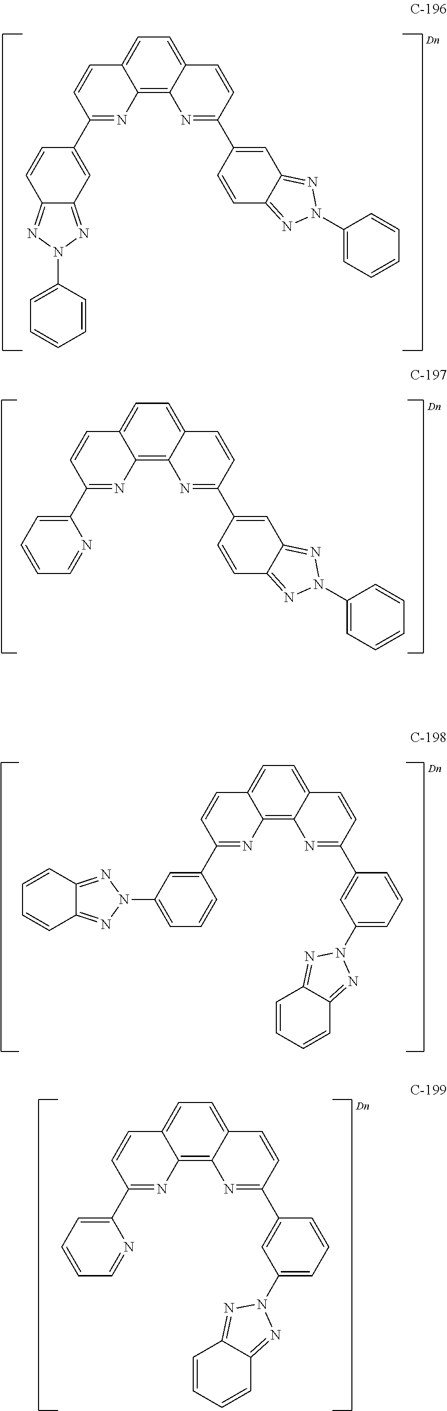 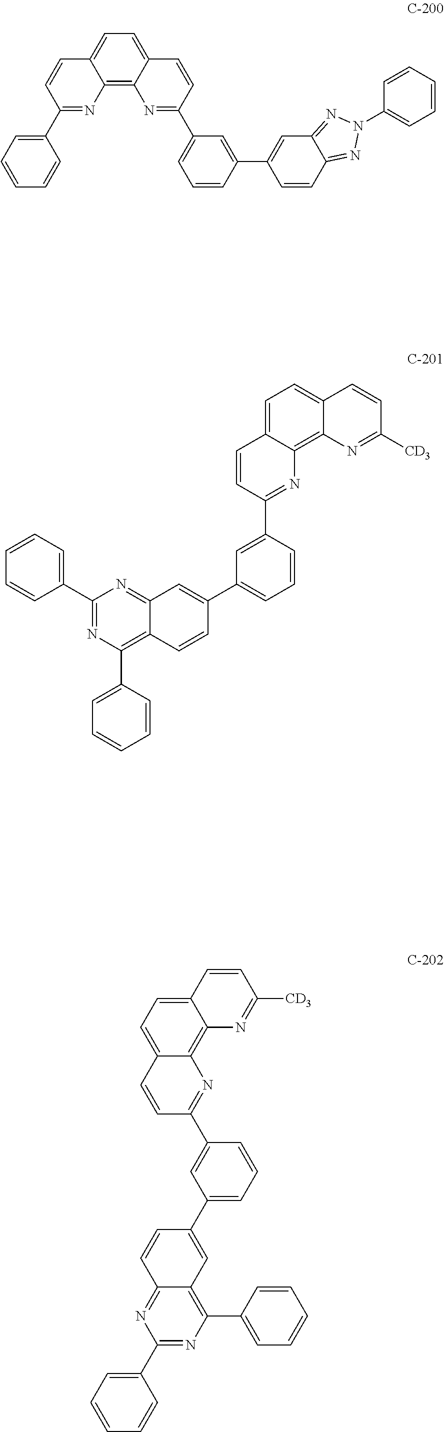 [SMILES: c1ccc(-c2ccc3ccc4ccc(-c5cccc(-c6ccc7nn(-c8ccccc8)nc7c6)c5)nc4c3n2)cc1 ; [2H]C([2H])([2H])c1ccc2ccc3ccc(-c4cccc(-c5ccc6c(-c7ccccc7)nc(-c7ccccc7)nc6c5)c4)nc3c2n1 ; [2H]C([2H])([2H])c1ccc2ccc3ccc(-c4cccc(-c5ccc6nc(-c7ccccc7)nc(-c7ccccc7)c6c5)c4)nc3c2n1] 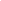 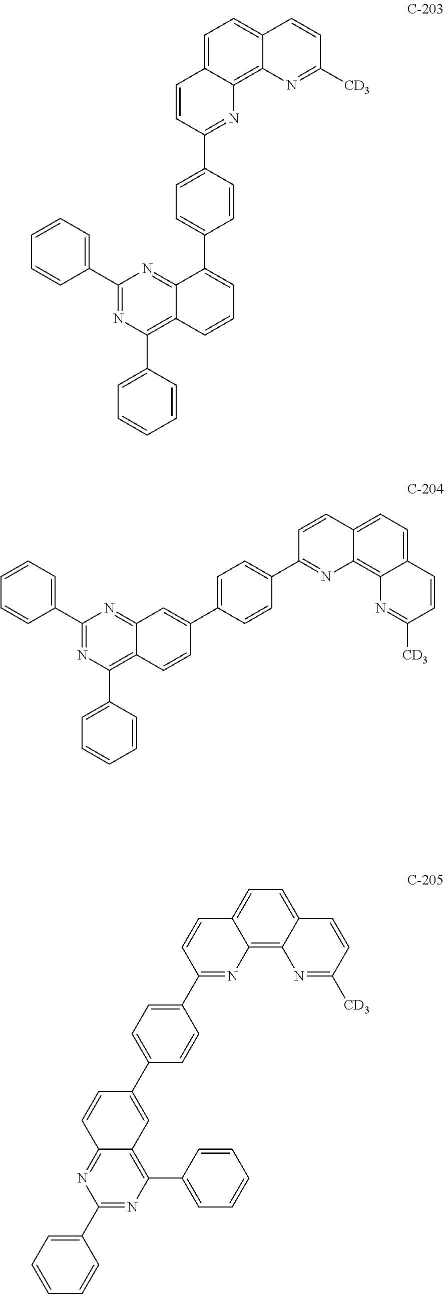 [SMILES: [2H]C([2H])([2H])c1ccc2ccc3ccc(-c4ccc(-c5cccc6c(-c7ccccc7)nc(-c7ccccc7)nc56)cc4)nc3c2n1 ; [2H]C([2H])([2H])c1ccc2ccc3ccc(-c4ccc(-c5ccc6c(-c7ccccc7)nc(-c7ccccc7)nc6c5)cc4)nc3c2n1 ; [2H]C([2H])([2H])c1ccc2ccc3ccc(-c4ccc(-c5ccc6nc(-c7ccccc7)nc(-c7ccccc7)c6c5)cc4)nc3c2n1] 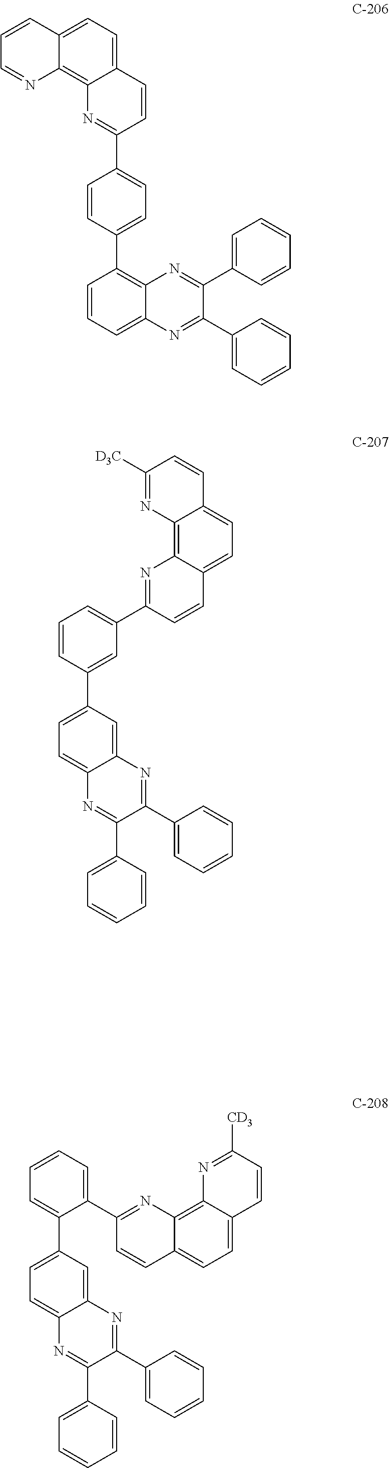 [SMILES: c1ccc(-c2nc3cccc(-c4ccc(-c5ccc6ccc7cccnc7c6n5)cc4)c3nc2-c2ccccc2)cc1 ; [2H]C([2H])([2H])c1ccc2ccc3ccc(-c4cccc(-c5ccc6nc(-c7ccccc7)c(-c7ccccc7)nc6c5)c4)nc3c2n1 ; [2H]C([2H])([2H])c1ccc2ccc3ccc(-c4ccccc4-c4ccc5nc(-c6ccccc6)c(-c6ccccc6)nc5c4)nc3c2n1] 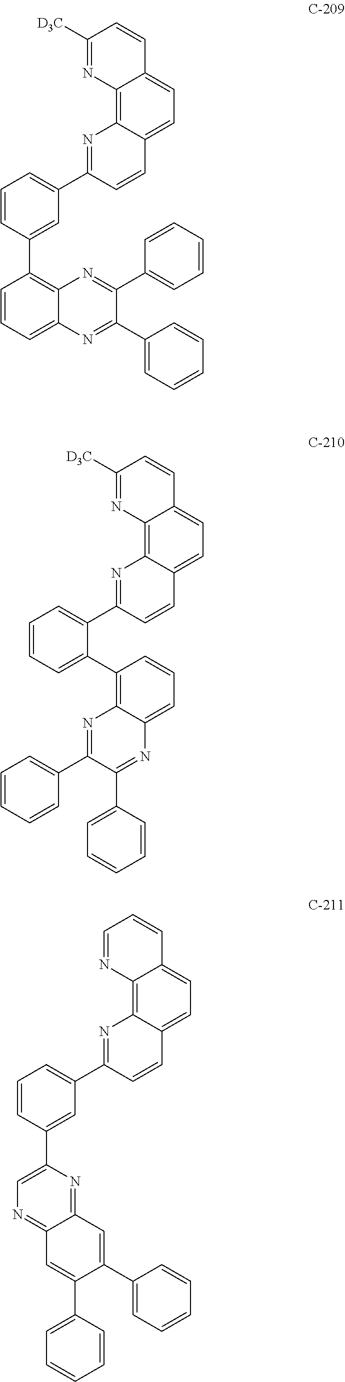 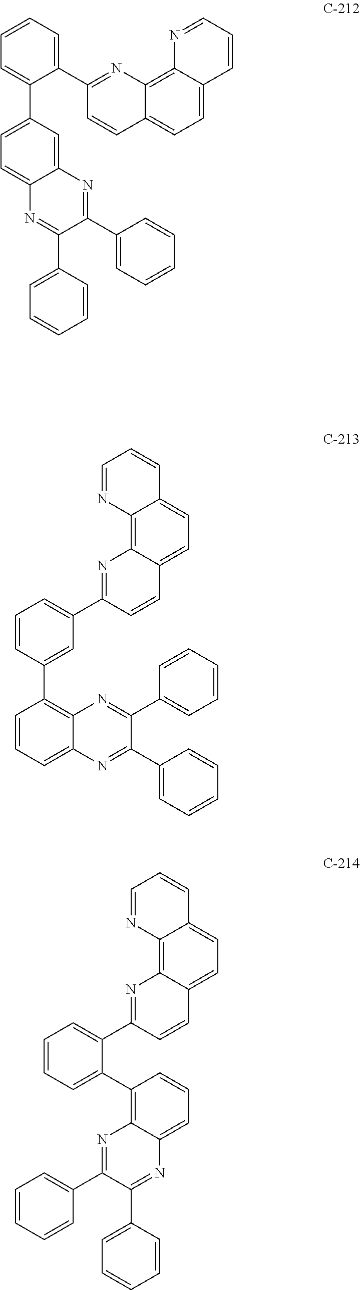 [SMILES: c1ccc(-c2nc3ccc(-c4ccccc4-c4ccc5ccc6cccnc6c5n4)cc3nc2-c2ccccc2)cc1 ; c1ccc(-c2nc3cccc(-c4cccc(-c5ccc6ccc7cccnc7c6n5)c4)c3nc2-c2ccccc2)cc1 ; c1ccc(-c2nc3cccc(-c4ccccc4-c4ccc5ccc6cccnc6c5n4)c3nc2-c2ccccc2)cc1] 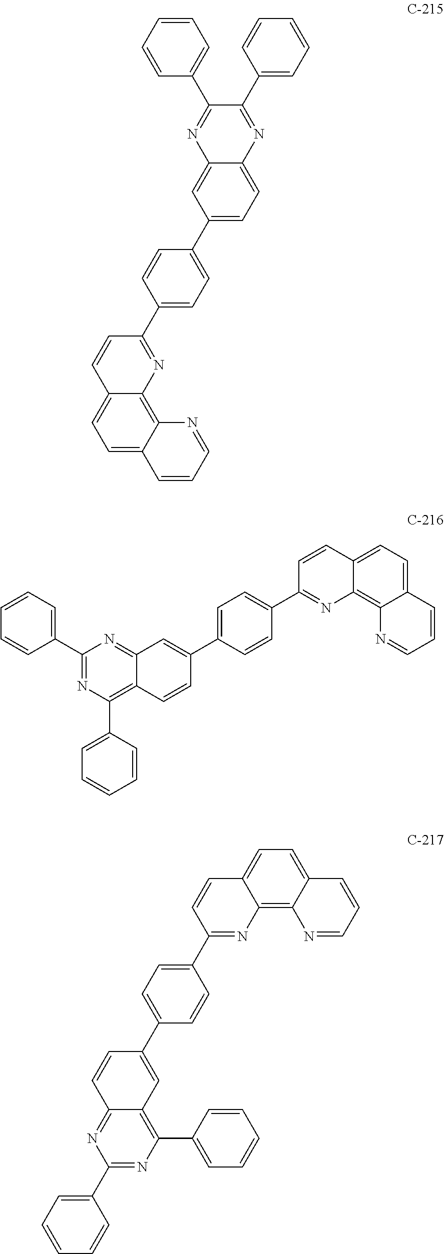 [SMILES: c1ccc(-c2nc3ccc(-c4ccc(-c5ccc6ccc7cccnc7c6n5)cc4)cc3nc2-c2ccccc2)cc1 ; c1ccc(-c2nc(-c3ccccc3)c3ccc(-c4ccc(-c5ccc6ccc7cccnc7c6n5)cc4)cc3n2)cc1 ; c1ccc(-c2nc(-c3ccccc3)c3cc(-c4ccc(-c5ccc6ccc7cccnc7c6n5)cc4)ccc3n2)cc1] 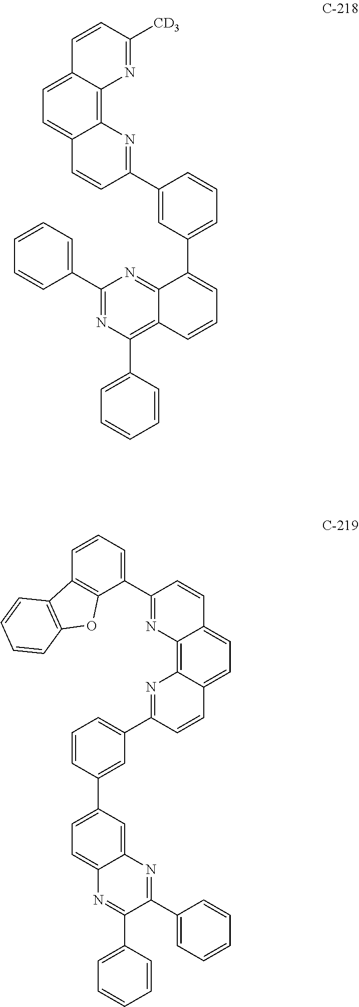 [SMILES: [2H]C([2H])([2H])c1ccc2ccc3ccc(-c4cccc(-c5cccc6c(-c7ccccc7)nc(-c7ccccc7)nc56)c4)nc3c2n1 ; c1ccc(-c2nc3ccc(-c4cccc(-c5ccc6ccc7ccc(-c8cccc9c8oc8ccccc89)nc7c6n5)c4)cc3nc2-c2ccccc2)cc1] 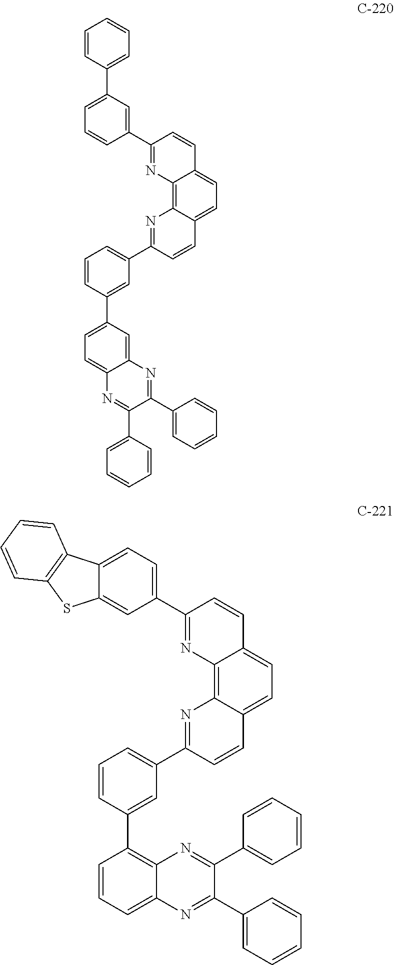 [SMILES: c1ccc(-c2cccc(-c3ccc4ccc5ccc(-c6cccc(-c7ccc8nc(-c9ccccc9)c(-c9ccccc9)nc8c7)c6)nc5c4n3)c2)cc1 ; c1ccc(-c2nc3cccc(-c4cccc(-c5ccc6ccc7ccc(-c8ccc9c(c8)sc8ccccc89)nc7c6n5)c4)c3nc2-c2ccccc2)cc1] 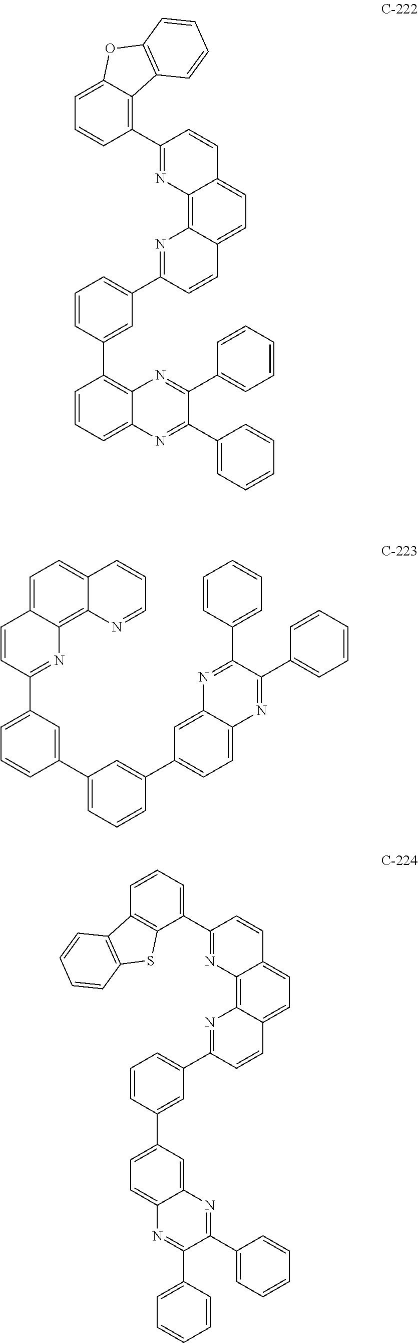 [SMILES: c1ccc(-c2nc3cccc(-c4cccc(-c5ccc6ccc7ccc(-c8cccc9oc%10ccccc%10c89)nc7c6n5)c4)c3nc2-c2ccccc2)cc1 ; c1ccc(-c2nc3ccc(-c4cccc(-c5cccc(-c6ccc7ccc8cccnc8c7n6)c5)c4)cc3nc2-c2ccccc2)cc1 ; c1ccc(-c2nc3ccc(-c4cccc(-c5ccc6ccc7ccc(-c8cccc9c8sc8ccccc89)nc7c6n5)c4)cc3nc2-c2ccccc2)cc1] 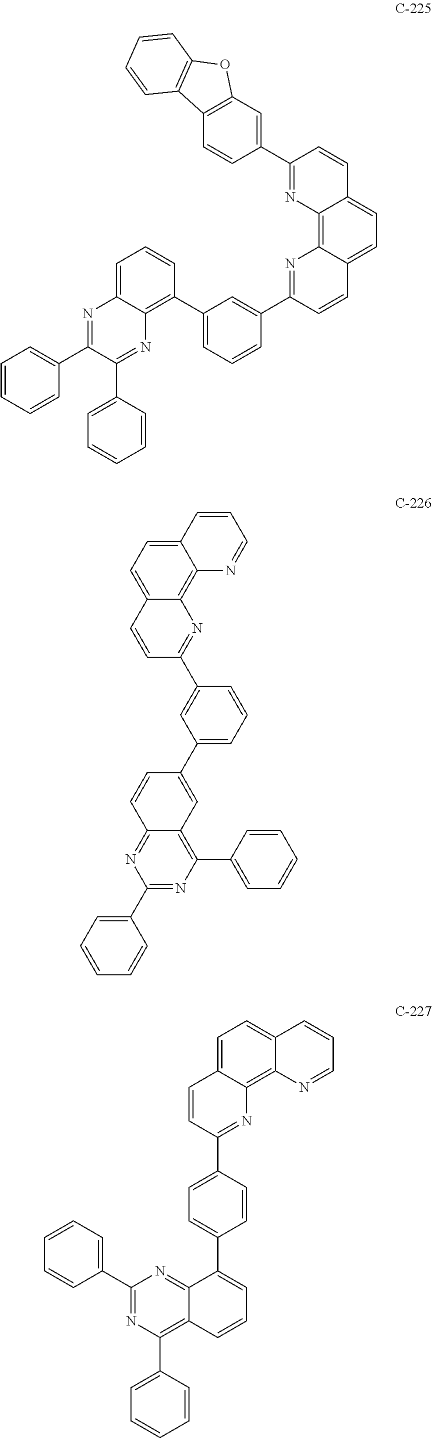 [SMILES: c1ccc(-c2nc3cccc(-c4cccc(-c5ccc6ccc7ccc(-c8ccc9c(c8)oc8ccccc89)nc7c6n5)c4)c3nc2-c2ccccc2)cc1 ; c1ccc(-c2nc(-c3ccccc3)c3cc(-c4cccc(-c5ccc6ccc7cccnc7c6n5)c4)ccc3n2)cc1 ; c1ccc(-c2nc(-c3ccccc3)c3cccc(-c4ccc(-c5ccc6ccc7cccnc7c6n5)cc4)c3n2)cc1] 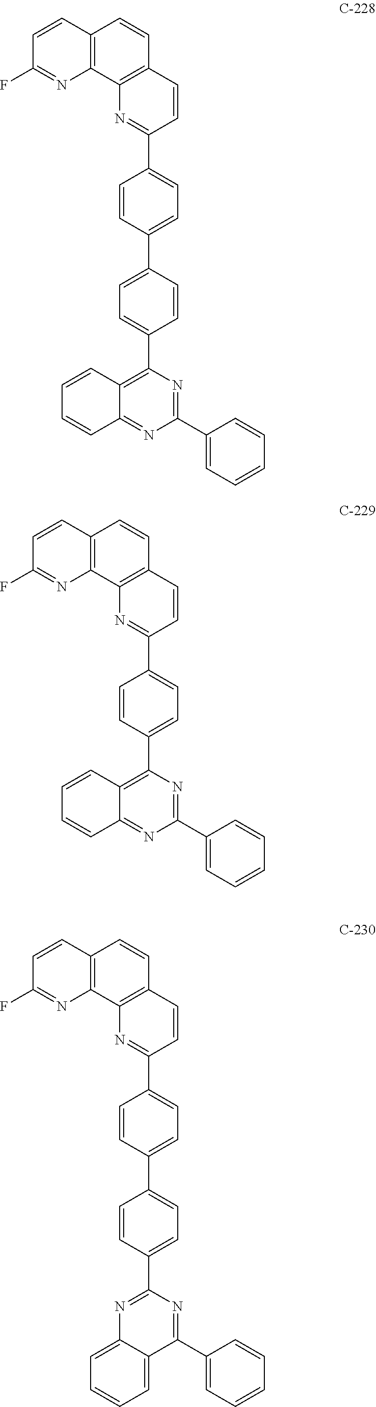 [SMILES: Fc1ccc2ccc3ccc(-c4ccc(-c5ccc(-c6nc(-c7ccccc7)nc7ccccc67)cc5)cc4)nc3c2n1 ; Fc1ccc2ccc3ccc(-c4ccc(-c5nc(-c6ccccc6)nc6ccccc56)cc4)nc3c2n1 ; Fc1ccc2ccc3ccc(-c4ccc(-c5ccc(-c6nc(-c7ccccc7)c7ccccc7n6)cc5)cc4)nc3c2n1] 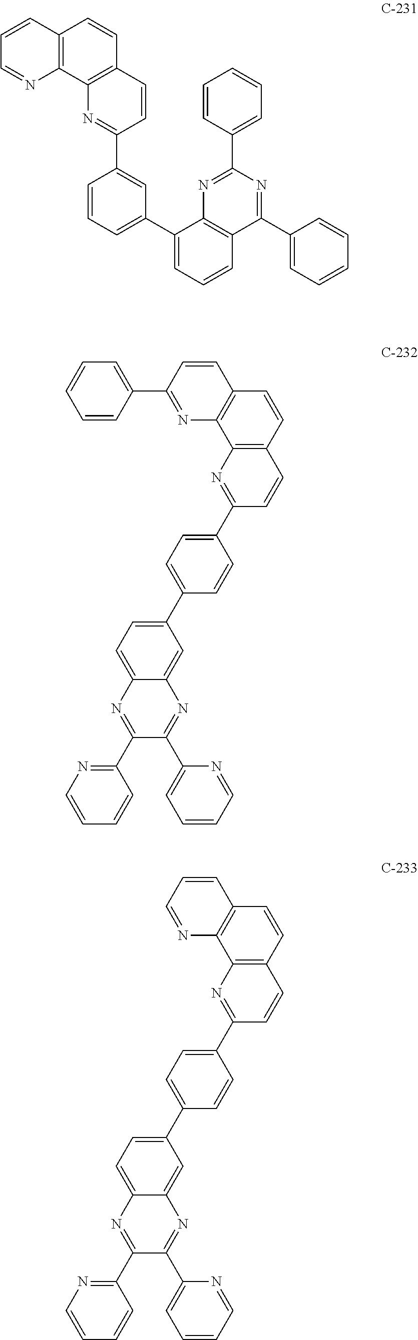 [SMILES: c1ccc(-c2nc(-c3ccccc3)c3cccc(-c4cccc(-c5ccc6ccc7cccnc7c6n5)c4)c3n2)cc1 ; c1ccc(-c2ccc3ccc4ccc(-c5ccc(-c6ccc7nc(-c8ccccn8)c(-c8ccccn8)nc7c6)cc5)nc4c3n2)cc1 ; c1ccc(-c2nc3ccc(-c4ccc(-c5ccc6ccc7cccnc7c6n5)cc4)cc3nc2-c2ccccn2)nc1] 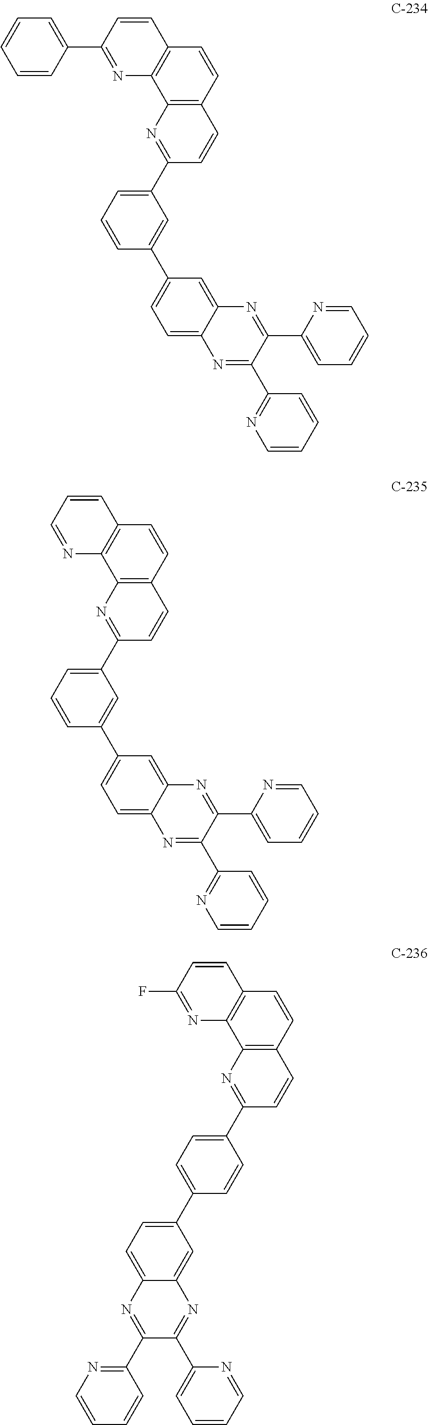 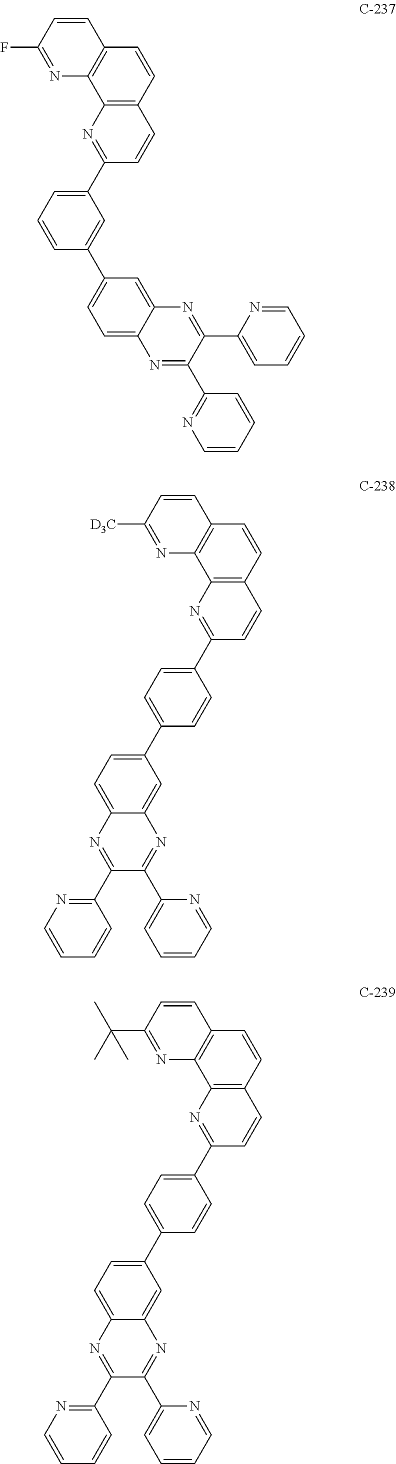 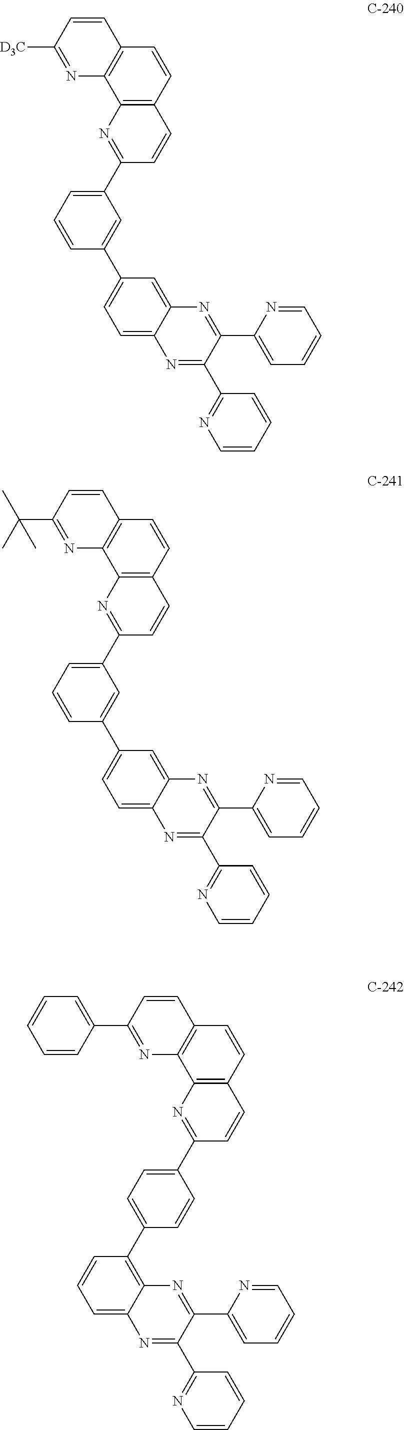 [SMILES: [2H]C([2H])([2H])c1ccc2ccc3ccc(-c4cccc(-c5ccc6nc(-c7ccccn7)c(-c7ccccn7)nc6c5)c4)nc3c2n1 ; CC(C)(C)c1ccc2ccc3ccc(-c4cccc(-c5ccc6nc(-c7ccccn7)c(-c7ccccn7)nc6c5)c4)nc3c2n1 ; c1ccc(-c2ccc3ccc4ccc(-c5ccc(-c6cccc7nc(-c8ccccn8)c(-c8ccccn8)nc67)cc5)nc4c3n2)cc1] 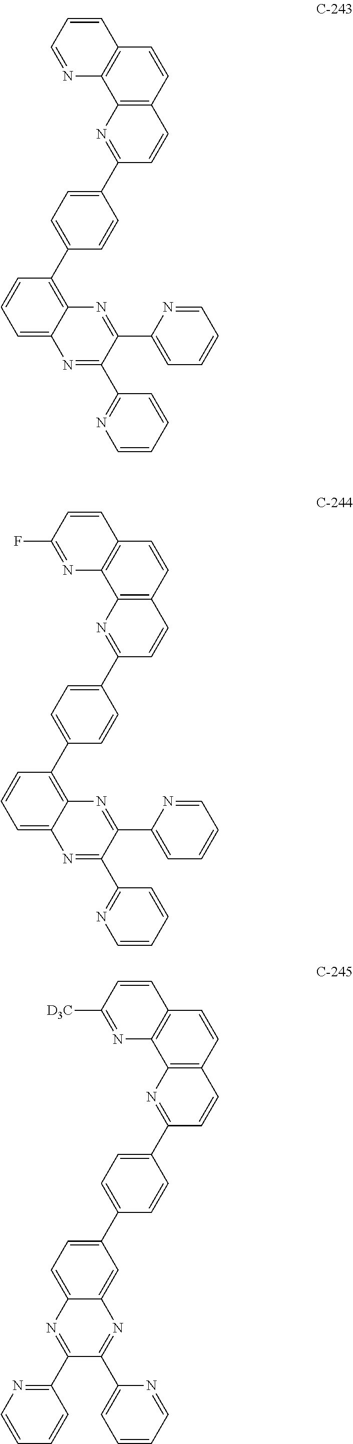 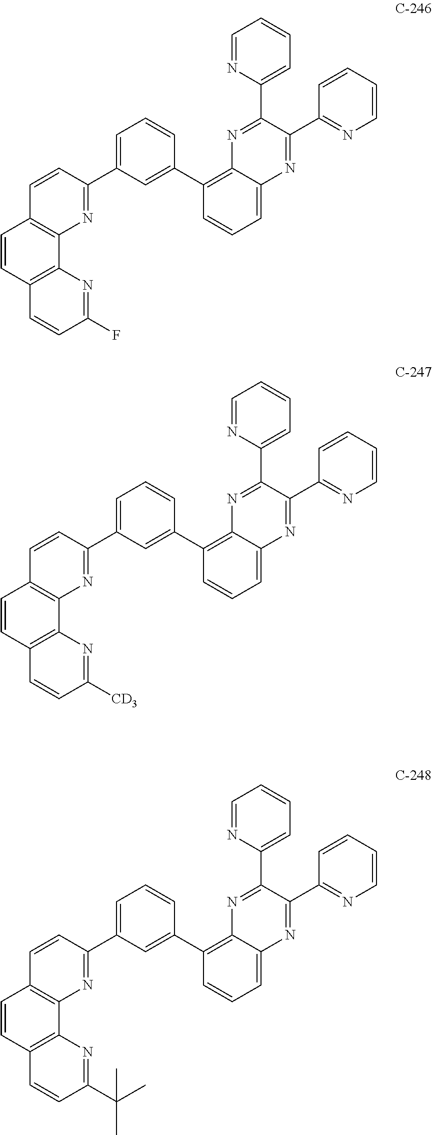 [SMILES: Fc1ccc2ccc3ccc(-c4cccc(-c5cccc6nc(-c7ccccn7)c(-c7ccccn7)nc56)c4)nc3c2n1 ; [2H]C([2H])([2H])c1ccc2ccc3ccc(-c4cccc(-c5cccc6nc(-c7ccccn7)c(-c7ccccn7)nc56)c4)nc3c2n1 ; CC(C)(C)c1ccc2ccc3ccc(-c4cccc(-c5cccc6nc(-c7ccccn7)c(-c7ccccn7)nc56)c4)nc3c2n1] 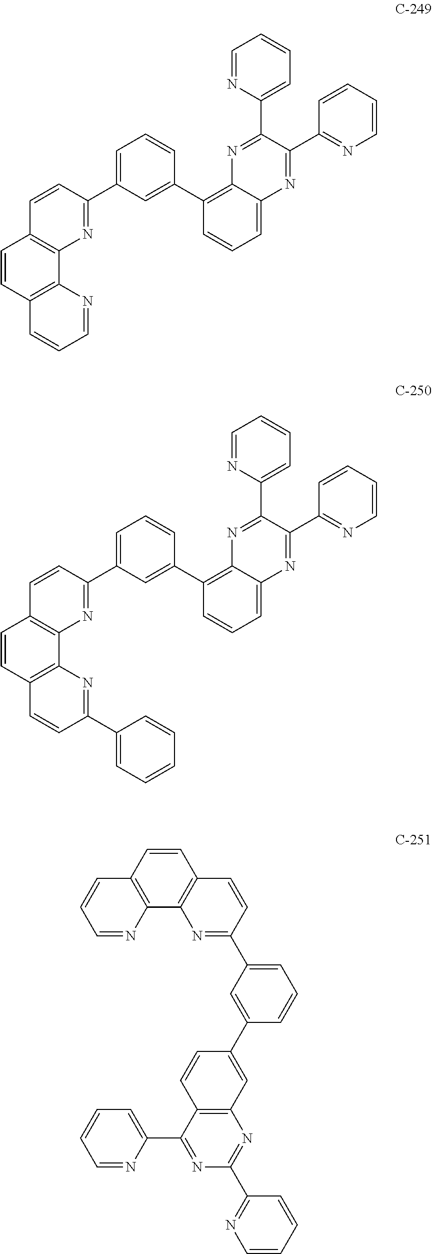 [SMILES: c1ccc(-c2nc3cccc(-c4cccc(-c5ccc6ccc7cccnc7c6n5)c4)c3nc2-c2ccccn2)nc1 ; c1ccc(-c2ccc3ccc4ccc(-c5cccc(-c6cccc7nc(-c8ccccn8)c(-c8ccccn8)nc67)c5)nc4c3n2)cc1 ; c1ccc(-c2nc(-c3ccccn3)c3ccc(-c4cccc(-c5ccc6ccc7cccnc7c6n5)c4)cc3n2)nc1] 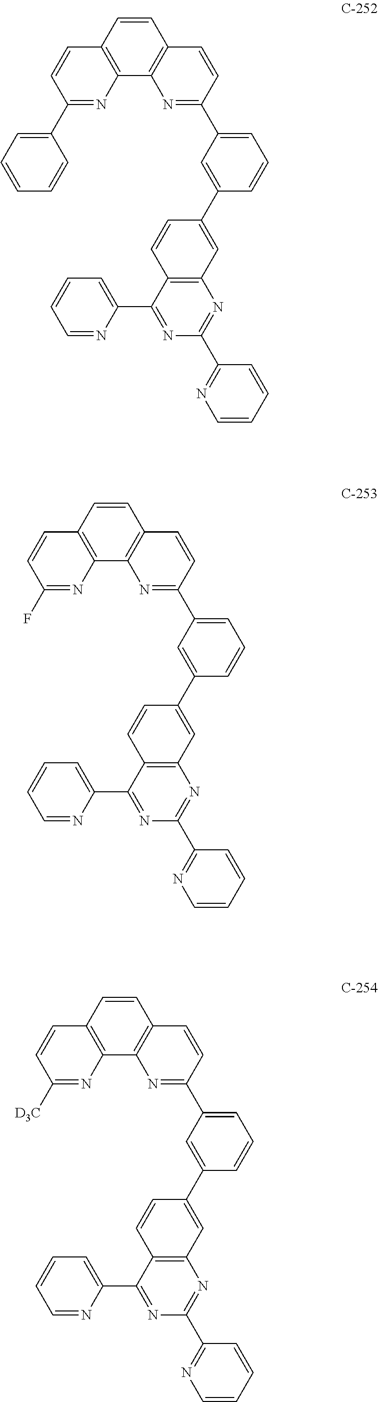 [SMILES: c1ccc(-c2ccc3ccc4ccc(-c5cccc(-c6ccc7c(-c8ccccn8)nc(-c8ccccn8)nc7c6)c5)nc4c3n2)cc1 ; Fc1ccc2ccc3ccc(-c4cccc(-c5ccc6c(-c7ccccn7)nc(-c7ccccn7)nc6c5)c4)nc3c2n1 ; [2H]C([2H])([2H])c1ccc2ccc3ccc(-c4cccc(-c5ccc6c(-c7ccccn7)nc(-c7ccccn7)nc6c5)c4)nc3c2n1] 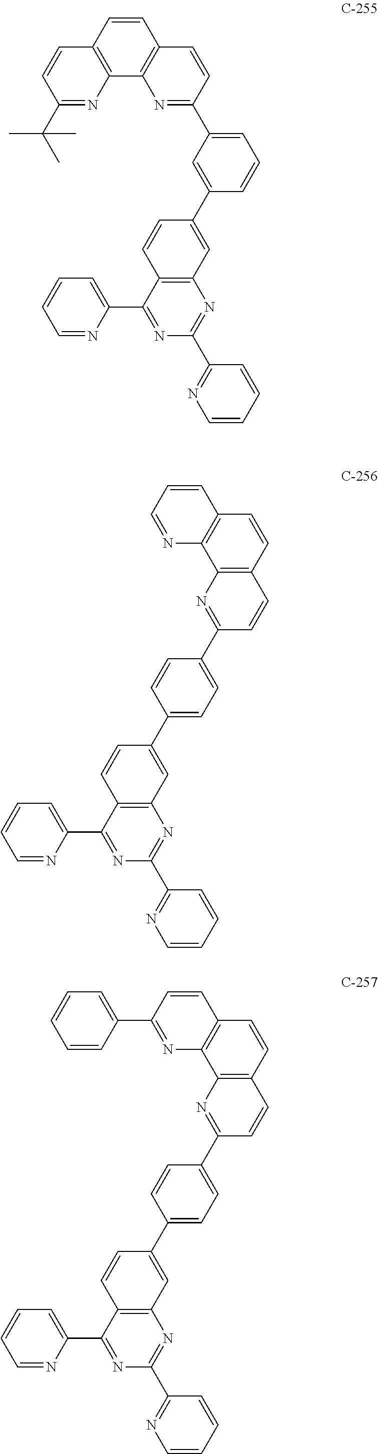 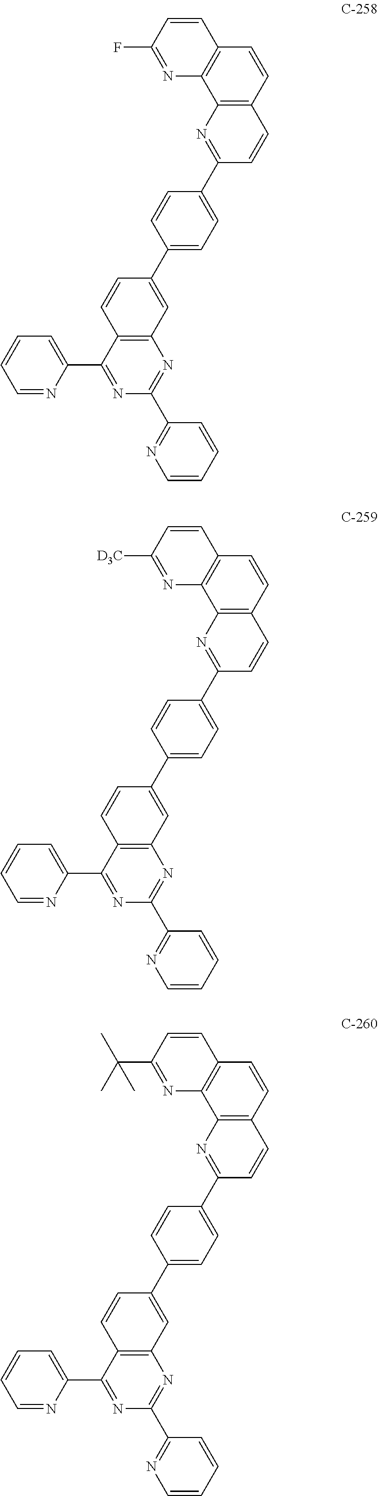 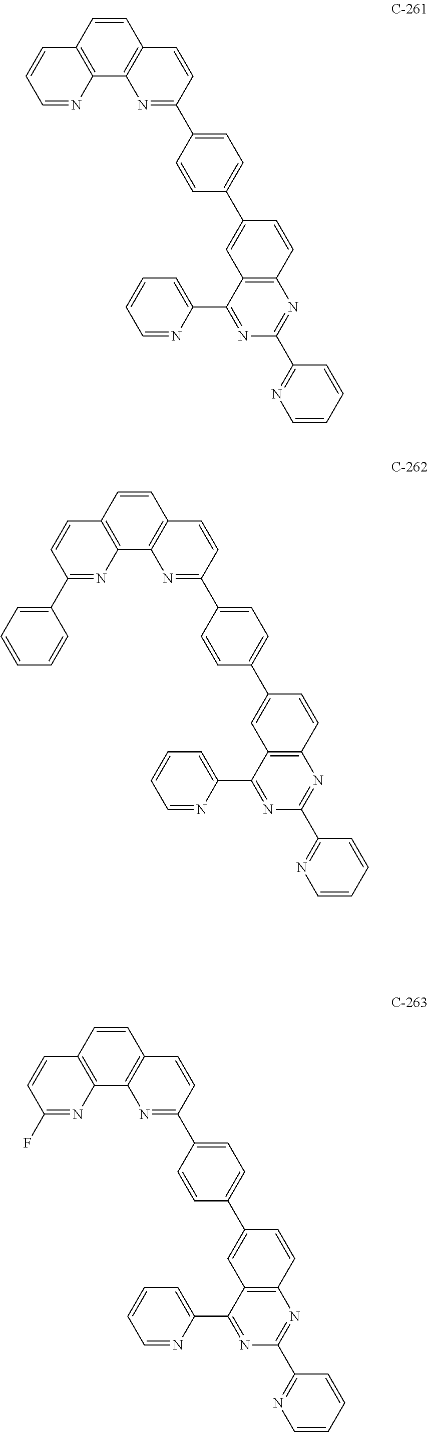 [SMILES: c1ccc(-c2nc(-c3ccccn3)c3cc(-c4ccc(-c5ccc6ccc7cccnc7c6n5)cc4)ccc3n2)nc1 ; c1ccc(-c2ccc3ccc4ccc(-c5ccc(-c6ccc7nc(-c8ccccn8)nc(-c8ccccn8)c7c6)cc5)nc4c3n2)cc1 ; Fc1ccc2ccc3ccc(-c4ccc(-c5ccc6nc(-c7ccccn7)nc(-c7ccccn7)c6c5)cc4)nc3c2n1] 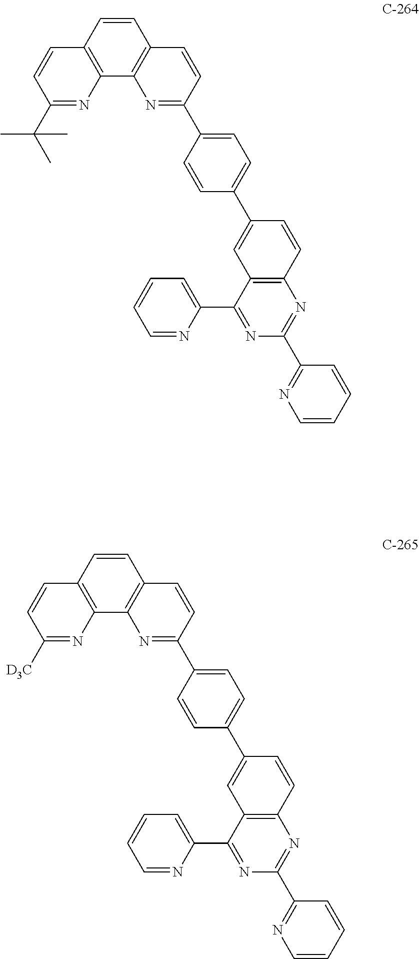 [SMILES: CC(C)(C)c1ccc2ccc3ccc(-c4ccc(-c5ccc6nc(-c7ccccn7)nc(-c7ccccn7)c6c5)cc4)nc3c2n1 ; [2H]C([2H])([2H])c1ccc2ccc3ccc(-c4ccc(-c5ccc6nc(-c7ccccn7)nc(-c7ccccn7)c6c5)cc4)nc3c2n1] 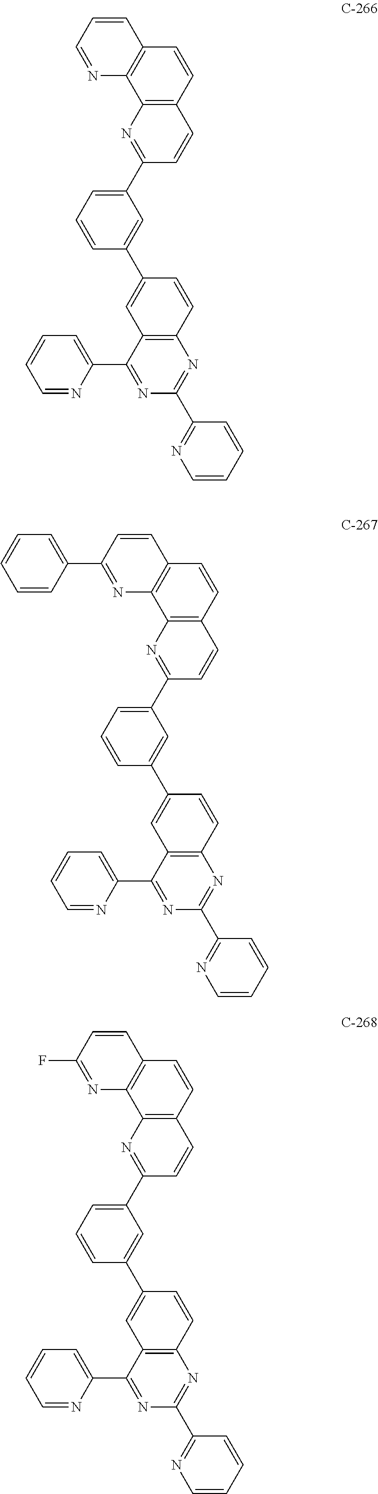 [SMILES: c1ccc(-c2nc(-c3ccccn3)c3cc(-c4cccc(-c5ccc6ccc7cccnc7c6n5)c4)ccc3n2)nc1 ; c1ccc(-c2ccc3ccc4ccc(-c5cccc(-c6ccc7nc(-c8ccccn8)nc(-c8ccccn8)c7c6)c5)nc4c3n2)cc1 ; Fc1ccc2ccc3ccc(-c4cccc(-c5ccc6nc(-c7ccccn7)nc(-c7ccccn7)c6c5)c4)nc3c2n1] 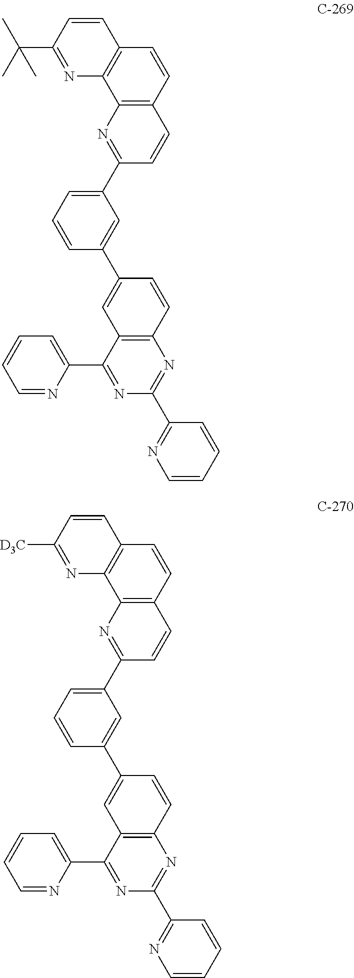 [SMILES: CC(C)(C)c1ccc2ccc3ccc(-c4cccc(-c5ccc6nc(-c7ccccn7)nc(-c7ccccn7)c6c5)c4)nc3c2n1 ; [2H]C([2H])([2H])c1ccc2ccc3ccc(-c4cccc(-c5ccc6nc(-c7ccccn7)nc(-c7ccccn7)c6c5)c4)nc3c2n1] 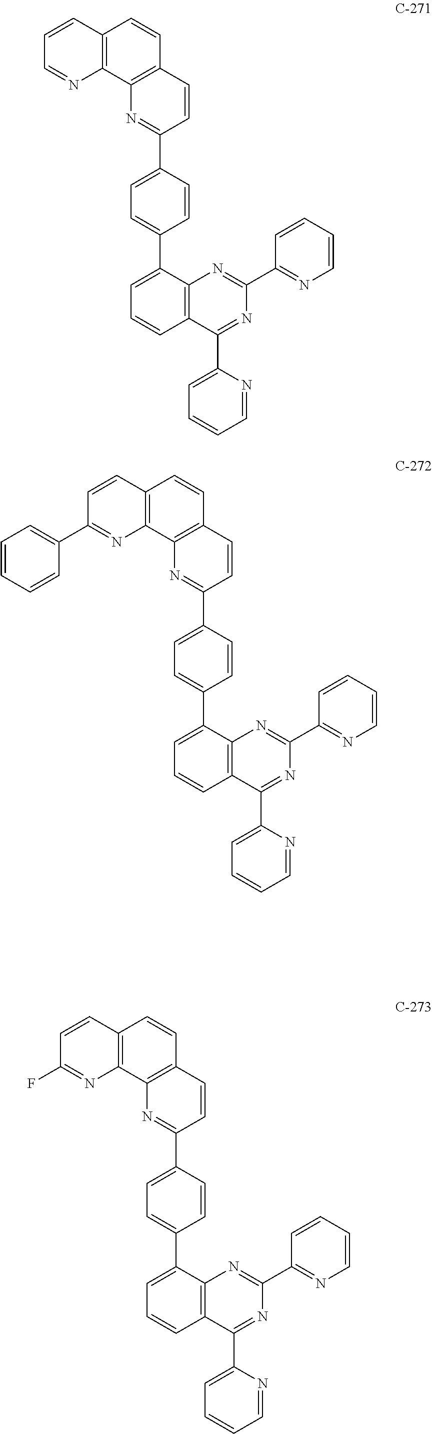 [SMILES: c1ccc(-c2nc(-c3ccccn3)c3cccc(-c4ccc(-c5ccc6ccc7cccnc7c6n5)cc4)c3n2)nc1 ; c1ccc(-c2ccc3ccc4ccc(-c5ccc(-c6cccc7c(-c8ccccn8)nc(-c8ccccn8)nc67)cc5)nc4c3n2)cc1 ; Fc1ccc2ccc3ccc(-c4ccc(-c5cccc6c(-c7ccccn7)nc(-c7ccccn7)nc56)cc4)nc3c2n1] 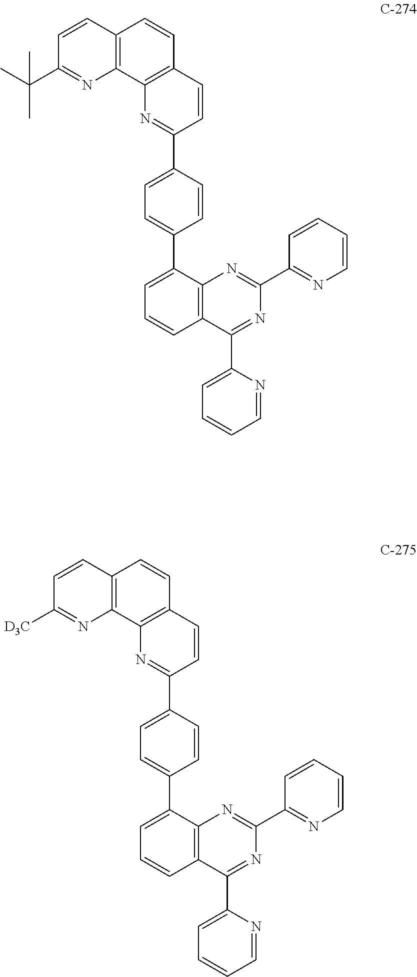 [SMILES: CC(C)(C)c1ccc2ccc3ccc(-c4ccc(-c5cccc6c(-c7ccccn7)nc(-c7ccccn7)nc56)cc4)nc3c2n1 ; [2H]C([2H])([2H])c1ccc2ccc3ccc(-c4ccc(-c5cccc6c(-c7ccccn7)nc(-c7ccccn7)nc56)cc4)nc3c2n1] 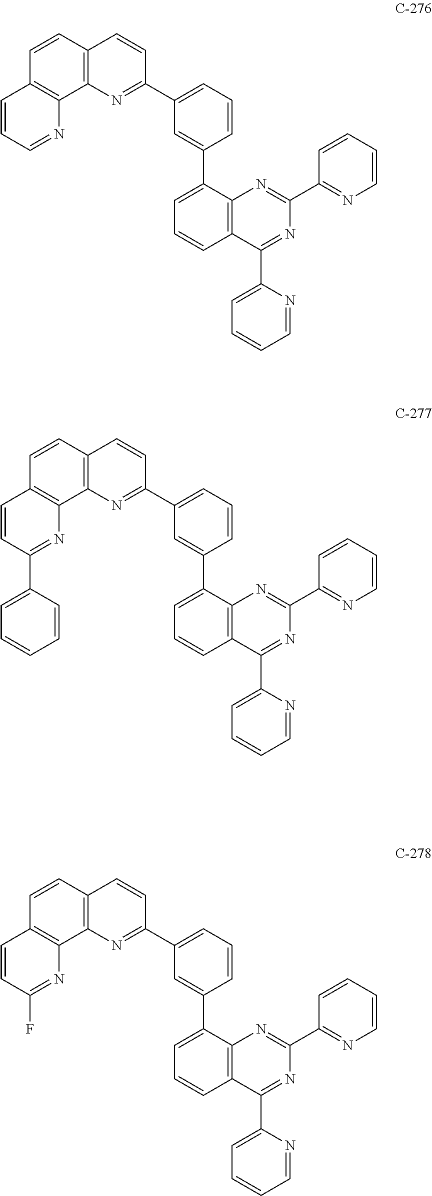 [SMILES: c1ccc(-c2nc(-c3ccccn3)c3cccc(-c4cccc(-c5ccc6ccc7cccnc7c6n5)c4)c3n2)nc1 ; c1ccc(-c2ccc3ccc4ccc(-c5cccc(-c6cccc7c(-c8ccccn8)nc(-c8ccccn8)nc67)c5)nc4c3n2)cc1 ; Fc1ccc2ccc3ccc(-c4cccc(-c5cccc6c(-c7ccccn7)nc(-c7ccccn7)nc56)c4)nc3c2n1] 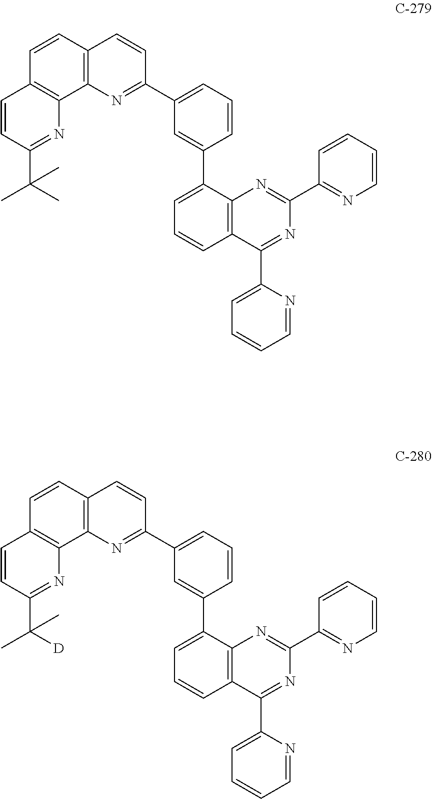 [SMILES: CC(C)(C)c1ccc2ccc3ccc(-c4cccc(-c5cccc6c(-c7ccccn7)nc(-c7ccccn7)nc56)c4)nc3c2n1 ; [2H]C(C)(C)c1ccc2ccc3ccc(-c4cccc(-c5cccc6c(-c7ccccn7)nc(-c7ccccn7)nc56)c4)nc3c2n1] 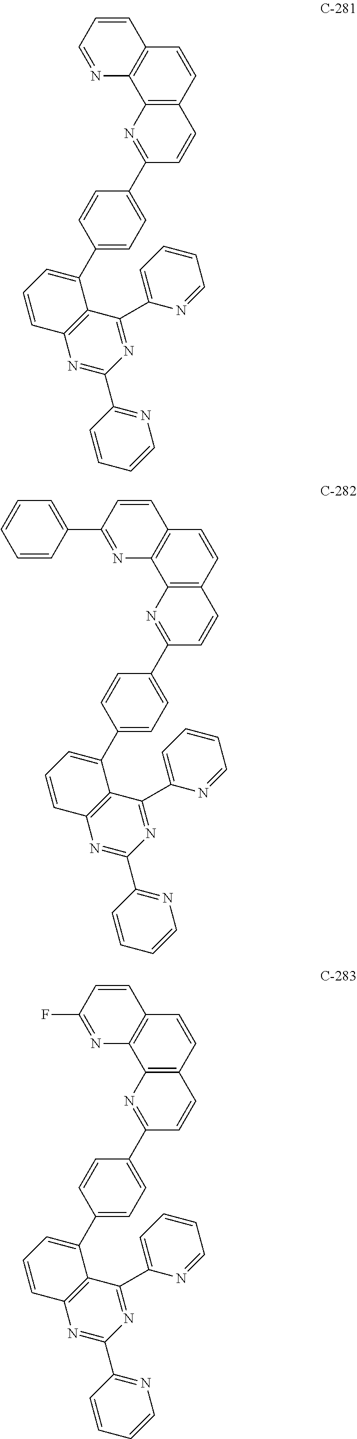 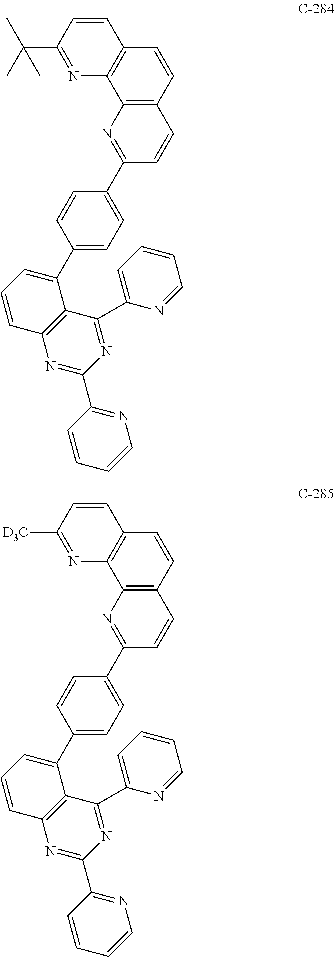 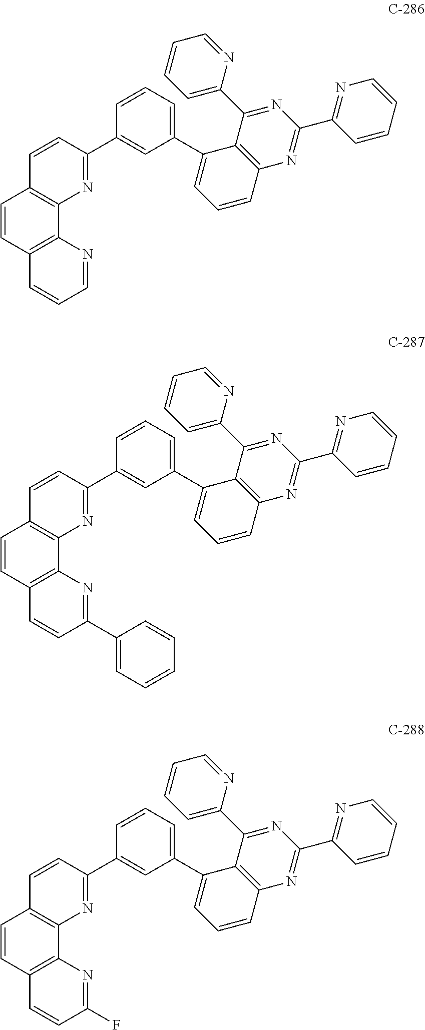 [SMILES: c1ccc(-c2nc(-c3ccccn3)c3c(-c4cccc(-c5ccc6ccc7cccnc7c6n5)c4)cccc3n2)nc1 ; c1ccc(-c2ccc3ccc4ccc(-c5cccc(-c6cccc7nc(-c8ccccn8)nc(-c8ccccn8)c67)c5)nc4c3n2)cc1 ; Fc1ccc2ccc3ccc(-c4cccc(-c5cccc6nc(-c7ccccn7)nc(-c7ccccn7)c56)c4)nc3c2n1] 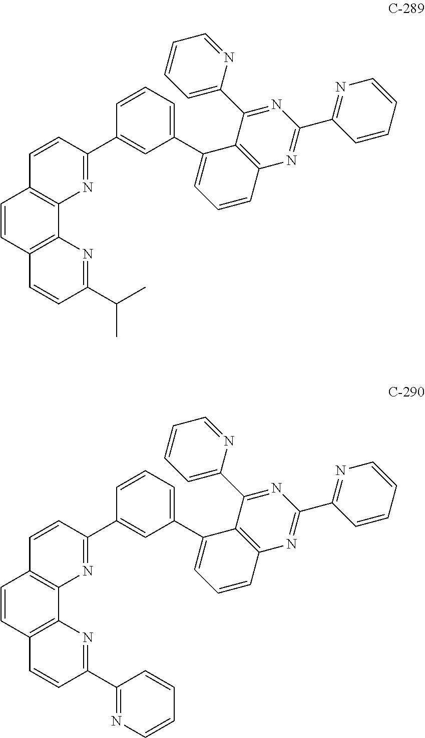 [SMILES: CC(C)c1ccc2ccc3ccc(-c4cccc(-c5cccc6nc(-c7ccccn7)nc(-c7ccccn7)c56)c4)nc3c2n1 ; c1ccc(-c2ccc3ccc4ccc(-c5cccc(-c6cccc7nc(-c8ccccn8)nc(-c8ccccn8)c67)c5)nc4c3n2)nc1] 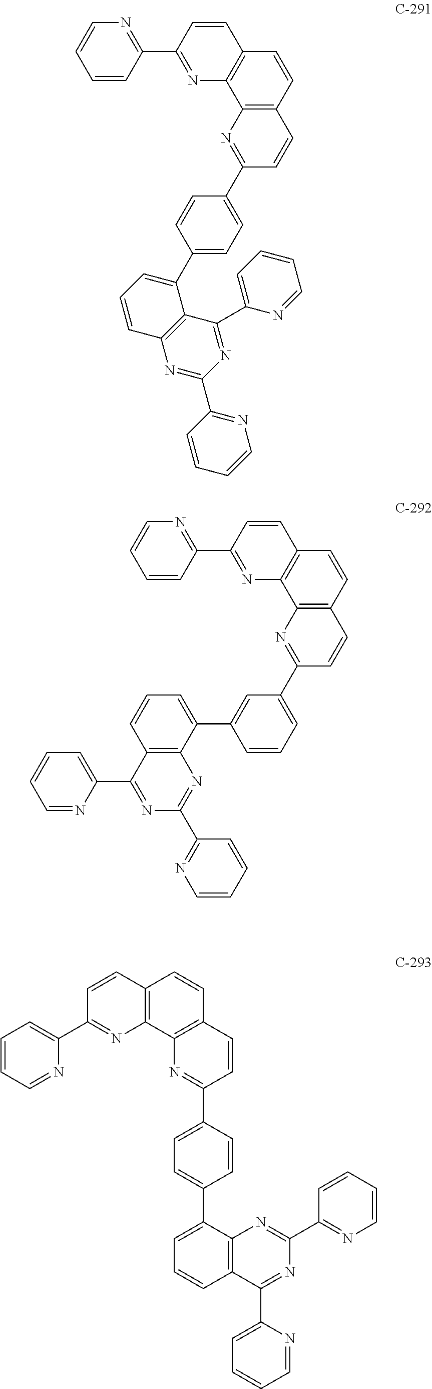 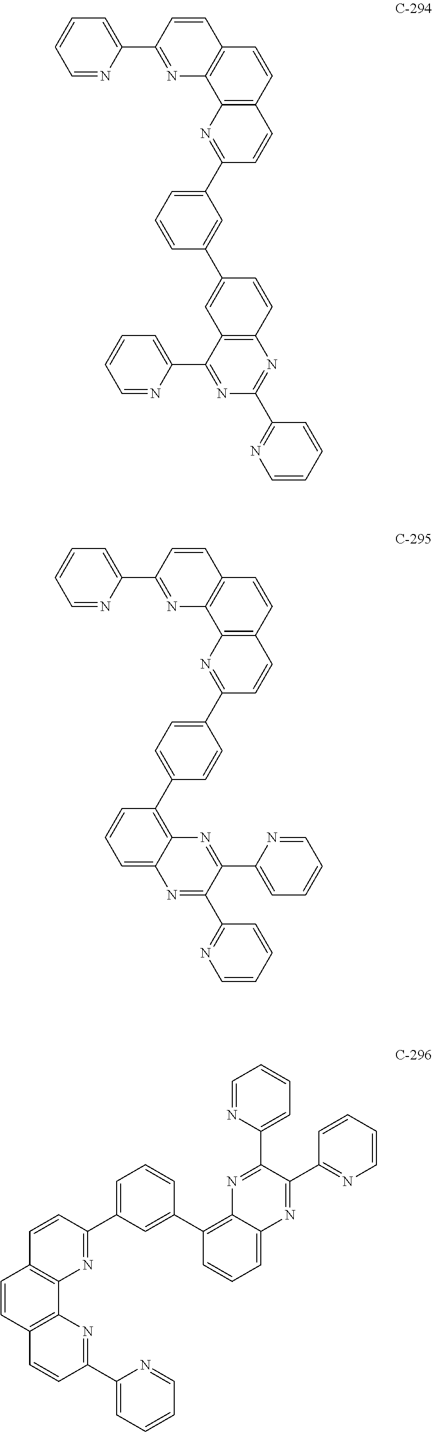 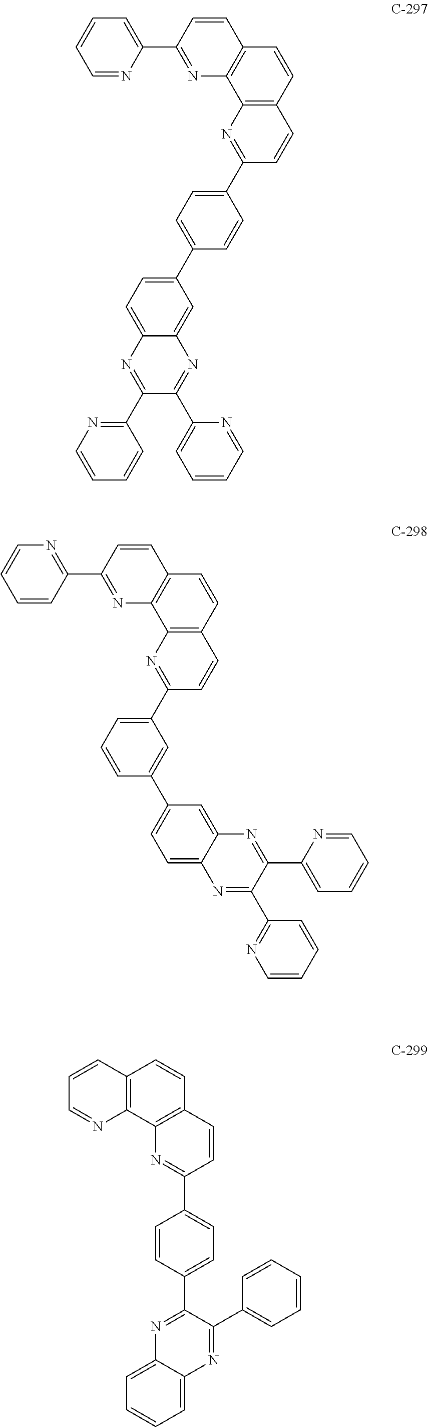 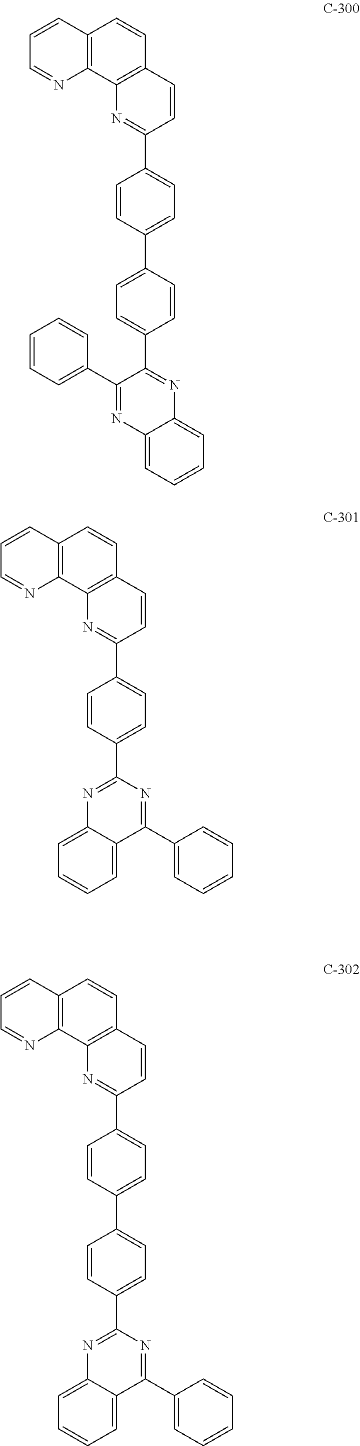 [SMILES: c1ccc(-c2nc3ccccc3nc2-c2ccc(-c3ccc(-c4ccc5ccc6cccnc6c5n4)cc3)cc2)cc1 ; c1ccc(-c2nc(-c3ccc(-c4ccc5ccc6cccnc6c5n4)cc3)nc3ccccc23)cc1 ; c1ccc(-c2nc(-c3ccc(-c4ccc(-c5ccc6ccc7cccnc7c6n5)cc4)cc3)nc3ccccc23)cc1] 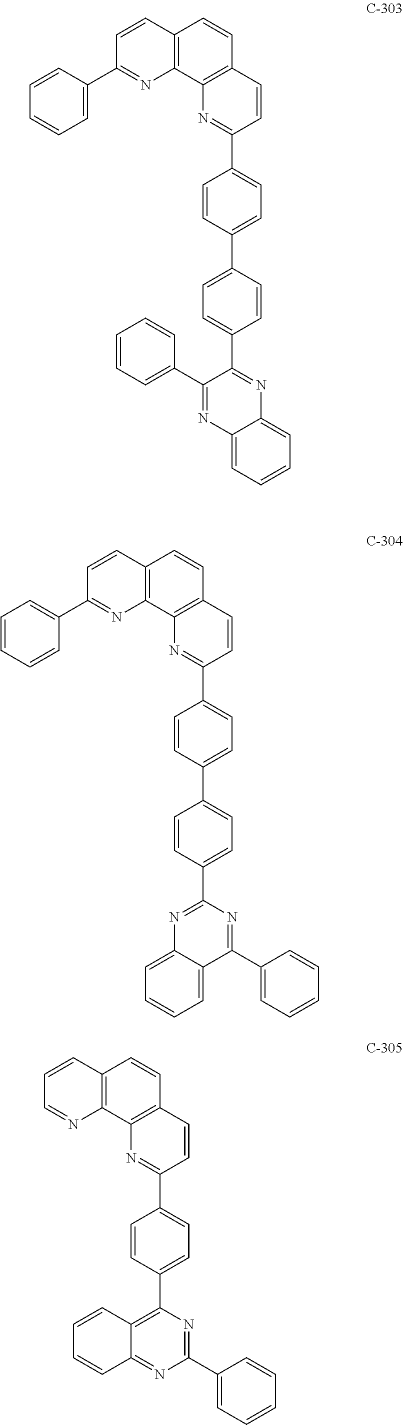 [SMILES: c1ccc(-c2ccc3ccc4ccc(-c5ccc(-c6ccc(-c7nc8ccccc8nc7-c7ccccc7)cc6)cc5)nc4c3n2)cc1 ; c1ccc(-c2ccc3ccc4ccc(-c5ccc(-c6ccc(-c7nc(-c8ccccc8)c8ccccc8n7)cc6)cc5)nc4c3n2)cc1 ; c1ccc(-c2nc(-c3ccc(-c4ccc5ccc6cccnc6c5n4)cc3)c3ccccc3n2)cc1] 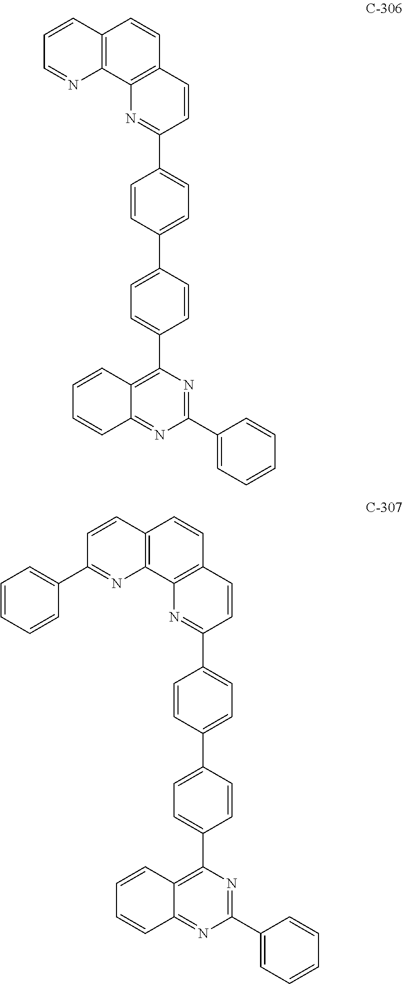 [SMILES: c1ccc(-c2nc(-c3ccc(-c4ccc(-c5ccc6ccc7cccnc7c6n5)cc4)cc3)c3ccccc3n2)cc1 ; c1ccc(-c2ccc3ccc4ccc(-c5ccc(-c6ccc(-c7nc(-c8ccccc8)nc8ccccc78)cc6)cc5)nc4c3n2)cc1] 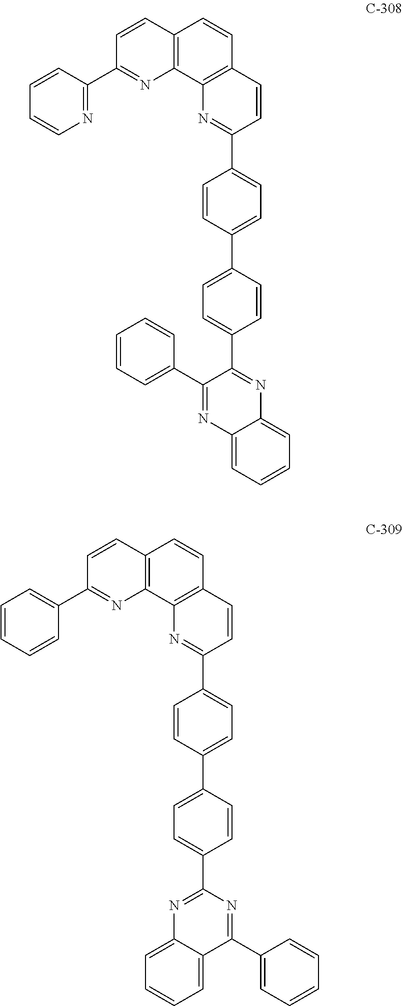 [SMILES: c1ccc(-c2nc3ccccc3nc2-c2ccc(-c3ccc(-c4ccc5ccc6ccc(-c7ccccn7)nc6c5n4)cc3)cc2)cc1 ; c1ccc(-c2ccc3ccc4ccc(-c5ccc(-c6ccc(-c7nc(-c8ccccc8)c8ccccc8n7)cc6)cc5)nc4c3n2)cc1] 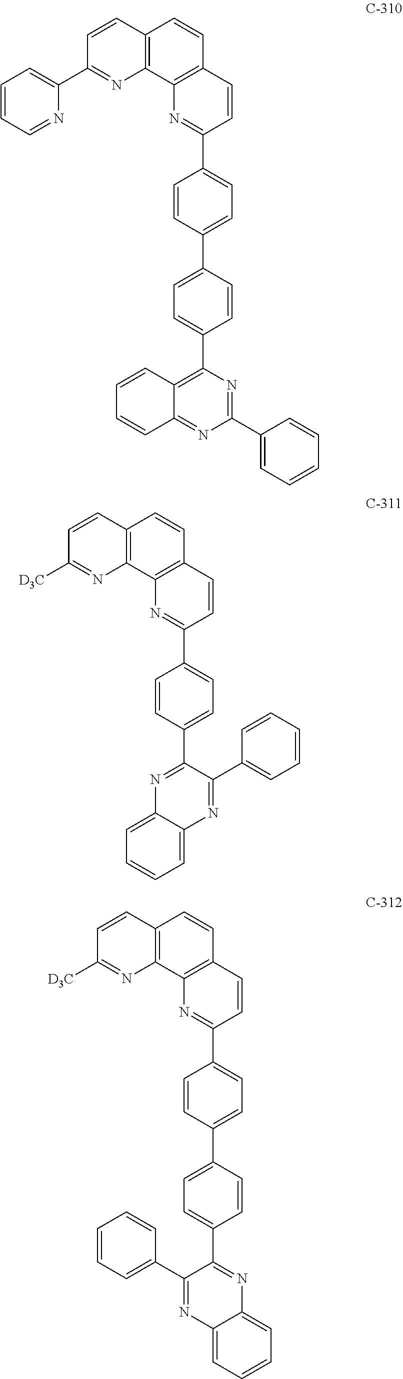 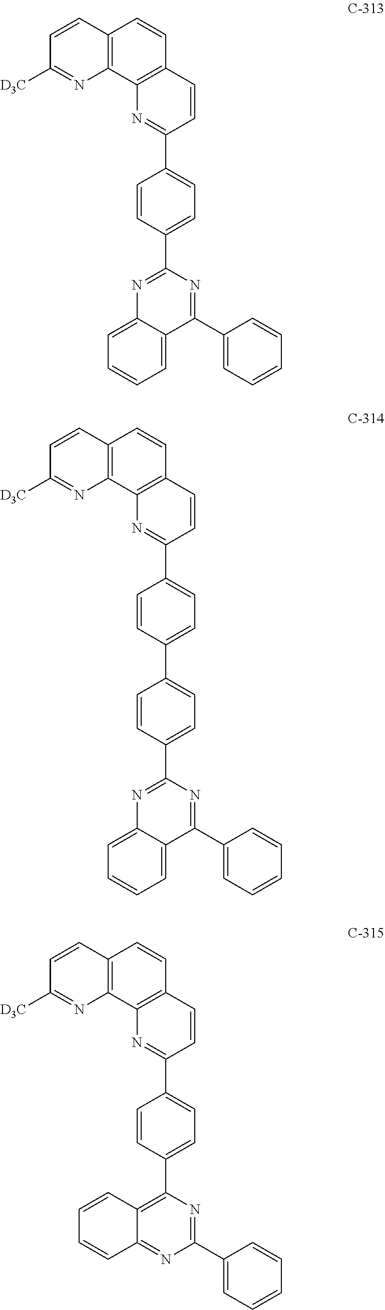 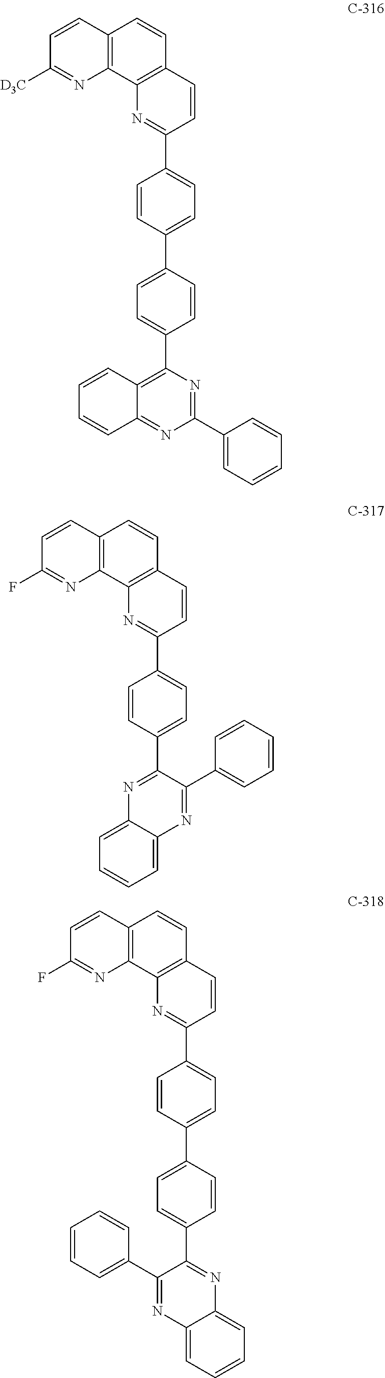 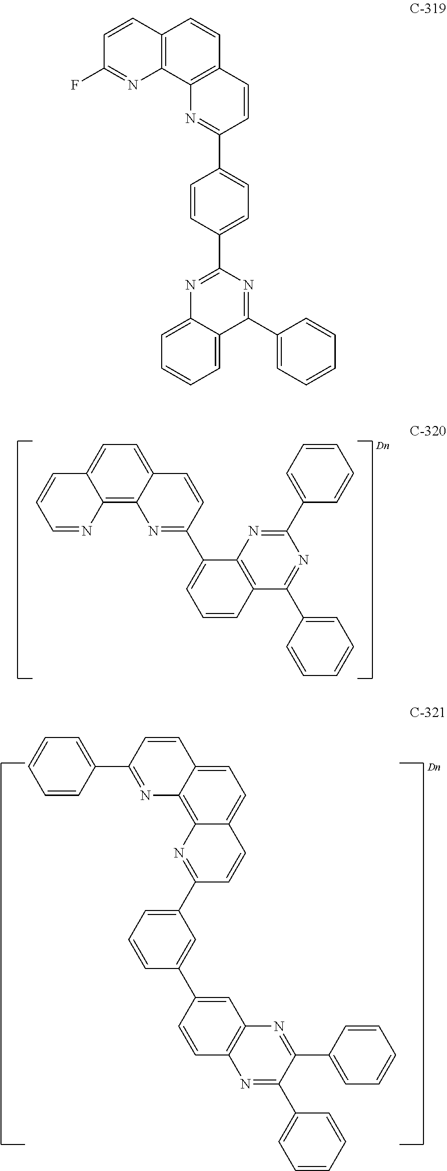 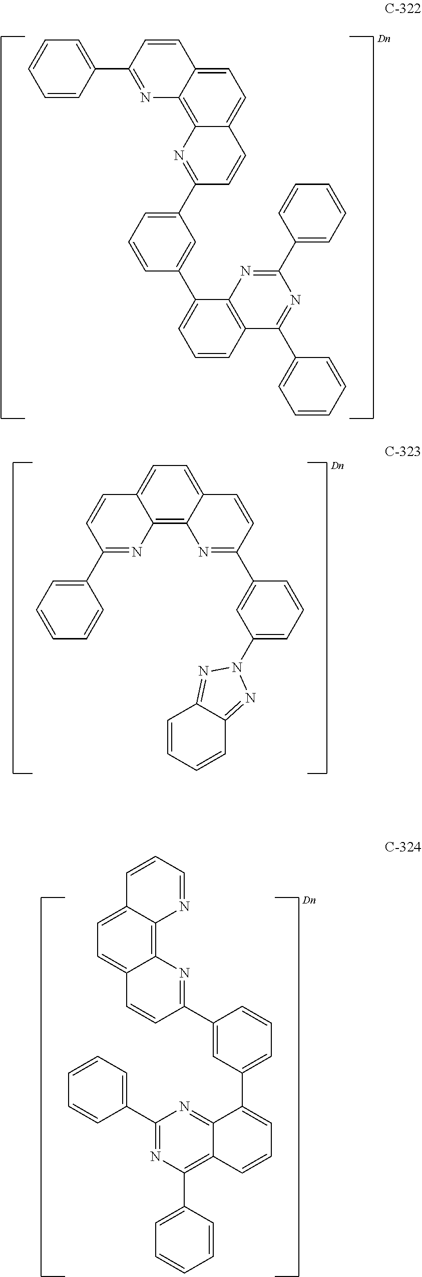 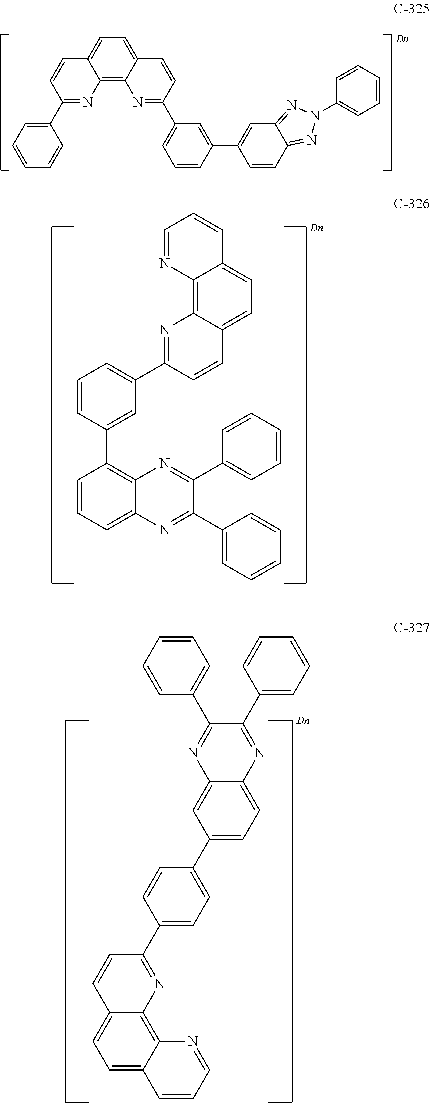 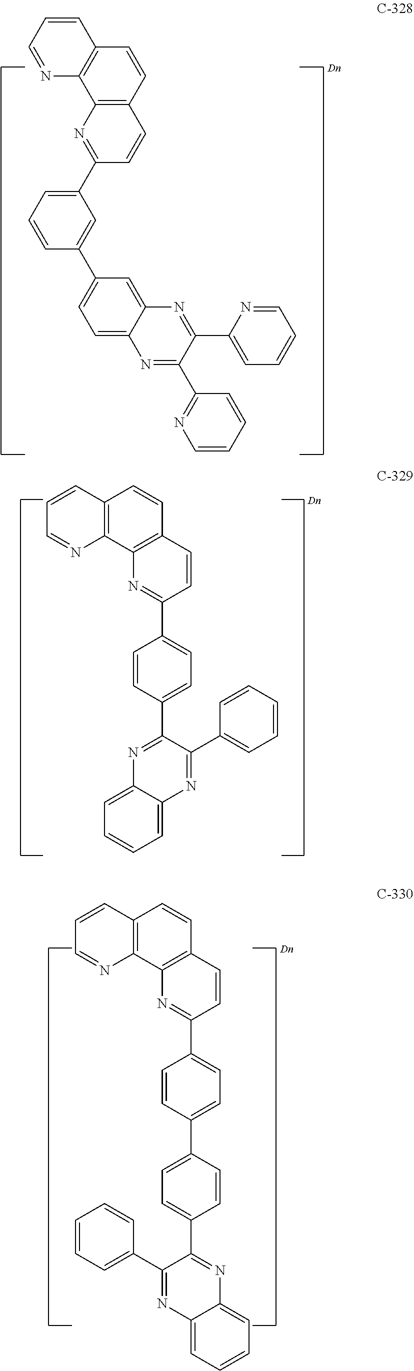 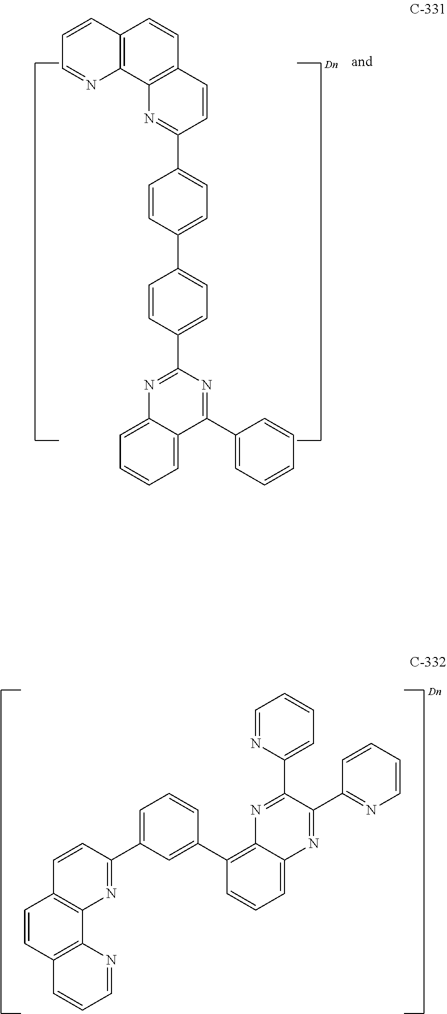
wherein Dₙ in the above compounds means that n number of hydrogens is replaced by deuterium, wherein n represents an integer of 1 or more, and the upper limit of n is determined by the number of hydrogens that can be substituted in each compound.

6. An organic electroluminescent material, comprising the compound represented by Formula 1 according to claim 1.

7. An organic electroluminescent device comprising a plurality of light-emitting units positioned between a first electrode and a second electrode; and at least one charge generation layer positioned between the adjacent light-emitting units, wherein the charge generation layer comprises a compound represented by Formula 1 according to claim 1.

8. The organic electroluminescent device according to claim 7, wherein at least one of the plurality of light-emitting units includes a first light-emitting layer and a second light-emitting layer that are adjacent to each other.

9. A compound selected from the following compounds:

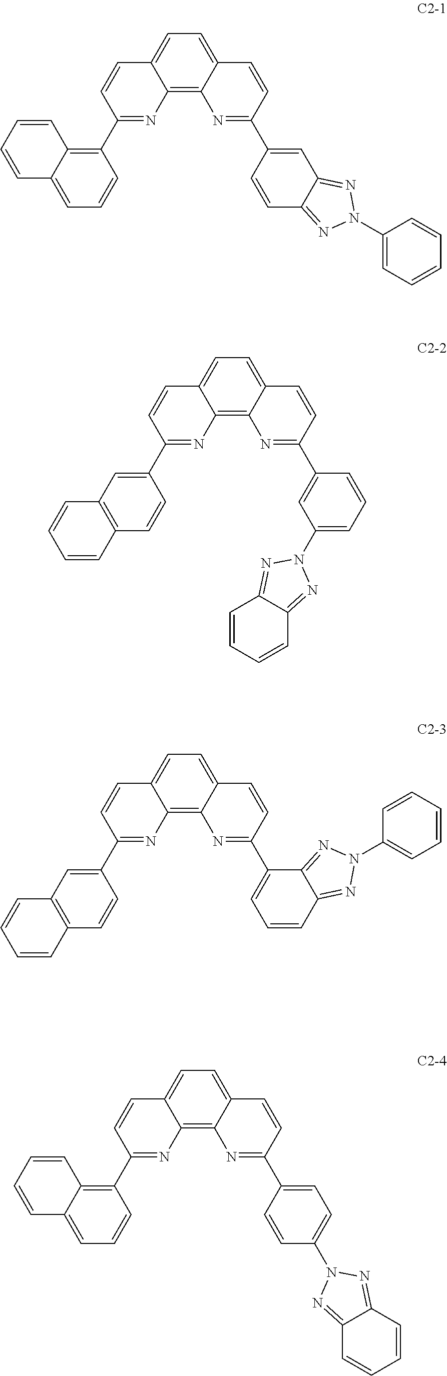 [SMILES: c1ccc(-n2nc3ccc(-c4ccc5ccc6ccc(-c7cccc8ccccc78)nc6c5n4)cc3n2)cc1 ; c1cc(-c2ccc3ccc4ccc(-c5ccc6ccccc6c5)nc4c3n2)cc(-n2nc3ccccc3n2)c1 ; c1ccc(-n2nc3cccc(-c4ccc5ccc6ccc(-c7ccc8ccccc8c7)nc6c5n4)c3n2)cc1 ; c1ccc2c(-c3ccc4ccc5ccc(-c6ccc(-n7nc8ccccc8n7)cc6)nc5c4n3)cccc2c1]

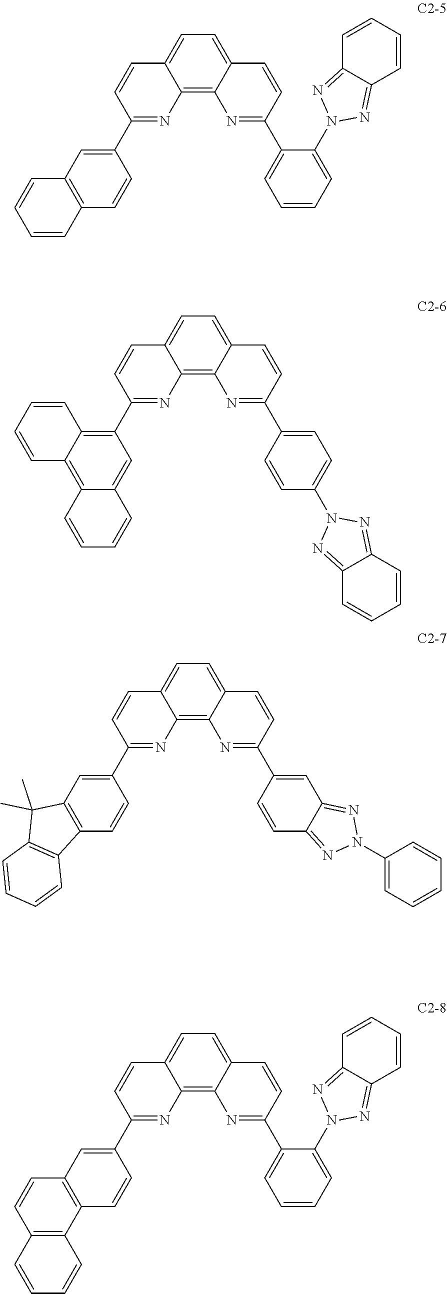 [SMILES: c1ccc(-n2nc3ccccc3n2)c(-c2ccc3ccc4ccc(-c5ccc6ccccc6c5)nc4c3n2)c1 ; c1ccc2c(c1)cc(-c1ccc3ccc4ccc(-c5ccc(-n6nc7ccccc7n6)cc5)nc4c3n1)c1ccccc12 ; CC1(C)c2ccccc2-c2ccc(-c3ccc4ccc5ccc(-c6ccc7nn(-c8ccccc8)nc7c6)nc5c4n3)cc21 ; c1ccc(-n2nc3ccccc3n2)c(-c2ccc3ccc4ccc(-c5ccc6c(ccc7ccccc76)c5)nc4c3n2)c1]

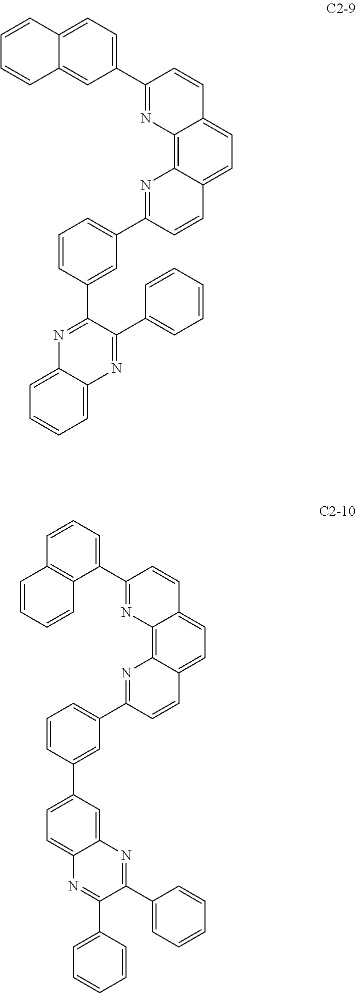 [SMILES: c1ccc(-c2nc3ccccc3nc2-c2cccc(-c3ccc4ccc5ccc(-c6ccc7ccccc7c6)nc5c4n3)c2)cc1 ; c1ccc(-c2nc3ccc(-c4cccc(-c5ccc6ccc7ccc(-c8cccc9ccccc89)nc7c6n5)c4)cc3nc2-c2ccccc2)cc1]

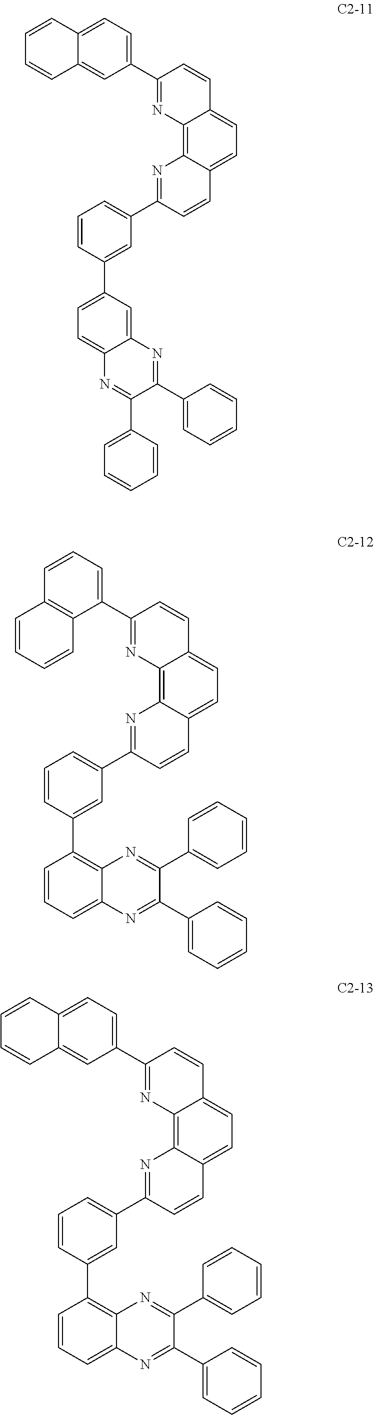 [SMILES: c1ccc(-c2nc3ccc(-c4cccc(-c5ccc6ccc7ccc(-c8ccc9ccccc9c8)nc7c6n5)c4)cc3nc2-c2ccccc2)cc1 ; c1ccc(-c2nc3cccc(-c4cccc(-c5ccc6ccc7ccc(-c8cccc9ccccc89)nc7c6n5)c4)c3nc2-c2ccccc2)cc1 ; c1ccc(-c2nc3cccc(-c4cccc(-c5ccc6ccc7ccc(-c8ccc9ccccc9c8)nc7c6n5)c4)c3nc2-c2ccccc2)cc1]

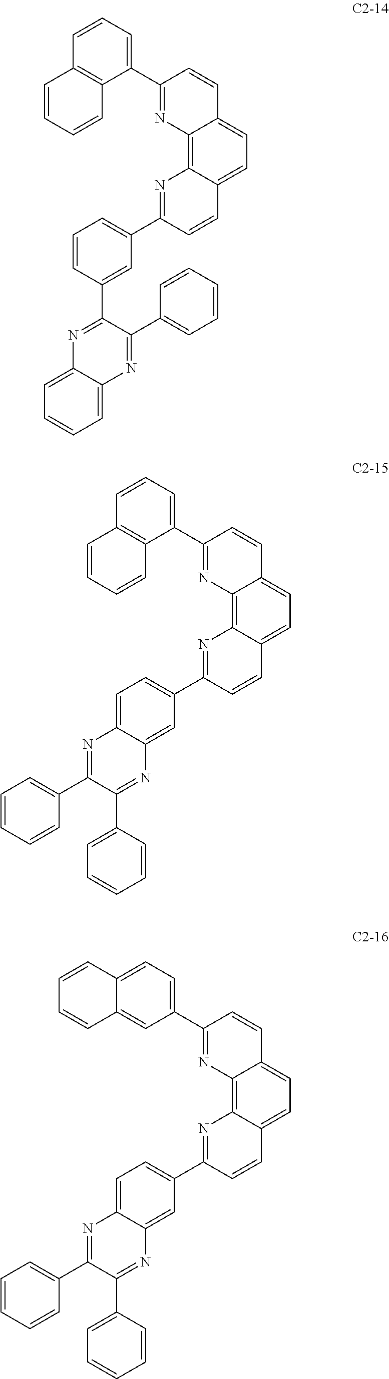 [SMILES: c1ccc(-c2nc3ccccc3nc2-c2cccc(-c3ccc4ccc5ccc(-c6cccc7ccccc67)nc5c4n3)c2)cc1 ; c1ccc(-c2nc3ccc(-c4ccc5ccc6ccc(-c7cccc8ccccc78)nc6c5n4)cc3nc2-c2ccccc2)cc1 ; c1ccc(-c2nc3ccc(-c4ccc5ccc6ccc(-c7ccc8ccccc8c7)nc6c5n4)cc3nc2-c2ccccc2)cc1]

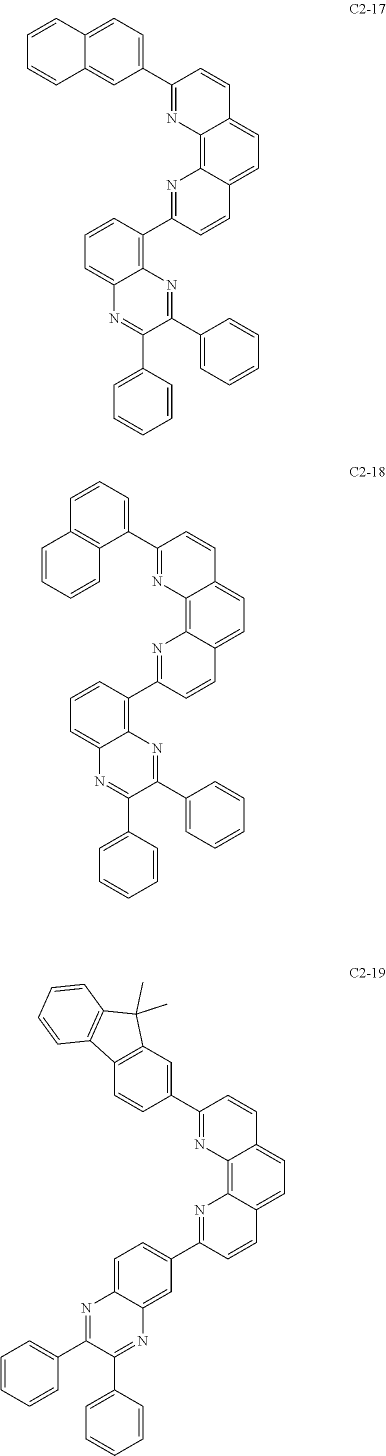 [SMILES: c1ccc(-c2nc3cccc(-c4ccc5ccc6ccc(-c7ccc8ccccc8c7)nc6c5n4)c3nc2-c2ccccc2)cc1 ; c1ccc(-c2nc3cccc(-c4ccc5ccc6ccc(-c7cccc8ccccc78)nc6c5n4)c3nc2-c2ccccc2)cc1 ; CC1(C)c2ccccc2-c2ccc(-c3ccc4ccc5ccc(-c6ccc7nc(-c8ccccc8)c(-c8ccccc8)nc7c6)nc5c4n3)cc21]

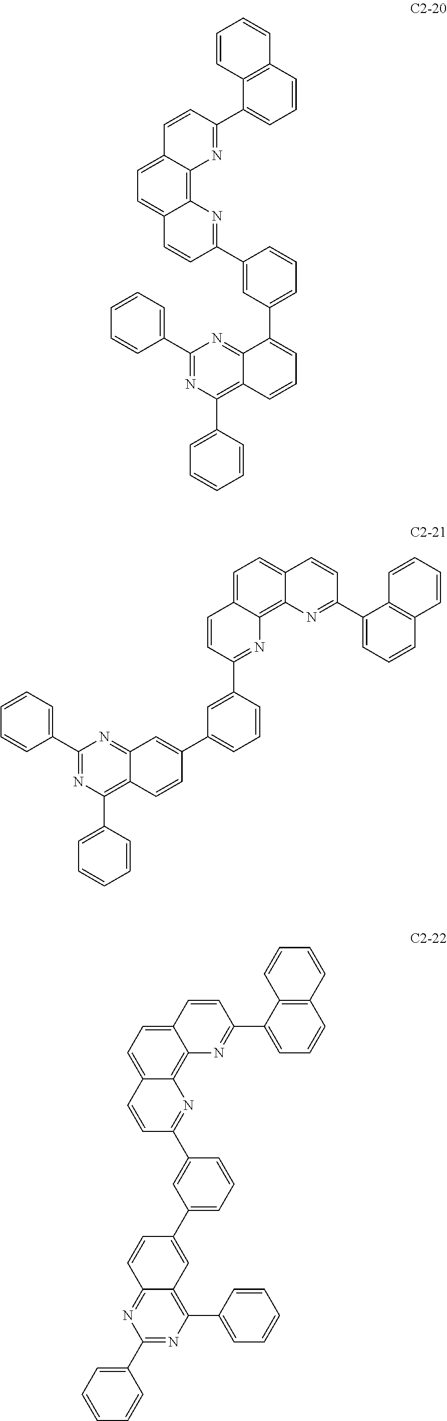 [SMILES: c1ccc(-c2nc(-c3ccccc3)c3cccc(-c4cccc(-c5ccc6ccc7ccc(-c8cccc9ccccc89)nc7c6n5)c4)c3n2)cc1 ; c1ccc(-c2nc(-c3ccccc3)c3ccc(-c4cccc(-c5ccc6ccc7ccc(-c8cccc9ccccc89)nc7c6n5)c4)cc3n2)cc1 ; c1ccc(-c2nc(-c3ccccc3)c3cc(-c4cccc(-c5ccc6ccc7ccc(-c8cccc9ccccc89)nc7c6n5)c4)ccc3n2)cc1]

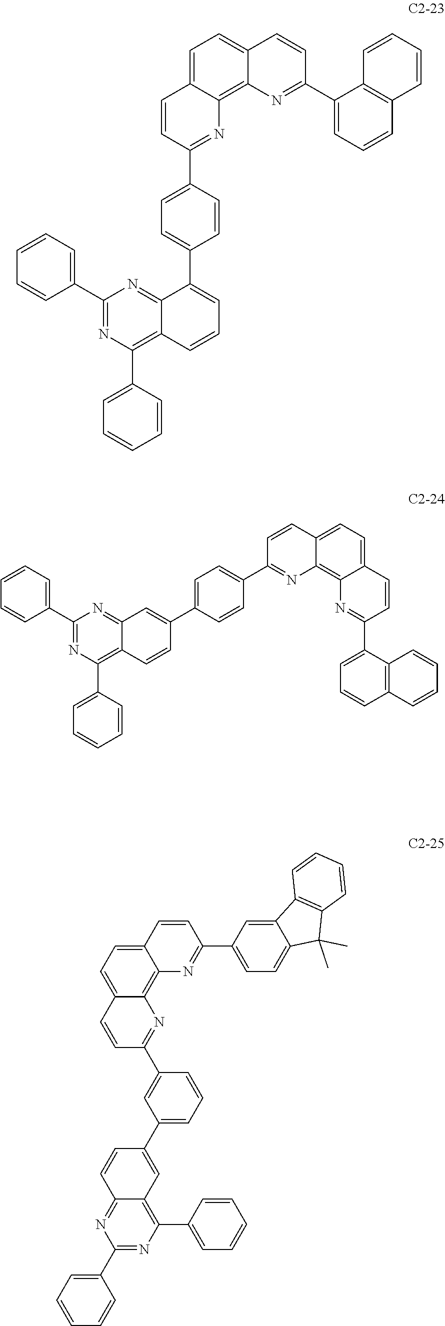 [SMILES: c1ccc(-c2nc(-c3ccccc3)c3cccc(-c4ccc(-c5ccc6ccc7ccc(-c8cccc9ccccc89)nc7c6n5)cc4)c3n2)cc1 ; c1ccc(-c2nc(-c3ccccc3)c3ccc(-c4ccc(-c5ccc6ccc7ccc(-c8cccc9ccccc89)nc7c6n5)cc4)cc3n2)cc1 ; CC1(C)c2ccccc2-c2cc(-c3ccc4ccc5ccc(-c6cccc(-c7ccc8nc(-c9ccccc9)nc(-c9ccccc9)c8c7)c6)nc5c4n3)ccc21]

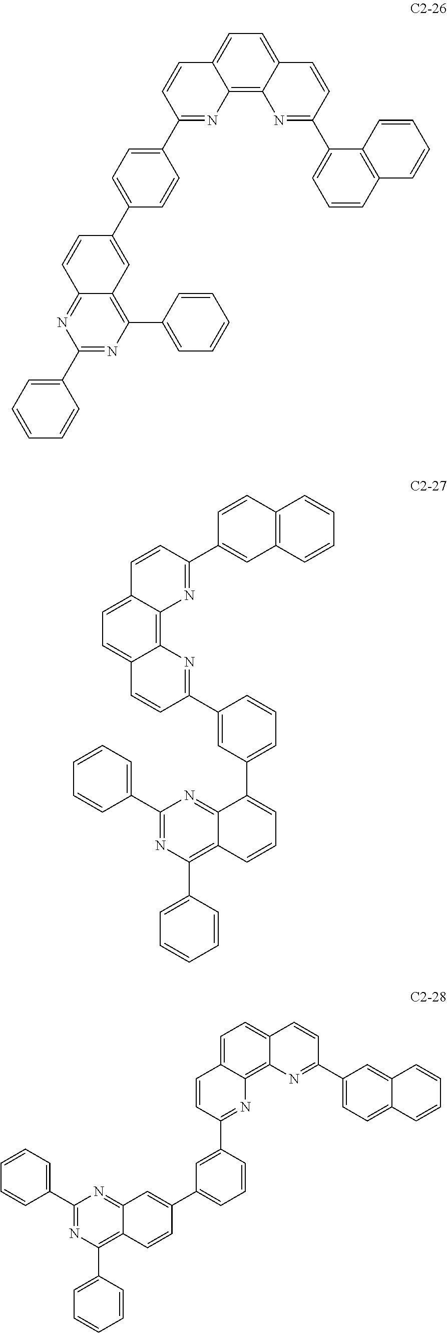 [SMILES: c1ccc(-c2nc(-c3ccccc3)c3cc(-c4ccc(-c5ccc6ccc7ccc(-c8cccc9ccccc89)nc7c6n5)cc4)ccc3n2)cc1 ; c1ccc(-c2nc(-c3ccccc3)c3cccc(-c4cccc(-c5ccc6ccc7ccc(-c8ccc9ccccc9c8)nc7c6n5)c4)c3n2)cc1 ; c1ccc(-c2nc(-c3ccccc3)c3ccc(-c4cccc(-c5ccc6ccc7ccc(-c8ccc9ccccc9c8)nc7c6n5)c4)cc3n2)cc1]

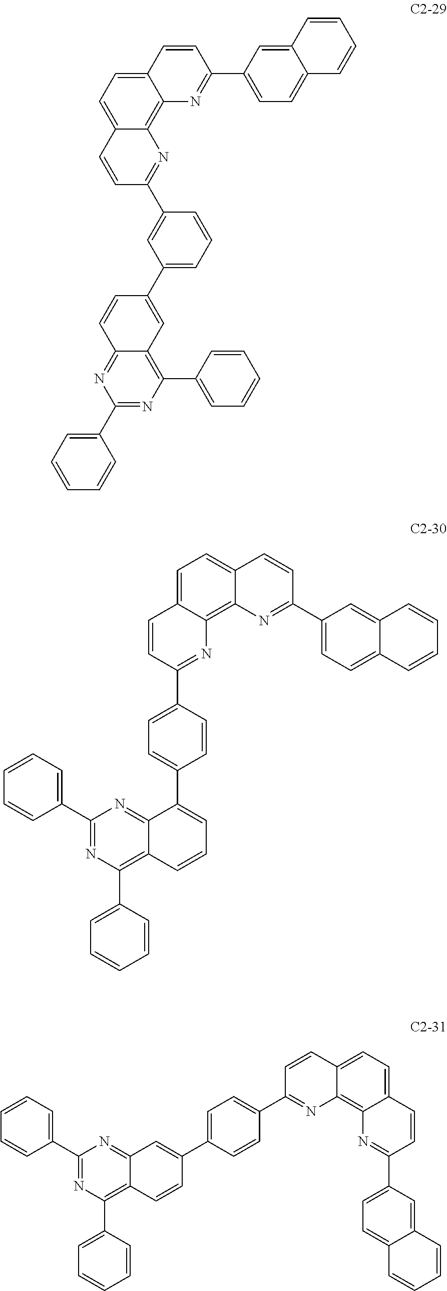 [SMILES: c1ccc(-c2nc(-c3ccccc3)c3cc(-c4cccc(-c5ccc6ccc7ccc(-c8ccc9ccccc9c8)nc7c6n5)c4)ccc3n2)cc1 ; c1ccc(-c2nc(-c3ccccc3)c3cccc(-c4ccc(-c5ccc6ccc7ccc(-c8ccc9ccccc9c8)nc7c6n5)cc4)c3n2)cc1 ; c1ccc(-c2nc(-c3ccccc3)c3ccc(-c4ccc(-c5ccc6ccc7ccc(-c8ccc9ccccc9c8)nc7c6n5)cc4)cc3n2)cc1]

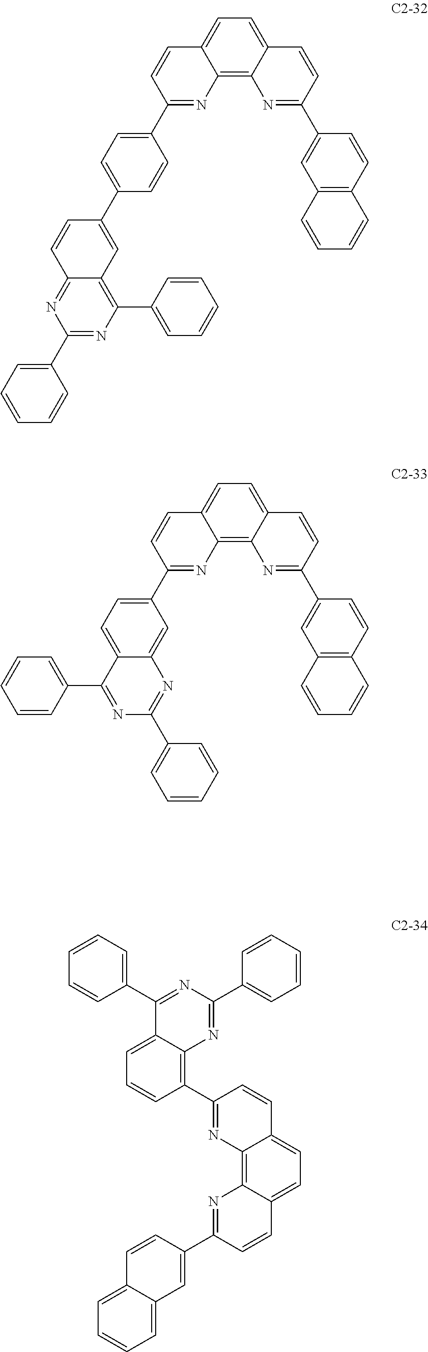 [SMILES: c1ccc(-c2nc(-c3ccccc3)c3cc(-c4ccc(-c5ccc6ccc7ccc(-c8ccc9ccccc9c8)nc7c6n5)cc4)ccc3n2)cc1 ; c1ccc(-c2nc(-c3ccccc3)c3ccc(-c4ccc5ccc6ccc(-c7ccc8ccccc8c7)nc6c5n4)cc3n2)cc1 ; c1ccc(-c2nc(-c3ccccc3)c3cccc(-c4ccc5ccc6ccc(-c7ccc8ccccc8c7)nc6c5n4)c3n2)cc1]

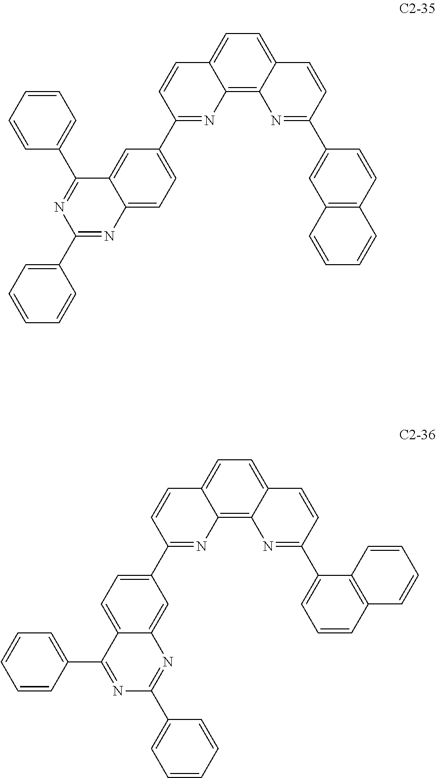 [SMILES: c1ccc(-c2nc(-c3ccccc3)c3cc(-c4ccc5ccc6ccc(-c7ccc8ccccc8c7)nc6c5n4)ccc3n2)cc1 ; c1ccc(-c2nc(-c3ccccc3)c3ccc(-c4ccc5ccc6ccc(-c7cccc8ccccc78)nc6c5n4)cc3n2)cc1]

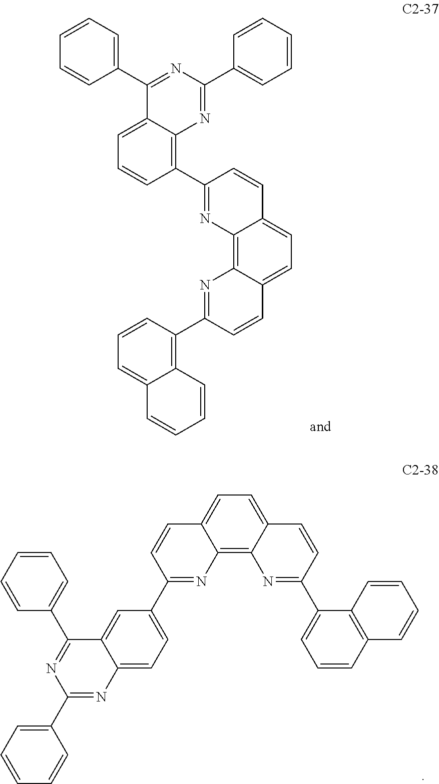 [SMILES: c1ccc(-c2nc(-c3ccccc3)c3cccc(-c4ccc5ccc6ccc(-c7cccc8ccccc78)nc6c5n4)c3n2)cc1 ; c1ccc(-c2nc(-c3ccccc3)c3cc(-c4ccc5ccc6ccc(-c7cccc8ccccc78)nc6c5n4)ccc3n2)cc1]

10. An organic electroluminescent material comprising a compound according to claim 9.

11. An organic electroluminescent device comprising a plurality of light-emitting units positioned between a first electrode and a second electrode; and at least one charge generation layer positioned between the adjacent light-emitting units, wherein the charge generation layer comprises a compound according to claim 9.

12. The organic electroluminescent device according to claim 11, wherein at least one of the plurality of light-emitting units includes a first light-emitting layer and a second light-emitting layer that are adjacent to each other.
